(19) United States
(12) Patent Application Publication
(10) Pub. No.: US 2024/0112754 A1
(43) Pub. Date: Apr. 4, 2024

(54) Crohn's Disease-Associated T Cell Receptor-Related Methods

(71) Applicant: Adaptive Biotechnologies Corporation, Seattle, WA (US)

(72) Inventors: Harlan S. Robins, Seattle, WA (US); Thomas M. Snyder, San Bruno, CA (US)

(21) Appl. No.: 18/276,805

(22) PCT Filed: Mar. 11, 2022
(86) PCT No.: PCT/US2022/019938

§ 371 Date: Aug. 10, 2023

Related U.S. Application Data
(60) Provisional application No. 63/160,213, filed on Mar. 12, 2021.

(51) Int. Cl. G16B 20/30; A61K 31/4402; A61K 31/573; A61K 31/58; A61K 31/606; C07K 14/705

(52) U.S. Cl. CPC G16B 20/30; A61K 31/4402; A61K 31/573; A61K 31/58; A61K 31/606; C07K 14/70503

(57) ABSTRACT

Provided are methods for assessing T cell receptor β chain complementary determining region 3 (TCRβ CDR3) sequences. In certain embodiments, prior to the assessing, the subject has been identified as having, or is suspected of having, inflammatory bowel disease (IBD). According to some embodiments, at the time of the assessing, the subject has one or more non-specific symptoms consistent with Crohn's disease. Also provided are methods comprising administering a Crohn's disease therapy to a subject identified as comprising T cells that express a T cell receptor β chain (TCRβ) comprising a TCRβ CDR3 sequence set forth in the present disclosure. Computer readable media and systems for assessing TCRβ CDR3 sequences are also provided.

### CROSS-REFERENCE TO RELATED APPLICATIONS

This application claims the benefit of U.S. Provisional Patent Application No. 63/160,213, filed Mar. 12, 2021, which application is incorporated herein by reference in its entirety.

### INTRODUCTION

Antigen-specific cellular immune responses are mediated by a diverse population of T cells and B cells, each bearing immune cell receptors (TCRs and BCRs, respectively) capable of recognizing a specific antigen (in the case of T cells, an antigen peptide bound to a particular major histocompatibility complex (MHC) molecule on the surface of host cells). Encounter with an antigen leads to the clonal expansion, activation, and maturation of T and B cells, resulting in effector populations of cytotoxic (CD8⁺ CTL) and helper (CD4⁺ ) T cells, or antibodies and memory B cells, respectively. The presence of antigen-specific effector cells is diagnostic of an immune response specific to that antigen.

Activated T cells proliferate by clonal expansion and reside in the memory T cell compartment for many years as a clonal population of cells (clones) with identical-by-descent rearranged TCR genes (Arstila T P, et al. A direct estimate of the human alpha/beta T cell receptor diversity, Science 286: 958-961).

The majority of TCR diversity resides in the beta chain of the TCR alpha/beta heterodimer. Immense diversity is generated by combining noncontiguous TCRβ variable (V), diversity (D), and joining (J) region gene segments, which collectively encode the CDR3 region, the primary region of the TCRβ locus for determining antigen specificity. Deletion and template-independent insertion of nucleotides during rearrangement at the Vβ-Dβ and Dβ-Jβ junctions further add to the potential diversity of receptors that can be encoded (Cabaniols J P, et al. Most alpha/beta T cell receptor diversity is due to terminal deoxynucleotidyl transferase, J Exp Med 194: 1385-1390, 2001). Typically, at a given point in time, an adult with a healthy immune system expresses approximately 10 million unique TCRβ chains on their 10¹² circulating T cells (Robins H S, et al. (2009) Comprehensive assessment of T-cell receptor beta-chain diversity in alpha/beta T cells, Blood 114: 4099-4107).

The human T-cell repertoire thus dynamically encodes exposure to disease-related antigens through rearrangements of their receptor-encoding genes and so provides an excellent basis for making diagnostic predictions. It has been demonstrated that TCRβ receptors in peripheral blood samples from human subjects can be employed to predict the status of exposure to a disease; i.e., based on the presence and abundance of such receptors in the training cohort (Emerson et al., Immunosequencing identifies signatures of cytomegalovirus exposure history and HLA-mediated effects on the T cell repertoire, Nature Genetics April 2017; doi: 10.1038/ng.3822).

Inflammatory bowel diseases (IBD) are chronic, relapsing inflammatory conditions that are immunologically mediated. IBD is believed to result from a pronounced immunologic response in genetically susceptible individuals, usually due to an environmental factor, such as gut commensals. IBD can be diagnosed at any age, but the majority of diagnoses are made between the ages of 20 and 30, with a second peak of IBD diagnoses occurring during the sixth or seventh decade of life. Due to the early onset of IBD, the severe symptoms associated with it, the natural unsettled course of disease, number of hospitalizations and the lack of a cure, IBD diagnosis has a significant impact on a patient's quality of life.

IBD is an umbrella term used to describe disorders that involve chronic inflammation of the digestive tract. Types of IBD include Crohn's disease (CD) and ulcerative colitis (UC). Crohn's disease is characterized by inflammation of the lining of the digestive tract, often involving the deeper layers of the digestive tract. Ulcerative colitis involves inflammation and sores (ulcers) along the superficial lining of the large intestine (colon) and rectum.

Both Crohn's disease and ulcerative colitis are characterized by diarrhea, fatigue, abdominal pain and cramping, rectal bleeding (blood in the stool), and unintended weight loss. When IBD predominantly involves the colon, differentiation between Crohn's disease and ulcerative colitis is especially challenging. Inaccurate diagnoses are estimated to occur in 30% of IBD patients. In the majority of cases, the diagnostic uncertainty arises from the overlap of clinical and histologic features, making Crohn's disease appear like ulcerative colitis. The differentiation between Crohn's disease and ulcerative colitis relies on an often inaccurate compilation of clinical, radiologic, endoscopic, and histopathologic interpretations.

An estimated 15% of IBD patients are indistinguishable following one or more of clinicial, radiologic, serologic and pathological tests and are labeled as “indeterminate colitis” (IC). Another 15% of the colonic IBD cases that undergo pouch surgery resulting from an initial ulcerative colitis diagnosis (based on the pathologist's initial designation of endoscopic biopsies and colectomy specimen) will have their ulcerative colitis diagnosis changed to Crohn's disease based on the postoperative follow-up when clinical and histopathological changes indicate development of Crohn's disease in the ileal pouch. One-half of these patients will require pouch excision or diversion.

Distinguishing between Crohn's disease and ulcerative colitis is important for informing appropriate therapy. For example, restorative proctocolectomy (RPC) should be contraindicated for Crohn's disease patients, whereas ileal pouch-anal anastomosis (IPAA) is standard acceptable treatment for patients with ulcerative colitis and indeterminate colitis who are predicted likely to develop ulcerative colitis.

There has been significant interest in the identification of biomarkers that can accurately distinguish Crohn's disease and ulcerative colitis. Investigations have been minimally successful at identifying biomarkers of potential relevance for distinguishing Crohn's disease and ulcerative colitis. Such biomarkers in serum include placenta growth factor-1 (PLGF-1), IL-7, TQRb1, and IL-12P40. In biopsies obtained from the mucosa, they include Rho GD1a, desmoglein, pleckstrin, VDAC (voltage-dependent anion channel), 3-hydroxy-3-methylglutaryl-coenzyme A reductase (HMG-CoA), and C10orf76. Biomarkers in stool include calprotectin, PMN-elastate, lactoferrin, and S100A12. Although the identification of these biomarkers represent an advancement in the field, they have not been shown to accurately form the basis for diagnosing Crohn's disease and/or delineating Crohn's disease and ulcerative colitis.

The present diagnostic deficiencies and potential morbidity from an incorrect diagnosis (e.g., unnecessary and/or inappropriate surgical interventions) underscore the need for new diagnostic approaches with improved sensitivity and specificity for Crohn's disease or sub-types thereof, permitting more accurate diagnosis of Crohn's disease and/or differential diagnosis between Crohn's disease and ulcerative colitis.

### SUMMARY

Provided are methods for assessing T cell receptor β chain complementary determining region 3 (TCRβ CDR3) sequences. In certain embodiments, prior to the assessing, the subject has been identified as having, or is suspected of having, inflammatory bowel disease (IBD). According to some embodiments, at the time of the assessing, the subject has one or more non-specific symptoms consistent with Crohn's disease. Also provided are methods comprising administering a Crohn's disease therapy to a subject identified as comprising T cells that express a T cell receptor β chain (TCRβ) comprising a TCRβ CDR3 sequence set forth in the present disclosure. Computer readable media and systems for assessing TCRβ CDR3 sequences are also provided.

### BRIEF DESCRIPTION OF THE FIGURES

FIG. 1: Data illustrating the identification of Crohn's disease-associated TCRβ sequences that distinguish Crohn's patients from controls (healthy adults).

FIG. 2: Data illustrating model performance on holdout data sets, in which a strong signal was observed only for Crohn's patients and not other healthy/disease groups tested.

FIG. 3: Schematic illustration and data demonstrating that the Crohn's disease-associated TCRβ sequences cluster and show convergent recombination in Crohn's patients and not controls.

FIG. 4: Data demonstrating overlap between blood and tissue samples in Crohn's disease-associated TCRβ sequences.

FIG. 5: Data illustrating the performance of classification model trained on large scale Crohn's disease data set, including high sensitivity in Crohn's, high specificity against unrelated conditions, and significant differences in signal in more complicated disease (stricturing/fistulating) and in ileal/ileocolonic locations over colonic location.

### DETAILED DESCRIPTION

Before the methods of the present disclosure are described in greater detail, it is to be understood that the methods are not limited to particular embodiments described, as such may, of course, vary. It is also to be understood that the terminology used herein is for the purpose of describing particular embodiments only, and is not intended to be limiting, since the scope of the methods will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the methods. The upper and lower limits of these smaller ranges may independently be included in the smaller ranges and are also encompassed within the methods, subject to any specifically excluded limit in the stated range. Where the stated range includes one or both of the limits, ranges excluding either or both of those included limits are also included in the methods.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which the methods belong. Although any methods similar or equivalent to those described herein can also be used in the practice or testing of the methods, representative illustrative methods are now described.

All publications and patents cited in this specification are herein incorporated by reference as if each individual publication or patent were specifically and individually indicated to be incorporated by reference and are incorporated herein by reference to disclose and describe the materials and/or methods in connection with which the publications are cited. The citation of any publication is for its disclosure prior to the filing date and should not be construed as an admission that the present methods are not entitled to antedate such publication, as the date of publication provided may be different from the actual publication date which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the methods, which are, for clarity, described in the context of separate embodiments, may also be provided in combination in a single embodiment. Conversely, various features of the methods, which are, for brevity, described in the context of a single embodiment, may also be provided separately or in any suitable sub-combination. All combinations of the embodiments are specifically embraced by the present disclosure and are disclosed herein just as if each and every combination was individually and explicitly disclosed, to the extent that such combinations embrace operable processes and/or compositions. In addition, all sub-combinations listed in the embodiments describing such variables are also specifically embraced by the present methods and are disclosed herein just as if each and every such sub-combination was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present methods. Any recited method can be carried out in the order of events recited or in any other order that is logically possible.

### Methods for Assessing TCRβ CDR3 Sequences

The present disclosure provides methods for assessing T cell receptor β chain complementary determining region 3 (TCRβ CDR3) sequences. In certain embodiments, the methods comprise assessing TCRβ CDR3 sequences determined from a sample obtained from a subject for the presence or absence of one or more TCRβ CDR3 sequences set forth in SEQ ID Nos:1-1996 herein, e.g., SEQ ID Nos:1-1281 herein. The inventors have determined that TCRs comprising such TCRβ CDR3 sequences are associated with Crohn's disease by being statistically more prevalent in individuals having Crohn's disease than those who do not have Crohn's disease. Accordingly, the methods of the present disclosure find use, for example, in predicting whether a subject has or does not have Crohn's disease. Prior to the assessing, the subject may have been identified as having, or is suspected of having, inflammatory bowel disease (IBD). In certain embodiments, for a subject identified as having IBD or suspected of having IBD, the methods find use in diagnosing the subject as having Crohn's disease. Such a diagnosis may be a differential diagnosis in which a subject exhibiting one or more non-specific symptoms consistent with Crohn's disease is diagnosed as having Crohn's disease and not another condition characterized by symptoms which overlap with those of Crohn's disease, including but not limited to, ulcerative colitis, irritable bowel syndrome, and/or celiac disease. Alternatively or additionally, such a diagnosis may be a differential diagnosis wherein the subject is diagnosed as having Crohn's disease with fistulation or structuring and not Crohn's disease without fistulation or structuring, or as having ileal/ileocolonic Crohn's disease and not colonic Crohn's disease. Details regarding the methods of the present disclosure will now be described.

According to some embodiments, the methods of the present disclosure are computer-implemented. By “computer-implemented” is meant at least one step of the method is implemented using one or more processors and one or more non-transitory computer-readable media. For example, in certain embodiments, provided are computer-implemented methods for assessing TCRβ CDR3 sequences, the methods being implemented using one or more processors and one or more non-transitory computer-readable media comprising instructions stored thereon, which when executed by the one or more processors, cause the one or more processors to assess TCRβ CDR3 sequences determined from a sample obtained from a subject for the presence or absence of one or more TCRβ CDR3 sequences set forth in SEQ ID Nos:1-1996 herein, e.g., SEQ ID Nos:1-1281 herein. The computer-implemented methods of the present disclosure may further comprise one or more steps that are not computer-implemented, e.g., obtaining a sample (e.g., a blood sample, gut tissue sample, or the like) from the subject, preparing the sample for immune repertoire nucleic acid sequencing, administering a Crohn's disease therapy to a subject diagnosed with Crohn's disease based on the assessment, and/or the like.

According to some embodiments, the subject has one or more non-specific symptoms consistent with Crohn's disease at the time of the assessing. Examples of such non-specific symptoms include, but are not limited to, diarrhea, fatigue, abdominal pain, abdominal cramping, rectal bleeding, unintended weight loss, and any combination thereof. As noted above, the methods of the present disclosure find use, e.g., in providing a differential diagnosis based on the assessing in which a subject who has one or more non-specific symptoms consistent with Crohn's disease is diagnosed as having Crohn's disease and not another condition characterized by symptoms which overlap with those of Crohn's disease, including but not limited to, ulcerative colitis, irritable bowel syndrome, and/or celiac disease.

According to some embodiments, the methods of the present disclosure find use in providing a differential diagnosis of the subject as having Crohn's disease with fistulation or structuring and not Crohn's disease without fistulation (abnormal passage between diseased loops of bowel, e.g.,) or structuring (narrowing of the bowel which may lead to bowel obstruction or changes in the caliber of the feces). See, e.g., Gasche et al., Inflamm. Bowel Dis., 6:8-15 (2000) In certain embodiments, the methods of the present disclosure find use in providing a differential diagnosis of the subject as having ileal/ileocolonic Crohn's disease and not colonic Crohn's disease. Details regarding ileal/ileocolonic Crohn's disease and colonic Crohn's disease may be found, e.g., in Atreya and Siegmund (2021) Nat Rev Gastroenterol Hepatol 18, 544-558.

As summarized above, the methods of the present disclosure comprise assessing the TCRβ CDR3 sequences determined from the sample obtained from the subject for the presence or absence of one or more TCRβ CDR3 sequences set forth in SEQ ID Nos:1-1996 set forth herein, e.g., SEQ ID Nos:1-1281 set forth herein. As noted above, in certain embodiments, the assessing step may be computer-implemented such that it is performed using one or more processors and one or more non-transitory computer-readable media comprising instructions stored thereon, which when executed by the one or more processors, cause the one or more processors to assess the determined TCRβ CDR3 sequences for the presence or absence of one or more TCRβ CDR3 sequences set forth in SEQ ID Nos:1-1996 (e.g., SEQ ID Nos:1-1281). For example, the instructions may cause the one or more processors to compare each of the determined TCRβ CDR3 sequences (e.g., each determined TCRβ CDR3 sequence or each unique determined TCRβ CDR3 sequence) stored on a computer-readable medium to a database comprising one or more TCRβ CDR3 sequences set forth in SEQ ID Nos:1-1996 (e.g., SEQ ID Nos:1-1281) stored on the same or a different computer-readable medium. According to some embodiments, the number of TCRβ CDR3 sequences determined from the sample obtained from the subject is from 1,000 to 2,000,000. For example, in certain embodiments, the number of determined TCRβ CDR3 sequences is 2,000,000 or fewer (e.g., 1,500,000 or fewer, 1,250,000 or fewer, 1,000,000 or fewer, 750,000 or fewer, or 500,000 or fewer), but 1,000 or more, 5,000 or more, 10,000 or more, 15,000 or more, 20,000 or more, 25,000 or more, 30,000 or more, 35,000 or more, 40,000 or more, 45,000 or more, 50,000 or more, 55,000 or more, 60,000 or more, 65,000 or more, 70,000 or more, 75,000 or more, 80,000 or more, 85,000 or more, 90,000 or more, 95,000 or more, or 100,000 or more. The number of TCRβ CDR3 sequences set forth in SEQ ID Nos:1-1996 (e.g., SEQ ID Nos:1-1281) to which the determined TCRβ CDR3 sequences is compared may vary. For example, the determined TCRβ CDR3 sequences may be compared to 1 or more, 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 75 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1000 or more, 1010 or more, 1020 or more, 1030 or more, or each of the TCRβ CDR3 sequences set forth in SEQ ID Nos:1-1996 (e.g., SEQ ID Nos:1-1281). When the determined TCRβ CDR3 sequences are compared to fewer than all of the TCRβ CDR3 sequences set forth in SEQ ID Nos:1-1996 (e.g., SEQ ID Nos:1-1281), the determined TCRβ CDR3 sequences may be compared to any desired number (e.g., as set forth above) and any desired combination of TCRβ CDR3 sequences set forth in SEQ ID Nos:1-1996, e.g., SEQ ID Nos:1-1281).

The methods of the present disclosure may include one or more additional steps based on the results of the assessing step. For example, if it is determined from the assessing step that none of the TCRβ CDR3 sequences set forth in SEQ ID Nos:1-1996 (e.g., SEQ ID Nos:1-1281) are present in the TCRβ CDR3 sequences determined from the sample obtained from the subject (e.g., a subject identified as having IB or suspected of having IBD, including but not limited to a subject exhibiting one or more non-specific symptoms consistent with Crohn's disease), then the methods may further comprise, e.g., identifying the subject as not having Crohn's disease. Also by way of example, if it is determined from the assessing step that one or more (e.g., 2 or more, 3 or more, 4 or more, 5 or more, or 10 or more) of the TCRβ CDR3 sequences set forth in SEQ ID Nos:1-1996 (e.g., SEQ ID Nos:1-1281) are present in the TCRβ CDR3 sequences determined from the sample obtained from the subject (e.g., a subject identified as having IB or suspected of having IBD, including but not limited to a subject exhibiting one or more non-specific symptoms consistent with Crohn's disease), then the methods may further comprise, e.g., predicting that the subject has Crohn's disease, diagnosing the subject as having Crohn's disease, identifying the subject as one who should be administered a Crohn's disease therapy, and/or administering a Crohn's disease therapy to the subject, e.g., administering to the subject one or more of any of the Crohn's disease therapies described elsewhere herein.

In certain embodiments, the methods further comprise subjecting the results of the assessing step to further analysis, such as subjecting the results of the assessing step to a model. For example, the methods may further comprise subjecting the results of the assessing step to a model in order to classify the subject as having Crohn's disease or not having Crohn's disease; and/or to classify the subject as having Crohn's disease and not having a non-Crohn's disease IBD, e.g., ulcerative colitis. One of ordinary skill in the art will appreciate that, with the benefit of the TCRβ CDR3 sequences set forth in SEQ ID Nos:1-1996 (e.g., SEQ ID Nos:1-1281) described herein, a variety of useful models may be applied to the results of the assessment. In one non-limiting example, the methods may further comprise subjecting the results of the assessing step to a two feature logistic regression with features representing the number of Crohn's disease-associated TCRβ CDR3 sequences determined from the sample and the total number of unique TCRβ CDR3 sequences determined from the sample. As demonstrated in the Experimental section below, such a model exhibits high specificity and sensitivity for Crohn's disease patients.

In certain embodiments, when the methods further comprise subjecting the results of the assessing step to a model for classification purposes (e.g., as described above), the model may take into account the number of unique Crohn's disease-associated TCRβ CDR3 sequences that are present in the TCRβ CDR3 sequences determined from the sample, e.g., where the greater the number of unique Crohn's disease-associated TCRβ CDR3 sequences, the more likely the model is to classify the subject as having Crohn's disease. According to some embodiments, the number of unique Crohn's disease-associated TCRβ CDR3 sequences is not a feature utilized by the model to classify the subject. In certain embodiments, the presence and/or frequency of one or more particular unique Crohn's disease-associated TCRβ CDR3 sequences is a feature(s) used by the model to classify the subject. For example, the presence and/or frequency of one or more particular unique Crohn's disease-associated TCRβ CDR3 sequences may be given relatively greater weight when classifying the subject as compared to the presence and/or frequency of one or more other unique Crohn's disease-associated TCRβ CDR3 sequences.

According to some embodiments, when a classification model weighs particular unique Crohn's disease-associated TCRβ CDR3 sequences differently than other unique Crohn's disease-associated TCRβ CDR3 sequences, the model may use convergent recombination to weigh the sequences differently. Different T cells can show convergent recombination where unique DNA sequences were formed in the recombination for a first T cell, a second T cell, a third T cell, etc., but where each leads to the same protein (CDR3+V-gene+J-gene) which is diagnostic for high likelihood of Crohn's disease. This convergent recombination may be more likely for certain Crohn's disease-associated TCRβ CDR3 sequences than others, and the model may take into account these aspects of the signal reflective of the interpretable biology of immune response. Accordingly, in some embodiments, sequences may be given differential weight based on convergent recombination.

In certain embodiments, prior to the assessing step, the methods may further include one or more steps for determining the TCRβ CDR3 sequences from the sample obtained from the subject. For example, the determining may include immunosequencing and evaluation of the T cell repertoire in the biological sample obtained from the subject, e.g., by high-throughput sequencing (HTS) as described elsewhere herein. The determining may be partially implemented using a computer. For example, the analysis of the raw sequencing data may be implemented by a computer. Extraction of DNA or RNA from the biological sample, amplification, and sequencing may be performed manually, using a machine, or a combination thereof. In certain embodiments, the methods may further comprise an initial step of obtaining the biological sample from the subject.

The biological sample (e.g., peripheral blood, gut tissue, and/or the like) may be obtained from a variety of subjects. Such subjects may be “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, non-human primates such as chimpanzees, and monkeys). In some embodiments, the subject is a human subject.

Biological samples of interest include those that comprise T cells, including but not limited to, whole blood samples, a fraction of whole blood comprising peripheral blood mononuclear cells (e.g., blood plasma), serum, a peripheral blood mononuclear cell (PBMC) sample, a gut tissue sample, urine, buffy coat, synovial fluid, bone marrow, cerebrospinal fluid, saliva, lymph fluid, seminal fluid, vaginal secretions, urethral secretions, exudate, transdermal exudates, pharyngeal exudates, nasal secretions, sputum, sweat, bronchoalveolar lavage, tracheal aspirations, fluid from joints, or vitreous fluid. T cells can also be obtained from biological samples which may be derived from, for example, solid tissue samples. T cells may be helper T cells (effector T cells or Th cells), cytotoxic T cells (CTLs), memory T cells, and regulatory T cells. In some embodiments, peripheral blood mononuclear cells (PBMC) are isolated by techniques known to those of skill in the art, e.g., by Ficoll-Hypaque® density gradient separation.

Nucleic acid, such as, genomic DNA or RNA may be extracted from lymphoid cells by methods known to those of skill in the art. Examples include using the QIAamp® DNA blood Mini Kit or a Qiagen DNeasy Blood extraction kit (both commercially available from Qiagen, Gaithersburg, Md., USA) to extract genomic DNA. In some embodiments, 100,000 to 200,000 cells may be used for analysis of diversity, i.e., about 0.6 to 1.2 μg DNA from diploid T cells. Using PBMCs as a source, the number of T cells can be estimated to be about 30% of total cells. Alternatively, total nucleic acid can be isolated from cells, including both genomic DNA and mRNA. In other embodiments, cDNA is transcribed from mRNA and then used as templates for amplification. The RNA molecules can be transcribed to cDNA using known reverse-transcription kits, such as the SMARTer™ Ultra Low RNA kit for Illumina sequencing (Clontech, Mountain View, Calif.) essentially according to the supplier's instructions.

### Immune Repertoire Sequencing (Multiplex PCR and High Throughput Sequencing)

According to some embodiments, TCRβ CDR3 sequences are determined from the sample obtained from the subject by immune cell receptor sequencing, e.g., immune repertoire sequencing.

By “T cell receptor” or “TCR” is meant a disulfide-linked membrane bound heterodimeric protein normally consisting of the highly variable α and β chains expressed as part of a complex with the invariant CD3 chain molecules. T cells expressing these two chains are referred to as α:β (or αβ) T cells, though a minority of T cells express an alternate receptor, formed by variable γ and a chains, referred as γσ T cells. TCR development occurs through a lymphocyte specific process of gene recombination, which assembles a final sequence from a large number of potential segments. This genetic recombination of TCR gene segments in somatic T cells occurs during the early stages of development in the thymus. The TCRα gene locus contains variable (V) and joining (J) gene segments (Vα and Jα), whereas the TCRβ locus contains a D gene segment in addition to Vβ and Jβ segments. Accordingly, the a chain is generated from VJ recombination and the β chain is involved in VDJ recombination. This is similar for the development of γδ TCRs, in which the TCRγ chain is involved in VJ recombination and the TCRδ gene is generated from VDJ recombination. The TCR α chain gene locus consists of 46 variable segments, 8 joining segments and the constant region. The TCR β chain gene locus consists of 48 variable segments followed by two diversity segments, 12 joining segments and two constant regions. The D and J segments are located within a relatively short 50 kb region while the variable genes are spread over a large region of 1.5 mega bases (TCRα) or 0.67 mega bases (TCRβ).

TCRβ CDR3 sequence determination may involve quantitative detection of sequences of substantially all possible TCR gene rearrangements that can be present in a sample containing lymphoid cell DNA.

Amplified nucleic acid molecules comprising rearranged TCR regions obtained from a biological sample are sequenced using high-throughput sequencing. In one embodiment, a multiplex PCR system is used to amplify rearranged TCR loci from genomic DNA as described in U.S. Pub. No. 2010/0330571, filed on Jun. 4, 2010, U.S. Pub. No. 2012/0058902, filed on Aug. 24, 2011, International App. No. PCT/US2013/062925, filed on Oct. 1, 2013, which is each incorporated by reference in its entirety.

To that end, multiplex PCR is performed using a set of forward primers that specifically hybridize to V segments and a set of reverse primers that specifically hybridize to the J segments of a TCR locus, where a multiplex PCR reaction using the primers allows amplification of all the possible VJ (and VDJ) combinations within a given population of T cells.

Exemplary V segment primers and J segment primers are described in US2012/0058902, US2010/033057, WO2010/151416, WO2011/106738, US2015/0299785, WO2012/027503, US2013/0288237, U.S. Pat. Nos. 9,181,590, 9,181,591, US2013/0253842, WO2013/188831, which are each herein incorporated by reference in their entireties.

A multiplex PCR system can be used to amplify rearranged immune cell receptor loci. In certain embodiments, the CDR3 region is amplified from a TCRB CDR3 region locus. A plurality of V-segment and J-segment primers are used to amplify substantially all (e.g., greater than 90%, 91%, 92%, 93%, 94%, 95%, 96%, 97%, 98% or 99%) rearranged immune cell receptor CDR3-encoding regions to produce a multiplicity of amplified rearranged DNA molecules. In certain embodiments, primers are designed so that each amplified rearranged DNA molecule is less than 600 nucleotides in length, thereby excluding amplification products from non-rearranged immune cell receptor loci.

In some embodiments, two pools of primers are used in a single, highly multiplexed PCR reaction. The “forward” pool of primers can include a plurality of V segment oligonucleotide primers and the reverse pool can include a plurality of J segment oligonucleotide primers. In some embodiments, there is a primer that is specific to (e.g., having a nucleotide sequence complementary to a unique sequence region of) each V region segment and to each J region segment in the respective TCR or Ig gene locus. In other embodiments, a primer can hybridize to one or more V segments or J segments, thereby reducing the number of primers required in the multiplex PCR. In certain embodiments, the J-segment primers anneal to a conserved sequence in the joining (“J”) segment.

Each primer can be designed such that a respective amplified DNA segment is obtained that includes a sequence portion of sufficient length to identify each J segment unambiguously based on sequence differences amongst known J-region encoding gene segments in the human genome database, and also to include a sequence portion to which a J-segment-specific primer can anneal for resequencing. This design of V- and J-segment-specific primers enables direct observation of a large fraction of the somatic rearrangements present in the immune cell receptor gene repertoire within the subject.

A multiplex PCR system can use at least 2, 3, 4, 5, 6, 7, 8, 9, 10, 11, 12, 13, 14, 15, 16, 17, 18, 19, 20, 21, 22, 23, 24, or 25, and in certain embodiments, at least 26, 27, 28, 29, 30, 31, 32, 33, 34, 35, 36, 37, 38, or 39, and in other embodiments at least 40, 41, 42, 43, 44, 45, 46, 47, 20 48, 49, 50, 51, 52, 53, 54, 55, 56, 57, 58, 59, 60, 65, 70, 75, 80, 85, or more forward primers, in which each forward primer specifically hybridizes to (i.e., is complementary to) a sequence corresponding to a V region segment. The multiplex PCR system also uses at least 2, 3, 4, 5, 6, or 7, and in certain embodiments, at least 8, 9, 10, 11, 12 or 13 reverse primers, or at least 14, 15, 16, 17, 18, 19, 20, 21, 22, 23, 24, or 25 or more reverse primers, in which each reverse primer specifically hybridizes to or is complementary to a sequence corresponding to a J region segment. Various combinations of V and J segment primers can be used to amplify the full diversity of TCR sequences in the immune cell receptor gene repertoire within the subject.

Further details on multiplex PCR system, including primer oligonucleotide sequences for amplifying TCR sequences are described in Robins et al., 2009 Blood 114, 4099; Robins et al., 2010 Sci. Translat. Med. 2:47ra64; Robins et al., 2011 J. Immunol. Meth. doi:10.1016/j.jim.2011.09. 001; Sherwood et al. 2011 Sci. Translat. Med. 3:90ra61; US2012/0058902, US2010/033057, WO/2010/151416, WO/2011/106738, US 2015/0299785, WO2012/027503, US2013/0288237, U.S. Pat. Nos. 9,181,590, 9,181,591, US2013/0253842, WO2013/188831, which is each incorporated herein by reference in its entirety.

Oligonucleotides or polynucleotides that are capable of specifically hybridizing or annealing to a target nucleic acid sequence by nucleotide base complementarity can do so under moderate to high stringency conditions. In one embodiment, suitable moderate to high stringency conditions for specific PCR amplification of a target nucleic acid sequence can be between 25 and 80 PCR cycles, with each cycle including a denaturation step (e.g., about 10-30 seconds (s) at greater than about 95° C.), an annealing step (e.g., about 10-30s at about 60-68° C.), and an extension step (e.g., about 10-60s at about 60-72° C.), optionally according to certain embodiments with the annealing and extension steps being combined to provide a two-step PCR. As would be recognized by the skilled person, other PCR reagents can be added or changed in the PCR reaction to increase specificity of primer annealing and amplification, such as altering the magnesium concentration, optionally adding DMSO, and/or the use of blocked primers, modified nucleotides, peptide-nucleic acids, and the like.

A primer may be a single-stranded DNA. The appropriate length of a primer depends on the intended use of the primer but typically ranges from 6 to 50 nucleotides, or in certain embodiments, from 15-35 nucleotides in length. Short primer molecules generally require cooler temperatures to form sufficiently stable hybrid complexes with the template. A primer need not reflect the exact sequence of the template nucleic acid, but must be sufficiently complementary to hybridize with the template. The design of suitable primers for the amplification of a given target sequence is well known in the art and described in the literature cited herein.

V- and J-segment primers are used to produce a plurality of amplicons from the multiplex PCR reaction. In certain embodiments, the amplicons range in size from 10, 20, 30, 40, 50, 75, 100, 200, 300, 400, 500, 600, 700, 800 or more nucleotides in length. In certain embodiments, the amplicons have a size between 20-600, 50-600, 20-400, or 50-400 nucleotides in length.

According to non-limiting theory, these embodiments exploit current understanding in the art (also described above) that once a T lymphocyte has rearranged its TCR-encoding genes, its progeny cells possess the same immune cell receptor-encoding gene rearrangement, thus giving rise to a clonal population (clones) that can be uniquely identified by the presence therein of rearranged (e.g., CDR3-encoding) V- and J-gene segments that can be amplified by a specific pairwise combination of V- and J-specific oligonucleotide primers as herein disclosed.

The V segment primers and J segment primers will preferably each include a second sequence at the 5′-end of the primer that is not complementary to the target V or J segment. The second sequence can comprise an oligonucleotide having a sequence that is selected from (i) a universal adaptor oligonucleotide sequence, and (ii) a sequencing platform-specific oligonucleotide sequence that is linked to and positioned 5′ to a first universal adaptor oligonucleotide sequence. Examples of universal adaptor oligonucleotide sequences can be pGEX forward and pGEX reverse adaptor sequences.

The resulting amplicons using the V-segment and J-segment primers described above include amplified V and J segments and the universal adaptor oligonucleotide sequences. The universal adaptor sequence can be complementary to an oligonucleotide sequence found in a tailing primer. Tailing primers can be used in a second PCR reaction to generate a second set of amplicons. In some embodiments, tailing primers can have the general formula (I):

5′-P--S--B--U-3′ (I),

where P comprises a sequencing platform-specific oligonucleotide, where S comprises a sequencing platform tag-containing oligonucleotide sequence; where B comprises an oligonucleotide barcode sequence and where the oligonucleotide barcode sequence can be used to identify a sample source, and where U comprises a sequence that is complementary to the universal adaptor oligonucleotide sequence or is the same as the universal adaptor oligonucleotide sequence.

Additional description about universal adaptor oligonucleotide sequences, barcodes, and tailing primers are found in WO2013/188831, which is incorporated by reference in its entirety.

Sequencing may be performed using any of a variety of available high throughput nucleic acid sequencing machines and systems. Illustrative sequencing systems include the Illumina iSeq 100, Miniseq, MiSeq series, NextSeq series (e.g., NextSeq 500 series, NextSeq 1000, NextSeq 2000), and NovaSeq sequencing systems (Illumina, Inc., San Diego, Calif.), the Pacific Biosciences Sequel (e.g., Sequel II) sequencing system (Pacific Biosciences, Menlo Park, Calif.), the Oxford Nanopore Technologies MinION™, GridIONx5™, PromethION™, or SmidgION™ nanopore-based sequencing systems (Oxford Nanopore Technologies, Oxford, UK), and other systems having similar capabilities.

In certain embodiments, sequencing is achieved using a set of sequencing platform-specific oligonucleotides that hybridize to a defined region within the amplified DNA molecules. The sequencing platform-specific oligonucleotides are designed to sequence amplicons, such that the V- and J-encoding gene segments can be uniquely identified by the sequences that are generated. See, e.g., US2012/0058902; US2010/033057; WO2011/106738; US2015/0299785; or WO2012/027503, which is each incorporated by reference in its entirety.

In some embodiments, the raw sequence data is preprocessed to remove errors in the primary sequence of each read and to compress the data. A nearest neighbor algorithm can be used to collapse the data into unique sequences by merging closely related sequences, to remove both PCR and sequencing errors. See, e.g., US2012/0058902; US2010/033057; WO2011/106738; US2015/0299785; or WO2012/027503, which is each incorporated by reference in its entirety.

Sequencing the multiplicity of amplified rearranged TCRβ CDR3-encoding region DNA molecules by high-throughput sequencing (HTS) can be used to produce a TCR clonotype profile comprising at least 10,000 TCR clonotype sequences of 20 to 400 nucleotides in length.

### Amplification Bias Control

Multiplex PCR assays can result in a bias in the total numbers of amplicons produced from a sample, given that certain primer sets may be more efficient in amplification than others. To overcome the problem of such biased utilization of subpopulations of amplification primers, methods can be used that provide a template composition for standardizing the amplification efficiencies of the members of an oligonucleotide primer set, where the primer set is capable of amplifying rearranged DNA encoding a plurality of TCRs in a biological sample that comprises DNA from lymphoid cells.

To that end, a template composition is used to standardize the various amplification efficiencies of the primer sets. The template composition can comprise a plurality of diverse template oligonucleotides of general formula (II):

5′-U1-B1-V-B2-R-J-B3-U2-3′ (II)

The constituent template oligonucleotides are diverse with respect to the nucleotide sequences of the individual template oligonucleotides. The individual template oligonucleotides can vary in nucleotide sequence considerably from one another as a function of significant sequence variability among the large number of possible TCR variable (V) and joining (J) region polynucleotides. Sequences of individual template oligonucleotide species can also vary from one another as a function of sequence differences in U1, U2, B (B1, B2 and B3) and R oligonucleotides that are included in a particular template within the diverse plurality of templates.

V is a polynucleotide comprising at least 20, 30, 60, 90, 120, 150, 180, or 210, and not more than 1000, 900, 800, 700, 600 or 500 contiguous nucleotides of an adaptive immune receptor variable (V) region encoding gene sequence, or the complement thereof, and in each of the plurality of template oligonucleotide sequences V comprises a unique oligonucleotide sequence.

J is a polynucleotide comprising at least 15-30, 31-60, 61-90, 91-120, or 120-150, and not more than 600, 500, 400, 300 or 200 contiguous nucleotides of an adaptive immune receptor joining (J) region encoding gene sequence, or the complement thereof, and in each of the plurality of template oligonucleotide sequences J comprises a unique oligonucleotide sequence.

U1 and U2 can be each either nothing or each comprise an oligonucleotide having, independently, a sequence that is selected from (i) a universal adaptor oligonucleotide sequence, and (ii) a sequencing platform-specific oligonucleotide sequence that is linked to and positioned 5′ to the universal adaptor oligonucleotide sequence.

B1, B2 and B3 can be each either nothing or each comprise an oligonucleotide B that comprises a first and a second oligonucleotide barcode sequence of 3, 4, 5, 6, 7, 8, 9, 10, 11, 12, 13, 14, 15, 16, 17, 18, 19, 20, 25, 30, 35, 40, 45, 50, 60, 70, 80, 90, 100, 200, 300, 400, 500, 600, 700, 800, 900 or 1000 contiguous nucleotides (including all integer values therebetween), wherein in each of the plurality of template oligonucleotide sequences B comprises a unique oligonucleotide sequence in which (i) the first barcode sequence uniquely identifies the unique V oligonucleotide sequence of the template oligonucleotide and (ii) the second barcode sequence uniquely identifies the unique J oligonucleotide sequence of the template oligonucleotide.

R can be either nothing or comprises a restriction enzyme recognition site that comprises an oligonucleotide sequence that is absent from V, J, U1, U2, B1, B2 and B3.

Methods are used with the template composition for determining non-uniform nucleic acid amplification potential among members of a set of oligonucleotide amplification primers that are capable of amplifying productively rearranged DNA encoding one or a plurality of TCRs in a biological sample that comprises DNA from lymphoid cells of a subject. The method can include the steps of: (a) amplifying DNA of a template composition for standardizing amplification efficiency of an oligonucleotide primer set in a multiplex polymerase chain reaction (PCR) that comprises: (i) the template composition (II) described above, wherein each template oligonucleotide in the plurality of template oligonucleotides is present in a substantially equimolar amount; (ii) an oligonucleotide amplification primer set that is capable of amplifying productively rearranged DNA encoding one or a plurality of TCRs in a biological sample that comprises DNA from lymphoid cells of a subject.

The primer set can include: (1) in substantially equimolar amounts, a plurality of V-segment oligonucleotide primers that are each independently capable of specifically hybridizing to at least one polynucleotide encoding a TCR V-region polypeptide or to the complement thereof, wherein each V-segment primer comprises a nucleotide sequence of at least 15 contiguous nucleotides that is complementary to at least one functional TCR V region-encoding gene segment and wherein the plurality of V-segment primers specifically hybridize to substantially all functional TCR V region-encoding gene segments that are present in the template composition, and (2) in substantially equimolar amounts, a plurality of J-segment oligonucleotide primers that are each independently capable of specifically hybridizing to at least one polynucleotide encoding a TCR J-region polypeptide or to the complement thereof, wherein each J-segment primer comprises a nucleotide sequence of at least 15 contiguous nucleotides that is complementary to at least one functional TCR J region-encoding gene segment and wherein the plurality of J-segment primers specifically hybridize to substantially all functional TCR J region-encoding gene segments that are present in the template composition.

The V-segment and J-segment oligonucleotide primers are capable of promoting amplification in said multiplex polymerase chain reaction (PCR) of substantially all template oligonucleotides in the template composition to produce a multiplicity of amplified template DNA molecules, said multiplicity of amplified template DNA molecules being sufficient to quantify diversity of the template oligonucleotides in the template composition, and wherein each amplified template DNA molecule in the multiplicity of amplified template DNA molecules is less than 1000, 900, 800, 700, 600, 500, 400, 300, 200, 100, 90, 80 or 70 nucleotides in length.

Methods for determining non-uniform nucleic acid amplification potential may further include: (b) sequencing all or a sufficient portion of each of said multiplicity of amplified template DNA molecules to determine, for each unique template DNA molecule in said multiplicity of amplified template DNA molecules, (i) a template-specific oligonucleotide DNA sequence and (ii) a relative frequency of occurrence of the template oligonucleotide; and (c) comparing the relative frequency of occurrence for each unique template DNA sequence from said template composition, wherein a non-uniform frequency of occurrence for one or more template DNA sequences indicates non-uniform nucleic acid amplification potential among members of the set of oligonucleotide amplification primers.

Further details concerning the aforementioned bias control methods are provided in US2013/0253842, U.S. Pat. No. 9,150,905, US2015/0203897, and WO2013/169957, which are incorporated by reference in their entireties.

### PCR Template Abundance Estimation

To estimate the average read coverage per input template in the multiplex PCR and sequencing approach, a set of synthetic TCR templates (as described above) can be used, comprising each combination of V.beta. and J.beta. gene segments. These synthetic molecules can be those described in general formula (II) above, and in US2013/0253842, U.S. Pat. No. 9,150,905, US2015/0203897, and WO2013/169957, which are incorporated by reference in their entireties.

These synthetic molecules can be included in each PCR reaction at very low concentration so that only some of the synthetic templates are observed. Using the known concentration of the synthetic template pool, the relationship between the number of observed unique synthetic molecules and the total number of synthetic molecules added to reaction can be simulated (this is very nearly one-to-one at the low concentrations that were used). The synthetic molecules allow calculation for each PCR reaction the mean number of sequencing reads obtained per molecule of PCR template, and an estimation of the number of T cells or B cells in the input material bearing each unique TCR rearrangement or Ig rearrangement, respectively.

TABLE 1
Crohn's Disease−Associated TCRβ CDR3 Amino Acid Sequences

| TCRβ CDR3 Amino | SEQ ID | | J Gene | Association |
|---|---|---|---|---|
| Acid Sequence | NO: | V Gene Segment | Segment | P-Value |
| CASSTDRAGELFF | 1 | TCRBV05-01 | TCRBJ02-02 | 7.59E−99 |
| CSAPGLGDEKLFF | 2 | TCRBV20-X | TCRBJ01-04 | 2.19E−97 |
| CATSRDLGTSYEQYF | 3 | TCRBV15-01 | TCRBJ02-07 | 2.30E−86 |
| CASSFSQETQYF | 4 | TCRBV05-04 | TCRBJ02-05 | 8.35E−86 |
| CASSSAGTANQPQHF | 5 | TCRBV06-05 | TCRBJ01-05 | 9.69E−86 |
| CASSSDRAGELFF | 6 | TCRBV05-01 | TCRBJ02-02 | 3.93E−81 |
| CASSSTGTANQPQHF | 7 | TCRBV06-05 | TCRBJ01-05 | 5.19E−76 |
| CAISRGGNQETQYF | 8 | TCRBV10-03 | TCRBJ02-05 | 6.86E−74 |
| CASSSDSPGELFF | 9 | TCRBV05-01 | TCRBJ02-02 | 4.58E−73 |
| CASSSDRPGELFF | 10 | TCRBV05-01 | TCRBJ02-02 | 4.47E−64 |
| CASSPTGTTNQPQHF | 11 | TCRBV06-05 | TCRBJ01-05 | 4.47E−62 |
| CASSQEPSGTYEQYF | 12 | TCRBV04-01 | TCRBJ02-07 | 5.32E−62 |
| CASSIGGGWGQPQHF | 13 | TCRBV19-01 | TCRBJ01-05 | 1.49E−58 |
| CASSQGQGEGYEQYF | 14 | TCRBV04-03 | TCRBJ02-07 | 2.10E−58 |
| CASSATGTANQPQHF | 15 | TCRBV06-X | TCRBJ01-05 | 2.02E−57 |
| CASRTGLNTGELFF | 16 | TCRBV27-01 | TCRBJ02-02 | 3.57E−57 |
| CSVEGGRPGELFF | 17 | TCRBV29-01 | TCRBJ02-02 | 4.52E−55 |
| CASSQGTEGYGYTF | 18 | TCRBV03-01/03-02 | TCRBJ01-02 | 2.13E−53 |
| CASRGEGNTEAFF | 19 | TCRBV12-03/12-04 | TCRBJ01-01 | 2.10E−51 |
| CASSLNPPGGYTF | 20 | TCRBV12-03/12-04 | TCRBJ01-02 | 4.91E−51 |
| CSATGLAGDSGANVLTF | 21 | TCRBV20-X | TCRBJ02-06 | 6.65E−49 |
| CASSRRDQYNEQFF | 22 | TCRBV19-01 | TCRBJ02-01 | 7.34E−49 |
| CASSFDRGENYGYTF | 23 | TCRBV27-01 | TCRBJ01-02 | 9.28E−49 |
| CASSLGDRVWETQYF | 24 | TCRBV05-06 | TCRBJ02-05 | 3.38E−48 |
| CASSDRDREGKLFF | 25 | TCRBV09-01 | TCRBJ01-04 | 1.31E−47 |
| CASSVDGTETYEQYF | 26 | TCRBV09-01 | TCRBJ02-07 | 3.64E−46 |
| CASSPPATVNTEAFF | 27 | TCRBV18-01 | TCRBJ01-01 | 1.23E−43 |
| CASSLAVGHLNTEAFF | 28 | TCRBV05-01 | TCRBJ01-01 | 1.77E−43 |
| CASSQEGQGFGNTIYF | 29 | TCRBV04-02 | TCRBJ01-03 | 2.19E−43 |
| CASSLALGHLNTEAFF | 30 | TCRBV05-01 | TCRBJ01-01 | 5.34E−42 |
| CASSLDGTGSEQYF | 31 | TCRBV07-09 | TCRBJ02-07 | 1.83E−40 |
| CASSPGAVNTEAFF | 32 | TCRBV07-06 | TCRBJ01-01 | 2.85E−40 |
| CASSVGGRALYNEQFF | 33 | TCRBV09-01 | TCRBJ02-01 | 4.63E−39 |
| CASRGGQGMTEAFF | 34 | TCRBV06-05 | TCRBJ01-01 | 6.59E−39 |
| CASSYGQGRNYGYTF | 35 | TCRBV05-01 | TCRBJ01-02 | 1.32E−38 |
| CASSEYSPLHF | 36 | TCRBV02-01 | TCRBJ01-06 | 8.22E−38 |
| CASSSGPIQETQYF | 37 | TCRBV07-03 | TCRBJ02-05 | 1.88E−36 |
| CASSSSGTANQPQHF | 38 | TCRBV06-05 | TCRBJ01-05 | 7.83E−36 |
| CASSSRQGRTGELFF | 39 | TCRBV28-01 | TCRBJ02-02 | 1.21E−35 |
| CSALGGVAEAFF | 40 | TCRBV20-X | TCRBJ01-01 | 1.84E−35 |
| CASSTDSPGELFF | 41 | TCRBV05-01 | TCRBJ02-02 | 4.91E−35 |
| CASSGDRPGELFF | 42 | TCRBV05-01 | TCRBJ02-02 | 4.91E−35 |
| CAISAGDSTDTQYF | 43 | TCRBV10-03 | TCRBJ02-03 | 5.15E−35 |
| CSAPGGGRDNSPLHF | 44 | TCRBV20-X | TCRBJ01-06 | 1.16E−34 |
| CASSLGSVNTEAFF | 45 | TCRBV07-06 | TCRBJ01-01 | 1.20E−34 |
| CSARDVASGANVLTF | 46 | TCRBV20-X | TCRBJ02-06 | 2.58E−34 |
| CASSLGTVDNSPLHF | 47 | TCRBV05-06 | TCRBJ01-06 | 1.00E−33 |
| CASSTDRPGELFF | 48 | TCRBV05-01 | TCRBJ02-02 | 5.07E−33 |
| CASSLAGLAGGPDTQYF | 49 | TCRBV07-02 | TCRBJ02-03 | 7.87E−33 |
| CASSLGAVNTEAFF | 50 | TCRBV07-08 | TCRBJ01-01 | 1.25E−32 |
| CSVWTGATDTQYF | 51 | TCRBV29-01 | TCRBJ02-03 | 1.57E−32 |
| CASRSTGYNQPQHF | 52 | TCRBV06-05 | TCRBJ01-05 | 2.10E−32 |
| CASSYGDRNSPLHF | 53 | TCRBV06-02/06-03 | TCRBJ01-06 | 7.93E−32 |
| CASRDVDTGELFF | 54 | TCRBV05-01 | TCRBJ02-02 | 1.14E−31 |
| CASSYGEGWNQPQHF | 55 | TCRBV06-02/06-03 | TCRBJ01-05 | 1.20E−31 |
| CASSYRQANNQPQHF | 56 | TCRBV06-02/06-03 | TCRBJ01-05 | 2.03E−31 |
| CASSPQINYGYTF | 57 | TCRBV07-02 | TCRBJ01-02 | 1.07E−30 |
| CASRDIDTGELFF | 58 | TCRBV05-01 | TCRBJ02-02 | 1.69E−30 |
| CASSLGDRAEAFF | 59 | TCRBV05-06 | TCRBJ01-01 | 1.92E−30 |
| CASRTGVNTGELFF | 60 | TCRBV27-01 | TCRBJ02-02 | 2.78E−30 |
| CASSQEGRSNTEAFF | 61 | TCRBV14-01 | TCRBJ01-01 | 5.76E−30 |
| CASSTDRTGELFF | 62 | TCRBV05-01 | TCRBJ02-02 | 8.44E−30 |
| CASSRGEDSPLHF | 63 | TCRBV28-01 | TCRBJ01-06 | 8.87E−30 |
| CASSLRGGGDYGYTF | 64 | TCRBV07-02 | TCRBJ01-02 | 9.69E−30 |
| CASRGSSGNQPQHF | 65 | TCRBV02-01 | TCRBJ01-05 | 1.12E−29 |
| CASSFNDRGNTEAFF | 66 | TCRBV27-01 | TCRBJ01-01 | 1.22E−29 |
| CASSLGDGEQFF | 67 | TCRBV11-02 | TCRBJ02-01 | 6.04E−29 |
| CASSRLSSYEQYF | 68 | TCRBV28-01 | TCRBJ02-07 | 6.33E−29 |
| CASSLVFGQSYEQYF | 69 | TCRBV11-02 | TCRBJ02-07 | 8.57E−29 |
| CASSPWGGETQYF | 70 | TCRBV04-02 | TCRBJ02-05 | 8.88E−29 |
| CASSPKDRGGEKLFF | 71 | TCRBV09-01 | TCRBJ01-04 | 9.33E−29 |
| CASSQWGGETQYF | 72 | TCRBV04-02 | TCRBJ02-05 | 1.69E−28 |
| CASSYGDRGNQPQHF | 73 | TCRBV06-02/06-03 | TCRBJ01-05 | 3.87E−28 |
| CASSQDPQETQYF | 74 | TCRBV14-01 | TCRBJ02-05 | 6.47E−28 |
| CSAWTASYNEQFF | 75 | TCRBV20-X | TCRBJ02-01 | 7.31E−28 |
| CASSPGAVNTEAFF | 32 | TCRBV07-X | TCRBJ01-01 | 8.97E−28 |
| CASSQADSTDTQYF | 76 | TCRBV04-01 | TCRBJ02-03 | 9.71E−28 |
| CASSPGSEGQPQHF | 77 | TCRBV18-01 | TCRBJ01-05 | 1.39E−27 |
| CASRIGEETQYF | 78 | TCRBV05-01 | TCRBJ02-05 | 1.75E−27 |
| CASSQEPSGIYEQYF | 79 | TCRBV04-01 | TCRBJ02-07 | 3.75E−27 |
| CASSERDRGYEQYF | 80 | TCRBV06-01 | TCRBJ02-07 | 4.37E−27 |
| CASSELDRAGNTIYF | 81 | TCRBV02-01 | TCRBJ01-03 | 4.76E−27 |
| CASSYGDKNSPLHF | 82 | TCRBV06-02/06-03 | TCRBJ01-06 | 8.37E−27 |
| CASSLAGLAGSPDTQYF | 83 | TCRBV07-02 | TCRBJ02-03 | 1.04E−26 |
| CASSGTGDGYTF | 84 | TCRBV19-01 | TCRBJ01-02 | 1.35E−26 |
| CASSIGSGWGQPQHF | 85 | TCRBV19-01 | TCRBJ01-05 | 2.55E−26 |
| CASRSEGNTEAFF | 86 | TCRBV12-03/12-04 | TCRBJ01-01 | 2.84E−26 |
| CASSRQGVGNQPQHF | 87 | TCRBV07-09 | TCRBJ01-05 | 3.89E−26 |
| CASRQGKGTEAFF | 88 | TCRBV06-X | TCRBJ01-01 | 4.68E−26 |
| CASSPSGGQGDTQYF | 89 | TCRBV05-01 | TCRBJ02-03 | 6.19E−26 |
| CASSSGTGRSNQPQHF | 90 | TCRBV11-02 | TCRBJ01-05 | 6.88E−26 |
| CASSLDLKNTGELFF | 91 | TCRBV11-02 | TCRBJ02-02 | 7.22E−26 |
| CASSYPSRGLETQYF | 92 | TCRBV06-02/06-03 | TCRBJ02-05 | 9.60E−26 |
| CASSNSSGANVLTF | 93 | TCRBV18-01 | TCRBJ02-06 | 1.05E−25 |
| CATSDLGVRGTGELFF | 94 | TCRBV24-01 | TCRBJ02-02 | 1.72E−25 |
| CASSFHRDRETQYF | 95 | TCRBV05-01 | TCRBJ02-05 | 2.05E−25 |
| CASSLIRADTQYF | 96 | TCRBV27-01 | TCRBJ02-03 | 2.13E−25 |
| CASSPSGGGEETQYF | 97 | TCRBV18-01 | TCRBJ02-05 | 2.34E−25 |
| CASSPPSGGRNTGELFF | 98 | TCRBV18-01 | TCRBJ02-02 | 2.96E−25 |
| CASSLDRGGSYNEQFF | 99 | TCRBV05-01 | TCRBJ02-01 | 3.37E−25 |
| CASSGRDGNTGELFF | 100 | TCRBV12-X | TCRBJ02-02 | 3.69E−25 |
| CASSFRLAGPQETQYF | 101 | TCRBV11-02 | TCRBJ02-05 | 4.73E−25 |
| CASSEGGLYGYTF | 102 | TCRBV10-02 | TCRBJ01-02 | 6.63E−25 |
| CASSGDSPGELFF | 103 | TCRBV05-01 | TCRBJ02-02 | 1.43E−24 |
| CASSPSGGLEETQYF | 104 | TCRBV18-01 | TCRBJ02-05 | 3.08E−24 |
| CASSTDRVGEAFF | 105 | TCRBV05-01 | TCRBJ01-01 | 3.72E−24 |
| CASSYSPTGATEAFF | 106 | TCRBV06-05 | TCRBJ01-01 | 3.84E−24 |
| CASSLGRSGSSYEQYF | 107 | TCRBV07-09 | TCRBJ02-07 | 5.02E−24 |
| CASSTDRSGELFF | 108 | TCRBV05-01 | TCRBJ02-02 | 6.32E−24 |
| CASRPDGYNQPQHF | 109 | TCRBV06-05 | TCRBJ01-05 | 6.32E−24 |
| CASHGNSPLHF | 110 | TCRBV06-05 | TCRBJ01-06 | 6.33E−24 |
| CASSLVPGEYGYTF | 111 | TCRBV07-09 | TCRBJ01-02 | 7.45E−24 |
| CASSPRTGSAETQYF | 112 | TCRBV19-01 | TCRBJ02-05 | 9.72E−24 |
| CSARDPREDTGELFF | 113 | TCRBV20-X | TCRBJ02-02 | 1.02E−23 |
| CASSPSPTANTEAFF | 114 | TCRBV06-05 | TCRBJ01-01 | 1.60E−23 |
| CASSEGDRGNQPQHF | 115 | TCRBV09-01 | TCRBJ01-05 | 1.76E−23 |
| CASSLTGTSGYNEQFF | 116 | TCRBV07-02 | TCRBJ02-01 | 1.86E−23 |
| CASSIGGGQYNEQFF | 117 | TCRBV19-01 | TCRBJ02-01 | 1.91E−23 |
| CASSLSRRYQETQYF | 118 | TCRBV27-01 | TCRBJ02-05 | 2.03E−23 |
| CASSPRTGGSYNSPLHF | 119 | TCRBV07-02 | TCRBJ01-06 | 2.07E−23 |
| CASSESKGGRNEQFF | 120 | TCRBV10-01 | TCRBJ02-01 | 2.29E−23 |
| CASSLARAGGPGDTQYF | 121 | TCRBV12-X | TCRBJ02-03 | 2.59E−23 |
| CASSVGLAGGGNEQFF | 122 | TCRBV07-09 | TCRBJ02-01 | 3.90E−23 |
| CASSLALAGGPDTQYF | 123 | TCRBV07-02 | TCRBJ02-03 | 4.48E−23 |
| CASSSRLAGAPDTQYF | 124 | TCRBV27-01 | TCRBJ02-03 | 4.51E−23 |
| CSARASGGPTYNEQFF | 125 | TCRBV20-X | TCRBJ02-01 | 5.11E−23 |
| CASSFRDRATEAFF | 126 | TCRBV11-02 | TCRBJ01-01 | 6.55E−23 |
| CATSFGTGGAGELFF | 127 | TCRBV15-01 | TCRBJ02-02 | 6.60E−23 |
| CASSFGANVLTF | 128 | TCRBV07-03 | TCRBJ02-06 | 1.48E−22 |
| CASSLADRVWETQYF | 129 | TCRBV05-06 | TCRBJ02-05 | 1.64E−22 |
| CASSLGGNSYQPQHF | 130 | TCRBV07-02 | TCRBJ01-05 | 1.68E−22 |
| CAISGGDSTDTQYF | 131 | TCRBV10-03 | TCRBJ02-03 | 1.70E−22 |
| CASSQDRAYNQPQHF | 132 | TCRBV04-02 | TCRBJ01-05 | 1.86E−22 |
| CASSPRGAGDTQYF | 133 | TCRBV07-02 | TCRBJ02-03 | 1.98E−22 |
| CASSPRTGTAETQYF | 134 | TCRBV19-01 | TCRBJ02-05 | 2.71E−22 |
| CASSPPDSPTNEKLFF | 135 | TCRBV18-01 | TCRBJ01-04 | 3.22E−22 |
| CSARGQGETGELFF | 136 | TCRBV20-X | TCRBJ02-02 | 3.37E−22 |
| CASSPQGTGKGNEQFF | 137 | TCRBV18-01 | TCRBJ02-01 | 4.96E−22 |
| CASRSPRGRNQPQHF | 138 | TCRBV28-01 | TCRBJ01-05 | 5.12E−22 |
| CASSYRQGARQPQHF | 139 | TCRBV06-02/06-03 | TCRBJ01-05 | 5.92E−22 |
| CASSLDRGENYGYTF | 140 | TCRBV27-01 | TCRBJ01-02 | 6.87E−22 |
| CASSLVSSGQYNEQFF | 141 | TCRBV07-09 | TCRBJ02-01 | 1.13E−21 |
| CSARDPGEDTGELFF | 142 | TCRBV20-X | TCRBJ02-02 | 1.20E−21 |
| CASRDADTGELFF | 143 | TCRBV05-01 | TCRBJ02-02 | 1.49E−21 |
| CSARNRSTDTQYF | 144 | TCRBV20-X | TCRBJ02-03 | 2.05E−21 |
| CSAIGTGSDEQYF | 145 | TCRBV20-X | TCRBJ02-07 | 2.22E−21 |
| CASRPPTGRNQPQHF | 146 | TCRBV28-01 | TCRBJ01-05 | 2.41E−21 |
| CASSPGSVNTEAFF | 147 | TCRBV07-06 | TCRBJ01-01 | 2.50E−21 |
| CSAPPPGVGNTIYF | 148 | TCRBV20-X | TCRBJ01-03 | 2.68E−21 |
| CASSPGSVNTEAFF | 147 | TCRBV07-X | TCRBJ01-01 | 3.03E−21 |
| CSATGAQADTQYF | 149 | TCRBV20-X | TCRBJ02-03 | 3.41E−21 |
| CSVKYGSGNTIYF | 150 | TCRBV29-01 | TCRBJ01-03 | 4.07E−21 |
| CASSLGGQGRNYGYTF | 151 | TCRBV05-04 | TCRBJ01-02 | 4.36E−21 |
| CASSLDRAETEAFF | 152 | TCRBV05-01 | TCRBJ01-01 | 4.75E−21 |
| CSVRGQLNTEAFF | 153 | TCRBV20-X | TCRBJ01-01 | 5.62E−21 |
| CASSPSGTTNQPQHF | 154 | TCRBV06-05 | TCRBJ01-05 | 5.92E−21 |
| CASSPAGGRNEQFF | 155 | TCRBV11-02 | TCRBJ02-01 | 7.05E−21 |
| CSVEGGRPGEQYF | 156 | TCRBV29-01 | TCRBJ02-07 | 7.45E−21 |
| CATKTASTDTQYF | 157 | TCRBV06-X | TCRBJ02-03 | 1.01E−20 |
| CASSLGDGEQYF | 158 | TCRBV11-02 | TCRBJ02-07 | 1.13E−20 |
| CASSLAPGGHTEAFF | 159 | TCRBV05-06 | TCRBJ01-01 | 1.14E−20 |
| CSVEFLGGADTQYF | 160 | TCRBV29-01 | TCRBJ02-03 | 1.25E−20 |
| CSATGTSDREQYF | 161 | TCRBV20-X | TCRBJ02-07 | 1.29E−20 |
| CASSYSPTANTEAFF | 162 | TCRBV06-05 | TCRBJ01-01 | 1.39E−20 |
| CASSLGGTGASGANVLTF | 163 | TCRBV07-02 | TCRBJ02-06 | 1.62E−20 |
| CASSFGRAANTGELFF | 164 | TCRBV05-01 | TCRBJ02-02 | 1.71E−20 |
| CASSPSAANNEKLFF | 165 | TCRBV18-01 | TCRBJ01-04 | 1.96E−20 |
| CASSFWQGRNTIYF | 166 | TCRBV05-06 | TCRBJ01-03 | 2.16E−20 |
| CASTSSGNTIYF | 167 | TCRBV07-08 | TCRBJ01-03 | 2.53E−20 |
| CSARWESFYNEQFF | 168 | TCRBV20-X | TCRBJ02-01 | 2.76E−20 |
| CASSYPSSGRETQYF | 169 | TCRBV06-02/06-03 | TCRBJ02-05 | 2.88E−20 |
| CSVEHRGRGKNIQYF | 170 | TCRBV29-01 | TCRBJ02-04 | 3.12E−20 |
| CSARGQGDGQPQHF | 171 | TCRBV20-X | TCRBJ01-05 | 3.31E−20 |
| CASSLVGRVYGYTF | 172 | TCRBV05-04 | TCRBJ01-02 | 4.36E−20 |
| CAISLGGNQETQYF | 173 | TCRBV10-03 | TCRBJ02-05 | 5.00E−20 |
| CASSGDRAGELFF | 174 | TCRBV05-01 | TCRBJ02-02 | 5.06E−20 |
| CASSATGTANQPQHF | 15 | TCRBV06-05 | TCRBJ01-05 | 5.45E−20 |
| CASSQTGTANQPQHF | 175 | TCRBV06-05 | TCRBJ01-05 | 5.62E−20 |
| CASRTGINTGELFF | 176 | TCRBV27-01 | TCRBJ02-02 | 6.32E−20 |
| CSARVGTGNNSPLHF | 177 | TCRBV20-X | TCRBJ01-06 | 8.65E−20 |
| CASSLKDRDGNTIYF | 178 | TCRBV07-08 | TCRBJ01-03 | 8.95E−20 |
| CASSYKGGADTQYF | 179 | TCRBV06-02/06-03 | TCRBJ02-03 | 1.01E−19 |
| CASSTDRAGEAFF | 180 | TCRBV05-01 | TCRBJ01-01 | 1.03E−19 |
| CASSHLRGSNEKLFF | 181 | TCRBV04-01 | TCRBJ01-04 | 1.03E−19 |
| CASSVGSRRLAGAYEQYF | 182 | TCRBV09-01 | TCRBJ02-07 | 1.30E−19 |
| CASSRDRNQETQYF | 183 | TCRBV05-05 | TCRBJ02-05 | 1.87E−19 |
| CASSWRLAGPQETQYF | 184 | TCRBV11-02 | TCRBJ02-05 | 2.00E−19 |
| CASSLEGFGELFF | 185 | TCRBV11-01 | TCRBJ02-02 | 2.45E−19 |
| CATSRDGGTVNSPLHF | 186 | TCRBV15-01 | TCRBJ01-06 | 2.46E−19 |
| CASRAEGNTEAFF | 187 | TCRBV12-03/12-04 | TCRBJ01-01 | 2.54E−19 |
| CASSYRAFYNEQFF | 188 | TCRBV06-02/06-03 | TCRBJ02-01 | 2.76E−19 |
| CAIQRGGDTQYF | 189 | TCRBV10-03 | TCRBJ02-03 | 4.60E−19 |
| CASSTRHEQYF | 190 | TCRBV04-02 | TCRBJ02-07 | 5.45E−19 |
| CSARVLAGGAGELFF | 191 | TCRBV20-X | TCRBJ02-02 | 7.45E−19 |
| CASSLDRGGGYNEQFF | 192 | TCRBV05-01 | TCRBJ02-01 | 1.08E−18 |
| CASSLAWTGMNTEAFF | 193 | TCRBV05-01 | TCRBJ01-01 | 1.30E−18 |
| CASSMGLGGSQPQHF | 194 | TCRBV19-01 | TCRBJ01-05 | 1.37E−18 |
| CASSPGRDSSYNEQFF | 195 | TCRBV18-01 | TCRBJ02-01 | 1.58E−18 |
| CASSTDRVGELFF | 196 | TCRBV05-01 | TCRBJ02-02 | 1.87E−18 |
| CASSLKGQGFSGANVLTF | 197 | TCRBV07-09 | TCRBJ02-06 | 1.90E−18 |
| CASRWTGIYGYTF | 198 | TCRBV07-02 | TCRBJ01-02 | 2.24E−18 |
| CASSVALRGALNTEAFF | 199 | TCRBV09-01 | TCRBJ01-01 | 2.59E−18 |
| CASSPGTVGNTIYF | 200 | TCRBV14-01 | TCRBJ01-03 | 2.60E−18 |
| CASSSRHEQFF | 201 | TCRBV04-02 | TCRBJ02-01 | 2.64E−18 |
| CASSESRGRRNTEAFF | 202 | TCRBV10-01 | TCRBJ01-01 | 3.27E−18 |
| CASSPPGDRNTDTQYF | 203 | TCRBV18-01 | TCRBJ02-03 | 3.59E−18 |
| CSATPGRIYGYTF | 204 | TCRBV20-X | TCRBJ01-02 | 3.67E−18 |
| CSARNRGPYEQYF | 205 | TCRBV20-X | TCRBJ02-07 | 3.69E−18 |
| CASSIRTGTAETQYF | 206 | TCRBV19-01 | TCRBJ02-05 | 3.86E−18 |
| CASSPRDREGKLFF | 207 | TCRBV09-01 | TCRBJ01-04 | 3.93E−18 |
| CAISKGGNQETQYF | 208 | TCRBV10-03 | TCRBJ02-05 | 4.78E−18 |
| CASSRGASGNTIYF | 209 | TCRBV05-01 | TCRBJ01-03 | 6.78E−18 |
| CASSPGTGRLNQPQHF | 210 | TCRBV18-01 | TCRBJ01-05 | 7.13E−18 |
| CSARDPRVDTGELFF | 211 | TCRBV20-X | TCRBJ02-02 | 1.18E−17 |
| CSAAGAQADTQYF | 212 | TCRBV20-X | TCRBJ02-03 | 1.27E−17 |
| CASSLEIQETQYF | 213 | TCRBV07-02 | TCRBJ02-05 | 1.28E−17 |
| CASSTRTGRNEKLFF | 214 | TCRBV04-01 | TCRBJ01-04 | 1.48E−17 |
| CASSASGTANQPQHF | 215 | TCRBV06-05 | TCRBJ01-05 | 1.62E−17 |
| CSARAGVAYNEQFF | 216 | TCRBV20-X | TCRBJ02-01 | 1.67E−17 |
| CASSVKDRGGEKLFF | 217 | TCRBV09-01 | TCRBJ01-04 | 2.20E−17 |
| CASSSDRSGELFF | 218 | TCRBV05-01 | TCRBJ02-02 | 2.52E−17 |
| CASSTRQGRTGELFF | 219 | TCRBV28-01 | TCRBJ02-02 | 3.34E−17 |
| CASSSRTGLGTEAFF | 220 | TCRBV12-X | TCRBJ01-01 | 3.34E−17 |
| CASSLGGQKNQPQHF | 221 | TCRBV05-06 | TCRBJ01-05 | 3.45E−17 |
| CASSLQQGRTEAFF | 222 | TCRBV11-02 | TCRBJ01-01 | 3.92E−17 |
| CASSLLSGGNYNEQFF | 223 | TCRBV11-02 | TCRBJ02-01 | 5.17E−17 |
| CASSLRDRDGNTIYF | 224 | TCRBV07-08 | TCRBJ01-03 | 5.26E−17 |
| CASSPPRGLAGGPGTDTQ | 225 | TCRBV18-01 | TCRBJ02-03 | 5.89E−17 |
| YF | | | | |
| CASSNRDREGKLFF | 226 | TCRBV09-01 | TCRBJ01-04 | 7.73E−17 |
| CSARDRVTGGSYNSPLHF | 227 | TCRBV20-X | TCRBJ01-06 | 8.61E−17 |
| CASSEKGDSGNTIYF | 228 | TCRBV06-01 | TCRBJ01-03 | 1.10E−16 |
| CASRLRDPSTDTQYF | 229 | TCRBV05-01 | TCRBJ02-03 | 1.22E−16 |
| CASSPRLYNEQFF | 230 | TCRBV14-01 | TCRBJ02-01 | 1.34E−16 |
| CSARDPRTDTGELFF | 231 | TCRBV20-X | TCRBJ02-02 | 1.60E−16 |
| CASSLVTGTMNTEAFF | 232 | TCRBV18-01 | TCRBJ01-01 | 1.99E−16 |
| CASSPSPTGNTEAFF | 233 | TCRBV06-05 | TCRBJ01-01 | 2.22E−16 |
| CASSLASGSRQETQYF | 234 | TCRBV05-04 | TCRBJ02-05 | 2.24E−16 |
| CASKRGEGSYEQYF | 235 | TCRBV28-01 | TCRBJ02-07 | 2.24E−16 |
| CASSFSVDSPLHF | 236 | TCRBV07-02 | TCRBJ01-06 | 2.24E−16 |
| CSVEGGQGNTDTQYF | 237 | TCRBV29-01 | TCRBJ02-03 | 2.39E−16 |
| CASSESAVYNSPLHF | 238 | TCRBV25-01 | TCRBJ01-06 | 2.65E−16 |
| CASSLAGQKNQPQHF | 239 | TCRBV05-06 | TCRBJ01-05 | 3.03E−16 |
| CASSPHLKETQYF | 240 | TCRBV27-01 | TCRBJ02-05 | 3.42E−16 |
| CASSERDRPYEQYF | 241 | TCRBV06-01 | TCRBJ02-07 | 3.61E−16 |
| CASSVAPRGGRSTDTQYF | 242 | TCRBV09-01 | TCRBJ02-03 | 3.62E−16 |
| CASSSMTSGTDTQYF | 243 | TCRBV11-03 | TCRBJ02-03 | 3.70E−16 |
| CASRTGGGNTEAFF | 244 | TCRBV05-05 | TCRBJ01-01 | 4.65E−16 |
| CASSATGNTIYF | 245 | TCRBV07-08 | TCRBJ01-03 | 4.95E−16 |
| CASSLAWIGMNTEAFF | 246 | TCRBV05-05 | TCRBJ01-01 | 5.46E−16 |
| CSARVLAGGSGELFF | 247 | TCRBV20-X | TCRBJ02-02 | 5.76E−16 |
| CASSIRTGSAETQYF | 248 | TCRBV19-01 | TCRBJ02-05 | 5.95E−16 |
| CASSTDRAGEQFF | 249 | TCRBV05-01 | TCRBJ02-01 | 5.96E−16 |
| CASSAQGVGNQPQHF | 250 | TCRBV07-09 | TCRBJ01-05 | 7.80E−16 |
| CSARLAGGPPSYNEQFF | 251 | TCRBV20-X | TCRBJ02-01 | 8.37E−16 |
| CASSPLAGGPRTDTQYF | 252 | TCRBV11-02 | TCRBJ02-03 | 8.67E−16 |
| CASSPLGGSNEKLFF | 253 | TCRBV09-01 | TCRBJ01-04 | 9.26E−16 |
| CASSHEGGETQYF | 254 | TCRBV04-02 | TCRBJ02-05 | 1.25E−15 |
| CASSPLLARETQYF | 255 | TCRBV14-01 | TCRBJ02-05 | 1.32E−15 |
| CASSFRGGGDYGYTF | 256 | TCRBV07-02 | TCRBJ01-02 | 1.35E−15 |
| CASSFAGPRSYNEQFF | 257 | TCRBV05-01 | TCRBJ02-01 | 1.52E−15 |
| CASSPKTGGSYNSPLHF | 258 | TCRBV07-02 | TCRBJ01-06 | 1.52E−15 |
| CASSSRLAGEVDTQYF | 259 | TCRBV27-01 | TCRBJ02-03 | 1.57E−15 |
| CASSRLAGNNEQFF | 260 | TCRBV07-08 | TCRBJ02-01 | 1.74E−15 |
| CASSPTSYSYEQYF | 261 | TCRBV05-04 | TCRBJ02-07 | 1.87E−15 |
| CASSHRHEQFF | 262 | TCRBV04-02 | TCRBJ02-01 | 2.30E−15 |
| CSVPRQGTDTQYF | 263 | TCRBV29-01 | TCRBJ02-03 | 2.49E−15 |
| CASSFLSGGNYNEQFF | 264 | TCRBV11-02 | TCRBJ02-01 | 2.57E−15 |
| CSATGTSDREQFF | 265 | TCRBV20-X | TCRBJ02-01 | 2.64E−15 |
| CASSYSVSPNQPQHF | 266 | TCRBV06-05 | TCRBJ01-05 | 3.27E−15 |
| CASSLTEGGQPQHF | 267 | TCRBV05-01 | TCRBJ01-05 | 3.50E−15 |
| CASSLGDRRGNTIYF | 268 | TCRBV11-02 | TCRBJ01-03 | 4.75E−15 |
| CASSGGQGETEAFF | 269 | TCRBV06-X | TCRBJ01-01 | 4.78E−15 |
| CASSPRDYEQYF | 270 | TCRBV04-02 | TCRBJ02-07 | 4.81E−15 |
| CASSPPGDRNSYEQYF | 271 | TCRBV18-01 | TCRBJ02-07 | 4.90E−15 |
| CASSPGTEGYGYTF | 272 | TCRBV03-01/03-02 | TCRBJ01-02 | 5.09E−15 |
| CASSLVFGENYGYTF | 273 | TCRBV11-02 | TCRBJ01-02 | 5.11E−15 |
| CASSLGTSYTDTQYF | 274 | TCRBV05-04 | TCRBJ02-03 | 5.36E−15 |
| CSAIRGSGELFF | 275 | TCRBV20-X | TCRBJ02-02 | 5.83E−15 |
| CASSLGQGEGYEQYF | 276 | TCRBV04-03 | TCRBJ02-07 | 5.84E−15 |
| CSARGVRREQYF | 277 | TCRBV20-X | TCRBJ02-07 | 5.98E−15 |
| CASRTPRGRNQPQHF | 278 | TCRBV28-01 | TCRBJ01-05 | 6.71E−15 |
| CASSQEGAGLATDTQYF | 279 | TCRBV04-01 | TCRBJ02-03 | 6.71E−15 |
| CASSFRLAGAPGYEQYF | 280 | TCRBV27-01 | TCRBJ02-07 | 6.71E−15 |
| CASTPGQGADGYTF | 281 | TCRBV06-05 | TCRBJ01-02 | 7.81E−15 |
| CASSPGTSGGARETQYF | 282 | TCRBV12-X | TCRBJ02-05 | 1.49E−14 |
| CASRRDREGNEQFF | 283 | TCRBV06-06 | TCRBJ02-01 | 1.49E−14 |
| CAIRGLAGGPTDTQYF | 284 | TCRBV10-03 | TCRBJ02-03 | 1.61E−14 |
| CAIGTGDSTDTQYF | 285 | TCRBV10-03 | TCRBJ02-03 | 1.66E−14 |
| CATSPTSNYGYTF | 286 | TCRBV15-01 | TCRBJ01-02 | 1.66E−14 |
| CASSPPAGGRNTGELFF | 287 | TCRBV18-01 | TCRBJ02-02 | 1.91E−14 |
| CASSPYTNTGELFF | 288 | TCRBV06-04 | TCRBJ02-02 | 2.22E−14 |
| CASSNRDSSPLHF | 289 | TCRBV11-02 | TCRBJ01-06 | 2.30E−14 |
| CASSPTSFSYEQYF | 290 | TCRBV05-04 | TCRBJ02-07 | 2.99E−14 |
| CSATGLADSGANVLTF | 291 | TCRBV20-X | TCRBJ02-06 | 3.31E−14 |
| CASSSGPTGELFF | 292 | TCRBV27-01 | TCRBJ02-02 | 3.53E−14 |
| CASSPQGTGKGETQYF | 293 | TCRBV18-01 | TCRBJ02-05 | 4.31E−14 |
| CASRRVADQPQHF | 294 | TCRBV02-01 | TCRBJ01-05 | 4.96E−14 |
| CASSFAGEDYEQYF | 295 | TCRBV12-X | TCRBJ02-07 | 5.04E−14 |
| CASSPETTVNTEAFF | 296 | TCRBV18-01 | TCRBJ01-01 | 5.20E−14 |
| CASSPTPSLNEQFF | 297 | TCRBV27-01 | TCRBJ02-01 | 5.24E−14 |
| CASSARDRGSGANVLTF | 298 | TCRBV06-01 | TCRBJ02-06 | 5.87E−14 |
| CASSLFGGIQETQYF | 299 | TCRBV27-01 | TCRBJ02-05 | 6.46E−14 |
| CASSLPPNEQFF | 300 | TCRBV27-01 | TCRBJ02-01 | 6.71E−14 |
| CASSPKGGVNTGELFF | 301 | TCRBV09-01 | TCRBJ02-02 | 6.83E−14 |
| CASSESKGGRNEQYF | 302 | TCRBV10-01 | TCRBJ02-07 | 7.43E−14 |
| CASSPQGQGKGQPQHF | 303 | TCRBV18-01 | TCRBJ01-05 | 8.50E−14 |
| CASSPFGTSYNEQFF | 304 | TCRBV27-01 | TCRBJ02-01 | 9.14E−14 |
| CASKAGGPGNEQFF | 305 | TCRBV02-01 | TCRBJ02-01 | 9.39E−14 |
| CASSYLGEQGNEQFF | 306 | TCRBV06-05 | TCRBJ02-01 | 1.00E−13 |
| CASSEGRGGPKETQYF | 307 | TCRBV02-01 | TCRBJ02-05 | 1.17E−13 |
| CSARRAGGPPSYNEQFF | 308 | TCRBV20-X | TCRBJ02-01 | 1.21E−13 |
| CASSTQQGRTEAFF | 309 | TCRBV11-02 | TCRBJ01-01 | 1.31E−13 |
| CSARSQGTDTQYF | 310 | TCRBV20-X | TCRBJ02-03 | 1.52E−13 |
| CASSKGLAGNYEQYF | 311 | TCRBV04-03 | TCRBJ02-07 | 1.59E−13 |
| CASSYGQGWNTEAFF | 312 | TCRBV06-02/06-03 | TCRBJ01-01 | 2.13E−13 |
| CASSYLGEQGYGYTF | 313 | TCRBV06-05 | TCRBJ01-02 | 2.23E−13 |
| CASSLAVGHMNTEAFF | 314 | TCRBV05-01 | TCRBJ01-01 | 2.95E−13 |
| CASSELRTGELFF | 315 | TCRBV10-02 | TCRBJ02-02 | 2.99E−13 |
| CASSYSVAPNQPQHF | 316 | TCRBV06-05 | TCRBJ01-05 | 3.02E−13 |
| CASSADRPGELFF | 317 | TCRBV05-01 | TCRBJ02-02 | 3.50E−13 |
| CSARWESYYNEQFF | 318 | TCRBV20-X | TCRBJ02-01 | 3.66E−13 |
| CASSRELADTQYF | 319 | TCRBV07-03 | TCRBJ02-03 | 3.87E−13 |
| CASSISPQGGPYEQYF | 320 | TCRBV19-01 | TCRBJ02-07 | 4.33E−13 |
| CASSIKRTGTEAFF | 321 | TCRBV19-01 | TCRBJ01-01 | 4.40E−13 |
| CAWSRTGRNTGELFF | 322 | TCRBV30-01 | TCRBJ02-02 | 4.82E−13 |
| CASSQETGVQETQYF | 323 | TCRBV04-02 | TCRBJ02-05 | 4.99E−13 |
| CASSVRTASSTDTQYF | 324 | TCRBV09-01 | TCRBJ02-03 | 5.06E−13 |
| CASRRHRNTEAFF | 325 | TCRBV12-03/12-04 | TCRBJ01-01 | 5.88E−13 |
| CASSPRTGTAETQYF | 134 | TCRBV07-02 | TCRBJ02-05 | 6.33E−13 |
| CASSFWQGRYEQYF | 326 | TCRBV05-06 | TCRBJ02-07 | 6.50E−13 |
| CASSQDIAGETQYF | 327 | TCRBV04-03 | TCRBJ02-05 | 6.71E−13 |
| CASRPGRDSGNTIYF | 328 | TCRBV12-X | TCRBJ01-03 | 7.15E−13 |
| CSAHTGEKLFF | 329 | TCRBV20-01 | TCRBJ01-04 | 8.67E−13 |
| CAIGGGDSTDTQYF | 330 | TCRBV10-03 | TCRBJ02-03 | 8.80E−13 |
| CASSLEIADTQYF | 331 | TCRBV07-02 | TCRBJ02-03 | 9.15E−13 |
| CASSPTGTANQPQHF | 332 | TCRBV06-X | TCRBJ01-05 | 9.19E−13 |
| CASSQGRERQPQHF | 333 | TCRBV04-01 | TCRBJ01-05 | 1.14E−12 |
| CASSLRGGDDYGYTF | 334 | TCRBV07-03 | TCRBJ01-02 | 1.19E−12 |
| CASSREGAGEQYF | 335 | TCRBV18-01 | TCRBJ02-07 | 1.24E−12 |
| CASSLVSQGQYNEQFF | 336 | TCRBV07-09 | TCRBJ02-01 | 1.45E−12 |
| CASSQESGGPLDTQYF | 337 | TCRBV03-01/03-02 | TCRBJ02-03 | 1.49E−12 |
| CASSPPWGTDTQYF | 338 | TCRBV19-01 | TCRBJ02-03 | 1.60E−12 |
| CASSLGGDSSYNEQFF | 339 | TCRBV07-02 | TCRBJ02-01 | 1.94E−12 |
| CASSFTWTGMNTEAFF | 340 | TCRBV05-05 | TCRBJ01-01 | 2.01E−12 |
| CASSQEGQGFGYGYTF | 341 | TCRBV04-02 | TCRBJ01-02 | 2.12E−12 |
| CASSQEGQGFGNEQFF | 342 | TCRBV04-02 | TCRBJ02-01 | 2.12E−12 |
| CASSYGANVLTF | 343 | TCRBV07-03 | TCRBJ02-06 | 2.16E−12 |
| CASSLLSTVNQPQHF | 344 | TCRBV28-01 | TCRBJ01-05 | 2.17E−12 |
| CSARLLAGGAGELFF | 345 | TCRBV20-X | TCRBJ02-02 | 2.63E−12 |
| CASSYGQGWNQPQHF | 346 | TCRBV06-02/06-03 | TCRBJ01-05 | 2.77E−12 |
| CAWRDLGGHTDTQYF | 347 | TCRBV30-01 | TCRBJ02-03 | 2.84E−12 |
| CASSLNQDNEQFF | 348 | TCRBV11-02 | TCRBJ02-01 | 2.94E−12 |
| CASSQEGQGWGNTIYF | 349 | TCRBV04-02 | TCRBJ01-03 | 3.08E−12 |
| CASSQESQGFGNTIYF | 350 | TCRBV04-02 | TCRBJ01-03 | 3.08E−12 |
| CAISVEGGGSYEQYF | 351 | TCRBV10-03 | TCRBJ02-07 | 3.36E−12 |
| CSARGTSGGGLTDTQYF | 352 | TCRBV20-X | TCRBJ02-03 | 4.18E−12 |
| CASSFRGSGQETQYF | 353 | TCRBV07-03 | TCRBJ02-05 | 4.32E−12 |
| CASSQALSGNTIYF | 354 | TCRBV14-01 | TCRBJ01-03 | 4.88E−12 |
| CSAHRGAGGPQETQYF | 355 | TCRBV20-X | TCRBJ02-05 | 4.94E−12 |
| CASSLGDRGRDGYTF | 356 | TCRBV07-06 | TCRBJ01-02 | 5.50E−12 |
| CSAKWGTDQPQHF | 357 | TCRBV20-X | TCRBJ01-05 | 5.72E−12 |
| CASSLDGTGSEQFF | 358 | TCRBV07-09 | TCRBJ02-01 | 5.72E−12 |
| CASSTDTPGELFF | 359 | TCRBV05-01 | TCRBJ02-02 | 5.72E−12 |
| CATRDTDTGELFF | 360 | TCRBV05-01 | TCRBJ02-02 | 5.73E−12 |
| CASSPSTANNEKLFF | 361 | TCRBV18-01 | TCRBJ01-04 | 6.10E−12 |
| CASIRLAGGADTQYF | 362 | TCRBV07-02 | TCRBJ02-03 | 6.79E−12 |
| CASSQWGGEKLFF | 363 | TCRBV04-02 | TCRBJ01-04 | 7.40E−12 |
| CASSQQGVNYGYTF | 364 | TCRBV04-03 | TCRBJ01-02 | 7.52E−12 |
| CASSQNLGREQYF | 365 | TCRBV14-01 | TCRBJ02-07 | 8.10E−12 |
| CASSVGPGQGVNTEAFF | 366 | TCRBV09-01 | TCRBJ01-01 | 8.14E−12 |
| CASTPTGNTIYF | 367 | TCRBV07-08 | TCRBJ01-03 | 8.24E−12 |
| CASSLNPGGGDGYTF | 368 | TCRBV07-03 | TCRBJ01-02 | 8.62E−12 |
| CSARDPSGDTGELFF | 369 | TCRBV20-X | TCRBJ02-02 | 8.80E−12 |
| CASSPTGTGVDGYTF | 370 | TCRBV05-01 | TCRBJ01-02 | 8.90E−12 |
| CASSRADFEAFF | 371 | TCRBV18-01 | TCRBJ01-01 | 9.31E−12 |
| CASSPPVGARNQPQHF | 372 | TCRBV19-01 | TCRBJ01-05 | 9.35E−12 |
| CASSEAYRDSSYEQYF | 373 | TCRBV06-01 | TCRBJ02-07 | 9.35E−12 |
| CASSLTDQSYNEQFF | 374 | TCRBV07-03 | TCRBJ02-01 | 1.14E−11 |
| CASSSENSPLHF | 375 | TCRBV06-06 | TCRBJ01-06 | 1.32E−11 |
| CASSPAGARNEQFF | 376 | TCRBV11-02 | TCRBJ02-01 | 1.60E−11 |
| CASSSQTGLNQPQHF | 377 | TCRBV07-06 | TCRBJ01-05 | 1.62E−11 |
| CASSQDLQRIYNEQFF | 378 | TCRBV04-01 | TCRBJ02-01 | 1.64E−11 |
| CASSPDGTGGEQYF | 379 | TCRBV07-09 | TCRBJ02-07 | 1.65E−11 |
| CASSGHPNTEAFF | 380 | TCRBV06-04 | TCRBJ01-01 | 1.70E−11 |
| CASSFNPSYEQYF | 381 | TCRBV07-X | TCRBJ02-07 | 1.75E−11 |
| CASSSSGARAFF | 382 | TCRBV27-01 | TCRBJ01-01 | 1.87E−11 |
| CSATGGSGNNEQFF | 383 | TCRBV20-X | TCRBJ02-01 | 1.88E−11 |
| CASSMGGGWGQPQHF | 384 | TCRBV19-01 | TCRBJ01-05 | 2.02E−11 |
| CAWTRGLSYEQYF | 385 | TCRBV30-01 | TCRBJ02-07 | 2.14E−11 |
| CASSYGDGWNQPQHF | 386 | TCRBV06-02/06-03 | TCRBJ01-05 | 2.19E−11 |
| CASSDSQNIQYF | 387 | TCRBV05-01 | TCRBJ02-04 | 2.21E−11 |
| CASSLTLGHLNTEAFF | 388 | TCRBV05-01 | TCRBJ01-01 | 2.41E−11 |
| CASSYSPSGRVGNIQYF | 389 | TCRBV06-X | TCRBJ02-04 | 2.61E−11 |
| CASSQGRGEQPQHF | 390 | TCRBV03-01/03-02 | TCRBJ01-05 | 2.61E−11 |
| CASSLGIGDNQPQHF | 391 | TCRBV07-09 | TCRBJ01-05 | 2.75E−11 |
| CASSQGGTGPDNEQFF | 392 | TCRBV14-01 | TCRBJ02-01 | 2.89E−11 |
| CASSISPMTGSNQPQHF | 393 | TCRBV19-01 | TCRBJ01-05 | 2.89E−11 |
| CASSSDKAGELFF | 394 | TCRBV05-01 | TCRBJ02-02 | 2.89E−11 |
| CASSLSGLAGERELFF | 395 | TCRBV27-01 | TCRBJ02-02 | 3.08E−11 |
| CASSGRQEINEKLFF | 396 | TCRBV06-X | TCRBJ01-04 | 3.40E−11 |
| CASSFRAEGEKLFF | 397 | TCRBV27-01 | TCRBJ01-04 | 3.87E−11 |
| CASSLAPGQGGEQYF | 398 | TCRBV05-05 | TCRBJ02-07 | 4.09E−11 |
| CASSLASGTGTYGYTF | 399 | TCRBV07-02 | TCRBJ01-02 | 4.14E−11 |
| CASSLGQHTGELFF | 400 | TCRBV06-02/06-03 | TCRBJ02-02 | 4.41E−11 |
| CASSADSPGELFF | 401 | TCRBV05-01 | TCRBJ02-02 | 4.96E−11 |
| CASSEDGRGADTQYF | 402 | TCRBV06-04 | TCRBJ02-03 | 5.01E−11 |
| CASSRSTAQETQYF | 403 | TCRBV05-05 | TCRBJ02-05 | 5.01E−11 |
| CASRAPRGRNQPQHF | 404 | TCRBV28-01 | TCRBJ01-05 | 5.02E−11 |
| CASTRTRRSYEQYF | 405 | TCRBV28-01 | TCRBJ02-07 | 5.02E−11 |
| CASSVALRGALNNEQFF | 406 | TCRBV09-01 | TCRBJ02-01 | 5.21E−11 |
| CASSTGGEQYF | 407 | TCRBV27-01 | TCRBJ02-07 | 6.59E−11 |
| CSVEGTRPGELFF | 408 | TCRBV29-01 | TCRBJ02-02 | 6.63E−11 |
| CASSRDFAYNEQFF | 409 | TCRBV27-01 | TCRBJ02-01 | 6.74E−11 |
| CASSPPTGAGTGELFF | 410 | TCRBV05-01 | TCRBJ02-02 | 7.14E−11 |
| CATSRDSKTQYF | 411 | TCRBV15-01 | TCRBJ02-05 | 7.33E−11 |
| CASMDRAGEKLFF | 412 | TCRBV19-01 | TCRBJ01-04 | 7.42E−11 |
| CASSLRTTGLNTEAFF | 413 | TCRBV05-01 | TCRBJ01-01 | 7.53E−11 |
| CASSPRTGRAETQYF | 414 | TCRBV04-03 | TCRBJ02-05 | 9.09E−11 |
| CSARRGVAYNEQFF | 415 | TCRBV20-X | TCRBJ02-01 | 9.46E−11 |
| CSARELAGGAGELFF | 416 | TCRBV20-X | TCRBJ02-02 | 9.58E−11 |
| CASSWKTDYEQYF | 417 | TCRBV05-05 | TCRBJ02-07 | 9.65E−11 |
| CASSLYPSLYEQYF | 418 | TCRBV07-09 | TCRBJ02-07 | 9.65E−11 |
| CASSEWGDDNQPQHF | 419 | TCRBV02-01 | TCRBJ01-05 | 1.17E−10 |
| CSARDPRQDTGELFF | 420 | TCRBV20-X | TCRBJ02-02 | 1.17E−10 |
| CSARGQGDHQPQHF | 421 | TCRBV20-X | TCRBJ01-05 | 1.18E−10 |
| CASSPPSAGRNTGELFF | 422 | TCRBV18-01 | TCRBJ02-02 | 1.20E−10 |
| CSARVGVAYNEQFF | 423 | TCRBV20-X | TCRBJ02-01 | 1.38E−10 |
| CASSLVFGESYEQYF | 424 | TCRBV11-02 | TCRBJ02-07 | 1.53E−10 |
| CASSTQGRDSPLHF | 425 | TCRBV07-08 | TCRBJ01-06 | 1.61E−10 |
| CASRRPQGRNQPQHF | 426 | TCRBV28-01 | TCRBJ01-05 | 1.61E−10 |
| CSATTSWNEQFF | 427 | TCRBV20-X | TCRBJ02-01 | 1.66E−10 |
| CASSLTGLIQETQYF | 428 | TCRBV27-01 | TCRBJ02-05 | 1.70E−10 |
| CASSSGTAGGSPLHF | 429 | TCRBV05-01 | TCRBJ01-06 | 1.76E−10 |
| CSVWGRATDTQYF | 430 | TCRBV29-01 | TCRBJ02-03 | 1.76E−10 |
| CAISQGDSTDTQYF | 431 | TCRBV10-03 | TCRBJ02-03 | 1.76E−10 |
| CASSAKDRGGEKLFF | 432 | TCRBV09-01 | TCRBJ01-04 | 1.78E−10 |
| CSVEFSGGADTQYF | 433 | TCRBV29-01 | TCRBJ02-03 | 1.98E−10 |
| CASSPKAGLTGELFF | 434 | TCRBV07-03 | TCRBJ02-02 | 2.22E−10 |
| CASSSELQETQYF | 435 | TCRBV07-02 | TCRBJ02-05 | 2.22E−10 |
| CSLEEGTDTQYF | 436 | TCRBV20-X | TCRBJ02-03 | 2.24E−10 |
| CASSLRGSGQETQYF | 437 | TCRBV07-03 | TCRBJ02-05 | 2.29E−10 |
| CASSLGASLDTQYF | 438 | TCRBV11-03 | TCRBJ02-03 | 2.31E−10 |
| CASSLITGTLNTEAFF | 439 | TCRBV18-01 | TCRBJ01-01 | 2.45E−10 |
| CSATGAGSDEQYF | 440 | TCRBV20-X | TCRBJ02-07 | 2.47E−10 |
| CSANLGQENTEAFF | 441 | TCRBV20-X | TCRBJ01-01 | 2.53E−10 |
| CSARPQGDNQPQHF | 442 | TCRBV20-X | TCRBJ01-05 | 2.70E−10 |
| CASRQAHGYTF | 443 | TCRBV05-01 | TCRBJ01-02 | 2.73E−10 |
| CASSPQGQLNTEAFF | 444 | TCRBV04-02 | TCRBJ01-01 | 2.84E−10 |
| CASRTGGARGYTF | 445 | TCRBV06-06 | TCRBJ01-02 | 3.02E−10 |
| CASSGGSGGPQETQYF | 446 | TCRBV02-01 | TCRBJ02-05 | 3.06E−10 |
| CSVEGGTGNTDTQYF | 447 | TCRBV29-01 | TCRBJ02-03 | 3.06E−10 |
| CASSERASGYNEQFF | 448 | TCRBV25-01 | TCRBJ02-01 | 3.23E−10 |
| CASSPQTGYEQYF | 449 | TCRBV12-03/12-04 | TCRBJ02-07 | 3.29E−10 |
| CASSTGTSYNSPLHF | 450 | TCRBV06-02/06-03 | TCRBJ01-06 | 3.42E−10 |
| CASSLEGKGDQPQHF | 451 | TCRBV07-08 | TCRBJ01-05 | 3.43E−10 |
| CASSGDLPGELFF | 452 | TCRBV05-01 | TCRBJ02-02 | 3.43E−10 |
| CASSSDEPEKLFF | 453 | TCRBV05-01 | TCRBJ01-04 | 3.43E−10 |
| CASSLAWTGMSYEQYF | 454 | TCRBV05-05 | TCRBJ02-07 | 3.43E−10 |
| CASSGDSAGELFF | 455 | TCRBV05-01 | TCRBJ02-02 | 3.77E−10 |
| CASSPPRGTSGGPNTGEL | 456 | TCRBV18-01 | TCRBJ02-02 | 3.93E−10 |
| FF | | | | |
| CSARALAGGAGELFF | 457 | TCRBV20-X | TCRBJ02-02 | 4.13E−10 |
| CASSPTGGARYEQYF | 458 | TCRBV09-01 | TCRBJ02-07 | 4.43E−10 |
| CASSTRTGSAETQYF | 459 | TCRBV19-01 | TCRBJ02-05 | 4.76E−10 |
| CASSTRTGTAETQYF | 460 | TCRBV19-01 | TCRBJ02-05 | 4.76E−10 |
| CASSQTFDEQYF | 461 | TCRBV14-01 | TCRBJ02-07 | 5.03E−10 |
| CATSRDYPQNSPLHF | 462 | TCRBV15-01 | TCRBJ01-06 | 5.03E−10 |
| CASSSDKPGELFF | 463 | TCRBV05-01 | TCRBJ02-02 | 5.38E−10 |
| CASSFVWTGMNTEAFF | 464 | TCRBV05-05 | TCRBJ01-01 | 5.38E−10 |
| CSARGNDYQPQHF | 465 | TCRBV20-X | TCRBJ01-05 | 5.38E−10 |
| CASSLISGTVNTEAFF | 466 | TCRBV18-01 | TCRBJ01-01 | 5.72E−10 |
| CASSSDNPGELFF | 467 | TCRBV05-01 | TCRBJ02-02 | 5.94E−10 |
| CASSPPGDRNTYEQYF | 468 | TCRBV18-01 | TCRBJ02-07 | 6.05E−10 |
| CASSLWQGRYEQYF | 469 | TCRBV05-06 | TCRBJ02-07 | 6.55E−10 |
| CASSLDAGPQETQYF | 470 | TCRBV11-02 | TCRBJ02-05 | 6.59E−10 |
| CASSRIAGDYNEQFF | 471 | TCRBV03-01/03-02 | TCRBJ02-01 | 6.59E−10 |
| CASSESGTRKETQYF | 472 | TCRBV10-02 | TCRBJ02-05 | 6.71E−10 |
| CASSEVFGGRETQYF | 473 | TCRBV06-01 | TCRBJ02-05 | 6.71E−10 |
| CASSYRLAGPQETQYF | 474 | TCRBV11-02 | TCRBJ02-05 | 6.71E−10 |
| CASSLDLKAGELFF | 475 | TCRBV11-02 | TCRBJ02-02 | 6.94E−10 |
| CASSLDGTGTEQYF | 476 | TCRBV07-09 | TCRBJ02-07 | 7.14E−10 |
| CASSLVRGHNSPLHF | 477 | TCRBV05-05 | TCRBJ01-06 | 7.31E−10 |
| CASSGDPPSSYNEQFF | 478 | TCRBV06-01 | TCRBJ02-01 | 7.40E−10 |
| CASSPDQNGYTF | 479 | TCRBV19-01 | TCRBJ01-02 | 7.40E−10 |
| CSAPPDSQSYNEQFF | 480 | TCRBV20-X | TCRBJ02-01 | 7.40E−10 |
| CASSVAQGDGGELFF | 481 | TCRBV09-01 | TCRBJ02-02 | 7.78E−10 |
| CASSVAPRGGRGTDTQYF | 482 | TCRBV09-01 | TCRBJ02-03 | 7.79E−10 |
| CASSSDLPGELFF | 483 | TCRBV05-01 | TCRBJ02-02 | 7.96E−10 |
| CASSLPGLLNEQFF | 484 | TCRBV28-01 | TCRBJ02-01 | 8.74E−10 |
| CASSLLGQRRGYTF | 485 | TCRBV05-06 | TCRBJ01-02 | 9.22E−10 |
| CASSLDLKVTEAFF | 486 | TCRBV11-02 | TCRBJ01-01 | 9.59E−10 |
| CASSEVFGGREKLFF | 487 | TCRBV06-01 | TCRBJ01-04 | 9.78E−10 |
| CASRGPRGRNQPQHF | 488 | TCRBV28-01 | TCRBJ01-05 | 9.78E−10 |
| CASSLKSGLGTGELFF | 489 | TCRBV07-03 | TCRBJ02-02 | 9.78E−10 |
| CASSESKGRRNTEAFF | 490 | TCRBV10-01 | TCRBJ01-01 | 9.78E−10 |
| CSATGWGTEAFF | 491 | TCRBV20-X | TCRBJ01-01 | 1.04E−09 |
| CASSLQRDSYEQYF | 492 | TCRBV05-05 | TCRBJ02-07 | 1.12E−09 |
| CASSLGNRVYQETQYF | 493 | TCRBV05-01 | TCRBJ02-05 | 1.12E−09 |
| CASSWGDEGYTF | 494 | TCRBV05-01 | TCRBJ01-02 | 1.20E−09 |
| CASSLAGSRSGANVLTF | 495 | TCRBV05-06 | TCRBJ02-06 | 1.28E−09 |
| CASSFRRNSGNTIYF | 496 | TCRBV05-06 | TCRBJ01-03 | 1.31E−09 |
| CASSLGGLAGDNEQFF | 497 | TCRBV05-05 | TCRBJ02-01 | 1.47E−09 |
| CASSRGREDQPQHF | 498 | TCRBV05-05 | TCRBJ01-05 | 1.59E−09 |
| CATSRDWGQNSPLHF | 499 | TCRBV15-01 | TCRBJ01-06 | 1.62E−09 |
| CASSLTAQSYNEQFF | 500 | TCRBV07-03 | TCRBJ02-01 | 1.66E−09 |
| CASSYGQGRSYGYTF | 501 | TCRBV05-01 | TCRBJ01-02 | 1.66E−09 |
| CAISEGSSGLETQYF | 502 | TCRBV10-03 | TCRBJ02-05 | 1.79E−09 |
| CASSRGGQGKNIQYF | 503 | TCRBV05-01 | TCRBJ02-04 | 1.79E−09 |
| CASSYGVTRGTEAFF | 504 | TCRBV06-05 | TCRBJ01-01 | 1.80E−09 |
| CASSLVRDRAQETQYF | 505 | TCRBV05-06 | TCRBJ02-05 | 1.87E−09 |
| CASSQGGQGIYEQYF | 506 | TCRBV03-01/03-02 | TCRBJ02-07 | 1.95E−09 |
| CASSPGQGRLNQPQHF | 507 | TCRBV18-01 | TCRBJ01-05 | 2.05E−09 |
| CAWRVQYNEQFF | 508 | TCRBV30-01 | TCRBJ02-01 | 2.20E−09 |
| CASSSGGGPGSPLHF | 509 | TCRBV05-05 | TCRBJ01-06 | 2.36E−09 |
| CASSLYPSVYEQYF | 510 | TCRBV07-09 | TCRBJ02-07 | 2.62E−09 |
| CASSFNGLMNTEAFF | 511 | TCRBV05-01 | TCRBJ01-01 | 2.79E−09 |
| CAWARGLSYEQYF | 512 | TCRBV30-01 | TCRBJ02-07 | 2.90E−09 |
| CASSLYGTGPYEQYF | 513 | TCRBV05-06 | TCRBJ02-07 | 2.91E−09 |
| CASSLRGIGDTQYF | 514 | TCRBV07-03 | TCRBJ02-03 | 2.94E−09 |
| CSARGQGDDQPQHF | 515 | TCRBV20-X | TCRBJ01-05 | 2.94E−09 |
| CASSLVGSRSGANVLTF | 516 | TCRBV05-06 | TCRBJ02-06 | 3.18E−09 |
| CASSGERPGELFF | 517 | TCRBV05-01 | TCRBJ02-02 | 3.74E−09 |
| CASSSGTSGRAPDTQYF | 518 | TCRBV27-01 | TCRBJ02-03 | 3.74E−09 |
| CASSLRGRGDLNTEAFF | 519 | TCRBV07-02 | TCRBJ01-01 | 3.74E−09 |
| CASSADQGVSGNTIYF | 520 | TCRBV09-01 | TCRBJ01-03 | 3.74E−09 |
| CASSLARTSGRNEQFF | 521 | TCRBV27-01 | TCRBJ02-01 | 3.74E−09 |
| CASSVPPDTQYF | 522 | TCRBV27-01 | TCRBJ02-03 | 4.25E−09 |
| CASRIPRGRNQPQHF | 523 | TCRBV28-01 | TCRBJ01-05 | 4.31E−09 |
| CASSSGSVNTEAFF | 524 | TCRBV07-06 | TCRBJ01-01 | 4.31E−09 |
| CASSLVPTGGGYNEQFF | 525 | TCRBV07-02 | TCRBJ02-01 | 4.31E−09 |
| CASRGSRGRNQPQHF | 526 | TCRBV28-01 | TCRBJ01-05 | 4.31E−09 |
| CASSLKWPGSYEQYF | 527 | TCRBV05-05 | TCRBJ02-07 | 4.68E−09 |
| CASSFSRGQETQYF | 528 | TCRBV11-02 | TCRBJ02-05 | 5.42E−09 |
| CASSWERGTEAFF | 529 | TCRBV07-09 | TCRBJ01-01 | 5.75E−09 |
| CASSPGDRADQPQHF | 530 | TCRBV05-01 | TCRBJ01-05 | 5.92E−09 |
| CASSPPTSGEYNEQFF | 531 | TCRBV05-04 | TCRBJ02-01 | 5.98E−09 |
| CASSPPNSGRVGTDTQYF | 532 | TCRBV18-01 | TCRBJ02-03 | 6.42E−09 |
| CASSQEGGRASTDTQYF | 533 | TCRBV04-03 | TCRBJ02-03 | 6.91E−09 |
| CASSDHLGSGNTIYF | 534 | TCRBV06-01 | TCRBJ01-03 | 7.11E−09 |
| CSAIRLAGGKDTQYF | 535 | TCRBV20-X | TCRBJ02-03 | 7.23E−09 |
| CASRNNEKLFF | 536 | TCRBV04-03 | TCRBJ01-04 | 7.73E−09 |
| CASSFWQGRNIQYF | 537 | TCRBV05-06 | TCRBJ02-04 | 7.73E−09 |
| CSARALAGGSGELFF | 538 | TCRBV20-X | TCRBJ02-02 | 8.17E−09 |
| CSARVVLGTQYF | 539 | TCRBV20-X | TCRBJ02-05 | 8.18E−09 |
| CASSTGEGNTIYF | 540 | TCRBV14-01 | TCRBJ01-03 | 8.25E−09 |
| CASSSGLSDSNQPQHF | 541 | TCRBV12-X | TCRBJ01-05 | 8.46E−09 |
| CSARELAGGSGELFF | 542 | TCRBV20-X | TCRBJ02-02 | 9.71E−09 |
| CSARGRGVEKLFF | 543 | TCRBV20-X | TCRBJ01-04 | 9.93E−09 |
| CASSLRGRRNQETQYF | 544 | TCRBV12-X | TCRBJ02-05 | 1.01E−08 |
| CSAPVGTAPSYEQYF | 545 | TCRBV20-X | TCRBJ02-07 | 1.03E−08 |
| CSAQGNNQPQHF | 546 | TCRBV29-01 | TCRBJ01-05 | 1.08E−08 |
| CATSRDREKGNTIYF | 547 | TCRBV15-01 | TCRBJ01-03 | 1.08E−08 |
| CASSNRDRGHEQYF | 548 | TCRBV06-05 | TCRBJ02-07 | 1.12E−08 |
| CASSTDKAGELFF | 549 | TCRBV05-01 | TCRBJ02-02 | 1.14E−08 |
| CASSPGTGRINQPQHF | 550 | TCRBV06-X | TCRBJ01-05 | 1.16E−08 |
| CASSPTLAGGRGEQFF | 551 | TCRBV18-01 | TCRBJ02-01 | 1.22E−08 |
| CAISESRGGNSNQPQHF | 552 | TCRBV10-03 | TCRBJ01-05 | 1.28E−08 |
| CASRLPRGRNQPQHF | 553 | TCRBV28-01 | TCRBJ01-05 | 1.30E−08 |
| CASSPVGGARYEQYF | 554 | TCRBV09-01 | TCRBJ02-07 | 1.37E−08 |
| CASGRLSSYEQYF | 555 | TCRBV28-01 | TCRBJ02-07 | 1.47E−08 |
| CASSPGTSRKTQYF | 556 | TCRBV28-01 | TCRBJ02-05 | 1.54E−08 |
| CSATGLAASGANVLTF | 557 | TCRBV20-X | TCRBJ02-06 | 1.60E−08 |
| CASSLAIGHLNTEAFF | 558 | TCRBV05-01 | TCRBJ01-01 | 1.61E−08 |
| CASSLFGGLQETQYF | 559 | TCRBV27-01 | TCRBJ02-05 | 1.86E−08 |
| CASSESQGSPLHF | 560 | TCRBV02-01 | TCRBJ01-06 | 1.86E−08 |
| CASSGQGRTYEQYF | 561 | TCRBV11-02 | TCRBJ02-07 | 1.87E−08 |
| CASSLSGLAGNPDTQYF | 562 | TCRBV07-02 | TCRBJ02-03 | 1.89E−08 |
| CASSENRWSNSPLHF | 563 | TCRBV06-01 | TCRBJ01-06 | 1.90E−08 |
| CSARDRITGGSYNSPLHF | 564 | TCRBV20-X | TCRBJ01-06 | 1.90E−08 |
| CASSLARAGGPEDTQYF | 565 | TCRBV12-X | TCRBJ02-03 | 1.90E−08 |
| CASSERDRGHEQYF | 566 | TCRBV06-01 | TCRBJ02-07 | 1.93E−08 |
| CASSPKTGDNTEAFF | 567 | TCRBV07-02 | TCRBJ01-01 | 1.96E−08 |
| CASSVAPRGGRATDTQYF | 568 | TCRBV09-01 | TCRBJ02-03 | 1.96E−08 |
| CASSQGGQEQETQYF | 569 | TCRBV03-01/03-02 | TCRBJ02-05 | 1.98E−08 |
| CATSNAVLNTEAFF | 570 | TCRBV15-01 | TCRBJ01-01 | 2.12E−08 |
| CASSFAKREAFF | 571 | TCRBV05-04 | TCRBJ01-01 | 2.12E−08 |
| CASSPGPPPNNEQFF | 572 | TCRBV18-01 | TCRBJ02-01 | 2.37E−08 |
| CASSLALAGGPDEQYF | 573 | TCRBV07-02 | TCRBJ02-07 | 2.41E−08 |
| CASRGQDPQETQYF | 574 | TCRBV05-01 | TCRBJ02-05 | 2.82E−08 |
| CSARVVLGTQYF | 539 | TCRBV20-X | TCRBJ02-03 | 2.90E−08 |
| CSADRTSGGRYNEQFF | 575 | TCRBV20-X | TCRBJ02-01 | 2.95E−08 |
| CASSGQGVGGYTF | 576 | TCRBV11-02 | TCRBJ01-02 | 3.11E−08 |
| CASSQGSVNTEAFF | 577 | TCRBV07-06 | TCRBJ01-01 | 3.43E−08 |
| CASHRLSNYGYTF | 578 | TCRBV28-01 | TCRBJ01-02 | 3.60E−08 |
| CASSESRSSYNEQFF | 579 | TCRBV07-03 | TCRBJ02-01 | 3.97E−08 |
| CSAGGTGGFF | 580 | TCRBV20-01 | TCRBJ02-01 | 4.05E−08 |
| CASSVGPGQGIYNEQFF | 581 | TCRBV09-01 | TCRBJ02-01 | 4.11E−08 |
| CASILRQDYGYTF | 582 | TCRBV02-01 | TCRBJ01-02 | 4.15E−08 |
| CASSPGTSLNIQYF | 583 | TCRBV28-01 | TCRBJ02-04 | 4.21E−08 |
| CASSTPPDTQYF | 584 | TCRBV27-01 | TCRBJ02-03 | 4.58E−08 |
| CASSQEGLAGGISYEQYF | 585 | TCRBV04-02 | TCRBJ02-07 | 4.77E−08 |
| CASSSTGGRSGELFF | 586 | TCRBV07-02 | TCRBJ02-02 | 4.77E−08 |
| CAWSTRTGVTYEQYF | 587 | TCRBV30-01 | TCRBJ02-07 | 4.77E−08 |
| CASRGLARHEQYF | 588 | TCRBV02-01 | TCRBJ02-07 | 4.77E−08 |
| CASSMIHMNTEAFF | 589 | TCRBV19-01 | TCRBJ01-01 | 5.25E−08 |
| CASSLAPGQGGGYTF | 590 | TCRBV05-05 | TCRBJ01-02 | 5.35E−08 |
| CARVLAGGGEQFF | 591 | TCRBV02-01 | TCRBJ02-01 | 5.82E−08 |
| CASSYGNPGQSYEQYF | 592 | TCRBV06-02/06-03 | TCRBJ02-07 | 6.29E−08 |
| CSAREGVAYNEQFF | 593 | TCRBV20-X | TCRBJ02-01 | 6.36E−08 |
| CASSIRAGTAETQYF | 594 | TCRBV19-01 | TCRBJ02-05 | 6.79E−08 |
| CASSIRSGTAETQYF | 595 | TCRBV19-01 | TCRBJ02-05 | 6.79E−08 |
| CAIRRPESTDTQYF | 596 | TCRBV10-03 | TCRBJ02-03 | 6.82E−08 |
| CASSLREVGETQYF | 597 | TCRBV04-01 | TCRBJ02-05 | 7.10E−08 |
| CASSSETSGRNEQFF | 598 | TCRBV07-09 | TCRBJ02-01 | 7.10E−08 |
| CASRPDRRNTEAFF | 599 | TCRBV03-01/03-02 | TCRBJ01-01 | 7.26E−08 |
| CASSYQDRGANYGYTF | 600 | TCRBV06-05 | TCRBJ01-02 | 7.30E−08 |
| CASSPRDYEQFF | 601 | TCRBV04-02 | TCRBJ02-01 | 7.69E−08 |
| CASSLESQRTDTQYF | 602 | TCRBV11-02 | TCRBJ02-03 | 7.96E−08 |
| CSARATGGPTYNEQFF | 603 | TCRBV20-X | TCRBJ02-01 | 8.39E−08 |
| CASSLGGTERYEQYF | 604 | TCRBV07-08 | TCRBJ02-07 | 8.39E−08 |
| CATSRDNSLQETQYF | 605 | TCRBV15-01 | TCRBJ02-05 | 8.56E−08 |
| CATSREGTGRNTEAFF | 606 | TCRBV15-01 | TCRBJ01-01 | 8.63E−08 |
| CASSLSWTGYEKLFF | 607 | TCRBV27-01 | TCRBJ01-04 | 8.79E−08 |
| CASSYSTGTGFRETQYF | 608 | TCRBV06-X | TCRBJ02-05 | 9.09E−08 |
| CASSQETGLDGYTF | 609 | TCRBV04-03 | TCRBJ01-02 | 9.19E−08 |
| CASTRGRGETQYF | 610 | TCRBV07-08 | TCRBJ02-05 | 9.36E−08 |
| CASSSRTGDSGNTIYF | 611 | TCRBV05-05 | TCRBJ01-03 | 9.36E−08 |
| CASSTRPDSNTGELFF | 612 | TCRBV19-01 | TCRBJ02-02 | 9.54E−08 |
| CASSRGQGEGYEQYF | 613 | TCRBV04-03 | TCRBJ02-07 | 9.54E−08 |
| CASGPTRPYEQYF | 614 | TCRBV19-01 | TCRBJ02-07 | 9.54E−08 |
| CASSQGQGEGYEQFF | 615 | TCRBV04-03 | TCRBJ02-01 | 9.54E−08 |
| CASSTDKTGELFF | 616 | TCRBV05-01 | TCRBJ02-02 | 9.54E−08 |
| CASSLEWDQPQHF | 617 | TCRBV05-05 | TCRBJ01-05 | 9.89E−08 |
| CASSPQGSAGNTIYF | 618 | TCRBV09-01 | TCRBJ01-03 | 9.94E−08 |
| CASSGRLADTGELFF | 619 | TCRBV12-X | TCRBJ02-02 | 1.06E−07 |
| CASSPQGTGKGYEQYF | 620 | TCRBV18-01 | TCRBJ02-07 | 1.12E−07 |
| CASRTGVGNQPQHF | 621 | TCRBV06-05 | TCRBJ01-05 | 1.25E−07 |
| CASSDRDSSPLHF | 622 | TCRBV11-02 | TCRBJ01-06 | 1.29E−07 |
| CASSPRQGTAETQYF | 623 | TCRBV19-01 | TCRBJ02-05 | 1.29E−07 |
| CASSASSGYYEQYF | 624 | TCRBV02-01 | TCRBJ02-07 | 1.29E−07 |
| CSAPLGTIQETQYF | 625 | TCRBV20-X | TCRBJ02-05 | 1.35E−07 |
| CASSVAARGALNTEAFF | 626 | TCRBV09-01 | TCRBJ01-01 | 1.41E−07 |
| CASSTGGEQFF | 627 | TCRBV27-01 | TCRBJ02-01 | 1.53E−07 |
| CASSLFGQNSGNTIYF | 628 | TCRBV12-X | TCRBJ01-03 | 1.62E−07 |
| CASSLVFGESYGYTF | 629 | TCRBV11-02 | TCRBJ01-02 | 1.65E−07 |
| CASSFVRDRETQYF | 630 | TCRBV05-01 | TCRBJ02-05 | 1.82E−07 |
| CASSPPAAGRNTGELFF | 631 | TCRBV18-01 | TCRBJ02-02 | 1.82E−07 |
| CATSRDSSNQETQYF | 632 | TCRBV15-01 | TCRBJ02-05 | 1.82E−07 |
| CASSQGRDKNIQYF | 633 | TCRBV14-01 | TCRBJ02-04 | 1.86E−07 |
| CASSQDTSGSYTGELFF | 634 | TCRBV04-01 | TCRBJ02-02 | 1.86E−07 |
| CASKGGTNEKLFF | 635 | TCRBV06-06 | TCRBJ01-04 | 1.89E−07 |
| CASSLQQGRSEAFF | 636 | TCRBV11-02 | TCRBJ01-01 | 1.96E−07 |
| CASSRTGGRGGELFF | 637 | TCRBV07-02 | TCRBJ02-02 | 1.96E−07 |
| CASSQGEREGHEQFF | 638 | TCRBV04-03 | TCRBJ02-01 | 1.96E−07 |
| CASRQQGPETQYF | 639 | TCRBV06-05 | TCRBJ02-05 | 1.97E−07 |
| CASSPPGARNEQFF | 640 | TCRBV11-02 | TCRBJ02-01 | 2.00E−07 |
| CASSLVGGISSYEQYF | 641 | TCRBV07-09 | TCRBJ02-07 | 2.14E−07 |
| CASSASGQGGYEQYF | 642 | TCRBV11-02 | TCRBJ02-07 | 2.26E−07 |
| CSAPPGGRDNSPLHF | 643 | TCRBV20-X | TCRBJ01-06 | 2.26E−07 |
| CASSYSGTGTLNQPQHF | 644 | TCRBV06-X | TCRBJ01-05 | 2.27E−07 |
| CASSLGGRQGRTEAFF | 645 | TCRBV07-03 | TCRBJ01-01 | 2.27E−07 |
| CSARTGTPNQETQYF | 646 | TCRBV20-X | TCRBJ02-05 | 2.30E−07 |
| CASSTDRPGEQFF | 647 | TCRBV05-01 | TCRBJ02-01 | 2.41E−07 |
| CASSLSYSHSGNTIYF | 648 | TCRBV12-X | TCRBJ01-03 | 2.41E−07 |
| CASSLGGLIDQPQHF | 649 | TCRBV12-X | TCRBJ01-05 | 2.41E−07 |
| CSGWGLATDTQYF | 650 | TCRBV29-01 | TCRBJ02-03 | 2.42E−07 |
| CASSPGTQKNIQYF | 651 | TCRBV18-01 | TCRBJ02-04 | 2.51E−07 |
| CASSGSGGNTEAFF | 652 | TCRBV04-03 | TCRBJ01-01 | 2.51E−07 |
| CASSPVGGGRYEQYF | 653 | TCRBV09-01 | TCRBJ02-07 | 2.51E−07 |
| CASSAFDQPQHF | 654 | TCRBV19-01 | TCRBJ01-05 | 2.86E−07 |
| CSARDPTGDTGELFF | 655 | TCRBV20-X | TCRBJ02-02 | 3.15E−07 |
| CASSIPPGEQYF | 656 | TCRBV27-01 | TCRBJ02-07 | 3.18E−07 |
| CASSPMKGYEQYF | 657 | TCRBV27-01 | TCRBJ02-07 | 3.22E−07 |
| CASRGDGSNTEAFF | 658 | TCRBV06-05 | TCRBJ01-01 | 3.31E−07 |
| CAWSVTGGLRNQPQHF | 659 | TCRBV30-01 | TCRBJ01-05 | 3.54E−07 |
| CASSIRGGTAETQYF | 660 | TCRBV19-01 | TCRBJ02-05 | 3.54E−07 |
| CASSARTGGAGQPQHF | 661 | TCRBV06-01 | TCRBJ01-05 | 3.61E−07 |
| CASREHSTDTQYF | 662 | TCRBV06-06 | TCRBJ02-03 | 3.62E−07 |
| CASSSRGAGGSYNEQFF | 663 | TCRBV06-02/06-03 | TCRBJ02-01 | 3.69E−07 |
| CASRGARGRNQPQHF | 664 | TCRBV28-01 | TCRBJ01-05 | 3.70E−07 |
| CASSQRTGGSYNSPLHF | 665 | TCRBV07-02 | TCRBJ01-06 | 3.70E−07 |
| CASRTARGRNQPQHF | 666 | TCRBV28-01 | TCRBJ01-05 | 3.70E−07 |
| CASSQLLARETQYF | 667 | TCRBV14-01 | TCRBJ02-05 | 3.87E−07 |
| CSARDAVSGTDTQYF | 668 | TCRBV20-X | TCRBJ02-03 | 4.13E−07 |
| CSARLGTGNNSPLHF | 669 | TCRBV20-X | TCRBJ01-06 | 4.26E−07 |
| CSVEFAGGADTQYF | 670 | TCRBV29-01 | TCRBJ02-03 | 4.39E−07 |
| CATSRDGGTINSPLHF | 671 | TCRBV15-01 | TCRBJ01-06 | 4.81E−07 |
| CSVGGPGGSNTEAFF | 672 | TCRBV29-01 | TCRBJ01-01 | 4.81E−07 |
| CASSYSGEQGNGYTF | 673 | TCRBV06-05 | TCRBJ01-02 | 4.81E−07 |
| CSALSGSSYNEQFF | 674 | TCRBV29-01 | TCRBJ02-01 | 4.95E−07 |
| CASSPPQRGSYNSPLHF | 675 | TCRBV18-01 | TCRBJ01-06 | 5.00E−07 |
| CSATSGTDDSPLHF | 676 | TCRBV20-X | TCRBJ01-06 | 5.09E−07 |
| CASSNPGDYNEQFF | 677 | TCRBV19-01 | TCRBJ02-01 | 5.31E−07 |
| CASSLQGRAGNTIYF | 678 | TCRBV13-01 | TCRBJ01-03 | 5.31E−07 |
| CATLQVGSYNEQFF | 679 | TCRBV19-01 | TCRBJ02-01 | 5.31E−07 |
| CASSLFGTGPYEQYF | 680 | TCRBV05-06 | TCRBJ02-07 | 5.48E−07 |
| CASSQVLAGVDEQFF | 681 | TCRBV14-01 | TCRBJ02-01 | 5.74E−07 |
| CASSSWGGETQYF | 682 | TCRBV04-02 | TCRBJ02-05 | 5.91E−07 |
| CASSFGSTETQYF | 683 | TCRBV11-02 | TCRBJ02-05 | 6.12E−07 |
| CASSLTTGGNSPLHF | 684 | TCRBV07-06 | TCRBJ01-06 | 6.17E−07 |
| CSARVRAGGPGELFF | 685 | TCRBV20-X | TCRBJ02-02 | 6.17E−07 |
| CASSARHLYGYTF | 686 | TCRBV19-01 | TCRBJ01-02 | 6.17E−07 |
| CASSEGGRTAYEQYF | 687 | TCRBV06-01 | TCRBJ02-07 | 6.28E−07 |
| CSAPKGASAYNEQFF | 688 | TCRBV20-X | TCRBJ02-01 | 6.34E−07 |
| CASSLGRPGGEQYF | 689 | TCRBV27-01 | TCRBJ02-07 | 6.62E−07 |
| CASSVEGSTHTEAFF | 690 | TCRBV09-01 | TCRBJ01-01 | 6.93E−07 |
| CRISGGVTEAFF | 691 | TCRBV20-X | TCRBJ01-01 | 6.93E−07 |
| CASSQGAQGFGNTIYF | 692 | TCRBV04-02 | TCRBJ01-03 | 7.15E−07 |
| CASSYDVVEQYF | 693 | TCRBV06-06 | TCRBJ02-07 | 7.81E−07 |
| CASSLGGGPGSPLHF | 694 | TCRBV05-05 | TCRBJ01-06 | 7.93E−07 |
| CASSYLPTGSNQPQHF | 695 | TCRBV06-02/06-03 | TCRBJ01-05 | 7.97E−07 |
| CASSSRLAGNPDTQYF | 696 | TCRBV27-01 | TCRBJ02-03 | 7.97E−07 |
| CSARDFTADNSPLHF | 697 | TCRBV20-X | TCRBJ01-06 | 8.08E−07 |
| CASSQELADTQYF | 698 | TCRBV07-03 | TCRBJ02-03 | 8.67E−07 |
| CASSSETSGANVLTF | 699 | TCRBV07-09 | TCRBJ02-06 | 8.68E−07 |
| CSVLRTSNTEAFF | 700 | TCRBV29-01 | TCRBJ01-01 | 8.71E−07 |
| CASSLEIADTQYF | 331 | TCRBV05-01 | TCRBJ02-03 | 9.13E−07 |
| CASGRTSGGETQYF | 701 | TCRBV12-05 | TCRBJ02-05 | 9.18E−07 |
| CSEDTDTQYF | 702 | TCRBV29-01 | TCRBJ02-03 | 9.24E−07 |
| CASSQVPVSSGNTIYF | 703 | TCRBV04-03 | TCRBJ01-03 | 9.42E−07 |
| CASSLAWTGLNTEAFF | 704 | TCRBV05-05 | TCRBJ01-01 | 9.87E−07 |
| CASSVGFRSSYNSPLHF | 705 | TCRBV09-01 | TCRBJ01-06 | 1.01E−06 |
| CASSVALRGALNYGYTF | 706 | TCRBV09-01 | TCRBJ01-02 | 1.02E−06 |
| CASSESDRSSQPQHF | 707 | TCRBV02-01 | TCRBJ01-05 | 1.06E−06 |
| CASSLDVSRNSPLHF | 708 | TCRBV07-02 | TCRBJ01-06 | 1.08E−06 |
| CASSALGDNSPLHF | 709 | TCRBV02-01 | TCRBJ01-06 | 1.09E−06 |
| CASSLEPIQPQHF | 710 | TCRBV05-05 | TCRBJ01-05 | 1.10E−06 |
| CASSLDRIHNSPLHF | 711 | TCRBV05-01 | TCRBJ01-06 | 1.10E−06 |
| CASSRDRGGSYNEQFF | 712 | TCRBV05-01 | TCRBJ02-01 | 1.16E−06 |
| CASSLDSGRPDTQYF | 713 | TCRBV11-03 | TCRBJ02-03 | 1.19E−06 |
| CASSLDQADTQYF | 714 | TCRBV11-02 | TCRBJ02-03 | 1.31E−06 |
| CASSHRQSETQYF | 715 | TCRBV14-01 | TCRBJ02-05 | 1.32E−06 |
| CASSNNEKLFF | 716 | TCRBV04-03 | TCRBJ01-04 | 1.53E−06 |
| CASSLTSGRAGSSYEQYF | 717 | TCRBV07-02 | TCRBJ02-07 | 1.62E−06 |
| CASSIGLAGGGNEQFF | 718 | TCRBV07-09 | TCRBJ02-01 | 1.62E−06 |
| CAWSTRTGTTYEQYF | 719 | TCRBV30-01 | TCRBJ02-07 | 1.63E−06 |
| CASSLHAGNTIYF | 720 | TCRBV13-01 | TCRBJ01-03 | 1.63E−06 |
| CASSPPPDRPTGELFF | 721 | TCRBV05-01 | TCRBJ02-02 | 1.78E−06 |
| CASSGQLSGNTIYF | 722 | TCRBV14-01 | TCRBJ01-03 | 1.83E−06 |
| CASSYGTSYNSPLHF | 723 | TCRBV06-02/06-03 | TCRBJ01-06 | 1.86E−06 |
| CASRSQGASYEQYF | 724 | TCRBV27-01 | TCRBJ02-07 | 1.87E−06 |
| CASSPDVVLETQYF | 725 | TCRBV18-01 | TCRBJ02-05 | 1.92E−06 |
| CASSSMTGGEKLFF | 726 | TCRBV05-06 | TCRBJ01-04 | 1.92E−06 |
| CASSTGAEQYF | 727 | TCRBV27-01 | TCRBJ02-07 | 1.99E−06 |
| CASSPREGRTDTQYF | 728 | TCRBV07-08 | TCRBJ02-03 | 2.06E−06 |
| CASSLTSSLETQYF | 729 | TCRBV27-01 | TCRBJ02-05 | 2.16E−06 |
| CASSVMQANTEAFF | 730 | TCRBV09-01 | TCRBJ01-01 | 2.16E−06 |
| CSAPDLRSYEQYF | 731 | TCRBV20-X | TCRBJ02-07 | 2.22E−06 |
| CASSYLGEQGNEQYF | 732 | TCRBV06-05 | TCRBJ02-07 | 2.43E−06 |
| CASSVRQDFSGNTIYF | 733 | TCRBV19-01 | TCRBJ01-03 | 2.43E−06 |
| CSASQELSNQPQHF | 734 | TCRBV20-X | TCRBJ01-05 | 2.63E−06 |
| CSARRQGETGELFF | 735 | TCRBV20-X | TCRBJ02-02 | 2.63E−06 |
| CASSPPGARNEQYF | 736 | TCRBV11-02 | TCRBJ02-07 | 2.81E−06 |
| CASSYDVVEAFF | 737 | TCRBV06-06 | TCRBJ01-01 | 2.81E−06 |
| CSARRTPPTEAFF | 738 | TCRBV20-X | TCRBJ01-01 | 2.82E−06 |
| CASSLTQANNYGYTF | 739 | TCRBV27-01 | TCRBJ01-02 | 3.19E−06 |
| CSVAGWGEKLFF | 740 | TCRBV29-01 | TCRBJ01-04 | 3.20E−06 |
| CSARTLAGGAGELFF | 741 | TCRBV20-X | TCRBJ02-02 | 3.20E−06 |
| CASSLQGQGAPGELFF | 742 | TCRBV07-02 | TCRBJ02-02 | 3.23E−06 |
| CASSPTNTGVEQYF | 743 | TCRBV18-01 | TCRBJ02-07 | 3.23E−06 |
| CSAKPLGGGYNEQFF | 744 | TCRBV20-X | TCRBJ02-01 | 3.23E−06 |
| CASSLPDRVNSPLHF | 745 | TCRBV18-01 | TCRBJ01-06 | 3.35E−06 |
| CASNNPGDTGELFF | 746 | TCRBV19-01 | TCRBJ02-02 | 3.35E−06 |
| CASSYGQGWTQPQHF | 747 | TCRBV06-02/06-03 | TCRBJ01-05 | 3.54E−06 |
| CASSQPGQGAREQYF | 748 | TCRBV11-02 | TCRBJ02-07 | 3.54E−06 |
| CASSLAGGGRENEQFF | 749 | TCRBV05-01 | TCRBJ02-01 | 3.77E−06 |
| CASSLAWRDYTDTQYF | 750 | TCRBV07-02 | TCRBJ02-03 | 3.78E−06 |
| CASSYSGEQGNEQFF | 75 | TCRBV06-05 | TCRBJ02-01 | 3.95E−06 |
| CSVRGVGENTEAFF | 752 | TCRBV29-01 | TCRBJ01-01 | 3.95E−06 |
| CASSEGVRGGYGYTF | 753 | TCRBV06-01 | TCRBJ01-02 | 4.10E−06 |
| CASSISRTENIQYF | 754 | TCRBV19-01 | TCRBJ02-04 | 4.10E−06 |
| CASSFRGQGARQPQHF | 755 | TCRBV28-01 | TCRBJ01-05 | 4.11E−06 |
| CATSTMGGTEAFF | 756 | TCRBV15-01 | TCRBJ01-01 | 4.11E−06 |
| CASSLDLKSTGELFF | 757 | TCRBV11-02 | TCRBJ02-02 | 4.23E−06 |
| CASSVVGAGEEKLFF | 758 | TCRBV09-01 | TCRBJ01-04 | 4.26E−06 |
| CATSREQGFSSYEQYF | 759 | TCRBV15-01 | TCRBJ02-07 | 4.29E−06 |
| CASSGGQGETEAFF | 269 | TCRBV06-05 | TCRBJ01-01 | 4.37E−06 |
| CASSVVPTEGEKLFF | 760 | TCRBV09-01 | TCRBJ01-04 | 4.37E−06 |
| CASSPHKQNTEAFF | 761 | TCRBV07-02 | TCRBJ01-01 | 4.91E−06 |
| CASSLEMADTQYF | 762 | TCRBV05-01 | TCRBJ02-03 | 5.03E−06 |
| CASSQVLGGLQETQYF | 763 | TCRBV14-01 | TCRBJ02-05 | 5.10E−06 |
| CASSPGSNSQPQHF | 764 | TCRBV05-01 | TCRBJ01-05 | 5.38E−06 |
| CASSPILARETQYF | 765 | TCRBV14-01 | TCRBJ02-05 | 5.38E−06 |
| CASSPGAQSQPQHF | 766 | TCRBV05-01 | TCRBJ01-05 | 5.38E−06 |
| CASSRSAGVGDTQYF | 767 | TCRBV19-01 | TCRBJ02-03 | 5.38E−06 |
| CASSFGREKLFF | 768 | TCRBV07-02 | TCRBJ01-04 | 5.48E−06 |
| CASSDHTGPGNTIYF | 769 | TCRBV06-01 | TCRBJ01-03 | 5.73E−06 |
| CSVEEGTDTQYF | 770 | TCRBV20-X | TCRBJ02-03 | 5.79E−06 |
| CASSSTGNSYEQYF | 771 | TCRBV13-01 | TCRBJ02-07 | 5.86E−06 |
| CASRRLAGDYEQYF | 772 | TCRBV02-01 | TCRBJ02-07 | 6.27E−06 |
| CASSPPDGPTNEKLFF | 773 | TCRBV18-01 | TCRBJ01-04 | 6.36E−06 |
| CSARSPGRYTGELFF | 774 | TCRBV20-X | TCRBJ02-02 | 6.36E−06 |
| CASSSRDGRTDTQYF | 775 | TCRBV07-08 | TCRBJ02-03 | 6.66E−06 |
| CASSQDFGGAGGYTF | 776 | TCRBV03-01/03-02 | TCRBJ01-02 | 6.66E−06 |
| CASSRTGQGNTIYF | 777 | TCRBV05-01 | TCRBJ01-03 | 6.77E−06 |
| CASSQGGARGETQYF | 778 | TCRBV14-01 | TCRBJ02-05 | 6.80E−06 |
| CASSLLGSRSGANVLTF | 779 | TCRBV05-06 | TCRBJ02-06 | 6.91E−06 |
| CASSSEVQETQYF | 780 | TCRBV07-03 | TCRBJ02-05 | 6.96E−06 |
| CASSYRVSNSPLHF | 781 | TCRBV07-02 | TCRBJ01-06 | 7.04E−06 |
| CASSLYGSGPYEQYF | 782 | TCRBV05-06 | TCRBJ02-07 | 7.62E−06 |
| CSARDNRADTGELFF | 783 | TCRBV20-X | TCRBJ02-02 | 7.62E−06 |
| CASSGAGGGETQYF | 784 | TCRBV05-05 | TCRBJ02-05 | 8.43E−06 |
| CASSLYPQVYEQYF | 785 | TCRBV07-09 | TCRBJ02-07 | 8.87E−06 |
| CASSHRTSGGPTGELFF | 786 | TCRBV14-01 | TCRBJ02-02 | 9.50E−06 |
| CASSPYTNQPQHF | 787 | TCRBV06-04 | TCRBJ01-05 | 9.59E−06 |
| CASSDWTSGYNEQFF | 788 | TCRBV25-01 | TCRBJ02-01 | 9.59E−06 |
| CSANGLAGVVGEQFF | 789 | TCRBV20-X | TCRBJ02-01 | 9.65E−06 |
| CASSRQREDQPQHF | 790 | TCRBV05-05 | TCRBJ01-05 | 9.65E−06 |
| CASRSSTGRNQPQHF | 791 | TCRBV28-01 | TCRBJ01-05 | 9.65E−06 |
| CASSGLTSGSRTDTQYF | 792 | TCRBV06-01 | TCRBJ02-03 | 9.65E−06 |
| CASSQEGFTDTQYF | 793 | TCRBV04-03 | TCRBJ02-03 | 9.95E−06 |
| CASSLAGVAGVDEQFF | 794 | TCRBV07-02 | TCRBJ02-01 | 9.97E−06 |
| CASSPLDTRYEQYF | 795 | TCRBV14-01 | TCRBJ02-07 | 1.08E−05 |
| CASSNPGDRNTIYF | 796 | TCRBV19-01 | TCRBJ01-03 | 1.08E−05 |
| CASSPGQISNTEAFF | 797 | TCRBV07-02 | TCRBJ01-01 | 1.08E−05 |
| CSVEGGRGNTDTQYF | 798 | TCRBV29-01 | TCRBJ02-03 | 1.09E−05 |
| CASSLLGGLSSGNTIYF | 799 | TCRBV05-06 | TCRBJ01-03 | 1.11E−05 |
| CASSSPGHWNTEAFF | 800 | TCRBV07-02 | TCRBJ01-01 | 1.12E−05 |
| CASSFWQGRHEQYF | 801 | TCRBV05-06 | TCRBJ02-07 | 1.12E−05 |
| CASSLYPSTYEQYF | 802 | TCRBV07-09 | TCRBJ02-07 | 1.12E−05 |
| CASSLWGARYYGYTF | 803 | TCRBV05-06 | TCRBJ01-02 | 1.16E−05 |
| CASSLGGGPLSEQFF | 804 | TCRBV05-05 | TCRBJ02-01 | 1.19E−05 |
| CASSYLGEQGQPQHF | 805 | TCRBV06-05 | TCRBJ01-05 | 1.22E−05 |
| CASSEGQGEGYEQYF | 806 | TCRBV04-03 | TCRBJ02-07 | 1.22E−05 |
| CASSYLGEQGNGYTF | 807 | TCRBV06-05 | TCRBJ01-02 | 1.22E−05 |
| CASSPQWAGSYEQYF | 808 | TCRBV19-01 | TCRBJ02-07 | 1.22E−05 |
| CASSSGTGEGYTF | 809 | TCRBV05-06 | TCRBJ01-02 | 1.23E−05 |
| CASSLWGGPDNEQFF | 810 | TCRBV05-05 | TCRBJ02-01 | 1.23E−05 |
| CASSAPSDEKLFF | 811 | TCRBV09-01 | TCRBJ01-04 | 1.32E−05 |
| CSARILAGGSGELFF | 812 | TCRBV20-X | TCRBJ02-02 | 1.37E−05 |
| CASSLSSSFPYNEQFF | 813 | TCRBV05-04 | TCRBJ02-01 | 1.42E−05 |
| CASSLGVRRGYTF | 814 | TCRBV12-03/12-04 | TCRBJ01-02 | 1.52E−05 |
| CATSRDLAESSYEQYF | 815 | TCRBV15-01 | TCRBJ02-07 | 1.60E−05 |
| CASSFWQGRNQPQHF | 816 | TCRBV05-06 | TCRBJ01-05 | 1.61E−05 |
| CASSPRWGTDTQYF | 817 | TCRBV12-X | TCRBJ02-03 | 1.62E−05 |
| CASSQGSTSGGRETQYF | 818 | TCRBV04-02 | TCRBJ02-05 | 1.62E−05 |
| CSARPGTGGLTDTQYF | 819 | TCRBV20-X | TCRBJ02-03 | 1.62E−05 |
| CASSLFGGVQETQYF | 820 | TCRBV27-01 | TCRBJ02-05 | 1.63E−05 |
| CASSLYGGLQETQYF | 821 | TCRBV27-01 | TCRBJ02-05 | 1.63E−05 |
| CASSLYGSLTDTQYF | 822 | TCRBV05-01 | TCRBJ02-03 | 1.67E−05 |
| CATKGGTNEKLFF | 823 | TCRBV06-06 | TCRBJ01-04 | 1.75E−05 |
| CASSQGRATGGGYTF | 824 | TCRBV03-01/03-02 | TCRBJ01-02 | 1.80E−05 |
| CASSSTGQGANEQFF | 825 | TCRBV07-03 | TCRBJ02-01 | 1.90E−05 |
| CASSPRLFNEQFF | 826 | TCRBV14-01 | TCRBJ02-01 | 1.92E−05 |
| CAISLEGGGSYEQYF | 827 | TCRBV10-03 | TCRBJ02-07 | 2.15E−05 |
| CASSLRRGVNTEAFF | 828 | TCRBV07-03 | TCRBJ01-01 | 2.18E−05 |
| CAWGPAGELFF | 829 | TCRBV30-01 | TCRBJ02-02 | 2.45E−05 |
| CASSYGNPGQTYEQYF | 830 | TCRBV06-02/06-03 | TCRBJ02-07 | 2.49E−05 |
| CASSLDRGAGYNEQFF | 831 | TCRBV05-01 | TCRBJ02-01 | 2.58E−05 |
| CATSREGTGVGTEAFF | 832 | TCRBV15-01 | TCRBJ01-01 | 2.60E−05 |
| CATSRDSSFQETQYF | 833 | TCRBV15-01 | TCRBJ02-05 | 2.65E−05 |
| CASSYGTVTEAFF | 834 | TCRBV06-02/06-03 | TCRBJ01-01 | 2.66E−05 |
| CSAVTRTYNEQFF | 835 | TCRBV20-X | TCRBJ02-01 | 2.73E−05 |
| CASSREIADTQYF | 836 | TCRBV05-01 | TCRBJ02-03 | 2.82E−05 |
| CASSFKGRRGYTF | 837 | TCRBV05-08 | TCRBJ01-02 | 2.82E−05 |
| CSVGEQGTDTQYF | 838 | TCRBV29-01 | TCRBJ02-03 | 2.93E−05 |
| CASSQGSLQETQYF | 839 | TCRBV18-01 | TCRBJ02-05 | 2.98E−05 |
| CASSPPGGRNEQFF | 840 | TCRBV11-02 | TCRBJ02-01 | 3.05E−05 |
| CASSLTGLVQETQYF | 841 | TCRBV27-01 | TCRBJ02-05 | 3.07E−05 |
| CASSESRGRAANEQFF | 842 | TCRBV10-01 | TCRBJ02-01 | 3.16E−05 |
| CASSLYVQASSYNSPLHF | 843 | TCRBV05-01 | TCRBJ01-06 | 3.16E−05 |
| CASSWRVAGPQETQYF | 844 | TCRBV11-02 | TCRBJ02-05 | 3.16E−05 |
| CASSERTGGSYNSPLHF | 845 | TCRBV07-02 | TCRBJ01-06 | 3.16E−05 |
| CSARDNLADTGELFF | 846 | TCRBV20-X | TCRBJ02-02 | 3.35E−05 |
| CSAIRTGGYQETQYF | 847 | TCRBV20-X | TCRBJ02-05 | 3.39E−05 |
| CASSQPGQVNEQFF | 848 | TCRBV19-01 | TCRBJ02-01 | 3.39E−05 |
| CASSRLAGAGETQYF | 849 | TCRBV07-X | TCRBJ02-05 | 3.54E−05 |
| CASSPGTSLNTQYF | 850 | TCRBV28-01 | TCRBJ02-03 | 3.65E−05 |
| CASSLVGGSSGANVLTF | 851 | TCRBV13-01 | TCRBJ02-06 | 3.74E−05 |
| CASSYRQGVRETQYF | 852 | TCRBV06-05 | TCRBJ02-05 | 3.77E−05 |
| CASSPLGAGTDTQYF | 853 | TCRBV06-06 | TCRBJ02-03 | 4.01E−05 |
| CASSPRGNEQFF | 854 | TCRBV09-01 | TCRBJ02-01 | 4.05E−05 |
| CASRSPGDYNEQFF | 855 | TCRBV19-01 | TCRBJ02-01 | 4.11E−05 |
| CASSESTGPLTDTQYP | 856 | TCRBV06-01 | TCRBJ02-03 | 4.15E−05 |
| CASSYGLAGVRAQYF | 857 | TCRBV06-02/06-03 | TCRBJ02-05 | 4.37E−05 |
| CASTPGQGSDGYTF | 858 | TCRBV06-05 | TCRBJ01-02 | 4.37E−05 |
| CAWSPGQAAGEKLFF | 859 | TCRBV30-01 | TCRBJ01-04 | 4.50E−05 |
| CASSLGLAGGRDIQYF | 860 | TCRBV04-01 | TCRBJ02-04 | 4.50E−05 |
| CASSIDSGTGDEAFF | 861 | TCRBV19-01 | TCRBJ01-01 | 4.51E−05 |
| CASSLNRINTEAFF | 862 | TCRBV28-01 | TCRBJ01-01 | 4.52E−05 |
| CASSLDGGRAPDTQYF | 863 | TCRBV11-03 | TCRBJ02-03 | 4.89E−05 |
| CASSPRDHEQYF | 864 | TCRBV04-02 | TCRBJ02-07 | 4.90E−05 |
| CSARKLAGGSGELFF | 865 | TCRBV20-X | TCRBJ02-02 | 4.91E−05 |
| CASSQEGLAGGITYEQYF | 866 | TCRBV04-02 | TCRBJ02-07 | 5.12E−05 |
| CASSPYGGGRYEQYF | 867 | TCRBV18-01 | TCRBJ02-07 | 5.15E−05 |
| CSAKDLGGPYNEQFF | 868 | TCRBV20-X | TCRBJ02-01 | 5.15E−05 |
| CATSSDTKTQYF | 869 | TCRBV15-01 | TCRBJ02-05 | 5.17E−05 |
| CASKGEEETQYF | 870 | TCRBV06-X | TCRBJ02-05 | 5.17E−05 |
| CASSPQGAQDTQYF | 871 | TCRBV07-09 | TCRBJ02-03 | 5.30E−05 |
| CASRGQDPNEKLFF | 872 | TCRBV05-01 | TCRBJ01-04 | 5.37E−05 |
| CASTLTGNTIYF | 873 | TCRBV07-08 | TCRBJ01-03 | 5.53E−05 |
| CSAPRQGTDTQYF | 874 | TCRBV29-01 | TCRBJ02-03 | 5.57E−05 |
| CSARAQGETGELFF | 875 | TCRBV20-X | TCRBJ02-02 | 5.60E−05 |
| CASRGGRDSGNTIYF | 876 | TCRBV12-X | TCRBJ01-03 | 5.88E−05 |
| CASSDVGRNNEQFF | 877 | TCRBV02-01 | TCRBJ02-01 | 6.04E−05 |
| CASSFLPGLGNEQFF | 878 | TCRBV07-02 | TCRBJ02-01 | 6.12E−05 |
| CASSPPDNPTNEKLFF | 879 | TCRBV18-01 | TCRBJ01-04 | 6.12E−05 |
| CSALRPGPGYTF | 880 | TCRBV20-X | TCRBJ01-02 | 6.12E−05 |
| CSVGQGADEAFF | 881 | TCRBV29-01 | TCRBJ01-01 | 6.24E−05 |
| CASSMGNGPYEQYF | 882 | TCRBV19-01 | TCRBJ02-07 | 6.42E−05 |
| CASRSPAGRNQPQHF | 883 | TCRBV28-01 | TCRBJ01-05 | 6.42E−05 |
| CASSLVGVPPDTQYF | 884 | TCRBV05-01 | TCRBJ02-03 | 6.58E−05 |
| CASSPGTSRRTQYF | 885 | TCRBV28-01 | TCRBJ02-05 | 7.13E−05 |
| CASTLGGGWGQPQHF | 886 | TCRBV19-01 | TCRBJ01-05 | 7.13E−05 |
| CASSLVNGNYGYTF | 887 | TCRBV07-02 | TCRBJ01-02 | 7.55E−05 |
| CASSTERAGELFF | 888 | TCRBV05-01 | TCRBJ02-02 | 7.58E−05 |
| CASSFVREGEKLFF | 889 | TCRBV12-X | TCRBJ01-04 | 7.79E−05 |
| CASSAYTNQPQHF | 890 | TCRBV06-04 | TCRBJ01-05 | 8.05E−05 |
| CSASLTSGRAADTQYF | 891 | TCRBV20-01 | TCRBJ02-03 | 8.22E−05 |
| CASSPVDSNYGYTF | 892 | TCRBV19-01 | TCRBJ01-02 | 8.22E−05 |
| CSAADWQETQYF | 893 | TCRBV20-X | TCRBJ02-05 | 8.85E−05 |
| CASSTRTGVRETQYF | 894 | TCRBV06-05 | TCRBJ02-05 | 9.01E−05 |
| CSAKGPGAEAFF | 895 | TCRBV20-X | TCRBJ01-01 | 9.46E−05 |
| CASGPSTSTDTQYF | 896 | TCRBV12-05 | TCRBJ02-03 | 9.55E−05 |
| CSAPSSGGPYEQYF | 897 | TCRBV20-X | TCRBJ02-07 | 9.94E−05 |
| CASSFEIQETQYF | 898 | TCRBV07-02 | TCRBJ02-05 | 1.02E−04 |
| CATSRDNSQQETQYF | 899 | TCRBV15-01 | TCRBJ02-05 | 1.09E−04 |
| CSAITRTYNEQFF | 900 | TCRBV20-X | TCRBJ02-01 | 1.12E−04 |
| CASSLAGGGLYEQYF | 901 | TCRBV05-08 | TCRBJ02-07 | 1.12E−04 |
| CASSSETGGRNEQFF | 902 | TCRBV07-09 | TCRBJ02-01 | 1.18E−04 |
| CASSDRTGTSYNEQFF | 903 | TCRBV06-04 | TCRBJ02-01 | 1.18E−04 |
| CASSLLGQSSGANVLTF | 904 | TCRBV05-06 | TCRBJ02-06 | 1.20E−04 |
| CASSLGTDPRETQYF | 905 | TCRBV11-02 | TCRBJ02-05 | 1.25E−04 |
| CAIRQDPYEQYF | 906 | TCRBV06-05 | TCRBJ02-07 | 1.26E−04 |
| CASSARGQGSYEQYF | 907 | TCRBV07-06 | TCRBJ02-07 | 1.26E−04 |
| CAWSPIDSLNTEAFF | 908 | TCRBV30-01 | TCRBJ01-01 | 1.33E−04 |
| CASSGTGDDYTF | 909 | TCRBV19-01 | TCRBJ01-02 | 1.47E−04 |
| CATTTTGDEQYF | 910 | TCRBV28-01 | TCRBJ02-07 | 1.47E−04 |
| CASSIIAGKGNQPQHF | 911 | TCRBV19-01 | TCRBJ01-05 | 1.47E−04 |
| CASNRWGQETQYF | 912 | TCRBV12-03/12-04 | TCRBJ02-05 | 1.56E−04 |
| CASSYGSAGTEAFF | 913 | TCRBV06-06 | TCRBJ01-01 | 1.58E−04 |
| CASRPQRNEQFF | 914 | TCRBV28-01 | TCRBJ02-01 | 1.61E−04 |
| CASSQGDRGATDTQYF | 915 | TCRBV04-01 | TCRBJ02-03 | 1.66E−04 |
| CASSQVSGFEQFF | 916 | TCRBV04-02 | TCRBJ02-01 | 1.81E−04 |
| CSARSQGETGELFF | 917 | TCRBV20-X | TCRBJ02-02 | 1.83E−04 |
| CASSLGSGGVGEQYF | 918 | TCRBV11-02 | TCRBJ02-07 | 1.97E−04 |
| CASRRDRGLYGYTF | 919 | TCRBV11-02 | TCRBJ01-02 | 2.02E−04 |
| CASRPGQGGDEQYF | 920 | TCRBV06-X | TCRBJ02-07 | 2.02E−04 |
| CASSIDSRDNEQFF | 921 | TCRBV19-01 | TCRBJ02-01 | 2.06E−04 |
| CASSGPAANEKLFF | 922 | TCRBV07-09 | TCRBJ01-04 | 2.06E−04 |
| CASRGTGSVNEQFF | 923 | TCRBV27-01 | TCRBJ02-01 | 2.06E−04 |
| CASGGTSSGANVLTF | 924 | TCRBV02-01 | TCRBJ02-06 | 2.24E−04 |
| CAISAGGNRETQYF | 925 | TCRBV10-03 | TCRBJ02-05 | 2.24E−04 |
| CASSSAVSGTEAFF | 926 | TCRBV05-01 | TCRBJ01-01 | 2.41E−04 |
| CASSPQDRYYGYTF | 927 | TCRBV14-01 | TCRBJ01-02 | 2.56E−04 |
| CASSLAATLYNEQFF | 928 | TCRBV07-09 | TCRBJ02-01 | 2.56E−04 |
| CATSRDLPESSYEQYF | 929 | TCRBV15-01 | TCRBJ02-07 | 2.56E−04 |
| CASSPLSGTVSYEQYF | 930 | TCRBV07-02 | TCRBJ02-07 | 2.56E−04 |
| CASSLDHRGRVTEAFF | 931 | TCRBV07-08 | TCRBJ01-01 | 2.72E−04 |
| CASSYLDRAHEQYF | 932 | TCRBV06-02/06-03 | TCRBJ02-07 | 2.72E−04 |
| CATHRLANYGYTF | 933 | TCRBV28-01 | TCRBJ01-02 | 2.81E−04 |
| CASSQSVGDTGELFF | 934 | TCRBV14-01 | TCRBJ02-02 | 2.94E−04 |
| CASSQRTSGIQETQYF | 935 | TCRBV04-02 | TCRBJ02-05 | 2.94E−04 |
| CASSPVASNEQFF | 936 | TCRBV12-03/12-04 | TCRBJ02-01 | 2.98E−04 |
| CASSLSGLAGAPDTQYF | 937 | TCRBV07-02 | TCRBJ02-03 | 3.01E−04 |
| CASSLGGAKDEQYF | 938 | TCRBV27-01 | TCRBJ02-07 | 3.20E−04 |
| CASSPGTSMKTQYF | 939 | TCRBV28-01 | TCRBJ02-05 | 3.22E−04 |
| CASSPGTSRRTQYF | 885 | TCRBV28-01 | TCRBJ02-03 | 3.22E−04 |
| CSAFQGAAGNTIYF | 940 | TCRBV20-X | TCRBJ01-03 | 3.33E−04 |
| CASRRGRDPDTQYF | 941 | TCRBV06-X | TCRBJ02-03 | 3.43E−04 |
| CASSVWGDDNQPQHF | 942 | TCRBV02-01 | TCRBJ01-05 | 3.55E−04 |
| CASSLAGVDTDTQYF | 943 | TCRBV07-07 | TCRBJ02-03 | 3.58E−04 |
| CSARDQGETGELFF | 944 | TCRBV20-X | TCRBJ02-02 | 3.63E−04 |
| CASSEFWGSTDTQYF | 945 | TCRBV02-01 | TCRBJ02-03 | 3.67E−04 |
| CASSDPAGANVLTF | 946 | TCRBV09-01 | TCRBJ02-06 | 3.70E−04 |
| CASSQDRTGGPLGNEQFF | 947 | TCRBV14-01 | TCRBJ02-01 | 3.79E−04 |
| CATRREIADTQYF | 948 | TCRBV27-01 | TCRBJ02-03 | 3.86E−04 |
| CASSSYTNTGELFF | 949 | TCRBV06-04 | TCRBJ02-02 | 3.89E−04 |
| CASSSGARQYF | 950 | TCRBV27-01 | TCRBJ02-07 | 3.89E−04 |
| CSARDRAGGPGELFF | 951 | TCRBV20-X | TCRBJ02-02 | 3.98E−04 |
| CASSLALSGNTIYF | 952 | TCRBV14-01 | TCRBJ01-03 | 4.01E−04 |
| CASSLEGGSGYTF | 953 | TCRBV11-01 | TCRBJ01-02 | 4.40E−04 |
| CSARVGGRPEETQYF | 954 | TCRBV20-X | TCRBJ02-05 | 4.43E−04 |
| CASSAPGDNYGYTF | 955 | TCRBV19-01 | TCRBJ01-02 | 4.50E−04 |
| CASSYRGVYEQYF | 956 | TCRBV12-03/12-04 | TCRBJ02-07 | 4.58E−04 |
| CSVPLQGTDTQYF | 957 | TCRBV29-01 | TCRBJ02-03 | 5.11E−04 |
| CASSGDRVSDGYTF | 958 | TCRBV05-01 | TCRBJ01-02 | 5.24E−04 |
| CASSFWQGRNEQFF | 959 | TCRBV05-06 | TCRBJ02-01 | 5.24E−04 |
| CASRYSGTGELFF | 960 | TCRBV02-01 | TCRBJ02-02 | 5.35E−04 |
| CASSGGDREDQPQHF | 961 | TCRBV09-01 | TCRBJ01-05 | 5.36E−04 |
| CASSIRTGVRETQYF | 962 | TCRBV06-05 | TCRBJ02-05 | 5.38E−04 |
| CASSPLQGRAGELFF | 963 | TCRBV07-09 | TCRBJ02-02 | 5.96E−04 |
| CSASVSRGSYEQYF | 964 | TCRBV20-01 | TCRBJ02-07 | 6.03E−04 |
| CASSSETSGRNEQYF | 965 | TCRBV07-09 | TCRBJ02-07 | 6.11E−04 |
| CASSSHSTYEQYF | 966 | TCRBV07-X | TCRBJ02-07 | 6.15E−04 |
| CASSVEEDRGLNEQFF | 967 | TCRBV09-01 | TCRBJ02-01 | 6.20E−04 |
| CASSSQTGLYGYTF | 968 | TCRBV05-06 | TCRBJ01-02 | 6.39E−04 |
| CASSQVGEKNIQYF | 969 | TCRBV19-01 | TCRBJ02-04 | 6.41E−04 |
| CSVEGGRVGELFF | 970 | TCRBV29-01 | TCRBJ02-02 | 6.53E−04 |
| CASSLRTGVREKLFF | 971 | TCRBV28-01 | TCRBJ01-04 | 6.57E−04 |
| CASSLWQARYEQYF | 972 | TCRBV05-06 | TCRBJ02-07 | 6.64E−04 |
| CASSLAGAPSEQFF | 973 | TCRBV11-02 | TCRBJ02-01 | 6.69E−04 |
| CASSARTVSGANVLTF | 974 | TCRBV07-02 | TCRBJ02-06 | 6.89E−04 |
| CASSPGTGFGNTEAFF | 975 | TCRBV03-01/03-02 | TCRBJ01-01 | 6.89E−04 |
| CASRREIADTQYF | 976 | TCRBV27-01 | TCRBJ02-03 | 7.09E−04 |
| CASSLKGAGSGANVLTF | 977 | TCRBV28-01 | TCRBJ02-06 | 7.10E−04 |
| CASSLAGGSLYEQYF | 978 | TCRBV05-08 | TCRBJ02-07 | 7.10E−04 |
| CASRPGGAPTEAFF | 979 | TCRBV28-01 | TCRBJ01-01 | 7.30E−04 |
| CASSLELVGGYEQYF | 980 | TCRBV07-08 | TCRBJ02-07 | 7.30E−04 |
| CASSSDRDSPYEQYF | 981 | TCRBV11-03 | TCRBJ02-07 | 7.30E−04 |
| CASSLLGVSYEQYF | 982 | TCRBV05-06 | TCRBJ02-07 | 7.46E−04 |
| CASSETWAGNTEAFF | 983 | TCRBV02-01 | TCRBJ01-01 | 7.62E−04 |
| CASSQETGLNEQYF | 984 | TCRBV04-03 | TCRBJ02-07 | 7.62E−04 |
| CASTPLNTGELFF | 985 | TCRBV05-05 | TCRBJ02-02 | 7.67E−04 |
| CASRRLVNTEAFF | 986 | TCRBV27-01 | TCRBJ01-01 | 7.70E−04 |
| CASSLGVSGGEQFF | 987 | TCRBV05-01 | TCRBJ02-01 | 7.79E−04 |
| CASSLELTGGYEQYF | 988 | TCRBV07-08 | TCRBJ02-07 | 7.81E−04 |
| CASSAQQGRTEAFF | 989 | TCRBV11-01 | TCRBJ01-01 | 7.83E−04 |
| CASSLITSGDTDTQYF | 990 | TCRBV07-09 | TCRBJ02-03 | 7.88E−04 |
| CASRWGFQETQYF | 991 | TCRBV02-01 | TCRBJ02-05 | 7.90E−04 |
| CASSFGGPGGQPQHF | 992 | TCRBV28-01 | TCRBJ01-05 | 8.19E−04 |
| CASGTWGSNQPQHF | 993 | TCRBV12-X | TCRBJ01-05 | 8.19E−04 |
| CASSERDRGFEQYF | 994 | TCRBV06-01 | TCRBJ02-07 | 8.45E−04 |
| CASSRSKQGDTEAFF | 995 | TCRBV19-01 | TCRBJ01-01 | 8.45E−04 |
| CSVWTGSSYNEQFF | 996 | TCRBV20-X | TCRBJ02-01 | 8.68E−04 |
| CASSLDSFTEAFF | 997 | TCRBV11-02 | TCRBJ01-01 | 8.89E−04 |
| CASSLFGAGPYEQYF | 998 | TCRBV05-06 | TCRBJ02-07 | 9.17E−04 |
| CSARILAGGAGELFF | 999 | TCRBV20-X | TCRBJ02-02 | 1.03E−03 |
| CAWNRGLDYEQYF | 1000 | TCRBV30-01 | TCRBJ02-07 | 1.06E−03 |
| CASSKTGQGNTIYF | 1001 | TCRBV05-01 | TCRBJ01-03 | 1.06E−03 |
| CASSPFTNQPQHF | 1002 | TCRBV06-04 | TCRBJ01-05 | 1.06E−03 |
| CASSSTGNTYEQYF | 1003 | TCRBV13-01 | TCRBJ02-07 | 1.06E−03 |
| CASSYGQGRYNEQFF | 1004 | TCRBV06-05 | TCRBJ02-01 | 1.06E−03 |
| CASSLFGSGPYEQYF | 1005 | TCRBV05-06 | TCRBJ02-07 | 1.07E−03 |
| CASSLLQRREQYF | 1006 | TCRBV07-02 | TCRBJ02-07 | 1.12E−03 |
| CASIQIGDTGELFF | 1007 | TCRBV19-01 | TCRBJ02-02 | 1.25E−03 |
| CASSIDSRDHEQYF | 1008 | TCRBV19-01 | TCRBJ02-07 | 1.25E−03 |
| CASSSGAQQFF | 1009 | TCRBV27-01 | TCRBJ02-01 | 1.25E−03 |
| CSASPRSGNQPQHF | 1010 | TCRBV20-01 | TCRBJ01-05 | 1.25E−03 |
| CASSLLSGTVSYEQYF | 1011 | TCRBV07-02 | TCRBJ02-07 | 1.25E−03 |
| CASSVRQGIREKLFF | 1012 | TCRBV06-X | TCRBJ01-04 | 1.26E−03 |
| CASREGGVGNQPQHF | 1013 | TCRBV06-X | TCRBJ01-05 | 1.31E−03 |
| CASSPPWGADTQYF | 1014 | TCRBV19-01 | TCRBJ02-03 | 1.32E−03 |
| CASSLLGQGRDGYTF | 1015 | TCRBV05-06 | TCRBJ01-02 | 1.37E−03 |
| CASSYRTGVRETQYF | 1016 | TCRBV06-05 | TCRBJ02-05 | 1.37E−03 |
| CASSDRDSGTGELFF | 1017 | TCRBV05-01 | TCRBJ02-02 | 1.39E−03 |
| CASSLREIGETQYF | 1018 | TCRBV04-01 | TCRBJ02-05 | 1.39E−03 |
| CASSLYPSIYEQYF | 1019 | TCRBV07-09 | TCRBJ02-07 | 1.39E−03 |
| CASSLSGAGAGEQYF | 1020 | TCRBV07-02 | TCRBJ02-07 | 1.39E−03 |
| CASSLESGTSGYNEQFF | 1021 | TCRBV05-06 | TCRBJ02-01 | 1.39E−03 |
| CASSQGEREGHEQYF | 1022 | TCRBV04-03 | TCRBJ02-07 | 1.39E−03 |
| CASSLYGGMNTGELFF | 1023 | TCRBV07-02 | TCRBJ02-02 | 1.45E−03 |
| CASSIRTGIRETQYF | 1024 | TCRBV06-05 | TCRBJ02-05 | 1.55E−03 |
| CASSYSAGEAQPQHF | 1025 | TCRBV06-02/06-03 | TCRBJ01-05 | 1.57E−03 |
| CASSLIFGQSYEQYF | 1026 | TCRBV11-02 | TCRBJ02-07 | 1.66E−03 |
| CSAHRQPGGETQYF | 1027 | TCRBV20-X | TCRBJ02-05 | 1.69E−03 |
| CASSSQTGLGGYTF | 1028 | TCRBV05-06 | TCRBJ01-02 | 1.77E−03 |
| CSAQPPSGGSYEQYF | 1029 | TCRBV20-X | TCRBJ02-07 | 1.77E−03 |
| CASSSLRDRSSYNSPLHF | 1030 | TCRBV06-X | TCRBJ01-06 | 1.77E−03 |
| CASTRAPGSSYEQYF | 1031 | TCRBV19-01 | TCRBJ02-07 | 1.81E−03 |
| CASSLDRGRGYNEQFF | 1032 | TCRBV05-01 | TCRBJ02-01 | 1.85E−03 |
| CSARGQGEVGELFF | 1033 | TCRBV20-X | TCRBJ02-02 | 1.90E−03 |
| CASRSPQGRNQPQHF | 1034 | TCRBV28-01 | TCRBJ01-05 | 1.95E−03 |
| CASSLDSTLAKNIQYF | 1035 | TCRBV05-06 | TCRBJ02-04 | 2.02E−03 |
| CAWSPPDSLNEKLFF | 1036 | TCRBV30-01 | TCRBJ01-04 | 2.07E−03 |
| CSARAQGEAGELFF | 1037 | TCRBV20-X | TCRBJ02-02 | 2.18E−03 |
| CASSTRQGVRETQYF | 1038 | TCRBV06-05 | TCRBJ02-05 | 2.19E−03 |
| CASSQDRGGAGNQPQHF | 1039 | TCRBV04-01 | TCRBJ01-05 | 2.20E−03 |
| CASSFRDNEQFF | 1040 | TCRBV11-01 | TCRBJ02-01 | 2.20E−03 |
| CASSQNLNYGYTF | 1041 | TCRBV06-04 | TCRBJ01-02 | 2.21E−03 |
| CASSYAGENNEQFF | 1042 | TCRBV06-05 | TCRBJ02-01 | 2.22E−03 |
| CASSQRQGVREKLFF | 1043 | TCRBV04-03 | TCRBJ01-04 | 2.23E−03 |
| CASSLTSGTSGSSYEQYF | 1044 | TCRBV07-02 | TCRBJ02-07 | 2.25E−03 |
| CASSRKGDEKLFF | 1045 | TCRBV04-01 | TCRBJ01-04 | 2.25E−03 |
| CSANRLSSYNSPLHF | 1046 | TCRBV20-X | TCRBJ01-06 | 2.27E−03 |
| CASSELPVTEAFF | 1047 | TCRBV06-01 | TCRBJ01-01 | 2.28E−03 |
| CATSDLTDRDGYTF | 1048 | TCRBV24-01 | TCRBJ01-02 | 2.38E−03 |
| CASSWRTGVRETQYF | 1049 | TCRBV06-05 | TCRBJ02-05 | 2.47E−03 |
| CASSLGTVASNQPQHF | 1050 | TCRBV05-05 | TCRBJ01-05 | 2.52E−03 |
| CASSFWQGRNTQYF | 1051 | TCRBV05-06 | TCRBJ02-03 | 2.53E−03 |
| CASSPLLERETQYF | 1052 | TCRBV14-01 | TCRBJ02-05 | 2.53E−03 |
| CASSFWQGRHIQYF | 1053 | TCRBV05-06 | TCRBJ02-04 | 2.53E−03 |
| CSARGQGEAGELFF | 1054 | TCRBV20-X | TCRBJ02-02 | 2.53E−03 |
| CASSIRQGVRETQYF | 1055 | TCRBV06-05 | TCRBJ02-05 | 2.57E−03 |
| CSAQKLSSYNSPLHF | 1056 | TCRBV20-X | TCRBJ01-06 | 2.70E−03 |
| CASSDHGQNTEAFF | 1057 | TCRBV06-01 | TCRBJ01-01 | 2.91E−03 |
| CASSPLAGSTNEQFF | 1058 | TCRBV28-01 | TCRBJ02-01 | 3.17E−03 |
| CATSRDPQGAKNEQFF | 1059 | TCRBV15-01 | TCRBJ02-01 | 3.17E−03 |
| CASSPPDREDQPQHF | 1060 | TCRBV28-01 | TCRBJ01-05 | 3.39E−03 |
| CSARPGGGVNQPQHF | 1061 | TCRBV20-X | TCRBJ01-05 | 3.39E−03 |
| CASSVRTGVRETQYF | 1062 | TCRBV06-X | TCRBJ02-05 | 3.58E−03 |
| CASSRQLAGGAYEQYF | 1063 | TCRBV04-01 | TCRBJ02-07 | 3.65E−03 |
| CASSLGGLVKETQYF | 1064 | TCRBV27-01 | TCRBJ02-05 | 3.65E−03 |
| CASSLLTSGTYEQYF | 1065 | TCRBV18-01 | TCRBJ02-07 | 3.74E−03 |
| CASSLDRRREQYF | 1066 | TCRBV27-01 | TCRBJ02-07 | 3.86E−03 |
| CASRGTGSTNEQFF | 1067 | TCRBV27-01 | TCRBJ02-01 | 3.97E−03 |
| CASSLGLAGSHNEQFF | 1068 | TCRBV11-03 | TCRBJ02-01 | 4.06E−03 |
| CASATGAEQYF | 1069 | TCRBV27-01 | TCRBJ02-07 | 4.07E−03 |
| CASSFKPVNTEAFF | 1070 | TCRBV07-09 | TCRBJ01-01 | 4.17E−03 |
| CASSLRTGVRETQYF | 1071 | TCRBV06-05 | TCRBJ02-05 | 4.23E−03 |
| CASSLAQGGQYEQYF | 1072 | TCRBV07-07 | TCRBJ02-07 | 4.28E−03 |
| CASSLTPGTSGSSYEQYF | 1073 | TCRBV07-02 | TCRBJ02-07 | 4.28E−03 |
| CSAGPPRVNTEAFF | 1074 | TCRBV20-X | TCRBJ01-01 | 4.33E−03 |
| CASSVEGGRGSTDTQYF | 1075 | TCRBV09-01 | TCRBJ02-03 | 4.46E−03 |
| CASSLRQGVREKLFF | 1076 | TCRBV06-05 | TCRBJ01-04 | 4.98E−03 |
| CASSLVFGTSYEQYF | 1077 | TCRBV11-02 | TCRBJ02-07 | 5.10E−03 |
| CASSQEGGRPSTDTQYF | 1078 | TCRBV04-03 | TCRBJ02-03 | 5.10E−03 |
| CASSSGSEGQPQHF | 1079 | TCRBV18-01 | TCRBJ01-05 | 5.26E−03 |
| CASSLTSGTSGTPYEQYF | 1080 | TCRBV07-02 | TCRBJ02-07 | 5.33E−03 |
| CASQGSHNEQFF | 1081 | TCRBV07-08 | TCRBJ02-01 | 5.35E−03 |
| CSARTLAGGSGELFF | 1082 | TCRBV20-X | TCRBJ02-02 | 5.39E−03 |
| CASSSYTNQPQHF | 1083 | TCRBV06-04 | TCRBJ01-05 | 5.43E−03 |
| CASSEGAPWQPQHF | 1084 | TCRBV10-02 | TCRBJ01-05 | 5.57E−03 |
| CASSLRQGIREKLFF | 1085 | TCRBV11-02 | TCRBJ01-04 | 5.67E−03 |
| CASSLARTGETQYF | 1086 | TCRBV05-04 | TCRBJ02-05 | 5.68E−03 |
| CASSGRTGGNTGELFF | 1087 | TCRBV07-09 | TCRBJ02-02 | 5.70E−03 |
| CASSSRGGVRETQYF | 1088 | TCRBV06-05 | TCRBJ02-05 | 5.82E−03 |
| CASTRVGSEAFF | 1089 | TCRBV06-05 | TCRBJ01-01 | 5.91E−03 |
| CASSLASVTDTQYF | 1090 | TCRBV27-01 | TCRBJ02-03 | 6.00E−03 |
| CASRTSGLSGANVLTF | 1091 | TCRBV06-X | TCRBJ02-06 | 6.07E−03 |
| CASGGTGGPYEQYF | 1092 | TCRBV28-01 | TCRBJ02-07 | 6.30E−03 |
| CASSLWSREQFF | 1093 | TCRBV28-01 | TCRBJ02-01 | 6.35E−03 |
| CASSSKDRGEQYF | 1094 | TCRBV06-06 | TCRBJ02-07 | 6.45E−03 |
| CASSLWGREANEKLFF | 1095 | TCRBV07-06 | TCRBJ01-04 | 6.45E−03 |
| CASSLRRGGMNTEAFF | 1096 | TCRBV06-02/06-03 | TCRBJ01-01 | 6.55E−03 |
| CASSSGAQQYF | 1097 | TCRBV27-01 | TCRBJ02-07 | 6.65E−03 |
| CASSDVGRSDEQFF | 1098 | TCRBV02-01 | TCRBJ02-01 | 6.70E−03 |
| CASSTKTGVRAGELFF | 1099 | TCRBV19-01 | TCRBJ02-02 | 6.72E−03 |
| CASTNTNYGYTF | 1100 | TCRBV07-02 | TCRBJ01-02 | 6.81E−03 |
| CASSVRQGVREKLFF | 1101 | TCRBV06-X | TCRBJ01-04 | 6.85E−03 |
| CASSTRTGIRETQYF | 1102 | TCRBV06-05 | TCRBJ02-05 | 6.96E−03 |
| CASSLRTGIRETQYF | 1103 | TCRBV06-05 | TCRBJ02-05 | 6.96E−03 |
| CSVGQQGTDTQYF | 1104 | TCRBV29-01 | TCRBJ02-03 | 7.13E−03 |
| CASSSGARQFF | 1105 | TCRBV27-01 | TCRBJ02-01 | 7.21E−03 |
| CATTRGRGETQYF | 1106 | TCRBV07-08 | TCRBJ02-05 | 7.21E−03 |
| CASSLGLAGGGNTIYF | 1107 | TCRBV07-09 | TCRBJ01-03 | 7.21E−03 |
| CASSAPWGTDTQYF | 1108 | TCRBV19-01 | TCRBJ02-03 | 7.30E−03 |
| CASSLYGFRTDTQYF | 1109 | TCRBV05-06 | TCRBJ02-03 | 7.32E−03 |
| CASSEAGGSNTQYF | 1110 | TCRBV06-04 | TCRBJ02-03 | 7.35E−03 |
| CSAREWRGRSSYNEQFF | 1111 | TCRBV20-X | TCRBJ02-01 | 7.35E−03 |
| CASSLTGTEGNSPLHF | 1112 | TCRBV05-01 | TCRBJ01-06 | 7.37E−03 |
| CASSGRTGPNTGELFF | 1113 | TCRBV07-09 | TCRBJ02-02 | 7.52E−03 |
| CASSSRQGVRETQYF | 1114 | TCRBV06-05 | TCRBJ02-05 | 7.84E−03 |
| CASSYGPREPQHF | 1115 | TCRBV06-02/06-03 | TCRBJ01-05 | 7.84E−03 |
| CASSSGAKQYF | 1116 | TCRBV27-01 | TCRBJ02-07 | 8.07E−03 |
| CASSRGTDPRETQYF | 1117 | TCRBV11-02 | TCRBJ02-05 | 8.09E−03 |
| CASSLWSREQYF | 1118 | TCRBV28-01 | TCRBJ02-07 | 8.17E−03 |
| CATSDKKNIQYF | 1119 | TCRBV24-01 | TCRBJ02-04 | 8.22E−03 |
| CASSEGGHTQYF | 1120 | TCRBV10-01 | TCRBJ02-03 | 8.25E−03 |
| CASKRTSSYNEQFF | 1121 | TCRBV06-06 | TCRBJ02-01 | 8.44E−03 |
| CASSRGSTETQYF | 1122 | TCRBV11-02 | TCRBJ02-05 | 8.64E−03 |
| CASSAGPTGELFF | 1123 | TCRBV27-01 | TCRBJ02-02 | 8.64E−03 |
| CASNNRDRGYEQYF | 1124 | TCRBV06-05 | TCRBJ02-07 | 8.64E−03 |
| CASSPRGGRGSYEQYF | 1125 | TCRBV11-02 | TCRBJ02-07 | 8.83E−03 |
| CASSRYREGPYEQYF | 1126 | TCRBV04-01 | TCRBJ02-07 | 9.30E−03 |
| CATSRDPDRAGANVLTF | 1127 | TCRBV15-01 | TCRBJ02-06 | 9.50E−03 |
| CASQGASTDTQYF | 1128 | TCRBV27-01 | TCRBJ02-03 | 9.69E−03 |
| CATSDRQNIQYF | 1129 | TCRBV24-01 | TCRBJ02-04 | 1.02E−02 |
| CASSRDSTLAKNIQYF | 1130 | TCRBV05-06 | TCRBJ02-04 | 1.05E−02 |
| CASSLTTGLAGSSYEQYF | 1131 | TCRBV07-02 | TCRBJ02-07 | 1.12E−02 |
| CASSQARTGRFGNTIYF | 1132 | TCRBV04-03 | TCRBJ01-03 | 1.12E−02 |
| CASKDRENIQYF | 1133 | TCRBV02-01 | TCRBJ02-04 | 1.15E−02 |
| CSATGQGSGQPQHF | 1134 | TCRBV20-X | TCRBJ01-05 | 1.16E−02 |
| CSARRQGEAGELFF | 1135 | TCRBV20-X | TCRBJ02-02 | 1.16E−02 |
| CSARGKREGLGEQFF | 1136 | TCRBV20-X | TCRBJ02-01 | 1.17E−02 |
| CASSPSLVRDEQFF | 1137 | TCRBV07-09 | TCRBJ02-01 | 1.17E−02 |
| CASSLTGGWGNEQFF | 1138 | TCRBV28-01 | TCRBJ02-01 | 1.18E−02 |
| CASSWRQGVRETQYF | 1139 | TCRBV06-05 | TCRBJ02-05 | 1.18E−02 |
| CASSTRTGVREKLFF | 1140 | TCRBV06-05 | TCRBJ01-04 | 1.28E−02 |
| CASSPSGRADNEQFF | 1141 | TCRBV27-01 | TCRBJ02-01 | 1.30E−02 |
| CASKDRDNSPLHF | 1142 | TCRBV06-X | TCRBJ01-06 | 1.31E−02 |
| CSARSRGGGPGELFF | 1143 | TCRBV20-X | TCRBJ02-02 | 1.33E−02 |
| CATSRDPDRSGANVLTF | 1144 | TCRBV15-01 | TCRBJ02-06 | 1.33E−02 |
| CSARQQGETGELFF | 1145 | TCRBV20-X | TCRBJ02-02 | 1.33E−02 |
| CASRDTRGEQYF | 1146 | TCRBV27-01 | TCRBJ02-07 | 1.40E−02 |
| CASSVRTGIREKLFF | 1147 | TCRBV06-X | TCRBJ01-04 | 1.42E−02 |
| CASSFRTGVRETQYF | 1148 | TCRBV06-05 | TCRBJ02-05 | 1.45E−02 |
| CSVDSPNEQFF | 1149 | TCRBV20-01 | TCRBJ02-01 | 1.47E−02 |
| CASSLDRQGRDYGYTF | 1150 | TCRBV07-09 | TCRBJ01-02 | 1.48E−02 |
| CASRLNEAFF | 1151 | TCRBV02-01 | TCRBJ01-01 | 1.49E−02 |
| CASISAGEKNIQYF | 1152 | TCRBV19-01 | TCRBJ02-04 | 1.49E−02 |
| CSANPSGVGETQYF | 1153 | TCRBV20-X | TCRBJ02-05 | 1.52E−02 |
| CASSPLADNEQFF | 1154 | TCRBV06-04 | TCRBJ02-01 | 1.62E−02 |
| CSARSQGEAGELFF | 1155 | TCRBV20-X | TCRBJ02-02 | 1.65E−02 |
| CASSVDSTLAKNIQYF | 1156 | TCRBV05-06 | TCRBJ02-04 | 1.68E−02 |
| CASSHRTGVRETQYF | 1157 | TCRBV06-05 | TCRBJ02-05 | 1.72E−02 |
| CASSLYGAGPYEQYF | 1158 | TCRBV05-06 | TCRBJ02-07 | 1.73E−02 |
| CASSLRTGVRETQYF | 1071 | TCRBV28-01 | TCRBJ02-05 | 1.73E−02 |
| CASSQESGNGETQYF | 1159 | TCRBV04-02 | TCRBJ02-05 | 1.77E−02 |
| CASSNRQGIRETQYF | 1160 | TCRBV06-05 | TCRBJ02-05 | 1.78E−02 |
| CASRDTRGTQYF | 1161 | TCRBV27-01 | TCRBJ02-05 | 1.80E−02 |
| CASSRELADTQYF | 319 | TCRBV07-08 | TCRBJ02-03 | 1.82E−02 |
| CASSNRQGVRETQYF | 1162 | TCRBV06-05 | TCRBJ02-05 | 1.88E−02 |
| CASSQASAAQETQYF | 1163 | TCRBV03-01/03-02 | TCRBJ02-05 | 1.88E−02 |
| CASRSFAEAFF | 1164 | TCRBV28-01 | TCRBJ01-01 | 1.90E−02 |
| CASSLASQRYSNQPQHF | 1165 | TCRBV07-08 | TCRBJ01-05 | 1.91E−02 |
| CASSPGVQSQPQHF | 1166 | TCRBV05-01 | TCRBJ01-05 | 1.92E−02 |
| CASSQGLRGGGETQYF | 1167 | TCRBV04-01 | TCRBJ02-05 | 2.00E−02 |
| CASSLMGSSSGANVLTF | 1168 | TCRBV05-06 | TCRBJ02-06 | 2.08E−02 |
| CASSIRQGVREKLFF | 1169 | TCRBV06-05 | TCRBJ01-04 | 2.08E−02 |
| CASSLGHPMNTEAFF | 1170 | TCRBV07-03 | TCRBJ01-01 | 2.11E−02 |
| CSAGPRSGNQPQHF | 1171 | TCRBV20-X | TCRBJ01-05 | 2.11E−02 |
| CASSWGRGTTGELFF | 1172 | TCRBV07-02 | TCRBJ02-02 | 2.12E−02 |
| CASSLGDYFTGELFF | 1173 | TCRBV11-02 | TCRBJ02-02 | 2.13E−02 |
| CSAIRTGGFQETQYF | 1174 | TCRBV20-X | TCRBJ02-05 | 2.14E−02 |
| CASSIRTGVREKLFF | 1175 | TCRBV06-05 | TCRBJ01-04 | 2.16E−02 |
| CASSQQGVGSNQPQHF | 1176 | TCRBV06-X | TCRBJ01-05 | 2.29E−02 |
| CASSPLGGGGETQYF | 1177 | TCRBV07-06 | TCRBJ02-05 | 2.32E−02 |
| CASSALAGETTDTQYF | 1178 | TCRBV09-01 | TCRBJ02-03 | 2.35E−02 |
| CASSSDRGLAPLHF | 1179 | TCRBV06-05 | TCRBJ01-06 | 2.40E−02 |
| CASSIRTGIREKLFF | 1180 | TCRBV06-05 | TCRBJ01-04 | 2.41E−02 |
| CASSLAPGQGGEQFF | 1181 | TCRBV05-05 | TCRBJ02-01 | 2.41E−02 |
| CASSLTPTSGEQYF | 1182 | TCRBV07-06 | TCRBJ02-07 | 2.47E−02 |
| CASSTGTRGETQYF | 1183 | TCRBV18-01 | TCRBJ02-05 | 2.47E−02 |
| CASSLSGQGGNTIYF | 1184 | TCRBV25-01 | TCRBJ01-03 | 2.51E−02 |
| CASSRTGKEDGYTF | 1185 | TCRBV03-01/03-02 | TCRBJ01-02 | 2.51E−02 |
| CASSPSGRDTDTQYF | 1186 | TCRBV06-02 | TCRBJ02-03 | 2.56E−02 |
| CASSFRQGVRETQYF | 1187 | TCRBV06-05 | TCRBJ02-05 | 2.59E−02 |
| CASSLDSSLAKNIQYF | 1188 | TCRBV05-06 | TCRBJ02-04 | 2.59E−02 |
| CSARAQGEVGELFF | 1189 | TCRBV20-X | TCRBJ02-02 | 2.60E−02 |
| CASSVDRGGSYNEQFF | 1190 | TCRBV05-01 | TCRBJ02-01 | 2.61E−02 |
| CASSDRTALEKLFF | 1191 | TCRBV02-01 | TCRBJ01-04 | 2.63E−02 |
| CSVPAQGTDTQYF | 1192 | TCRBV29-01 | TCRBJ02-03 | 2.64E−02 |
| CASSTRTGIREKLFF | 1193 | TCRBV06-05 | TCRBJ01-04 | 2.69E−02 |
| CSAGRQGTTGELFF | 1194 | TCRBV20-X | TCRBJ02-02 | 2.74E−02 |
| CASSIRQGIRETQYF | 1195 | TCRBV06-05 | TCRBJ02-05 | 2.87E−02 |
| CSARGPGETGELFF | 1196 | TCRBV20-X | TCRBJ02-02 | 2.92E−02 |
| CASSSDTTLAKNIQYF | 1197 | TCRBV05-06 | TCRBJ02-04 | 3.09E−02 |
| CAISRGRGYEQYF | 1198 | TCRBV10-03 | TCRBJ02-07 | 3.14E−02 |
| CASRYQNEQFF | 1199 | TCRBV02-01 | TCRBJ02-01 | 3.20E−02 |
| CASSIDSTLAKNIQYF | 1200 | TCRBV05-06 | TCRBJ02-04 | 3.21E−02 |
| CASSLYGSADTQYF | 1201 | TCRBV27-01 | TCRBJ02-03 | 3.26E−02 |
| CASSKRGANYGYTF | 1202 | TCRBV02-01 | TCRBJ01-02 | 3.36E−02 |
| CASSLREGSGETQYF | 1203 | TCRBV05-01 | TCRBJ02-05 | 3.39E−02 |
| CASSKGPTGNTIYF | 1204 | TCRBV05-01 | TCRBJ01-03 | 3.39E−02 |
| CASSVRQGIRETQYF | 1205 | TCRBV06-X | TCRBJ02-05 | 3.44E−02 |
| CATLQAGGHNEQFF | 1206 | TCRBV19-01 | TCRBJ02-01 | 3.44E−02 |
| CSARSRAGGPGELFF | 1207 | TCRBV20-X | TCRBJ02-02 | 3.47E−02 |
| CASGLAGGPGTGELFF | 1208 | TCRBV06-06 | TCRBJ02-02 | 3.47E−02 |
| CASSASGGRYNEQFF | 1209 | TCRBV06-04 | TCRBJ02-01 | 3.87E−02 |
| CASSLRRDSQPQHF | 1210 | TCRBV07-08 | TCRBJ01-05 | 3.88E−02 |
| CASSERAGVRETQYF | 1211 | TCRBV06-01 | TCRBJ02-05 | 3.98E−02 |
| CSARKLAGGAGELFF | 1212 | TCRBV20-X | TCRBJ02-02 | 4.02E−02 |
| CSARSKREGLGEQFF | 1213 | TCRBV20-X | TCRBJ02-01 | 4.30E−02 |
| CASSPYGQGGDEQYF | 1214 | TCRBV18-01 | TCRBJ02-07 | 4.46E−02 |
| CSARPQGDDQPQHF | 1215 | TCRBV20-X | TCRBJ01-05 | 4.46E−02 |
| CASSYRGGIRETQYF | 1216 | TCRBV06-05 | TCRBJ02-05 | 4.46E−02 |
| CATSDRKNIQYF | 1217 | TCRBV24-01 | TCRBJ02-04 | 4.56E−02 |
| CSARGQGEGGELFF | 1218 | TCRBV20-X | TCRBJ02-02 | 4.60E−02 |
| CASSRLAGMEETQYF | 1219 | TCRBV14-01 | TCRBJ02-05 | 4.64E−02 |
| CASSLRGGVREKLFF | 1220 | TCRBV06-05 | TCRBJ01-04 | 4.64E−02 |
| CASRRKDSYSNQPQHF | 1221 | TCRBV19-01 | TCRBJ01-05 | 4.64E−02 |
| CASSSDVGGQPQHF | 1222 | TCRBV19-01 | TCRBJ01-05 | 4.64E−02 |
| CSGRGQGETGELFF | 1223 | TCRBV20-X | TCRBJ02-02 | 4.64E−02 |
| CSVEGEGNTDTQYF | 1224 | TCRBV29-01 | TCRBJ02-03 | 4.70E−02 |
| CASSPDGGGEQYF | 1225 | TCRBV04-02 | TCRBJ02-07 | 4.88E−02 |
| CASSSPGRGGGNQPQHF | 1226 | TCRBV28-01 | TCRBJ01-05 | 4.89E−02 |
| CASRDSKNIQYF | 1227 | TCRBV02-01 | TCRBJ02-04 | 4.99E−02 |
| CATSVTGVQETQYF | 1228 | TCRBV24-01 | TCRBJ02-05 | 5.45E−02 |
| CASSPRTNEQYF | 1229 | TCRBV07-09 | TCRBJ02-07 | 5.86E−02 |
| CASSYRTGIRETQYF | 1230 | TCRBV06-05 | TCRBJ02-05 | 5.90E−02 |
| CASSSGALQYF | 1231 | TCRBV27-01 | TCRBJ02-07 | 6.00E−02 |
| CASSTRSGVRETQYF | 1232 | TCRBV06-05 | TCRBJ02-05 | 6.13E−02 |
| CASSYRGGVRETQYF | 1233 | TCRBV06-05 | TCRBJ02-05 | 6.49E−02 |
| CASSSGVLAKNIQYF | 1234 | TCRBV05-06 | TCRBJ02-04 | 6.64E−02 |
| CASKAGVTDTQYF | 1235 | TCRBV10-01 | TCRBJ02-03 | 6.72E−02 |
| CASSGRGGNTGELFF | 1236 | TCRBV07-09 | TCRBJ02-02 | 7.31E−02 |
| CASSLIFGESYEQYF | 1237 | TCRBV11-02 | TCRBJ02-07 | 7.64E−02 |
| CASSQDTTAYNEQFF | 1238 | TCRBV03-01/03-02 | TCRBJ02-01 | 7.77E−02 |
| CASSIRQGIREKLFF | 1239 | TCRBV06-05 | TCRBJ01-04 | 7.91E−02 |
| CSATPGGRDGYTF | 1240 | TCRBV20-X | TCRBJ01-02 | 8.01E−02 |
| CASSTELQETQYF | 1241 | TCRBV07-02 | TCRBJ02-05 | 8.06E−02 |
| CASSLVGGGTRYGYTF | 1242 | TCRBV05-06 | TCRBJ01-02 | 8.18E−02 |
| CASSNPGDRDTQYF | 1243 | TCRBV19-01 | TCRBJ02-03 | 8.40E−02 |
| CASSTRQGIRETQYF | 1244 | TCRBV06-05 | TCRBJ02-05 | 8.44E−02 |
| CSARERAGGPGELFF | 1245 | TCRBV20-X | TCRBJ02-02 | 8.77E−02 |
| CASSWDSGANVLTF | 1246 | TCRBV07-06 | TCRBJ02-06 | 8.79E−02 |
| CASSPDQSNEQFF | 1247 | TCRBV05-01 | TCRBJ02-01 | 8.79E−02 |
| CASSLRGGVRETQYF | 1248 | TCRBV06-05 | TCRBJ02-05 | 8.93E−02 |
| CASSSGALQFF | 1249 | TCRBV27-01 | TCRBJ02-01 | 9.27E−02 |
| CASSLSTSFPYNEQFF | 1250 | TCRBV05-04 | TCRBJ02-01 | 9.46E−02 |
| CSARVPSGSGTGELFF | 1251 | TCRBV20-X | TCRBJ02-02 | 9.51E−02 |
| CASSLRTGVREKLFF | 971 | TCRBV06-05 | TCRBJ01-04 | 1.06E−01 |
| CASSYRSGVRETQYF | 1252 | TCRBV06-05 | TCRBJ02-05 | 1.06E−01 |
| CASGRGNEQFF | 1253 | TCRBV11-01 | TCRBJ02-01 | 1.14E−01 |
| CSARAWGGRSSYNEQFF | 1254 | TCRBV20-X | TCRBJ02-01 | 1.16E−01 |
| CASSSPGGPYEQYF | 1255 | TCRBV06-05 | TCRBJ02-07 | 1.17E−01 |
| CASSYRQGIRETQYF | 1256 | TCRBV06-05 | TCRBJ02-05 | 1.25E−01 |
| CAWSGLGVGETQYF | 1257 | TCRBV30-01 | TCRBJ02-05 | 1.28E−01 |
| CASTQQGAGEKLFF | 1258 | TCRBV07-09 | TCRBJ01-04 | 1.30E−01 |
| CASSLRQGIRETQYF | 1259 | TCRBV06-05 | TCRBJ02-05 | 1.31E−01 |
| CASSLRQGVRETQYF | 1260 | TCRBV06-05 | TCRBJ02-05 | 1.38E−01 |
| CSARDQGEGGELFF | 1261 | TCRBV20-X | TCRBJ02-02 | 1.39E−01 |
| CASSVRQGVRETQYF | 1262 | TCRBV06-X | TCRBJ02-05 | 1.42E−01 |
| CSARKQGETGELFF | 1263 | TCRBV20-X | TCRBJ02-02 | 1.47E−01 |
| CASSTRGGVRETQYF | 1264 | TCRBV06-05 | TCRBJ02-05 | 1.49E−01 |
| CASSVRTGVRETQYF | 1062 | TCRBV06-05 | TCRBJ02-05 | 1.51E−01 |
| CSASIGYTDTQYF | 1265 | TCRBV20-01 | TCRBJ02-03 | 1.51E−01 |
| CASSLRRGESYNSPLHF | 1266 | TCRBV05-06 | TCRBJ01-06 | 1.54E−01 |
| CASSQRQGVRETQYF | 1267 | TCRBV06-05 | TCRBJ02-05 | 1.63E−01 |
| CASSPKTGGLMNTEAFF | 1268 | TCRBV18-01 | TCRBJ01-01 | 1.73E−01 |
| CASSYWDRYEQYF | 1269 | TCRBV06-02/06-03 | TCRBJ02-07 | 1.81E−01 |
| CASSLEAGFQETQYF | 1270 | TCRBV05-05 | TCRBJ02-05 | 2.01E−01 |
| CASSYRPGVRETQYF | 1271 | TCRBV06-05 | TCRBJ02-05 | 2.02E−01 |
| CSVTGGAGNTEAFF | 1272 | TCRBV20-X | TCRBJ01-01 | 2.07E−01 |
| CASSLAAGTGGNQPQHF | 1273 | TCRBV11-02 | TCRBJ01-05 | 2.12E−01 |
| CASSLVDSGNYEQYF | 1274 | TCRBV07-08 | TCRBJ02-07 | 2.12E−01 |
| CASSLSTGEPQHF | 1275 | TCRBV07-02 | TCRBJ01-05 | 2.12E−01 |
| CASSPRVAGDLNEQFF | 1276 | TCRBV09-01 | TCRBJ02-01 | 2.18E−01 |
| CASSLFTKNIQYF | 1277 | TCRBV13-01 | TCRBJ02-04 | 2.22E−01 |
| CASSLGGFSGNTIYF | 1278 | TCRBV05-05 | TCRBJ01-03 | 2.28E−01 |
| CASNTWGAGNTIYF | 1279 | TCRBV12-X | TCRBJ01-03 | 2.51E−01 |
| CASSNRGGVRETQYF | 1280 | TCRBV06-05 | TCRBJ02-05 | 2.52E−01 |
| CASSLRTGIREKLFF | 1281 | TCRBV06-05 | TCRBJ01-04 | 3.29E−01 |

TABLE 2
Additional Crohn's Disease-Associated TCRβ CDR3 Amino Acid Sequences

| TCRβ CDR3 Amino Acid | SEQ ID | | |
|---|---|---|---|
| Sequence | NO: | V Gene Segment | J Gene Segment |
| CASSPTGGTDTEAFF | 1282 | TCRBV07-03 | TCRBJ01-01 |
| CASSNPGEKNIQYF | 1283 | TCRBV19-01 | TCRBJ02-04 |
| CASRPPRGRNQPQHF | 1284 | TCRBV28-01 | TCRBJ01-05 |
| CASSLGTAGSYNSPLHF | 1285 | TCRBV05-06 | TCRBJ01-06 |
| CASSLGTAGSYNSPLHF | 1285 | TCRBV05-04 | TCRBJ01-06 |
| CASSLDLRGAEAFF | 1286 | TCRBV11-02 | TCRBJ01-01 |
| CASSLDLRGAEAFF | 1286 | TCRBV11-X | TCRBJ01-01 |
| CASSYSGLAGVYNEQFF | 1287 | TCRBV06-X | TCRBJ02-01 |
| CASSYSGLAGVYNEQFF | 1287 | TCRBV06-02/06-03 | TCRBJ02-01 |
| CSARLLAGGSGELFF | 1288 | TCRBV20-X | TCRBJ02-02 |
| CASSLGRAGSYNSPLHF | 1289 | TCRBV05-06 | TCRBJ01-06 |
| CASRGGQGNTEAFF | 1290 | TCRBV06-05 | TCRBJ01-01 |
| CASSLVGGQADTQYF | 1291 | TCRBV11-02 | TCRBJ02-03 |
| CAISDSDRGYQPQHF | 1292 | TCRBV10-03 | TCRBJ01-05 |
| CSARGQGDNQPQHF | 1293 | TCRBV20-X | TCRBJ01-05 |
| CASSLEGNSPLHF | 1294 | TCRBV27-01 | TCRBJ01-06 |
| CASSWTSGRAGYNEQFF | 1295 | TCRBV05-01 | TCRBJ02-01 |
| CASSEADRAGNTIYF | 1296 | TCRBV02-01 | TCRBJ01-03 |
| CASSSPGDKNIQYF | 1297 | TCRBV19-01 | TCRBJ02-04 |
| CASSMPGEKNIQYF | 1298 | TCRBV19-01 | TCRBJ02-04 |
| CASSLGGGRSGYTF | 1299 | TCRBV04-01 | TCRBJ01-02 |
| CASSSGQAGSYNSPLHF | 1300 | TCRBV05-04 | TCRBJ01-06 |
| CSARVIDSYEQYF | 1301 | TCRBV20-X | TCRBJ02-07 |
| CASSQPGDKNIQYF | 1302 | TCRBV19-01 | TCRBJ02-04 |
| CASSYSPPSGIYNEQFF | 1303 | TCRBV06-02/06-03 | TCRBJ02-01 |
| CASSQEFTEAFF | 1304 | TCRBV14-01 | TCRBJ01-01 |
| CASSLDQGSNQPQHF | 1305 | TCRBV07-02 | TCRBJ01-05 |
| CASSYSGLAGIYNEQFF | 1306 | TCRBV06-X | TCRBJ02-01 |
| CASRPSTGRNQPQHF | 1307 | TCRBV28-01 | TCRBJ01-05 |
| CASSSRHEQYF | 1308 | TCRBV04-02 | TCRBJ02-07 |
| CASSIDRVHNSPLHF | 1309 | TCRBV05-01 | TCRBJ01-06 |
| CASSEGRADTYEQYF | 1310 | TCRBV06-01 | TCRBJ02-07 |
| CAISEKGQGYEQYF | 1311 | TCRBV10-03 | TCRBJ02-07 |
| CASSQDGGYQETQYF | 1312 | TCRBV14-01 | TCRBJ02-05 |
| CASSYSPLAGVYNEQFF | 1313 | TCRBV06-X | TCRBJ02-01 |
| CASSLDRGSGYNEQFF | 1314 | TCRBV05-01 | TCRBJ02-01 |
| CASRTGTGNGNTIYF | 1315 | TCRBV10-02 | TCRBJ01-03 |
| CASSYGRLAGGYEQYF | 1316 | TCRBV06-05 | TCRBJ02-07 |
| CASSNPGDKNIQYF | 1317 | TCRBV19-01 | TCRBJ02-04 |
| CASSDVGRTDTQYF | 1318 | TCRBV02-01 | TCRBJ02-03 |
| CASSLRDKNYGYTF | 1319 | TCRBV07-02 | TCRBJ01-02 |
| CASSLDRVHNSPLHF | 1320 | TCRBV05-01 | TCRBJ01-06 |
| CASSQEGIRGLMNTEAFF | 1321 | TCRBV03-01/03-02 | TCRBJ01-01 |
| CASSWTSGRLSYNEQFF | 1322 | TCRBV05-01 | TCRBJ02-01 |
| CASSRELQETQYF | 1323 | TCRBV07-02 | TCRBJ02-05 |
| CASSRELQETQYF | 1323 | TCRBV07-03 | TCRBJ02-05 |
| CSATSGTDNSPLHF | 1324 | TCRBV20-X | TCRBJ01-06 |
| CASSLYVAASTDTQYF | 1325 | TCRBV05-05 | TCRBJ02-03 |
| CASSLELQETQYF | 1326 | TCRBV07-02 | TCRBJ02-05 |
| CASSLELQETQYF | 1326 | TCRBV07-03 | TCRBJ02-05 |
| CASSLGDRADQPQHF | 1327 | TCRBV05-01 | TCRBJ01-05 |
| CASSPGGGPGSPLHF | 1328 | TCRBV05-05 | TCRBJ01-06 |
| CSVEGQGAVETQYF | 1329 | TCRBV29-01 | TCRBJ02-05 |
| CASSYSRVYNEQFF | 1330 | TCRBV06-05 | TCRBJ02-01 |
| CASSYSRVYNEQFF | 1330 | TCRBV06-06 | TCRBJ02-01 |
| CASSLRPDNTEAFF | 1331 | TCRBV05-06 | TCRBJ01-01 |
| CASSLVWTGRSYEQYF | 1332 | TCRBV11-02 | TCRBJ02-07 |
| CASSQGTGDTYEQYF | 1333 | TCRBV14-01 | TCRBJ02-07 |
| CSAPDLSSYEQYF | 1334 | TCRBV20-X | TCRBJ02-07 |
| CASRASSGNQPQHF | 1335 | TCRBV02-01 | TCRBJ01-05 |
| CASRDTDTGELFF | 1336 | TCRBV05-01 | TCRBJ02-02 |
| CASSIRGGSYSNQPQHF | 1337 | TCRBV19-01 | TCRBJ01-05 |
| CASSPLTSGPYNEQFF | 1338 | TCRBV07-03 | TCRBJ02-01 |
| CASSQDWVSNQPQHF | 1339 | TCRBV04-01 | TCRBJ01-05 |
| CASSETGTGNEKLFF | 1340 | TCRBV06-01 | TCRBJ01-04 |
| CASSSSGTGYQPQHF | 1341 | TCRBV28-01 | TCRBJ01-05 |
| CASSAFTNQPQHF | 1342 | TCRBV06-04 | TCRBJ01-05 |
| CASRTSGGLETQYF | 1343 | TCRBV27-01 | TCRBJ02-05 |
| CASSLGPPPNNEQFF | 1344 | TCRBV18-01 | TCRBJ02-01 |
| CSALEGAAKNIQYF | 1345 | TCRBV20-X | TCRBJ02-04 |
| CASSQDPAGGVNEQFF | 1346 | TCRBV04-03 | TCRBJ02-01 |
| CASSTREGEQYF | 1347 | TCRBV02-01 | TCRBJ02-07 |
| CASSRVRGGNTEAFF | 1348 | TCRBV19-01 | TCRBJ01-01 |
| CASSLGTGNNEKLFF | 1349 | TCRBV07-07 | TCRBJ01-04 |
| CASRTPGLHNEQFF | 1350 | TCRBV28-01 | TCRBJ02-01 |
| CASRSQGDGQPQHF | 1351 | TCRBV12-X | TCRBJ01-05 |
| CASSYFGRGTDEQYF | 1352 | TCRBV06-02/06-03 | TCRBJ02-07 |
| CASGGHSGNTIYF | 1353 | TCRBV12-05 | TCRBJ01-03 |
| CASSFTVDSPLHF | 1354 | TCRBV07-02 | TCRBJ01-06 |
| CASSLGKTSGGQETQYF | 1355 | TCRBV05-01 | TCRBJ02-05 |
| CSARDTRTGGSEQYF | 1356 | TCRBV20-X | TCRBJ02-07 |
| CASSRTGSEQPQHF | 1357 | TCRBV19-01 | TCRBJ01-05 |
| CASRTSGDTGELFF | 1358 | TCRBV19-01 | TCRBJ02-02 |
| CASSPVAGGSQPQHF | 1359 | TCRBV18-01 | TCRBJ01-05 |
| CASSEDPGGQPQHF | 1360 | TCRBV02-01 | TCRBJ01-05 |
| CASSQGDIRWGYTF | 1361 | TCRBV14-01 | TCRBJ01-02 |
| CASRVRRDEAFF | 1362 | TCRBV28-01 | TCRBJ01-01 |
| CASSYTPREKLFF | 1363 | TCRBV06-02/06-03 | TCRBJ01-04 |
| CASSPRDRADGYTF | 1364 | TCRBV06-05 | TCRBJ01-02 |
| CASSLGEAGSYNSPLHF | 1365 | TCRBV05-06 | TCRBJ01-06 |
| CSAEQGGVHEQYF | 1366 | TCRBV20-X | TCRBJ02-07 |
| CASSLWGGRAYEQYF | 1367 | TCRBV07-08 | TCRBJ02-07 |
| CASSSMGDNQPQHF | 1368 | TCRBV12-X | TCRBJ01-05 |
| CASSQEVTYNQPQHF | 1369 | TCRBV03-01/03-02 | TCRBJ01-05 |
| CASSVGDGLGPQHF | 1370 | TCRBV09-01 | TCRBJ01-05 |
| CASRTSGGTGELFF | 1371 | TCRBV14-01 | TCRBJ02-02 |
| CASSFQQGAGYTF | 1372 | TCRBV12-03/12-04 | TCRBJ01-02 |
| CASSPGQAGSYNSPLHF | 1373 | TCRBV05-06 | TCRBJ01-06 |
| CASRGAGRYNEQFF | 1374 | TCRBV06-X | TCRBJ02-01 |
| CAISDGGFSNQPQHF | 1375 | TCRBV10-03 | TCRBJ01-05 |
| CAISDSGRGVQPQHF | 1376 | TCRBV10-03 | TCRBJ01-05 |
| CASSFTGNSNYGYTF | 1377 | TCRBV06-05 | TCRBJ01-02 |
| CASSLMRGGNTIYF | 1378 | TCRBV27-01 | TCRBJ01-03 |
| CASSSSGRRTDTQYF | 1379 | TCRBV11-03 | TCRBJ02-03 |
| CASRAGRGTDTQYF | 1380 | TCRBV27-01 | TCRBJ02-03 |
| CASSVDGPNTEAFF | 1381 | TCRBV05-01 | TCRBJ01-01 |
| CAWSAIDSLNQPQHF | 1382 | TCRBV30-01 | TCRBJ01-05 |
| LSSFEKPLPAVP | 1383 | TCRBV06-08 | TCRBJ02-07 |
| CASSSTVDSPLHF | 1384 | TCRBV07-02 | TCRBJ01-06 |
| CSANLAGAVNEQFF | 1385 | TCRBV20-X | TCRBJ02-01 |
| CASSQELQETQYF | 1386 | TCRBV07-02 | TCRBJ02-05 |
| CASSPRLGNEKLFF | 1387 | TCRBV05-06 | TCRBJ01-04 |
| CASSLTGGRTDTQYF | 1388 | TCRBV05-05 | TCRBJ02-03 |
| CASSPTGGTQETQYF | 1389 | TCRBV07-03 | TCRBJ02-05 |
| CASTRGQGNGYTF | 1390 | TCRBV19-01 | TCRBJ01-02 |
| CASRSDRLNYGYTF | 1391 | TCRBV19-01 | TCRBJ01-02 |
| CSARDDRTGSEAFF | 1392 | TCRBV20-X | TCRBJ01-01 |
| CASSLVVGGDNSPLHF | 1393 | TCRBV05-01 | TCRBJ01-06 |
| CASSQGGGRGSPLHF | 1394 | TCRBV04-02 | TCRBJ01-06 |
| CASSLNDRGNTEAFF | 1395 | TCRBV27-01 | TCRBJ01-01 |
| CASSLNDRGNTEAFF | 1395 | TCRBV13-01 | TCRBJ01-01 |
| CASSIRSGSAETQYF | 1396 | TCRBV19-01 | TCRBJ02-05 |
| CASSFDPSSYNEQFF | 1397 | TCRBV11-X | TCRBJ02-01 |
| CSASLGRGAYNEQFF | 1398 | TCRBV20-01 | TCRBJ02-01 |
| CASSHFPTEAFF | 1399 | TCRBV14-01 | TCRBJ01-01 |
| CASSVEGNNEQFF | 1400 | TCRBV06-X | TCRBJ02-01 |
| CASSLAGDLLYGYTF | 1401 | TCRBV05-01 | TCRBJ01-02 |
| CASSLANGNSGNTIYF | 1402 | TCRBV07-09 | TCRBJ01-03 |
| CASSPGRAGSYNSPLHF | 1403 | TCRBV05-04 | TCRBJ01-06 |
| CASRRRGRTDTQYF | 1404 | TCRBV28-01 | TCRBJ02-03 |
| CASSEEAGGPETQYF | 1405 | TCRBV06-04 | TCRBJ02-05 |
| CASSEDRVGGYTF | 1406 | TCRBV07-08 | TCRBJ01-02 |
| CASSASGRVNNEQFF | 1407 | TCRBV09-01 | TCRBJ02-01 |
| CASTYLDRGNYGYTF | 1408 | TCRBV28-01 | TCRBJ01-02 |
| CSARDQTSGRDTQYF | 1409 | TCRBV20-X | TCRBJ02-03 |
| CASSSASTNEKLFF | 1410 | TCRBV07-02 | TCRBJ01-04 |
| CAWTLQYNEQFF | 1411 | TCRBV30-01 | TCRBJ02-01 |
| CASSAATTNEKLFF | 1412 | TCRBV05-01 | TCRBJ01-04 |
| CASSAPQGRGYTF | 1413 | TCRBV02-01 | TCRBJ01-02 |
| CASSPRTGGAGQPQHF | 1414 | TCRBV06-X | TCRBJ01-05 |
| CASSQARDAEAFF | 1415 | TCRBV03-01/03-02 | TCRBJ01-01 |
| CASSLLNKNTEAFF | 1416 | TCRBV07-09 | TCRBJ01-01 |
| CASSRLVRDTQYF | 1417 | TCRBV07-02 | TCRBJ02-03 |
| CSAGDRVYNSPLHF | 1418 | TCRBV20-X | TCRBJ01-06 |
| CASSEYRQNTEAFF | 1419 | TCRBV10-01 | TCRBJ01-01 |
| CASSFQPSYEQYF | 1420 | TCRBV11-02 | TCRBJ02-07 |
| CASSRLVTDTQYF | 1421 | TCRBV07-06 | TCRBJ02-03 |
| CASSRLVTDTQYF | 1421 | TCRBV07-X | TCRBJ02-03 |
| CASSPGQWGYTF | 1422 | TCRBV14-01 | TCRBJ01-02 |
| CASSQPGQTYEQYF | 1423 | TCRBV19-01 | TCRBJ02-07 |
| CSNGYTF | 1424 | TCRBV29-01 | TCRBJ01-02 |
| CASSNPGQVNIQYF | 1425 | TCRBV19-01 | TCRBJ02-04 |
| CASSPPGGSPLHF | 1426 | TCRBV11-02 | TCRBJ01-06 |
| CASSPMGRTDTQYF | 1427 | TCRBV18-01 | TCRBJ02-03 |
| CAISVTGEDTEAFF | 1428 | TCRBV10-03 | TCRBJ01-01 |
| CASSLGAVNEKLFF | 1429 | TCRBV07-06 | TCRBJ01-04 |
| CASSPEDSGTDTQYF | 1430 | TCRBV18-01 | TCRBJ02-03 |
| CASSLQGAVTEAFF | 1431 | TCRBV13-01 | TCRBJ01-01 |
| CASSYDREHSPLHF | 1432 | TCRBV06-05 | TCRBJ01-06 |
| CATSDRGGTEAFF | 1433 | TCRBV03-01/03-02 | TCRBJ01-01 |
| CASSFRERYEQYF | 1434 | TCRBV06-05 | TCRBJ02-07 |
| CASSSWDRSYGYTF | 1435 | TCRBV27-01 | TCRBJ01-02 |
| CSARVRPLSTDTQYF | 1436 | TCRBV20-X | TCRBJ02-03 |
| CSTKSSYNSPLHF | 1437 | TCRBV20-X | TCRBJ01-06 |
| CASSFSTGGEQYF | 1438 | TCRBV07-06 | TCRBJ02-07 |
| CASSPRGDSSGNTIYF | 1439 | TCRBV07-08 | TCRBJ01-03 |
| CASSRTSGSRYEQYF | 1440 | TCRBV14-01 | TCRBJ02-07 |
| CASSGRDGGTEAFF | 1441 | TCRBV05-01 | TCRBJ01-01 |
| CASSLGAGYLDTQYF | 1442 | TCRBV05-01 | TCRBJ02-03 |
| CASTGPSGNTIYF | 1443 | TCRBV03-01/03-02 | TCRBJ01-03 |
| CSARKSGTSSYNEQFF | 1444 | TCRBV20-X | TCRBJ02-01 |
| CSGDRATNTEAFF | 1445 | TCRBV29-01 | TCRBJ01-01 |
| CASSLGGGWGQPQHF | 1446 | TCRBV19-01 | TCRBJ01-05 |
| CASTGRTENTEAFF | 1447 | TCRBV12-X | TCRBJ01-01 |
| CASLDRDTGELFF | 1448 | TCRBV10-01 | TCRBJ02-02 |
| CAGGPSSYEQYF | 1449 | TCRBV30-01 | TCRBJ02-07 |
| CASSPLGVDNSPLHF | 1450 | TCRBV09-01 | TCRBJ01-06 |
| CASSGVGYLAKNIQYF | 1451 | TCRBV04-01 | TCRBJ02-04 |
| CASSPGTSGSYNSPLHF | 1452 | TCRBV05-06 | TCRBJ01-06 |
| CASSYSTGQEQYF | 1453 | TCRBV06-06 | TCRBJ02-07 |
| CSVAMDSNTEAFF | 1454 | TCRBV29-01 | TCRBJ01-01 |
| CASSRGQGAAGANVLTF | 1455 | TCRBV07-09 | TCRBJ02-06 |
| CSVGLVGEAFF | 1456 | TCRBV29-01 | TCRBJ01-01 |
| CASRAPTGRNQPQHF | 1457 | TCRBV28-01 | TCRBJ01-05 |
| CASSLARRANTEAFF | 1458 | TCRBV11-02 | TCRBJ01-01 |
| CSVVSSRGQPQHF | 1459 | TCRBV29-01 | TCRBJ01-05 |
| CASSGAGFLAKNIQYF | 1460 | TCRBV04-01 | TCRBJ02-04 |
| CSARDTGGKKLFF | 1461 | TCRBV20-X | TCRBJ01-04 |
| CASRDQGAGNTEAFF | 1462 | TCRBV06-04 | TCRBJ01-01 |
| CASSLARGLGGELFF | 1463 | TCRBV12-X | TCRBJ02-02 |
| CATSRDNSNNEKLFF | 1464 | TCRBV15-01 | TCRBJ01-04 |
| CASSLDLAASNYGYTF | 1465 | TCRBV11-02 | TCRBJ01-02 |
| CASSLAPGVAGSSYEQYF | 1466 | TCRBV07-02 | TCRBJ02-07 |
| CASSWQGGGYTF | 1467 | TCRBV07-X | TCRBJ01-02 |
| CASSWQGGGYTF | 1467 | TCRBV07-02 | TCRBJ01-02 |
| CASSPLAGALSYEQYF | 1468 | TCRBV09-01 | TCRBJ02-07 |
| CATSRDYSQNSPLHF | 1469 | TCRBV15-01 | TCRBJ01-06 |
| CATSRERVSNTEAFF | 1470 | TCRBV15-01 | TCRBJ01-01 |
| CASSPLADRYEQYF | 1471 | TCRBV07-02 | TCRBJ02-07 |
| CAISVEAAGNEQFF | 1472 | TCRBV10-03 | TCRBJ02-01 |
| CASSLDGRLTDTQYF | 1473 | TCRBV07-03 | TCRBJ02-03 |
| CASSPRFYNEQFF | 1474 | TCRBV14-01 | TCRBJ02-01 |
| CASSPPGRPNTEAFF | 1475 | TCRBV04-03 | TCRBJ01-01 |
| CASSFRRGESYNSPLHF | 1476 | TCRBV05-06 | TCRBJ01-06 |
| CASSFPTGANTEAFF | 1477 | TCRBV12-X | TCRBJ01-01 |
| CASSQPGEKNIQYF | 1478 | TCRBV19-01 | TCRBJ02-04 |
| CAWLGTNYGYTF | 1479 | TCRBV30-01 | TCRBJ01-02 |
| CSVGAGGTNEKLFF | 1480 | TCRBV29-01 | TCRBJ01-04 |
| CASSLRFNTEAFF | 1481 | TCRBV11-03 | TCRBJ01-01 |
| CSAKDRGESNQPQHF | 1482 | TCRBV20-X | TCRBJ01-05 |
| CSARQGSNGYTF | 1483 | TCRBV20-X | TCRBJ01-02 |
| CASSYPGNSGNTIYF | 1484 | TCRBV06-02/06-03 | TCRBJ01-03 |
| CASSYGAPNSGNTIYF | 1485 | TCRBV06-05 | TCRBJ01-03 |
| CASTPGDRGNQPQHF | 1486 | TCRBV28-01 | TCRBJ01-05 |
| CASSPLVGGSYNEQFF | 1487 | TCRBV09-01 | TCRBJ02-01 |
| CASSTGYSGNTIYF | 1488 | TCRBV04-03 | TCRBJ01-03 |
| CASSHRQGAYEKLFF | 1489 | TCRBV03-01/03-02 | TCRBJ01-04 |
| CATSSRGRGNQPQHF | 1490 | TCRBV15-01 | TCRBJ01-05 |
| CASSRRAITEAFF | 1491 | TCRBV19-01 | TCRBJ01-01 |
| CASRVGAGDTQYF | 1492 | TCRBV12-03/12-04 | TCRBJ02-03 |
| CASSNPGVKNIQYF | 1493 | TCRBV19-01 | TCRBJ02-04 |
| CASSVSGTANQPQHF | 1494 | TCRBV06-X | TCRBJ01-05 |
| CASSQDKRQGGTEAFF | 1495 | TCRBV04-01 | TCRBJ01-01 |
| CSAPGRSDTEAFF | 1496 | TCRBV20-X | TCRBJ01-01 |
| CASSLWDTNSPLHF | 1497 | TCRBV28-01 | TCRBJ01-06 |
| CASSEGRADSYEQYF | 1498 | TCRBV06-01 | TCRBJ02-07 |
| CASSPWGGEKLFF | 1499 | TCRBV04-02 | TCRBJ01-04 |
| CSARQLAGGSGELFF | 1500 | TCRBV20-X | TCRBJ02-02 |
| CASSVQKGANQPQHF | 1501 | TCRBV06-X | TCRBJ01-05 |
| CASSLLPGGADTQYF | 1502 | TCRBV11-02 | TCRBJ02-03 |
| CASREPGGLTEAFF | 1503 | TCRBV02-01 | TCRBJ01-01 |
| CSVLLAGGKETQYF | 1504 | TCRBV29-01 | TCRBJ02-05 |
| CASSHTGYGYTF | 1505 | TCRBV07-02 | TCRBJ01-02 |
| CASSLGVSGGEQYF | 1506 | TCRBV05-01 | TCRBJ02-07 |
| CATRDGDTGELFF | 1507 | TCRBV05-01 | TCRBJ02-02 |
| CSVGRQETEAFF | 1508 | TCRBV29-01 | TCRBJ01-01 |
| CASSLARSRTEAFF | 1509 | TCRBV05-06 | TCRBJ01-01 |
| CASSLAGAPSEQYF | 1510 | TCRBV11-02 | TCRBJ02-07 |
| CASSLGYEGYTF | 1511 | TCRBV12-03/12-04 | TCRBJ01-02 |
| CASSLGLGRPTGELFF | 1512 | TCRBV07-02 | TCRBJ02-02 |
| CASSLRGPRDTEAFF | 1513 | TCRBV07-03 | TCRBJ01-01 |
| CSARLGTVSGYTF | 1514 | TCRBV20-X | TCRBJ01-02 |
| CASSLWDRDTEAFF | 1515 | TCRBV09-01 | TCRBJ01-01 |
| CATLQVGAKNIQYF | 1516 | TCRBV19-01 | TCRBJ02-04 |
| CSARVLDSYEQYF | 1517 | TCRBV20-X | TCRBJ02-07 |
| CAWSRQAGGYTF | 1518 | TCRBV30-01 | TCRBJ01-02 |
| CASRGQGDGQPQHF | 1519 | TCRBV12-X | TCRBJ01-05 |
| CASSSPGAKNIQYF | 1520 | TCRBV19-01 | TCRBJ02-04 |
| CASSQPKRGTYEQYF | 1521 | TCRBV03-01/03-02 | TCRBJ02-07 |
| CASSRAGGGQETQYF | 1522 | TCRBV10-02 | TCRBJ02-05 |
| CASSRTGTPLHF | 1523 | TCRBV05-06 | TCRBJ01-06 |
| CASSLDLIAYEQYF | 1524 | TCRBV07-02 | TCRBJ02-07 |
| CASSPDRGQGDTQYF | 1525 | TCRBV05-08 | TCRBJ02-03 |
| CASSIDPGQTYYGYTF | 1526 | TCRBV19-01 | TCRBJ01-02 |
| CSAPRPSGREETQYF | 1527 | TCRBV20-X | TCRBJ02-05 |
| CASSYREGNSPLHF | 1528 | TCRBV13-01 | TCRBJ01-06 |
| CSVRAGVRNEQFF | 1529 | TCRBV29-01 | TCRBJ02-01 |
| CASSVTGGQAEAFF | 1530 | TCRBV09-01 | TCRBJ01-01 |
| CAISQSSNYGYTF | 1531 | TCRBV10-03 | TCRBJ01-02 |
| CSALPRTGGGNTIYF | 1532 | TCRBV20-X | TCRBJ01-03 |
| CASSRTGLDNEQFF | 1533 | TCRBV06-02/06-03 | TCRBJ02-01 |
| CSAREGTSEHNEQFF | 1534 | TCRBV20-X | TCRBJ02-01 |
| CASSLTPTGGGQPQHF | 1535 | TCRBV05-01 | TCRBJ01-05 |
| CASSLSPGGPTGELFF | 1536 | TCRBV27-01 | TCRBJ02-02 |
| CASSRTGTTTDTQYF | 1537 | TCRBV05-04 | TCRBJ02-03 |
| CSARDAGRYNEKLFF | 1538 | TCRBV20-X | TCRBJ01-04 |
| CASSPTAFYNSPLHF | 1539 | TCRBV09-01 | TCRBJ01-06 |
| CASSLDSLGNTEAFF | 1540 | TCRBV11-03 | TCRBJ01-01 |
| CATRDLGEKLFF | 1541 | TCRBV06-05 | TCRBJ01-04 |
| CASSRTEYNSPLHF | 1542 | TCRBV06-04 | TCRBJ01-06 |
| CSARDPRQGRSYGYTF | 1543 | TCRBV20-X | TCRBJ01-02 |
| CASSTRQGVREKLFF | 1544 | TCRBV06-05 | TCRBJ01-04 |
| CASSRKGRNTEAFF | 1545 | TCRBV06-05 | TCRBJ01-01 |
| CASSPGGGTAYEQYF | 1546 | TCRBV11-03 | TCRBJ02-07 |
| CASSPDREAHEQYF | 1547 | TCRBV28-01 | TCRBJ02-07 |
| CSASMDRDQPQHF | 1548 | TCRBV20-X | TCRBJ01-05 |
| CASSRAGTKYNEQFF | 1549 | TCRBV07-02 | TCRBJ02-01 |
| CASSQAGRETEAFF | 1550 | TCRBV14-01 | TCRBJ01-01 |
| CASTRQDGNTIYF | 1551 | TCRBV06-05 | TCRBJ01-03 |
| CASSYGEQYGYTF | 1552 | TCRBV06-02/06-03 | TCRBJ01-02 |
| CAIRGSSSSYNEQFF | 1553 | TCRBV10-03 | TCRBJ02-01 |
| CASSQGEDYGYTF | 1554 | TCRBV07-08 | TCRBJ01-02 |
| CASKRGASGANVLTF | 1555 | TCRBV19-01 | TCRBJ02-06 |
| CASTPGQLNTEAFF | 1556 | TCRBV14-01 | TCRBJ01-01 |
| CASRDRGESQPQHF | 1557 | TCRBV19-01 | TCRBJ01-05 |
| CATSDSGLAGGAETQYF | 1558 | TCRBV24-01 | TCRBJ02-05 |
| CASSSGVAGVQETQYF | 1559 | TCRBV07-09 | TCRBJ02-05 |
| CSASDHSPLHF | 1560 | TCRBV20-01 | TCRBJ01-06 |
| CASSLEPTSGGETQYF | 1561 | TCRBV05-01 | TCRBJ02-05 |
| CSARADHPRETQYF | 1562 | TCRBV20-X | TCRBJ02-05 |
| CASQRLANYGYTF | 1563 | TCRBV28-01 | TCRBJ01-02 |
| CASSRSPREGYTF | 1564 | TCRBV18-01 | TCRBJ01-02 |
| CASSQVFGQETQYF | 1565 | TCRBV14-01 | TCRBJ02-05 |
| CASSLPGTGGNTEAFF | 1566 | TCRBV12-X | TCRBJ01-01 |
| CASSYLNYYGYTF | 1567 | TCRBV06-02/06-03 | TCRBJ01-02 |
| CASNTQGANSPLHF | 1568 | TCRBV19-01 | TCRBJ01-06 |
| CASSLYPALYEQYF | 1569 | TCRBV07-09 | TCRBJ02-07 |
| CASSRQRGSSYNEQFF | 1570 | TCRBV07-09 | TCRBJ02-01 |
| CATSDLTRSEQFF | 1571 | TCRBV24-01 | TCRBJ02-01 |
| CASSPAGVGLTDTQYF | 1572 | TCRBV07-02 | TCRBJ02-03 |
| CASSFGDKNYGYTF | 1573 | TCRBV12-X | TCRBJ01-02 |
| CASSQDPGRGTQYF | 1574 | TCRBV03-01/03-02 | TCRBJ02-03 |
| CASTIQGIQPQHF | 1575 | TCRBV28-01 | TCRBJ01-05 |
| CASSQAGNIYGYTF | 1576 | TCRBV04-01 | TCRBJ01-02 |
| CASSRVGPNYGYTF | 1577 | TCRBV28-01 | TCRBJ01-02 |
| CASRIRGGAYEQYF | 1578 | TCRBV27-01 | TCRBJ02-07 |
| CASSPSGAAYSPLHF | 1579 | TCRBV06-05 | TCRBJ01-06 |
| CASREVSGNTIYF | 1580 | TCRBV04-01 | TCRBJ01-03 |
| CASSPAGDRNSYEQYF | 1581 | TCRBV18-01 | TCRBJ02-07 |
| CASTQGGSGTEAFF | 1582 | TCRBV02-01 | TCRBJ01-01 |
| CASSQELGLYNEQFF | 1583 | TCRBV04-02 | TCRBJ02-01 |
| CSASWRTGGTEAFF | 1584 | TCRBV20-01 | TCRBJ01-01 |
| CASSPPAGGNTEAFF | 1585 | TCRBV07-08 | TCRBJ01-01 |
| CASKGTQGNQPQHF | 1586 | TCRBV06-05 | TCRBJ01-05 |
| CAISDRALQPQHF | 1587 | TCRBV10-03 | TCRBJ01-05 |
| CASSKDRDYNEQFF | 1588 | TCRBV18-01 | TCRBJ02-01 |
| CSVGRTGGFGYTF | 1589 | TCRBV29-01 | TCRBJ01-02 |
| CSGKGTTNEKLFF | 1590 | TCRBV20-X | TCRBJ01-04 |
| CAISAPGPNYGYTF | 1591 | TCRBV10-03 | TCRBJ01-02 |
| CASSFGDSSYGYTF | 1592 | TCRBV05-08 | TCRBJ01-02 |
| CASSLWQARNTIYF | 1593 | TCRBV05-06 | TCRBJ01-03 |
| CASTGSFSGNTIYF | 1594 | TCRBV09-01 | TCRBJ01-03 |
| CASSQVRGRDTDTQYF | 1595 | TCRBV04-01 | TCRBJ02-03 |
| CSASWASNEKLFF | 1596 | TCRBV20-X | TCRBJ01-04 |
| CASSLGGNTYQPQHF | 1597 | TCRBV07-02 | TCRBJ01-05 |
| CAWNRRRETQYF | 1598 | TCRBV30-01 | TCRBJ02-05 |
| CASRKLIYGYTF | 1599 | TCRBV28-01 | TCRBJ01-02 |
| CATSRDRQVPGYTF | 1600 | TCRBV15-01 | TCRBJ01-02 |
| CSARDPDPSSYNSPLHF | 1601 | TCRBV20-X | TCRBJ01-06 |
| CASSLELLAGVETQYF | 1602 | TCRBV07-09 | TCRBJ02-05 |
| CASSQVRTGANEKLFF | 1603 | TCRBV04-03 | TCRBJ01-04 |
| CASSIRTGIVYEQYF | 1604 | TCRBV19-01 | TCRBJ02-07 |
| CASSTPYNEQFF | 1605 | TCRBV06-01 | TCRBJ02-01 |
| CASSPPQREGETQYF | 1606 | TCRBV18-01 | TCRBJ02-05 |
| CSVEEFRGDGYTF | 1607 | TCRBV29-01 | TCRBJ01-02 |
| CASSVGLYEQYF | 1608 | TCRBV06-02 | TCRBJ02-07 |
| CASNLAGDADTQYF | 1609 | TCRBV05-01 | TCRBJ02-03 |
| CSAPSGSGNTQYF | 1610 | TCRBV20-X | TCRBJ02-03 |
| CASRPPGQKNIQYF | 1611 | TCRBV12-X | TCRBJ02-04 |
| CASSLAYYSNQPQHF | 1612 | TCRBV07-02 | TCRBJ01-05 |
| CASRSTAGSYEQYF | 1613 | TCRBV05-01 | TCRBJ02-07 |
| CASSGAAGTDTQYF | 1614 | TCRBV06-01 | TCRBJ02-03 |
| CASGGLPQHF | 1615 | TCRBV19-01 | TCRBJ01-05 |
| CASSTTGGENYGYTF | 1616 | TCRBV28-01 | TCRBJ01-02 |
| CASSRWTGLNTEAFF | 1617 | TCRBV11-01 | TCRBJ01-01 |
| CASSTTANEQFF | 1618 | TCRBV06-06 | TCRBJ02-01 |
| CASSYAGQGFNQPQHF | 1619 | TCRBV06-02/06-03 | TCRBJ01-05 |
| CASSRPGQKNTEAFF | 1620 | TCRBV02-01 | TCRBJ01-01 |
| CASSLYSRVYEQYF | 1621 | TCRBV05-04 | TCRBJ02-07 |
| CASRGTGSTQPQHF | 1622 | TCRBV27-01 | TCRBJ01-05 |
| CASSLGASNNQPQHF | 1623 | TCRBV11-02 | TCRBJ01-05 |
| CASSRGTGEIYGYTF | 1624 | TCRBV03-01/03-02 | TCRBJ01-02 |
| CASSPDGGIQAFF | 1625 | TCRBV18-01 | TCRBJ01-01 |
| CASGLERDSNQPQHF | 1626 | TCRBV12-05 | TCRBJ01-05 |
| CASSDSLAGAGEQFF | 1627 | TCRBV02-01 | TCRBJ02-01 |
| CSASALVSGNTIYF | 1628 | TCRBV20-01 | TCRBJ01-03 |
| CSARDRGLGTNTGELFF | 1629 | TCRBV20-X | TCRBJ02-02 |
| CASSRLGEAGNTIYF | 1630 | TCRBV05-06 | TCRBJ01-03 |
| CASSLEVYGTEAFF | 1631 | TCRBV05-08 | TCRBJ01-01 |
| CASSSPGTLNYGYTF | 1632 | TCRBV05-01 | TCRBJ01-02 |
| CASRRQGAVTEAFF | 1633 | TCRBV06-X | TCRBJ01-01 |
| CASSPETAPSYEQYF | 1634 | TCRBV18-01 | TCRBJ02-07 |
| CSAMTGPYGYTF | 1635 | TCRBV29-01 | TCRBJ01-02 |
| CASSLSAWNYGYTF | 1636 | TCRBV27-01 | TCRBJ01-02 |
| CASSPLPGGDEKLFF | 1637 | TCRBV05-01 | TCRBJ01-04 |
| CASSYEVTNYGYTF | 1638 | TCRBV06-06 | TCRBJ01-02 |
| CASSQGTSVGELFF | 1639 | TCRBV04-02 | TCRBJ02-02 |
| CASSSQGTSTEAFF | 1640 | TCRBV18-01 | TCRBJ01-01 |
| CASSQGLAGVLSDTQYF | 1641 | TCRBV04-01 | TCRBJ02-03 |
| CASSLIGGRITGELFF | 1642 | TCRBV07-09 | TCRBJ02-02 |
| CASSFGGDMNTEAFF | 1643 | TCRBV05-06 | TCRBJ01-01 |
| CASGPGGGRGYTF | 1644 | TCRBV12-05 | TCRBJ01-02 |
| CASSASEGSSYNSPLHF | 1645 | TCRBV09-01 | TCRBJ01-06 |
| CASSLGQGEEAFF | 1646 | TCRBV11-03 | TCRBJ01-01 |
| CASGKGGADEKLFF | 1647 | TCRBV02-01 | TCRBJ01-04 |
| CASSFQGAGDGYTF | 1648 | TCRBV28-01 | TCRBJ01-02 |
| CASSSSGLLEAFF | 1649 | TCRBV12-03/12-04 | TCRBJ01-01 |
| CASSLGAGGEGGYTF | 1650 | TCRBV07-02 | TCRBJ01-02 |
| CASSFWDSLNYGYTF | 1651 | TCRBV12-X | TCRBJ01-02 |
| CASSAGRLEKLFF | 1652 | TCRBV28-01 | TCRBJ01-04 |
| CASSRRTGGSTDTQYF | 1653 | TCRBV21-01 | TCRBJ02-03 |
| CASSYRVWTEAFF | 1654 | TCRBV12-03/12-04 | TCRBJ01-01 |
| CASSQSNYGYTF | 1655 | TCRBV11-X | TCRBJ01-02 |
| CASSFNRGHSYEQYF | 1656 | TCRBV06-02/06-03 | TCRBJ02-07 |
| CASSLRPPGDTQYF | 1657 | TCRBV03-01/03-02 | TCRBJ02-03 |
| CASSFPVGNSPLHF | 1658 | TCRBV27-01 | TCRBJ01-06 |
| CASSRISGPSSYEQYF | 1659 | TCRBV11-02 | TCRBJ02-07 |
| CATRSGLGSNEQFF | 1660 | TCRBV24-01 | TCRBJ02-01 |
| CSASPNRGGGNQPQHF | 1661 | TCRBV20-X | TCRBJ01-05 |
| CATSREKAPQHF | 1662 | TCRBV15-01 | TCRBJ01-05 |
| CASSAGTAGSYNSPLHF | 1663 | TCRBV05-06 | TCRBJ01-06 |
| CASSYSGYYEAFF | 1664 | TCRBV06-05 | TCRBJ01-01 |
| CASTPIDSLNTEAFF | 1665 | TCRBV02-01 | TCRBJ01-01 |
| CASSQGRQGDTGELFF | 1666 | TCRBV04-01 | TCRBJ02-02 |
| CSARWRGIYEQYF | 1667 | TCRBV20-X | TCRBJ02-07 |
| CASQIQGLNTEAFF | 1668 | TCRBV07-09 | TCRBJ01-01 |
| CASSYHRDRETQYF | 1669 | TCRBV05-01 | TCRBJ02-05 |
| CASKRDGGEKLFF | 1670 | TCRBV02-01 | TCRBJ01-04 |
| CASSLPRTGGNTIYF | 1671 | TCRBV03-01/03-02 | TCRBJ01-03 |
| CASSYSNPRSYNSPLHF | 1672 | TCRBV06-X | TCRBJ01-06 |
| CASSLWGEGYGYTF | 1673 | TCRBV06-05 | TCRBJ01-02 |
| CAWTQQGTNEKLFF | 1674 | TCRBV30-01 | TCRBJ01-04 |
| CASSSGQFNQPQHF | 1675 | TCRBV11-X | TCRBJ01-05 |
| CASSLGTGNIQYF | 1676 | TCRBV11-X | TCRBJ02-04 |
| CASSNPGANTEAFF | 1677 | TCRBV07-09 | TCRBJ01-01 |
| CASSSWTSGRYEQYF | 1678 | TCRBV07-03 | TCRBJ02-07 |
| CASSRGGQRYNSPLHF | 1679 | TCRBV05-01 | TCRBJ01-06 |
| CASSEYLAGDTGELFF | 1680 | TCRBV06-01 | TCRBJ02-02 |
| CASSPTGLIYEQYF | 1681 | TCRBV14-01 | TCRBJ02-07 |
| CASSLSGAASPLHF | 1682 | TCRBV12-X | TCRBJ01-06 |
| CASSLRVEGADTQYF | 1683 | TCRBV12-X | TCRBJ02-03 |
| CASRTGGGLNSPLHF | 1684 | TCRBV19-01 | TCRBJ01-06 |
| CSARRNLNNEQFF | 1685 | TCRBV20-X | TCRBJ02-01 |
| CASSQVGRGPNEQFF | 1686 | TCRBV04-01 | TCRBJ02-01 |
| CATSRNRGGYQPQHF | 1687 | TCRBV15-01 | TCRBJ01-05 |
| CASSKGLHYGYTF | 1688 | TCRBV28-01 | TCRBJ01-02 |
| CSAKGDRMNTEAFF | 1689 | TCRBV20-X | TCRBJ01-01 |
| CASNMGSYGYTF | 1690 | TCRBV19-01 | TCRBJ01-02 |
| CSVEVRGYTEAFF | 1691 | TCRBV29-01 | TCRBJ01-01 |
| CASSEQTGEAFF | 1692 | TCRBV25-01 | TCRBJ01-01 |
| CASSPYRTGPYEQYF | 1693 | TCRBV07-08 | TCRBJ02-07 |
| CASRGTSGGGTQYF | 1694 | TCRBV12-X | TCRBJ02-05 |
| CASSSEGGYNQPQHF | 1695 | TCRBV28-01 | TCRBJ01-05 |
| CASSFLGHNSPLHF | 1696 | TCRBV05-06 | TCRBJ01-06 |
| CASSYSTALQETQYF | 1697 | TCRBV06-06 | TCRBJ02-05 |
| CASSSPETGTNSPLHF | 1698 | TCRBV05-01 | TCRBJ01-06 |
| CASSPGAGPPYEQYF | 1699 | TCRBV05-01 | TCRBJ02-07 |
| CATSQGGYNSPLHF | 1700 | TCRBV15-01 | TCRBJ01-06 |
| CASSPGQNHNEQFF | 1701 | TCRBV05-01 | TCRBJ02-01 |
| CASRRDTLASPLHF | 1702 | TCRBV27-01 | TCRBJ01-06 |
| CASRSEGASTEAFF | 1703 | TCRBV05-01 | TCRBJ01-01 |
| CASSRPREQYF | 1704 | TCRBV05-08 | TCRBJ02-07 |
| CASVREQYF | 1705 | TCRBV28-01 | TCRBJ02-07 |
| CASSYRDRDAYEQYF | 1706 | TCRBV07-08 | TCRBJ02-07 |
| CASSGGDRGRSPLHF | 1707 | TCRBV05-04 | TCRBJ01-06 |
| CAISADRAGAEAFF | 1708 | TCRBV10-03 | TCRBJ01-01 |
| CASSPTTGLNQPQHF | 1709 | TCRBV06-05 | TCRBJ01-05 |
| CASSRQFNTGELFF | 1710 | TCRBV14-01 | TCRBJ02-02 |
| CASSQDAGKGYGYTF | 1711 | TCRBV04-01 | TCRBJ01-02 |
| CASSPGTAGSYNSPLHF | 1712 | TCRBV05-04 | TCRBJ01-06 |
| CASSQYSASTDTQYF | 1713 | TCRBV14-01 | TCRBJ02-03 |
| CASRLDTSTDTQYF | 1714 | TCRBV05-08 | TCRBJ02-03 |
| CASSFRPGDYGYTF | 1715 | TCRBV11-02 | TCRBJ01-02 |
| CASSQALTSDEQFF | 1716 | TCRBV03-01/03-02 | TCRBJ02-01 |
| CASSFGDRGGNYGYTF | 1717 | TCRBV12-X | TCRBJ01-02 |
| CASHRGLGTEAFF | 1718 | TCRBV06-06 | TCRBJ01-01 |
| CATSDRGLLGETQYF | 1719 | TCRBV24-01 | TCRBJ02-05 |
| CASSTQGVTYEQYF | 1720 | TCRBV02-01 | TCRBJ02-07 |
| CASRAGTSTGELFF | 1721 | TCRBV06-06 | TCRBJ02-02 |
| CASSPPLRDRAYNEQFF | 1722 | TCRBV07-09 | TCRBJ02-01 |
| CASNRPAGSGANVLTF | 1723 | TCRBV06-X | TCRBJ02-06 |
| CASSPLRGSYGYTF | 1724 | TCRBV07-08 | TCRBJ01-02 |
| CSAPRGGAYYNEQFF | 1725 | TCRBV20-X | TCRBJ02-01 |
| CASSLDPSPNTEAFF | 1726 | TCRBV11-X | TCRBJ01-01 |
| CASSLSGQLNYGYTF | 1727 | TCRBV05-04 | TCRBJ01-02 |
| CASRDHNYGYTF | 1728 | TCRBV06-04 | TCRBJ01-02 |
| CASSPLSSSGANVLTF | 1729 | TCRBV07-03 | TCRBJ02-06 |
| CASSSPNAEAFF | 1730 | TCRBV28-01 | TCRBJ01-01 |
| CASSQDPSGVTEAFF | 1731 | TCRBV04-02 | TCRBJ01-01 |
| CASSTSGTDNQPQHF | 1732 | TCRBV07-02 | TCRBJ01-05 |
| CASSLGPQGQYF | 1733 | TCRBV05-06 | TCRBJ02-03 |
| CASSLGQGGSQPQHF | 1734 | TCRBV07-06 | TCRBJ01-05 |
| CASSEWGLRSYEQYF | 1735 | TCRBV06-01 | TCRBJ02-07 |
| CSAPRTSGGRSTDTQYF | 1736 | TCRBV20-X | TCRBJ02-03 |
| CASSYLRAGNTDTQYF | 1737 | TCRBV06-05 | TCRBJ02-03 |
| CASSRTADNSPLHF | 1738 | TCRBV28-01 | TCRBJ01-06 |
| CASSLSFGGNSPLHF | 1739 | TCRBV05-01 | TCRBJ01-06 |
| CASSELAGLYGYTF | 1740 | TCRBV06-01 | TCRBJ01-02 |
| CASSFPGTNTDTQYF | 1741 | TCRBV05-04 | TCRBJ02-03 |
| CASSPPVNYGYTF | 1742 | TCRBV19-01 | TCRBJ01-02 |
| CASKDTNTEAFF | 1743 | TCRBV05-01 | TCRBJ01-01 |
| CASSPRTGINSPLHF | 1744 | TCRBV11-02 | TCRBJ01-06 |
| CASSPRLAEAFF | 1745 | TCRBV12-03/12-04 | TCRBJ01-01 |
| CASSQELQGLYGYTF | 1746 | TCRBV04-03 | TCRBJ01-02 |
| CASSPRLDRGYGYTF | 1747 | TCRBV18-01 | TCRBJ01-02 |
| CASISTGTYGYTF | 1748 | TCRBV06-05 | TCRBJ01-02 |
| CASSESPGYGYTF | 1749 | TCRBV02-01 | TCRBJ01-02 |
| CAWSVQGSRNQPQHF | 1750 | TCRBV30-01 | TCRBJ01-05 |
| CASSMGQGAGAGELFF | 1751 | TCRBV19-01 | TCRBJ02-02 |
| CASSYSPPSGVYNEQFF | 1752 | TCRBV06-02/06-03 | TCRBJ02-01 |
| CAWSVRPGRNTEAFF | 1753 | TCRBV30-01 | TCRBJ01-01 |
| CASSLGVGWETQYF | 1754 | TCRBV07-09 | TCRBJ02-05 |
| CASSLTSGRHEQYF | 1755 | TCRBV11-02 | TCRBJ02-07 |
| CASSLDRGVTEAFF | 1756 | TCRBV05-08 | TCRBJ01-01 |
| CATSRRTGRNSPLHF | 1757 | TCRBV15-01 | TCRBJ01-06 |
| CASSSGTGGSGNTIYF | 1758 | TCRBV07-03 | TCRBJ01-03 |
| CASSLDTTLAKNIQYF | 1759 | TCRBV05-06 | TCRBJ02-04 |
| CASSERLTGELFF | 1760 | TCRBV09-01 | TCRBJ02-02 |
| CASSQHQKLFF | 1761 | TCRBV14-01 | TCRBJ01-04 |
| CASTKQGQPQHF | 1762 | TCRBV19-01 | TCRBJ01-05 |
| CSAPGESTEAFF | 1763 | TCRBV20-X | TCRBJ01-01 |
| CASSPAIRDTEAFF | 1764 | TCRBV18-01 | TCRBJ01-01 |
| CSVEETGGNQPQHF | 1765 | TCRBV29-01 | TCRBJ01-05 |
| CASSQEGGIYGYTF | 1766 | TCRBV04-02 | TCRBJ01-02 |
| CASSQDRGSSSGNTIYF | 1767 | TCRBV04-03 | TCRBJ01-03 |
| CSATQGLETQYF | 1768 | TCRBV20-X | TCRBJ02-05 |
| CASSYGASGGLTDTQYF | 1769 | TCRBV06-02/06-03 | TCRBJ02-03 |
| CSVDGQGNTDTQYF | 1770 | TCRBV29-01 | TCRBJ02-03 |
| CAWSVRDSLNEKLFF | 1771 | TCRBV30-01 | TCRBJ01-04 |
| CASSPTGVAGNTIYF | 1772 | TCRBV09-01 | TCRBJ01-03 |
| CASSPPVGDNSPLHF | 1773 | TCRBV18-01 | TCRBJ01-06 |
| CASRRDRGNEKLFF | 1774 | TCRBV05-04 | TCRBJ01-04 |
| CATSREKGPNTEAFF | 1775 | TCRBV15-01 | TCRBJ01-01 |
| CASSLRRGLGYGYTF | 1776 | TCRBV05-06 | TCRBJ01-02 |
| CASSVEDSGGYNEQFF | 1777 | TCRBV09-01 | TCRBJ02-01 |
| CASSASGGYYEQYF | 1778 | TCRBV02-01 | TCRBJ02-07 |
| CASSLRSSGGVDNEQFF | 1779 | TCRBV11-03 | TCRBJ02-01 |
| CASSQSPTSYEQYF | 1780 | TCRBV03-01/03-02 | TCRBJ02-07 |
| CASSLAPLAGGNEQFF | 1781 | TCRBV07-09 | TCRBJ02-01 |
| CASSQDRRSYEQYF | 1782 | TCRBV05-01 | TCRBJ02-07 |
| CASSLAGTGRADTQYF | 1783 | TCRBV07-02 | TCRBJ02-03 |
| CASSFAQETQYF | 1784 | TCRBV05-04 | TCRBJ02-05 |
| CASSSNRVAYEQYF | 1785 | TCRBV06-05 | TCRBJ02-07 |
| CSASLAGVAKNIQYF | 1786 | TCRBV20-01 | TCRBJ02-04 |
| CASSTMTSGSYEQYF | 1787 | TCRBV19-01 | TCRBJ02-07 |
| CASSQQLAGEQYF | 1788 | TCRBV03-01/03-02 | TCRBJ02-07 |
| CASRGLEKLFF | 1789 | TCRBV25-01 | TCRBJ01-04 |
| CASSYPTGATYEQYF | 1790 | TCRBV06-02/06-03 | TCRBJ02-07 |
| CASSYRESNQPQHF | 1791 | TCRBV07-02 | TCRBJ01-05 |
| CSVELGTGGNYGYTF | 1792 | TCRBV29-01 | TCRBJ01-02 |
| CASSRQGQGNTIYF | 1793 | TCRBV05-01 | TCRBJ01-03 |
| CASSLGQRKAFF | 1794 | TCRBV28-01 | TCRBJ01-01 |
| CSARFQVDQPQHF | 1795 | TCRBV20-X | TCRBJ01-05 |
| CASSLDVYNQPQHF | 1796 | TCRBV12-X | TCRBJ01-05 |
| CASSDSDSQETQYF | 1797 | TCRBV02-01 | TCRBJ02-05 |
| CASSLAATNNNEQFF | 1798 | TCRBV05-01 | TCRBJ02-01 |
| CSAGRGTGGVNQPQHF | 1799 | TCRBV20-X | TCRBJ01-05 |
| CASSRLGPDTQYF | 1800 | TCRBV05-05 | TCRBJ02-03 |
| CASSLSEVNTEAFF | 1801 | TCRBV07-03 | TCRBJ01-01 |
| CASSQNRENEKLFF | 1802 | TCRBV03-01/03-02 | TCRBJ01-04 |
| CSAHRSNTEAFF | 1803 | TCRBV20-X | TCRBJ01-01 |
| CASSQESTASGNTIYF | 1804 | TCRBV04-02 | TCRBJ01-03 |
| CASRDVGETQYF | 1805 | TCRBV19-01 | TCRBJ02-05 |
| CASVQGASNYGYTF | 1806 | TCRBV05-04 | TCRBJ01-02 |
| CASSNPGDRNEQFF | 1807 | TCRBV19-01 | TCRBJ02-01 |
| CASRHRGHNQPQHF | 1808 | TCRBV06-05 | TCRBJ01-05 |
| CSAGQGKGEQYF | 1809 | TCRBV20-X | TCRBJ02-07 |
| CASSPLAGVYEQYF | 1810 | TCRBV06-02/06-03 | TCRBJ02-07 |
| CASTPPPGNQPQHF | 1811 | TCRBV28-01 | TCRBJ01-05 |
| CASSDNWGTEAFF | 1812 | TCRBV02-01 | TCRBJ01-01 |
| CASRDGDTGELFF | 1813 | TCRBV05-01 | TCRBJ02-02 |
| CAWSRQHNEQFF | 1814 | TCRBV30-01 | TCRBJ02-01 |
| CASSPRQVFSGNTIYF | 1815 | TCRBV09-01 | TCRBJ01-03 |
| CSVEDLAASSYNEQFF | 1816 | TCRBV29-01 | TCRBJ02-01 |
| CARGGQPQHF | 1817 | TCRBV06-05 | TCRBJ01-05 |
| CASSPTAYSYEQYF | 1818 | TCRBV05-04 | TCRBJ02-07 |
| CASSLRTGLRETQYF | 1819 | TCRBV27-01 | TCRBJ02-05 |
| CASSQEILDTQYF | 1820 | TCRBV04-03 | TCRBJ02-03 |
| CASSMEGSRGYTF | 1821 | TCRBV19-01 | TCRBJ01-02 |
| CAISEWDRGMNTEAFF | 1822 | TCRBV10-03 | TCRBJ01-01 |
| CSARSQGRYTGELFF | 1823 | TCRBV20-X | TCRBJ02-02 |
| CASSPYRVNTGELFF | 1824 | TCRBV07-08 | TCRBJ02-02 |
| CASSPQDRVNQPQHF | 1825 | TCRBV12-X | TCRBJ01-05 |
| CSAREQGETGELFF | 1826 | TCRBV20-X | TCRBJ02-02 |
| CASSDSAEGGELFF | 1827 | TCRBV06-04 | TCRBJ02-02 |
| CASRVDSHQPQHF | 1828 | TCRBV06-05 | TCRBJ01-05 |
| CASSSGTAGSYNSPLHF | 1829 | TCRBV05-06 | TCRBJ01-06 |
| CASSPRSGRGETQYF | 1830 | TCRBV19-01 | TCRBJ02-05 |
| CASRGAVQPQHF | 1831 | TCRBV28-01 | TCRBJ01-05 |
| CASRADRRNTIYF | 1832 | TCRBV06-05 | TCRBJ01-03 |
| CATSRGTGASTGELFF | 1833 | TCRBV15-01 | TCRBJ02-02 |
| CASSSGQDSPLHF | 1834 | TCRBV05-05 | TCRBJ01-06 |
| CASSQDRRGGNYEQFF | 1835 | TCRBV04-03 | TCRBJ02-01 |
| CASSQVDSPNTGELFF | 1836 | TCRBV03-01/03-02 | TCRBJ02-02 |
| CASRRQGGAYEQYF | 1837 | TCRBV07-02 | TCRBJ02-07 |
| CASRRGTVSTDTQYF | 1838 | TCRBV02-01 | TCRBJ02-03 |
| CASSLGDRGQTNEKLFF | 1839 | TCRBV07-09 | TCRBJ01-04 |
| CASSGQVDNEQFF | 1840 | TCRBV05-01 | TCRBJ02-01 |
| CASSEARKGNTEAFF | 1841 | TCRBV02-01 | TCRBJ01-01 |
| CSARDHEAGGYNEQFF | 1842 | TCRBV20-X | TCRBJ02-01 |
| CASSQEHEGYGYTF | 1843 | TCRBV03-01/03-02 | TCRBJ01-02 |
| CSVGTGLASYEQYF | 1844 | TCRBV29-01 | TCRBJ02-07 |
| CSARPLRDRTYGYTF | 1845 | TCRBV20-X | TCRBJ01-02 |
| CASSIEGTENEKLFF | 1846 | TCRBV19-01 | TCRBJ01-04 |
| CASSVEGASREKLFF | 1847 | TCRBV09-01 | TCRBJ01-04 |
| CASSPPSHEQFF | 1848 | TCRBV03-01/03-02 | TCRBJ02-01 |
| CASSDPGTGGYGYTF | 1849 | TCRBV28-01 | TCRBJ01-02 |
| CASSQERSTNYGYTF | 1850 | TCRBV04-01 | TCRBJ01-02 |
| CASSGRTGGNYEQYF | 1851 | TCRBV19-01 | TCRBJ02-07 |
| CASSDLTSGNTIYF | 1852 | TCRBV19-01 | TCRBJ01-03 |
| CASSLVDSSYEQYF | 1853 | TCRBV11-X | TCRBJ02-07 |
| CASSFLARTGELFF | 1854 | TCRBV27-01 | TCRBJ02-02 |
| CASSLGHRGTGELFF | 1855 | TCRBV11-03 | TCRBJ02-02 |
| CASSPRLAGETQYF | 1856 | TCRBV07-03 | TCRBJ02-05 |
| CASSPLGRQETQYF | 1857 | TCRBV05-06 | TCRBJ02-05 |
| CASSLKGRHYGYTF | 1858 | TCRBV28-01 | TCRBJ01-02 |
| CASSSPTGGGQPQHF | 1859 | TCRBV12-X | TCRBJ01-05 |
| CASSPRGEGETQYF | 1860 | TCRBV28-01 | TCRBJ02-05 |
| CASSLLATAYEQYF | 1861 | TCRBV05-01 | TCRBJ02-07 |
| CASSHGVDTEAFF | 1862 | TCRBV14-01 | TCRBJ01-01 |
| CSATGLPNSPLHF | 1863 | TCRBV20-X | TCRBJ01-06 |
| CASSLVGTGGLAKNIQYF | 1864 | TCRBV28-01 | TCRBJ02-04 |
| CASSWSRSYEQYF | 1865 | TCRBV07-02 | TCRBJ02-07 |
| CASTLGLYNSPLHF | 1866 | TCRBV28-01 | TCRBJ01-06 |
| CASVGQGNGYTF | 1867 | TCRBV25-01 | TCRBJ01-02 |
| CASSSRYLNTEAFF | 1868 | TCRBV05-01 | TCRBJ01-01 |
| CASSIFPGQGLETQYF | 1869 | TCRBV19-01 | TCRBJ02-05 |
| CASSIGKQGDQPQHF | 1870 | TCRBV19-01 | TCRBJ01-05 |
| CASSLEGTVTDTQYF | 1871 | TCRBV11-01 | TCRBJ02-03 |
| CASSPTRTGMNTEAFF | 1872 | TCRBV28-01 | TCRBJ01-01 |
| CASRAGGSSPLHF | 1873 | TCRBV12-03/12-04 | TCRBJ01-06 |
| CASSPRQGQGNQPQHF | 1874 | TCRBV06-X | TCRBJ01-05 |
| CASSSPGGNNEQFF | 1875 | TCRBV19-01 | TCRBJ02-01 |
| CSARPGRNGYTF | 1876 | TCRBV20-X | TCRBJ01-02 |
| CASGPGDIQYF | 1877 | TCRBV12-03/12-04 | TCRBJ02-04 |
| CATSLFSYNEQFF | 1878 | TCRBV15-01 | TCRBJ02-01 |
| CASSVQGNIQYF | 1879 | TCRBV06-X | TCRBJ02-04 |
| CASSSDRLLSPLHF | 1880 | TCRBV07-06 | TCRBJ01-06 |
| CASKRGTANQETQYF | 1881 | TCRBV12-X | TCRBJ02-05 |
| CASNPPGGGYTF | 1882 | TCRBV28-01 | TCRBJ01-02 |
| CASNRGIQETQYF | 1883 | TCRBV03-01/03-02 | TCRBJ02-05 |
| CASSSPGEKNIQYF | 1884 | TCRBV19-01 | TCRBJ02-04 |
| CASSYSRGNHEQYF | 1885 | TCRBV06-05 | TCRBJ02-07 |
| CASSYSPPAGIYNEQFF | 1886 | TCRBV06-02/06-03 | TCRBJ02-01 |
| CASSFGGSGGETQYF | 1887 | TCRBV05-01 | TCRBJ02-05 |
| CASSYSPLAGIYNEQFF | 1888 | TCRBV06-X | TCRBJ02-01 |
| CASSPQQGYEQYF | 1889 | TCRBV07-02 | TCRBJ02-07 |
| CASSYSGAATNEKLFF | 1890 | TCRBV05-01 | TCRBJ01-04 |
| CASRLGASNQPQHF | 1891 | TCRBV07-08 | TCRBJ01-05 |
| CASSLEQYNSPLHF | 1892 | TCRBV07-09 | TCRBJ01-06 |
| CASSLVMGPWNEQFF | 1893 | TCRBV07-06 | TCRBJ02-01 |
| CAIRYSDVYNEQFF | 1894 | TCRBV10-03 | TCRBJ02-01 |
| CASSKTDNYGYTF | 1895 | TCRBV05-01 | TCRBJ01-02 |
| CASGLGAYEQYF | 1896 | TCRBV06-05 | TCRBJ02-07 |
| CASRTGTTSYEQYF | 1897 | TCRBV27-01 | TCRBJ02-07 |
| CASSLSADYQPQHF | 1898 | TCRBV27-01 | TCRBJ01-05 |
| CASSEAQRGSYNEQFF | 1899 | TCRBV06-01 | TCRBJ02-01 |
| CASSYSRDGQETQYF | 1900 | TCRBV06-05 | TCRBJ02-05 |
| CSARDDRVGNTIYF | 1901 | TCRBV20-X | TCRBJ01-03 |
| CASRGTLRNTEAFF | 1902 | TCRBV04-01 | TCRBJ01-01 |
| CSARDQAYQPQHF | 1903 | TCRBV20-X | TCRBJ01-05 |
| CASSQPGDTDTQYF | 1904 | TCRBV19-01 | TCRBJ02-03 |
| CASSSGPAGGYTF | 1905 | TCRBV27-01 | TCRBJ01-02 |
| CASSAWTATNEKLFF | 1906 | TCRBV06-X | TCRBJ01-04 |
| CASSYDRAGGTEAFF | 1907 | TCRBV06-05 | TCRBJ01-01 |
| CASSPTSTNEKLFF | 1908 | TCRBV04-01 | TCRBJ01-04 |
| CASSYGQPSNYGYTF | 1909 | TCRBV06-02/06-03 | TCRBJ01-02 |
| CASSRVNEQYF | 1910 | TCRBV06-05 | TCRBJ02-07 |
| CASSAWTDQPQHF | 1911 | TCRBV03-01/03-02 | TCRBJ01-05 |
| CASSPTTTDTQYF | 1912 | TCRBV07-08 | TCRBJ02-03 |
| CASSPLGTGGDTQYF | 1913 | TCRBV18-01 | TCRBJ02-03 |
| CAWMRGLSYEQYF | 1914 | TCRBV30-01 | TCRBJ02-07 |
| CASSQDRQSGQPQHF | 1915 | TCRBV03-01/03-02 | TCRBJ01-05 |
| CAWRLQYNEQFF | 1916 | TCRBV30-01 | TCRBJ02-01 |
| CASSLSGHNGYTF | 1917 | TCRBV12-03/12-04 | TCRBJ01-02 |
| CAISERGARDTQYF | 1918 | TCRBV10-03 | TCRBJ02-03 |
| CASTTAGGPRELFF | 1919 | TCRBV09-01 | TCRBJ02-02 |
| CASRKDRGSNQPQHF | 1920 | TCRBV28-01 | TCRBJ01-05 |
| CASSLSGTGTNYGYTF | 1921 | TCRBV28-01 | TCRBJ01-02 |
| CASSPVVTEAFF | 1922 | TCRBV07-09 | TCRBJ01-01 |
| CASSSSGGGNEKLFF | 1923 | TCRBV05-04 | TCRBJ01-04 |
| CASSALDSNYGYTF | 1924 | TCRBV19-01 | TCRBJ01-02 |
| CASSLQEKLFF | 1925 | TCRBV19-01 | TCRBJ01-04 |
| CASSGTGYYGYTF | 1926 | TCRBV27-01 | TCRBJ01-02 |
| CASSPPQGSSYNSPLHF | 1927 | TCRBV06-06 | TCRBJ01-06 |
| CSALRGSDTQYF | 1928 | TCRBV20-X | TCRBJ02-03 |
| CSATSGRARELFF | 1929 | TCRBV20-X | TCRBJ02-02 |
| CASSPRTGNTIYF | 1930 | TCRBV28-01 | TCRBJ01-03 |
| CASSHGLMNTEAFF | 1931 | TCRBV03-01/03-02 | TCRBJ01-01 |
| CASSRGLAGNYEQYF | 1932 | TCRBV04-03 | TCRBJ02-07 |
| CASSLKRGMNTEAFF | 1933 | TCRBV27-01 | TCRBJ01-01 |
| CASSQVPGYSGNTIYF | 1934 | TCRBV14-01 | TCRBJ01-03 |
| CASSPTAGSTEAFF | 1935 | TCRBV18-01 | TCRBJ01-01 |
| CASSKGQGNTEAFF | 1936 | TCRBV28-01 | TCRBJ01-01 |
| CASSQGTGTSGYTF | 1937 | TCRBV04-01 | TCRBJ01-02 |
| CSARDGRGGDGYTF | 1938 | TCRBV20-X | TCRBJ01-02 |
| CASSLMGANVLTF | 1939 | TCRBV12-03/12-04 | TCRBJ02-06 |
| CSAPTGRGSYNEQFF | 1940 | TCRBV20-X | TCRBJ02-01 |
| CASSEDRAGNTEAFF | 1941 | TCRBV02-01 | TCRBJ01-01 |
| CASSEDRVDGYTF | 1942 | TCRBV02-01 | TCRBJ01-02 |
| CASSQEPPYEQYF | 1943 | TCRBV14-01 | TCRBJ02-07 |
| CASSRDRGRSTEAFF | 1944 | TCRBV18-01 | TCRBJ01-01 |
| CASSSTGRPNTGELFF | 1945 | TCRBV05-01 | TCRBJ02-02 |
| CASSDRGKNQPQHF | 1946 | TCRBV06-01 | TCRBJ01-05 |
| CASSKEVQETQYF | 1947 | TCRBV07-03 | TCRBJ02-05 |
| CASSPNLGGYEQYF | 1948 | TCRBV05-01 | TCRBJ02-07 |
| CASSAHGNTEAFF | 1949 | TCRBV02-01 | TCRBJ01-01 |
| CASSLAIGYGYTF | 1950 | TCRBV07-08 | TCRBJ01-02 |
| CASSFASETQYF | 1951 | TCRBV05-04 | TCRBJ02-05 |
| CASSSATGNQPQHF | 1952 | TCRBV11-02 | TCRBJ01-05 |
| CASSLEFNEKLFF | 1953 | TCRBV28-01 | TCRBJ01-04 |
| CASSLRAASNTEAFF | 1954 | TCRBV05-01 | TCRBJ01-01 |
| CASSQASEGYGYTF | 1955 | TCRBV03-01/03-02 | TCRBJ01-02 |
| CASSQGTRNSPLHF | 1956 | TCRBV04-03 | TCRBJ01-06 |
| CASRETGNTEAFF | 1957 | TCRBV07-08 | TCRBJ01-01 |
| CSAGGSSGYNEQFF | 1958 | TCRBV20-X | TCRBJ02-01 |
| CASSIGVLRTEAFF | 1959 | TCRBV19-01 | TCRBJ01-01 |
| CASSFRGKANYGYTF | 1960 | TCRBV27-01 | TCRBJ01-02 |
| CASSSQVGYEQYF | 1961 | TCRBV12-03/12-04 | TCRBJ02-07 |
| CASSPSGPNEQFF | 1962 | TCRBV11-03 | TCRBJ02-01 |
| CASSETGGSNQPQHF | 1963 | TCRBV11-02 | TCRBJ01-05 |
| CASSLRQGDSEQYF | 1964 | TCRBV11-03 | TCRBJ02-07 |
| CSASGDLYNEQFF | 1965 | TCRBV20-01 | TCRBJ02-01 |
| CASSFGGRWETQYF | 1966 | TCRBV05-06 | TCRBJ02-05 |
| CASSQVGDKNIQYF | 1967 | TCRBV19-01 | TCRBJ02-04 |
| CASSLVTGGLGYTF | 1968 | TCRBV07-09 | TCRBJ01-02 |
| CASSRQPSGNTIYF | 1969 | TCRBV05-01 | TCRBJ01-03 |
| CASSLDPGGRAYEQYF | 1970 | TCRBV05-01 | TCRBJ02-07 |
| CASSPLDRVTEAFF | 1971 | TCRBV14-01 | TCRBJ01-01 |
| CASSGEGSYNSPLHF | 1972 | TCRBV12-X | TCRBJ01-06 |
| CSALRTSGRANEQFF | 1973 | TCRBV20-X | TCRBJ02-01 |
| CSAQFGGNQPQHF | 1974 | TCRBV20-X | TCRBJ01-05 |
| CASSPDRGYNSPLHF | 1975 | TCRBV06-04 | TCRBJ01-06 |
| CSARVDRLNYGYTF | 1976 | TCRBV20-X | TCRBJ01-02 |
| CASSIQGSHYEQYF | 1977 | TCRBV19-01 | TCRBJ02-07 |
| CASSLTGLGNQPQHF | 1978 | TCRBV09-01 | TCRBJ01-05 |
| CSAVRDSLYGYTF | 1979 | TCRBV20-X | TCRBJ01-02 |
| CSARDRAGFNSPLHF | 1980 | TCRBV20-X | TCRBJ01-06 |
| CASSPIFSNQPQHF | 1981 | TCRBV12-X | TCRBJ01-05 |
| CASSLERGQGQPQHF | 1982 | TCRBV07-02 | TCRBJ01-05 |
| CSARGAAHQPQHF | 1983 | TCRBV20-X | TCRBJ01-05 |
| CASSEGPPAGELFF | 1984 | TCRBV09-01 | TCRBJ02-02 |
| CASSPGQSGSYNSPLHF | 1985 | TCRBV05-04 | TCRBJ01-06 |
| CASSLGGDTYEQYF | 1986 | TCRBV05-06 | TCRBJ02-07 |
| CASSPVTNYGYTF | 1987 | TCRBV19-01 | TCRBJ01-02 |
| CASSQQLSGNTIYF | 1988 | TCRBV14-01 | TCRBJ01-03 |
| CASSLTDRRDSPLHF | 1989 | TCRBV05-01 | TCRBJ01-06 |
| CAWSFRGSYEQYF | 1990 | TCRBV30-01 | TCRBJ02-07 |
| CASSSRQGGSNQPQHF | 1991 | TCRBV07-02 | TCRBJ01-05 |
| CASRSEYSNQPQHF | 1992 | TCRBV28-01 | TCRBJ01-05 |
| CSVLAGVETQYF | 1993 | TCRBV20-X | TCRBJ02-05 |
| CASSPPAGDHEKLFF | 1994 | TCRBV18-01 | TCRBJ01-04 |
| CASSEHHSNQPQHF | 1995 | TCRBV10-02 | TCRBJ01-05 |
| CASSPDRGFNQPQHF | 1996 | TCRBV02-01 | TCRBJ01-05 |

In Tables 1 and 2 herein, the amino acid sequence represents the TCRβ CDR3 segment of the TCR, while V ##-#4 or J #4-#4 refers to a standard two level coding system [family]-[gene] for a particular part of the human genome that can be used as part of a TCR rearrangement formed in response to antigen exposure. The first two digits reflect a member of a family and the second two digits reflect a particular gene from within that family if present. So, by way of example, TCRBV06 would indicate a match of sequence to a specific family of variable (V) chain sequences where TCRBV06-05 indicates a more precise identification to a specific gene from within a family of variable chain sequences.

Identities of these V- and J-gene sequences can be found at the international ImMunoGeneTics information system (http://www.imgt.org), including at www.imgt.org/download/V-QUEST/IMGT_V-QUEST_reference_directory/Homo_sapiens/TR/TRBV.fasta.

### Therapeutic Methods

Also provided by the present disclosure are therapeutic methods. According to some embodiments, provided are methods comprising administering a Crohn's disease therapy to a subject identified as comprising T cells that express a T cell receptor β chain (TCRβ) comprising a TCRβ CDR3 sequence set forth in SEQ ID Nos:1-1996, e.g., SEQ ID Nos:1-1281. In certain embodiments, the methods comprise administering a Crohn's disease therapy to a subject identified as comprising T cells that express two or more (e.g., two or more unique) TCRβ comprising a TCRβ CDR3 sequence set forth in SEQ ID Nos:1-1996, e.g., SEQ ID Nos:1-1281. According to some embodiments, the methods comprise administering a Crohn's disease therapy to a subject identified using a model/classifier as described elsewhere herein as having Crohn's disease. Such models include, but are not limited to, those that employ a two feature logistic regression with features representing the number of Crohn's disease-associated TCRβ CDR3 sequences determined from the sample and the total number of unique TCRβ CDR3 sequences determined from the sample. As demonstrated in the Experimental section below, such a model exhibits high specificity and sensitivity for Crohn's disease patients. In certain embodiments, the model may take into account the number of unique Crohn's disease-associated TCRβ CDR3 sequences that are present in the TCRβ CDR3 sequences determined from the sample, e.g., where the greater the number of unique Crohn's disease-associated TCRβ CDR3 sequences, the more likely the model is to classify the subject as having Crohn's disease. According to some embodiments, the number of unique Crohn's disease-associated TCRβ CDR3 sequences is not a feature utilized by the model to classify the subject. In certain embodiments, the presence and/or frequency of one or more particular unique Crohn's disease-associated TCRβ CDR3 sequences is a feature(s) used by the model to classify the subject. For example, the presence and/or frequency of one or more particular unique Crohn's disease-associated TCRβ CDR3 sequences may be given relatively greater weight when classifying the subject as compared to the presence and/or frequency of one or more other unique Crohn's disease-associated TCRβ CDR3 sequences. According to some embodiments, when a classification model weighs particular unique Crohn's disease-associated TCRβ CDR3 sequences differently than other unique Crohn's disease-associated TCRβ CDR3 sequences, the model may use convergent recombination to weigh the sequences differently, as described elsewhere herein.

Any suitable Crohn's disease therapy may be administered to a subject identified as described above. Crohn's disease therapies are known and may vary depending upon the age of the patient, stage of the disease, and/or the like. In certain embodiments, the Crohn's disease therapy comprises administering a therapeutically effective amount of an anti-inflammatory drug to the subject. Non-limiting examples of anti-inflammatory drugs that find use in treating Crohn's disease include corticosteroids (e.g., prednisone, budesonide, or a combination thereof) and 5-aminosalicylates, e.g., sulfasalazine, mesalamine, or a combination thereof. According to some embodiments, the Crohn's disease therapy comprises administering a therapeutically effective amount of an immunosuppressant to the subject. Immunosuppressants that find use in treating Crohn's disease include, but are not limited to, azathioprine, mercaptopurine, methotrexate, or any combination thereof. In certain embodiments, the Crohn's disease therapy comprises administering a therapeutically effective amount of a monoclonal antibody to the subject. Non-limiting examples of monoclonal antibodies that find use in treating Crohn's disease include natalizumab, vedolizumab, infliximab, adalimumab, certolizumab pegol, ustekinumab, or any combination thereof. According to some embodiments, the Crohn's disease therapy comprises administering a therapeutically effective amount of an antibiotic (e.g., ciprofloxacin, metronidazole, or a combination thereof) to the subject. In certain embodiments, the Crohn's disease therapy comprises surgery. According to some embodiments, the surgery is adapted for Crohn's disease and not ulcerative colitis. In certain embodiments, the surgery is adapted for Crohn's disease and not irritable bowel syndrome. According to some embodiments, the surgery is adapted for Crohn's disease and not celiac disease. In certain embodiments, the surgery is adapted for Crohn's disease with fistulation or structuring and not Crohn's disease without fistulation or structuring. According to some embodiments, the surgery is adapted for ileal/ileocolonic Crohn's disease and not colonic Crohn's disease.

In certain embodiments, prior to administering the Crohn's disease therapy to the subject, the methods comprise identifying the subject as having Crohn's disease and not ulcerative colitis based upon the subject being identified as comprising T cells that express one or more TCRβ comprising one or more TCRβ CDR3 sequences set forth in SEQ ID Nos:1-1996 or SEQ ID Nos:1-1281. According to some embodiments, the Crohn's disease therapy (e.g., medication and/or surgery) is a therapy adapted for Crohn's disease and not ulcerative colitis.

According to some embodiments, prior to administering the Crohn's disease therapy to the subject, the methods comprise identifying the subject as having Crohn's disease and not irritable bowel syndrome based upon the subject being identified as comprising T cells that express one or more TCRβ comprising one or more TCRβ CDR3 sequences set forth in SEQ ID Nos:1-1996 or SEQ ID Nos:1-1281. In certain embodiments, the Crohn's disease therapy (e.g., medication and/or surgery) is a therapy adapted for Crohn's disease and not irritable bowel syndrome.

In certain embodiments, prior to administering the Crohn's disease therapy to the subject, the methods comprise identifying the subject as having Crohn's disease and not celiac disease based upon the subject being identified as comprising T cells that express one or more TCRβ comprising one or more TCRβ CDR3 sequences set forth in SEQ ID Nos:1-1996 or SEQ ID Nos:1-1281. According to some embodiments, the Crohn's disease therapy (e.g., medication and/or surgery) is a therapy adapted for Crohn's disease and not celiac disease.

According to some embodiments, prior to administering the Crohn's disease therapy to the subject, the methods comprise identifying the subject as having Crohn's disease with fistulation or structuring and not Crohn's disease without fistulation or structuring based upon the subject being identified as comprising T cells that express one or more TCRβ comprising one or more TCRβ CDR3 sequences set forth in SEQ ID Nos:1-1996 or SEQ ID Nos:1-1281. In certain embodiments, the Crohn's disease therapy (e.g., medication and/or surgery) is a therapy adapted for Crohn's disease with fistulation or structuring and not Crohn's disease without fistulation or structuring.

In certain embodiments, prior to administering the Crohn's disease therapy to the subject, the methods comprise identifying the subject as having ileal/ileocolonic Crohn's disease and not colonic Crohn's disease based upon the subject being identified as comprising T cells that express one or more TCRβ comprising one or more TCRβ CDR3 sequences set forth in SEQ ID Nos:1-1996 or SEQ ID Nos:1-1281. According to some embodiments, the Crohn's disease therapy (e.g., medication and/or surgery) is a therapy adapted for ileal/ileocolonic Crohn's disease and not colonic Crohn's disease.

A variety of therapies for treatment of Crohn's disease (including specific treatment of Crohn's disease or a subtype thereof and not ulcerative colitis, irritable bowel syndrome, or celiac disease; and including specific treatment of a particular Crohn's disease subtype, e.g., Crohn's disease with fistulation or structuring and not Crohn's disease without fistulation or structuring, or ileal/ileocolonic Crohn's disease and not colonic Crohn's disease) are known and described, e.g., in Gade et al. (2020) Cureus 12(5):e8351; Cushing & Higgins (2021) JAMA 325(1):69-80; Shi & Ng (2018) J Gastroenterol. 53(9): 989-998; and Sulz et al. (2020) Digestion 101 Suppl 1:43-57 (DOI: 10.1159/000506364); the disclosures of which are incorporated herein by reference in their entireties for all purposes.

According to some embodiments, the methods are effective in treating the Crohn's disease of the individual. By “treat” or “treatment” is meant at least an amelioration of the symptoms associated with the Crohn's disease (e.g., diarrhea, fatigue, abdominal pain, abdominal cramping, rectal bleeding, unintended weight loss, or any combination thereof), where amelioration is used in a broad sense to refer to at least a reduction in the magnitude of a parameter, e.g., symptom, associated with the Crohn's disease being treated. As such, treatment also includes situations where the Crohn's disease, or at least symptoms associated therewith, are completely inhibited, e.g., prevented from happening, or stopped, e.g., terminated, such that the individual no longer suffers from the Crohn's disease, or at least the symptoms that characterize the Crohn's disease.

Dosing may be dependent on severity and responsiveness of the disease state to be treated. Optimal dosing schedules can be calculated from measurements of drug accumulation in the body of the individual. The administering physician can determine optimum dosages, dosing methodologies and repetition rates. Optimum dosages may vary depending on the relative potency of individual therapeutic agents, and can generally be estimated based on EC₅₀s found to be effective in in vitro and in vivo animal models, etc. In general, dosage is from about 0.01 μg to about 100 g per kg of body weight, and may be given once or more daily, weekly, monthly or yearly. In certain aspects, the dosage is from about 1 μg/kg to 100 mg/kg or more, depending on the factors mentioned above. The treating physician can estimate repetition rates for dosing based on measured residence times and concentrations of the therapeutic agent in bodily fluids or tissues. Following successful treatment, it may be desirable to have the subject undergo maintenance therapy to prevent the recurrence of the disease state, where the therapeutic agent is administered in maintenance doses, ranging from about 0.01 μg to about 100 g per kg of body weight, once or more daily, to once every several months, once every six months, once every year, or at any other suitable frequency.

The therapeutic methods of the present disclosure may include administering a single type of therapeutic agent to the subject, or may include administering two or more types of therapeutic agents to the subject separately or by administration of a cocktail of different therapeutic agents. For example, in certain embodiments, two or more therapeutic agents that find use in treating Crohn's disease described elsewhere herein (e.g., anti-inflammatory drug, immunosuppressant, monoclonal antibody, and/or antibiotic) may be administered to the subject, e.g., two or more, three or more, four or more, or five or more of such therapeutic agents.

The one or more therapeutic agents may be administered to the subject using any available method and route suitable for drug delivery, including in vivo and ex vivo methods, as well as systemic and localized routes of administration. Conventional and pharmaceutically acceptable routes of administration include oral and parenteral routes of administration. Parenteral routes of administration of interest include, but are not limited to, injection (e.g., intravenous, intra-arterial, local, subcutaneous, or intramuscular injection), intranasal, intra-tracheal, intradermal, topical application, ocular, nasal, and other parenteral routes of administration. Routes of administration may be combined, if desired, or adjusted depending upon the therapeutic agent and/or the desired effect. The therapeutic agent may be administered in a single dose or in multiple doses. In some embodiments, the therapeutic agent is administered intravenously. In some embodiments, the therapeutic agent is administered by injection, e.g., for systemic delivery (e.g., intravenous infusion) or to a local site.

A “therapeutically effective amount” or “efficacious amount” refers to the amount of a therapeutic agent that, when administered to a mammal or other subject for treating a disease, is sufficient to effect such treatment for the disease. The “therapeutically effective amount” will vary depending on the therapeutic agent, the disease and its severity and the age, weight, etc., of the subject to be treated.

In some embodiments, the Crohn's disease therapy is an adoptive cell therapy. Non-limiting examples of adoptive cell therapies include those involving administering to the subject an effective amount of recombinant cells (e.g., recombinant immune cells such as T cells) that express a T cell receptor comprising a Crohn's disease-associated TCRβ CDR3 sequence identified as being present in TCRs expressed by T cells in the subject. Similar to CAR therapies, TCR therapies modify the patient's T lymphocytes ex vivo before being administered back into the patient's body. The target antigens identified by CAR-T cell therapy are all cell surface proteins, while TCR-T cell therapy can recognize intracellular antigen fragments presented by MHC molecules, so TCR-T cell therapy has a wider range of targets. Approaches for TCR therapy are known and described in, e.g., Zhang et al. (2019) Technol Cancer Res Treat. 18:1533033819831068; Govers et al. (2010) Trends in Molecular Medicine 16(2):77-87; Zhao et al. (2019) Front. Immunol. 10:2250.

Nucleic acids that encode a T cell receptor β chain comprising a TCRβ CDR3 sequence set forth in SEQ ID Nos:1-1996 (e.g., SEQ ID Nos:1-1281) are also provided. For example, in certain embodiments, provided is an expression vector comprising a nucleic acid sequence that encodes a T cell receptor β chain comprising a TCRβ CDR3 sequence set forth in SEQ ID Nos:1-1996 (e.g., SEQ ID Nos:1-1281) operably linked to a nucleic acid expression control sequence. A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

In order to express a desired T cell receptor β chain comprising a TCRβ CDR3 sequence set forth in SEQ ID Nos:1-1996 (e.g., SEQ ID Nos:1-1281), a nucleotide sequence encoding the T cell receptor β chain can be inserted into an appropriate vector, e.g., using recombinant DNA techniques known in the art. Exemplary viral vectors include, without limitation, retrovirus (including lentivirus), adenovirus, adeno-associated virus, herpesvirus (e.g., herpes simplex virus), poxvirus, papillomavirus, and papovavirus (e.g., SV40). Illustrative examples of expression vectors include, but are not limited to pClneo vectors (Promega) for expression in mammalian cells; pLenti4/V 5-DEST™, pLenti6/V 5-DEST™, murine stem cell virus (MSCV), MSGV, moloney murine leukemia virus (MMLV), and pLenti6.2/V5-GW/lacZ (Invitrogen) for lentivirus-mediated gene transfer and expression in mammalian cells. In certain embodiments, a nucleic acid sequence encoding the T cell receptor β chain may be ligated into any such expression vectors for the expression of the T cell receptor β chain in mammalian cells.

Expression control sequences, control elements, or regulatory sequences present in an expression vector are those non-translated regions of the vector—origin of replication, selection cassettes, promoters, enhancers, translation initiation signals (Shine Dalgamo sequence or Kozak sequence), introns, a polyadenylation sequence, 5′ and 3′ untranslated regions, and/or the like—which interact with host cellular proteins to carry out transcription and translation. Such elements may vary in their strength and specificity. Depending on the vector system and host utilized, any number of suitable transcription and translation elements, including ubiquitous promoters and inducible promoters may be used.

Components of the expression vector are operably linked such that they are in a relationship permitting them to function in their intended manner. In some embodiments, the term refers to a functional linkage between a nucleic acid expression control sequence (such as a promoter, and/or enhancer) and a second polynucleotide sequence, e.g., a nucleic acid encoding the T cell receptor β chain, where the expression control sequence directs transcription of the nucleic acid encoding the T cell receptor β chain.

In some embodiments, the expression vector is an episomal vector or a vector that is maintained extrachromosomally. As used herein, the term “episomal” refers to a vector that is able to replicate without integration into the host cell's chromosomal DNA and without gradual loss from a dividing host cell also meaning that said vector replicates extrachromosomally or episomally. Such a vector may be engineered to harbor the sequence coding for the origin of DNA replication or “ori” from an alpha, beta, or gamma herpesvirus, an adenovirus, SV40, a bovine papilloma virus, a yeast, or the like. The host cell may include a viral replication transactivator protein that activates the replication. Alpha herpes viruses have a relatively short reproductive cycle, variable host range, efficiently destroy infected cells and establish latent infections primarily in sensory ganglia. Illustrative examples of alpha herpes viruses include HSV 1, HSV 2, and VZV. Beta herpesviruses have long reproductive cycles and a restricted host range. Infected cells often enlarge. Non-limiting examples of beta herpes viruses include CMV, HHV-6 and HHV-7. Gamma-herpesviruses are specific for either T or B lymphocytes, and latency is often demonstrated in lymphoid tissue. Illustrative examples of gamma herpes viruses include EBV and HHV-8.

Also provided are recombinant cells that comprise any of the expression vectors of the present disclosure comprising a nucleic acid that encodes a T cell receptor β chain comprising a TCRβ CDR3 sequence set forth in SEQ ID Nos:1-1996, e.g., SEQ ID Nos:1-1281. In certain aspects, provided are cells that express a TCR comprising a T cell receptor β chain comprising a TCRβ CDR3 sequence set forth in SEQ ID Nos:1-1996 (e.g., SEQ ID Nos:1-1281) on the surface of the cell.

In some embodiments, the cells of the present disclosure are eukaryotic cells. Eukaryotic cells of interest include, but are not limited to, yeast cells, insect cells, mammalian cells, and the like. Mammalian cells of interest include, e.g., murine cells, non-human primate cells, human cells, and the like.

“Recombinant host cells,” “host cells,” “cells,” “cell lines,” “cell cultures,” and other such terms denoting microorganisms or higher eukaryotic cell lines, refer to cells which can be, or have been, used as recipients for a recombinant vector or other transferred DNA, and include the progeny of the cell which has been transfected. Host cells may be cultured as unicellular or multicellular entities (e.g., tissue, organs, or organoids) including an expression vector of the present disclosure.

In one aspect, the cells provided herein include immune cells. Non-limiting examples of recombinant immune cells which may include any of the expression vectors of the present disclosure include T cells, B cells, natural killer (NK) cells, macrophages, monocytes, neutrophils, dendritic cells, mast cells, basophils, and eosinophils. In some embodiments, the immune cell is a T cell. Examples of T cells include naive T cells (T_{N}), cytotoxic T cells (T_{CTL}), memory T cells (T_{MEM}), T memory stem cells (T_{SCM}), central memory T cells (T_{CM}), effector memory T cells (T_{EM}), tissue resident memory T cells (T_{RM}), effector T cells (T_{EFF}), regulatory T cells (T_{REGs}), helper T cells (T_{H}, T_{H}1, T_{H}2, T_{H}17) CD4+ T cells, CD8+ T cells, virus-specific T cells, alpha beta T cells (T_{αβ}), and gamma delta T cells (T_{γδ}). In another aspect, the cells provided herein comprise stem cells, e.g., an embryonic stem cell or an adult stem cell.

Also provided are methods of making the cells of the present disclosure. In some embodiments, such methods include transfecting or transducing cells with a nucleic acid or expression vector of the present disclosure, e.g., an expression vector comprising a nucleic acid that encodes a T cell receptor β chain comprising a TCRβ CDR3 sequence set forth in SEQ ID Nos:1-1996, e.g., SEQ ID Nos:1-1281. The term “transfection” or “transduction” is used to refer to the introduction of foreign DNA into a cell. A cell has been “transfected” when exogenous DNA has been introduced inside the cell membrane. A number of transfection techniques are generally known in the art. See, e.g., Sambrook et al. (2001) Molecular Cloning, a laboratory manual, 3^{rd} edition, Cold Spring Harbor Laboratories, New York, Davis et al. (1995) Basic Methods in Molecular Biology, 2nd edition, McGraw- Hill, and Chu et al. (1981) Gene 13:197. Such techniques can be used to introduce one or more exogenous DNA moieties into suitable host cells. The term refers to both stable and transient uptake of the genetic material.

In some embodiments, a cell of the present disclosure is produced by transfecting the cell with a viral vector encoding the T cell receptor β chain comprising a TCRβ CDR3 sequence set forth in SEQ ID Nos:1-1996, e.g., SEQ ID Nos:1-1281. In some embodiments, such methods include activating a population of T cells (e.g., T cells obtained from an individual to whom a TCR T cell therapy will be administered), stimulating the population of T cells to proliferate, and transducing the T cell with a viral vector encoding the T cell receptor β chain comprising a TCRβ CDR3 sequence set forth in SEQ ID Nos:1-1996, e.g., SEQ ID Nos:1-1281. In some embodiments, the T cells are transduced with a retroviral vector, e.g., a gamma retroviral vector or a lentiviral vector, encoding the T cell receptor β chain comprising a TCRβ CDR3 sequence set forth in SEQ ID Nos:1-1996, e.g., SEQ ID Nos:1-1281. In some embodiments, the T cells are transduced with a lentiviral vector encoding the T cell receptor β chain comprising a TCRβ CDR3 sequence set forth in SEQ ID Nos:1-1996, e.g., SEQ ID Nos:1-1281.

Cells of the present disclosure may be autologous/autogeneic (“self”) or non-autologous (“non-self,” e.g., allogeneic, syngeneic or xenogeneic). “Autologous” as used herein, refers to cells from the same individual. “Allogeneic” as used herein refers to cells of the same species that differ genetically from the cell in comparison. “Syngeneic,” as used herein, refers to cells of a different individual that are genetically identical to the cell in comparison. In some embodiments, the cells are T cells obtained from a mammal. In some embodiments, the mammal is a primate. In some embodiments, the primate is a human.

T cells may be obtained from a number of sources including, but not limited to, peripheral blood, peripheral blood mononuclear cells, bone marrow, lymph node tissue, cord blood, thymus tissue, tissue from a site of infection, ascites, pleural effusion, spleen tissue, and tumors. In certain embodiments, T cells can be obtained from a unit of blood collected from an individual using any number of known techniques such as sedimentation, e.g., FICOLL™ separation.

In some embodiments, an isolated or purified population of T cells is used. In some embodiments, T_{CTL} and T_{H} lymphocytes are purified from PBMCs. In some embodiments, the T_{CTL} and T_{H} lymphocytes are sorted into naïve (T_{N}), memory (T_{MEM}), and effector (T_{EFF}) T cell subpopulations either before or after activation, expansion, and/or genetic modification. Suitable approaches for such sorting are known and include, e.g., magnetic-activated cell sorting (MACS), where TN are CD45RA⁺ CD62L⁺ CD95⁻; TSCM are CD45RA⁺ CD62L⁺ CD95⁺; TCM are CD45RO⁺ CD62L⁺ CD95⁺; and TEM are CD45RO⁺ CD62L⁻ CD95⁺. An example approach for such sorting is described in Wang et al. (2016) Blood 127(24):2980-90.

A specific subpopulation of T cells expressing one or more of the following markers: CD3, CD4, CD8, CD28, CD45RA, CD45RO, CD62, CD127, and HLA-DR can be further isolated by positive or negative selection techniques. In some embodiments, a specific subpopulation of T cells, expressing one or more of the markers selected from the group consisting of CD62L, CCR7, CD28, CD27, CD122, CD127, CD197; or CD38 or CD62L, CD127, CD197, and CD38, is further isolated by positive or negative selection techniques. In some embodiments, the manufactured T cell compositions do not express one or more of the following markers: CD57, CD244, CD 160, PD-1, CTLA4, TIM3, and LAG3. In some embodiments, the manufactured T cell compositions do not substantially express one or more of the following markers: CD57, CD244, CD 160, PD-1, CTLA4, TIM3, and LAG3.

In order to achieve therapeutically effective doses of T cell compositions, the T cells may be subjected to one or more rounds of stimulation, activation and/or expansion. T cells can be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; and 6,867,041, each of which is incorporated herein by reference in its entirety for all purposes. In some embodiments, T cells are activated and expanded for about 1 to 21 days, e.g., about 5 to 21 days. In some embodiments, T cells are activated and expanded for about 1 day to about 4 days, about 1 day to about 3 days, about 1 day to about 2 days, about 2 days to about 3 days, about 2 days to about 4 days, about 3 days to about 4 days, or about 1 day, about 2 days, about 3 days, or about 4 days prior to introduction of a nucleic acid (e.g., expression vector) encoding the polypeptide into the T cells.

In some embodiments, T cells are activated and expanded for about 6 hours, about 12 hours, about 18 hours or about 24 hours prior to introduction of a nucleic acid (e.g., expression vector) encoding the T cell receptor β chain comprising a TCRβ CDR3 sequence set forth in SEQ ID Nos:1-1996 (e.g., SEQ ID Nos:1-1281) into the T cells. In some embodiments, T cells are activated at the same time that a nucleic acid (e.g., an expression vector) encoding the T cell receptor β chain is introduced into the T cells.

In some embodiments, conditions appropriate for T cell culture include an appropriate media (e.g., Minimal Essential Media or RPMI Media 1640 or, X-vivo 15, (Lonza)) and one or more factors necessary for proliferation and viability including, but not limited to serum (e.g., fetal bovine or human serum), interleukin-2 (IL-2), insulin, IFN-γ, IL-4, IL-7, IL-21, GM-CSF, IL-10, IL-12, IL-15, TGFβ, and TNF-α or any other additives suitable for the growth of cells known to the skilled artisan. Further illustrative examples of cell culture media include, but are not limited to RPMI 1640, Clicks, AEVI-V, DMEM, MEM, a-MEM, F-12, X-Vivo 15, and X-Vivo 20, Optimizer, with added amino acids, sodium pyruvate, and vitamins, either serum-free or supplemented with an appropriate amount of serum (or plasma) or a defined set of hormones, and/or an amount of cytokine(s) sufficient for the growth and expansion of T cells.

In some embodiments, the nucleic acid (e.g., an expression vector) encoding the T cell receptor β chain is introduced into the cell (e.g., a T cell) by microinjection, transfection, lipofection, heat-shock, electroporation, transduction, gene gun, microinjection, DEAE-dextran-mediated transfer, and the like. In some embodiments, the nucleic acid (e.g., expression vector) encoding the T cell receptor β chain is introduced into the cell (e.g., a T cell) by AAV transduction. The AAV vector may comprise ITRs from AAV2, and a serotype from any one of AAV1, AAV2, AAV3, AAV4, AAV5, AAV6, AAV7, AAV8, AAV9, or AAV 10. In some embodiments, the AAV vector comprises ITRs from AAV2 and a serotype from AAV6. In some embodiments, the nucleic acid (e.g., expression vector) encoding the T cell receptor β chain is introduced into the cell (e.g., a T cell) by lentiviral transduction. The lentiviral vector backbone may be derived from HIV-1, HIV-2, visna-maedi virus (VMV) virus, caprine arthritis-encephalitis virus (CAEV), equine infectious anemia virus (EIAV), feline immunodeficiency virus (FIV), bovine immune deficiency virus (BIV), or simian immunodeficiency virus (SIV). The lentiviral vector may be integration competent or an integrase deficient lentiviral vector (TDLV). In one embodiment, IDLV vectors including an HIV-based vector backbone (i.e., HIV cis-acting sequence elements) are employed.

### Computer-Readable Media and Systems

Also provided by the present disclosure are computer-readable media and systems.

In certain aspects, provided are one or more computer-readable media having stored thereon one or more TCRβ CDR3 sequences set forth in SEQ ID Nos:1-1996, e.g., SEQ ID Nos:1-1281. The number of TCRβ CDR3 sequences set forth in SEQ ID Nos:1-1996 (e.g., SEQ ID Nos:1-1281) stored on the one or more computer-readable media may vary. For example, the one or more computer-readable media may have stored thereon 1 or more, 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 75 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1000 or more, 1100 or more, 1200 or more, or each of the TCRβ CDR3 sequences set forth in SEQ ID Nos:1-1996, e.g., SEQ ID Nos:1-1281. When fewer than all of the TCRβ CDR3 sequences set forth in SEQ ID Nos:1-1996 (e.g., SEQ ID Nos:1-1281) are stored on the one or more computer-readable media, the one or more computer-readable media may have stored thereon any desired number (e.g., as set forth above) and combination of TCRβ CDR3 sequences set forth in SEQ ID Nos:1-1996, e.g., SEQ ID Nos:1-1281. In some embodiments, the one or more computer-readable media may have stored thereon 1996 or fewer, 1281 or fewer, 1000 or fewer, 950 or fewer, 900 or fewer, 850 or fewer, 800 or fewer, 750 or fewer, 700 or fewer, 650 or fewer, 600 or fewer, 550 or fewer, 500 or fewer, 450 or fewer, 400 or fewer, 350 or fewer, 300 or fewer, 250 or fewer, 200 or fewer, 190 or fewer, 180 or fewer, 170 or fewer, 160 or fewer, 150 or fewer, 140 or fewer, 130 or fewer, 120 or fewer, 110 or fewer, 100 or fewer, 90 or fewer, 80 or fewer, 70 or fewer, 60 or fewer, 50 or fewer, 40 or fewer, 30 or fewer, 20 or fewer, or 10 or fewer of the TCRβ CDR3 sequences set forth in SEQ ID Nos:1-1996 (e.g., SEQ ID Nos:1-1281), in any desired combination.

Also provided are systems for assessing TCRβ CDR3 sequences. According to some embodiments, provided are systems for assessing TCRβ CDR3 sequences, such systems comprising one or more processors and one or more computer-readable media. The one or more computer-readable media comprise instructions stored thereon, which when executed by the one or more processors, cause the one or more processors to assess TCRβ CDR3 sequences determined from a sample obtained from a subject (e.g., a subject identified as having IB or suspected of having IBD, including but not limited to a subject exhibiting one or more non-specific symptoms consistent with Crohn's disease) for the presence or absence of one or more TCRβ CDR3 sequences set forth in SEQ ID Nos:1-1996, e.g., SEQ ID Nos:1-1281. According to some embodiments, the number of TCRβ CDR3 sequences determined from the sample obtained from the subject is from 1,000 to 2,000,000. For example, in certain embodiments, the number of determined TCRβ CDR3 sequences is 2,000,000 or fewer (e.g., 1,500,000 or fewer, 1,250,000 or fewer, 1,000,000 or fewer, 750,000 or fewer, or 500,000 or fewer), but 1,000 or more, 5,000 or more, 10,000 or more, 15,000 or more, 20,000 or more, 25,000 or more, 30,000 or more, 35,000 or more, 40,000 or more, 45,000 or more, 50,000 or more, 55,000 or more, 60,000 or more, 65,000 or more, 70,000 or more, 75,000 or more, 80,000 or more, 85,000 or more, 90,000 or more, 95,000 or more, or 100,000 or more. The number of TCRβ CDR3 sequences set forth in SEQ ID Nos:1-1996 (e.g., SEQ ID Nos:1-1281) to which the determined TCRβ CDR3 sequences is compared may vary. For example, the determined TCRβ CDR3 sequences may be compared to 1 or more, 2 or more, 3 or more, 4 or more, 5 or more, 6 or more, 7 or more, 8 or more, 9 or more, 10 or more, 15 or more, 20 or more, 25 or more, 30 or more, 35 or more, 40 or more, 45 or more, 50 or more, 75 or more, 100 or more, 150 or more, 200 or more, 250 or more, 300 or more, 350 or more, 400 or more, 450 or more, 500 or more, 550 or more, 600 or more, 650 or more, 700 or more, 750 or more, 800 or more, 850 or more, 900 or more, 950 or more, 1000 or more, 1010 or more, 1020 or more, 1030 or more, or each of the TCRβ CDR3 sequences set forth in SEQ ID Nos:1-1996, e.g., SEQ ID Nos:1-1281. When the determined TCRβ CDR3 sequences are compared to fewer than all of the TCRβ CDR3 sequences set forth in SEQ ID Nos:1-1996 (e.g., SEQ ID Nos:1-1281), the determined TCRβ CDR3 sequences may be compared to any desired number (e.g., as set forth above) and combination of TCRβ CDR3 sequences set forth in SEQ ID Nos:1-1996, e.g., SEQ ID Nos:1-1281.

The one or more computer-readable media may further comprise instructions stored thereon, which when executed by the one or more processors, cause the one or more processors to perform one or more additional steps based on the results of the assessing step. For example, if it is determined from the assessing step that none of the TCRβ CDR3 sequences set forth in SEQ ID Nos:1-1996 (e.g., SEQ ID Nos:1-1281) are present in the TCRβ CDR3 sequences determined from the sample obtained from the subject, then the instructions may further cause the one or more processors to, e.g., identify the subject as not having Crohn's disease, identify the subject as one who should not be administered a Crohn's disease therapy, and/or the like. Also, by way of example, if it is determined from the assessing step that one or more (e.g., 2 or more, 3 or more, 4 or more, 5 or more, or 10 or more) of the TCRβ CDR3 sequences set forth in SEQ ID Nos:1-1996 (e.g., SEQ ID Nos:1-1281) are present in the TCRβ CDR3 sequences determined from the sample obtained from the subject (e.g., a subject identified as having IB or suspected of having IBD, including but not limited to a subject exhibiting one or more non-specific symptoms consistent with Crohn's disease), then the instructions may further cause the one or more processors to, e.g., predict that the subject has Crohn's disease, diagnose the subject as having Crohn's disease, identify the subject as one who should be administered a Crohn's disease therapy, and/or the like.

In certain embodiments, the one or more computer-readable media may further comprise instructions stored thereon, which when executed by the one or more processors, cause the one or more processors to subject the results of the assessing step to further analysis, such as subjecting the results of the assessing step to a model. For example, the instructions may cause the one or more processors to subject the results of the assessing step to a model in order to classify the subject as having Crohn's disease or not having Crohn's disease; and/or to classify the subject as having Crohn's disease and not having a non-Crohn's disease IBD, e.g., ulcerative colitis. One of ordinary skill in the art will appreciate that, with the benefit of the TCRβ CDR3 sequences set forth in SEQ ID Nos:1-1996 (e.g., SEQ ID Nos:1-1281) described herein, a variety of useful models may be applied to the results of the assessment. In one non-limiting example, the instructions may cause the one or more processors to subject the results of the assessing step to a two feature logistic regression with features representing the number of Crohn's disease-associated TCRβ CDR3 sequences determined from the sample and the total number of unique TCRβ CDR3 sequences determined from the sample. As demonstrated in the Experimental section below, such a model exhibits high specificity and sensitivity for Crohn's disease.

In certain embodiments, when the one or more computer-readable media further comprise instructions stored thereon, which when executed by the one or more processors, cause the one or more processors to subject the results of the assessing step to a model for classification purposes (e.g., as described above), the model may take into account the number of unique Crohn's disease-associated TCRβ CDR3 sequences that are present in the TCRβ CDR3 sequences determined from the sample, e.g., where the greater the number of unique Crohn's disease-associated TCRβ CDR3 sequences, the more likely the model is to classify the subject as having Crohn's disease. According to some embodiments, the number of unique Crohn's disease-associated TCRβ CDR3 sequences is not a feature utilized by the model to classify the subject. In certain embodiments, the presence and/or frequency of one or more particular unique Crohn's disease-associated TCRβ CDR3 sequences is a feature(s) used by the model to classify the subject. For example, the presence and/or frequency of one or more particular unique Crohn's disease-associated TCRβ CDR3 sequences may be given relatively greater weight when classifying the subject as compared to the presence and/or frequency of one or more other unique Crohn's disease-associated TCRβ CDR3 sequences.

A variety of processor-based systems may be employed to implement the embodiments of the present disclosure. Such systems may include system architecture wherein the components of the system are in electrical communication with each other using a bus. System architecture can include a processing unit (CPU or processor), as well as a cache, that are variously coupled to the system bus. The bus couples various system components including system memory, (e.g., read only memory (ROM) and random access memory (RAM), to the processor.

System architecture can include a cache of high-speed memory connected directly with, in close proximity to, or integrated as part of the processor. System architecture can copy data from the memory and/or the storage device to the cache for quick access by the processor. In this way, the cache can provide a performance boost that avoids processor delays while waiting for data. These and other modules can control or be configured to control the processor to perform various actions. Other system memory may be available for use as well. Memory can include multiple different types of memory with different performance characteristics. Processor can include any general purpose processor and a hardware module or software module, such as first, second and third modules stored in the storage device, configured to control the processor as well as a special-purpose processor where software instructions are incorporated into the actual processor design. The processor may essentially be a completely self-contained computing system, containing multiple cores or processors, a bus, memory controller, cache, etc. A multi-core processor may be symmetric or asymmetric.

To enable user interaction with the computing system architecture, an input device can represent any number of input mechanisms, such as a microphone for speech, a touch-sensitive screen for gesture or graphical input, keyboard, mouse, motion input, speech and so forth. An output device can also be one or more of a number of output mechanisms. In some instances, multimodal systems can enable a user to provide multiple types of input to communicate with the computing system architecture. A communications interface can generally govern and manage the user input and system output. There is no restriction on operating on any particular hardware arrangement and therefore the basic features here may easily be substituted for improved hardware or firmware arrangements as they are developed.

The storage device is typically a non-volatile memory and can be a hard disk or other types of computer-readable media which can store data that are accessible by a computer, such as magnetic cassettes, flash memory cards, solid state memory devices, digital versatile disks, cartridges, random access memories (RAMs), read only memory (ROM), and hybrids thereof.

The storage device can include software modules for controlling the processor. Other hardware or software modules are contemplated. The storage device can be connected to the system bus. In one aspect, a hardware module that performs a particular function can include the software component stored in a computer-readable medium in connection with the necessary hardware components, such as the processor, bus, output device, and so forth, to carry out various functions of the disclosed technology.

Embodiments within the scope of the present disclosure may also include tangible and/or non-transitory computer-readable storage media or devices for carrying or having computer- executable instructions or data structures stored thereon. Such tangible computer-readable storage devices can be any available device that can be accessed by a general purpose or special purpose computer, including the functional design of any special purpose processor as described above. By way of example, and not limitation, such tangible computer-readable devices can include RAM, ROM, EEPROM, CD-ROM or other optical disk storage, magnetic disk storage or other magnetic storage devices, or any other device which can be used to carry or store desired program code in the form of computer-executable instructions, data structures, or processor chip design. When information or instructions are provided via a network or another communications connection (either hardwired, wireless, or combination thereof) to a computer, the computer properly views the connection as a computer-readable medium. Thus, any such connection is properly termed a computer-readable medium. Combinations of the above should also be included within the scope of the computer-readable storage devices.

Computer-executable instructions include, for example, instructions and data which cause a general purpose computer, special purpose computer, or special purpose processing device to perform a certain function or group of functions. Computer-executable instructions also include program modules that are executed by computers in stand-alone or network environments. Generally, program modules include routines, programs, components, data structures, objects, and the functions inherent in the design of special-purpose processors, etc. that perform tasks or implement abstract data types. Computer-executable instructions, associated data structures, and program modules represent examples of the program code means for executing steps of the methods disclosed herein. The particular sequence of such executable instructions or associated data structures represents examples of corresponding acts for implementing the functions described in such steps.

Other embodiments of the disclosure may be practiced in network computing environments with many types of computer system configurations, including personal computers, hand-held devices, multi-processor systems, microprocessor-based or programmable consumer electronics, network PCs, minicomputers, mainframe computers, and the like. Embodiments may also be practiced in distributed computing environments where tasks are performed by local and remote processing devices that are linked (either by hardwired links, wireless links, or by a combination thereof) through a communications network. In a distributed computing environment, program modules may be located in both local and remote memory storage devices.

Notwithstanding the appended claims, the present disclosure is also defined by the following embodiments:
1. A computer-implemented method for assessing T cell receptor β chain complementary determining region 3 (TCRβ CDR3) sequences, the method comprising:
   assessing TCRβ CDR3 sequences determined from a sample obtained from a subject for the presence or absence of one or more TCRβ CDR3 sequences set forth in SEQ ID Nos:1-1996.
2. The computer-implemented method of embodiment 1, comprising assessing TCRβ CDR3 sequences determined from the sample obtained from the subject for the presence or absence of one or more TCRβ CDR3 sequences set forth in SEQ ID Nos:1-1281.
3. The computer-implemented method of embodiment 1 or embodiment 2, wherein prior to the assessing, the subject has been identified as having, or is suspected of having, inflammatory bowel disease (IBD).
4. The computer-implemented method of any one of embodiments 1 to 3, wherein the subject has one or more non-specific symptoms consistent with Crohn's disease at the time of the assessing.
5. The computer-implemented method of embodiment 4, wherein the one or more non-specific symptoms are selected from the group consisting of: diarrhea, fatigue, abdominal pain, abdominal cramping, rectal bleeding, unintended weight loss, and any combination thereof.
6. The computer-implemented method of any one of embodiments 1 to 5, wherein the TCRβ CDR3 sequences determined from the sample obtained from the subject comprise 10,000 or more TCRβ CDR3 sequences.
7. The computer-implemented method of any one of embodiments 1 to 6, wherein the TCRβ CDR3 sequences determined from the sample obtained from the subject were determined by performing amplification and high throughput sequencing of genomic DNA present in the sample obtained from the subject.
8. The computer-implemented method of any one of embodiments 1 to 7, wherein the sample obtained from the subject is a peripheral blood sample.
9. The computer-implemented method of any one of embodiments 1 to 7, wherein the sample obtained from the subject is a gut tissue sample.
10. A method comprising administering a Crohn's disease therapy to a subject identified as comprising T cells that express a T cell receptor β chain (TCRβ) comprising a TCRβ CDR3 sequence set forth in SEQ ID Nos:1-1996.
11. The method of embodiment 10, comprising administering the Crohn's disease therapy to a subject identified as comprising T cells that express a TCRβ comprising a TCRβ CDR3 sequence set forth in SEQ ID Nos:1-1281.
12. The method of embodiment 10 or embodiment 11, wherein the Crohn's disease therapy comprises administering a therapeutically effective amount of an anti-inflammatory drug to the subject.
13. The method of embodiment 12, wherein the Crohn's disease therapy comprises administering a therapeutically effective amount of a corticosteroid to the subject.
14. The method of embodiment 13, wherein the corticosteroid is prednisone, budesonide, or a combination thereof.
15. The method of any one of embodiments 10 to 14, wherein the Crohn's disease therapy comprises administering a therapeutically effective amount of a 5-aminosalicylate to the subject.
16. The method of embodiment 15, wherein the 5-aminosalicylate is sulfasalazine, mesalamine, or a combination thereof.
17. The method of any one of embodiments 10 to 16, wherein the Crohn's disease therapy comprises administering a therapeutically effective amount of an immunosuppressant to the subject.
18. The method of embodiment 17, wherein the immunosuppressant is azathioprine, mercaptopurine, methotrexate, or any combination thereof.
19. The method of any one of embodiments 10 to 18, wherein the Crohn's disease therapy comprises administering a therapeutically effective amount of a monoclonal antibody to the subject.
20. The method of embodiment 19, wherein the monoclonal antibody is natalizumab, vedolizumab, infliximab, adalimumab, certolizumab pegol, ustekinumab, or any combination thereof.
21. The method of any one of embodiments 10 to 20, wherein the Crohn's disease therapy comprises administering a therapeutically effective amount of an antibiotic to the subject.
22. The method of embodiment 10 or embodiment 11, further comprising, prior to administering the Crohn's disease therapy to the subject, identifying the subject as having Crohn's disease and not ulcerative colitis based upon the subject being identified as comprising T cells that express one or more TCRβ comprising one or more TCRβ CDR3 sequences set forth in SEQ ID Nos:1-1996 or SEQ ID Nos:1-1281.
23. The method of embodiment 10 or embodiment 11, further comprising, prior to administering the Crohn's disease therapy to the subject, identifying the subject as having Crohn's disease and not irritable bowel syndrome based upon the subject being identified as comprising T cells that express one or more TCRβ comprising one or more TCRβ CDR3 sequences set forth in SEQ ID Nos:1-1996 or SEQ ID Nos:1-1281.
24. The method of embodiment 10 or embodiment 11, further comprising, prior to administering the Crohn's disease therapy to the subject, identifying the subject as having Crohn's disease and not celiac disease based upon the subject being identified as comprising T cells that express one or more TCRβ comprising one or more TCRβ CDR3 sequences set forth in SEQ ID Nos:1-1996 or SEQ ID Nos:1-1281.
25. The method according to any one of embodiments 22 to 24, wherein the Crohn's disease therapy is a therapy adapted for Crohn's disease and not ulcerative colitis, irritable bowel syndrome, and/or celiac disease.
26. The method of embodiment 10 or embodiment 11, further comprising, prior to administering the Crohn's disease therapy to the subject, identifying the subject as having Crohn's disease with fistulation or structuring and not Crohn's disease without fistulation or structuring based upon the subject being identified as comprising T cells that express one or more TCRβ comprising one or more TCRβ CDR3 sequences set forth in SEQ ID Nos:1-1996 or SEQ ID Nos:1-1281.
27. The method of embodiment 26, wherein the Crohn's disease therapy is a therapy adapted for Crohn's disease with fistulation or structuring and not Crohn's disease without fistulation or structuring.
28. The method of embodiment 10 or embodiment 11, further comprising, prior to administering the Crohn's disease therapy to the subject, identifying the subject as having ileal/ileocolonic Crohn's disease and not colonic Crohn's disease based upon the subject being identified as comprising T cells that express one or more TCRβ comprising one or more TCRβ CDR3 sequences set forth in SEQ ID Nos:1-1996 or SEQ ID Nos:1-1281.
29. The method of embodiment 28, wherein the Crohn's disease therapy is a therapy adapted for ileal/ileocolonic Crohn's disease and not colonic Crohn's disease.
30. A non-transitory computer readable medium having stored thereon one or more TCRβ CDR3 sequences set forth in SEQ ID Nos:1-1996 or SEQ ID Nos:1-1281.
31. The non-transitory computer readable medium of embodiment 30 having stored thereon 5 or more TCRβ CDR3 sequences set forth in SEQ ID Nos:1-1996 or SEQ ID Nos:1-1281.
32. The non-transitory computer readable medium of embodiment 30 having stored thereon 10 or more TCRβ CDR3 sequences set forth in SEQ ID Nos:1-1996 or SEQ ID Nos:1-1281.
33. The non-transitory computer readable medium of embodiment 30 having stored thereon 100 or more TCRβ CDR3 sequences set forth in SEQ ID Nos:1-1996 or SEQ ID Nos:1-1281.
34. A system for assessing TCRβ CDR3 sequences, comprising:
   one or more processors; and
   one or more non-transitory computer-readable media comprising instructions stored thereon, which when executed by the one or more processors, cause the one or more processors to assess TCRβ CDR3 sequences determined from a sample obtained from a subject for the presence or absence of one or more TCRβ CDR3 sequences set forth in SEQ ID Nos:1-1996 or SEQ ID Nos:1-1281.
35. The system of embodiment 34, wherein the TCRβ CDR3 sequences determined from the sample obtained from the subject comprise 10,000 or more TCRβ CDR3 sequences.
36. The system of embodiment 34 or embodiment 35, wherein the instructions, when executed by the one or more processors, cause the one or more processors to assess the determined TCRβ sequences for the presence or absence of 5 or more TCRβ CDR3 sequences set forth in SEQ ID Nos:1-1996 or SEQ ID Nos:1-1281.
37. The system of embodiment 34 or embodiment 35, wherein the instructions, when executed by the one or more processors, cause the one or more processors to assess the determined TCRβ sequences for the presence or absence of 10 or more TCRβ CDR3 sequences set forth in SEQ ID Nos:1-1996 or SEQ ID Nos:1-1281.
38. The system of embodiment 34 or embodiment 35, wherein the instructions, when executed by the one or more processors, cause the one or more processors to assess the determined TCRβ sequences for the presence or absence of 100 or more TCRβ CDR3 sequences set forth in SEQ ID Nos:1-1996 or SEQ ID Nos:1-1281.

The following examples are offered by way of illustration and not by way of limitation.

### EXPERIMENTAL

### Example 1—Prediction of Crohn's Disease from TCR Repertoire Data

Described in this example is the identification of Crohn's disease-associated TCRβ sequences, sometimes referred to herein as “enhanced sequences for Crohn's disease”, “enhanced sequences”, or the like.

T-cell receptor repertoires were generated by immunosequencing of whole blood samples, peripheral blood mononuclear cells (PBMC), or buffy coat preparations of blood. Briefly, genomic DNA was extracted from the blood or cell samples using standard extraction kits. As much as 18 μg of genomic DNA was then input into a multiplex PCR reaction to amplify the CDR3 regions of TCRβ chains followed by high-throughput sequencing (the immunoSEQ Assay as described above).

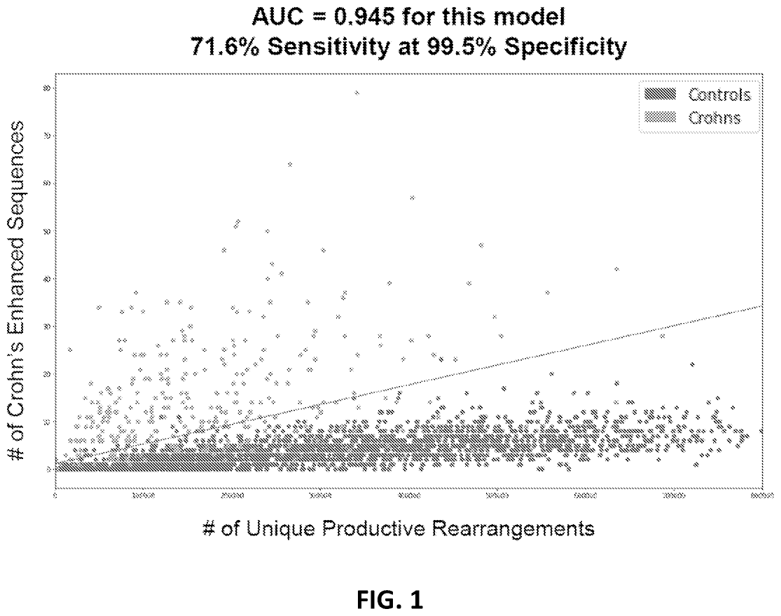

An initial diagnostic model to predict Crohn's Disease from TCR repertoire data was run on blood samples from patients undergoing Crohn's-related surgery from a cohort from Hopital Saint Louis (Paris). The cases in training included 362 Crohn's patients prior to surgical intervention. The controls in train included >1000 healthy adults from other data sets (healthy blood donors collected via contract research organization and other studies). The model first uses one-tailed Fisher's exact tests to identify unique TCR sequences that are elevated in the Crohn's case samples versus the controls. Unique sequences are identified by their V gene, J gene, and TCRβ CDR3 amino acid sequences. A two-feature logistic regression was then performed with dependent variables E and N, where E is the number of unique TCRβ DNA sequences that encode an enhanced sequence and N is the total number of unique TCRβ DNA sequences in that subject. As schematically illustrated in FIG. 1, hundreds of Crohn's disease-associated TCRβ sequences that distinguish Crohn's patients from controls were identified and used in the logistic regression (AUC=0.945 for the model; 71.6% sensitivity at 99.5% specificity).

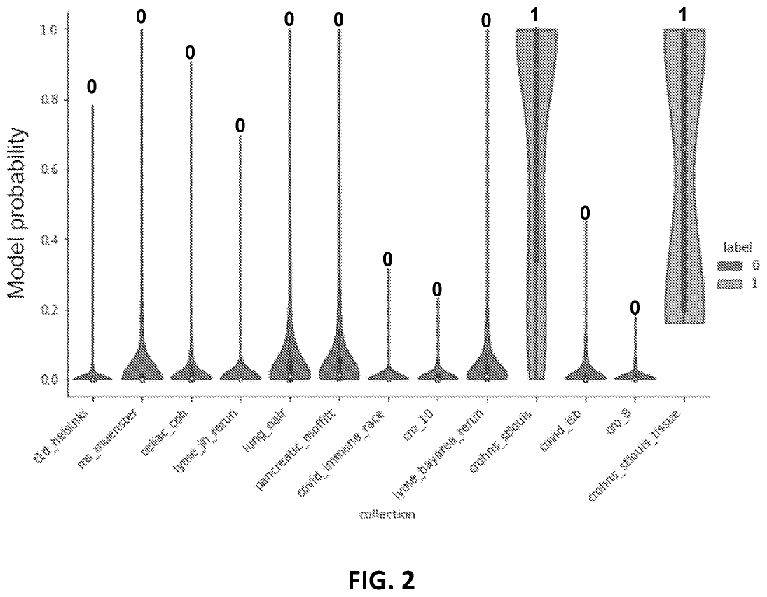

Model performance was then tested on Holdout Data sets not used in training. High model scores (strong signal) were observed only for Crohn's patients and not other healthy/disease groups tested, as shown in FIG. 2.

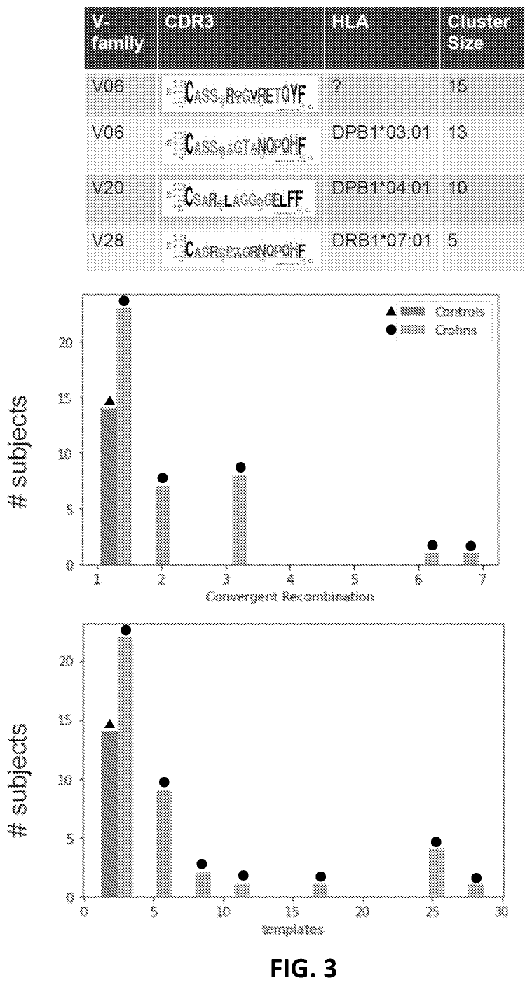

It was determined that the Crohn's disease-associated TCRβ sequences exhibit clusters associated with class II MHC, and high convergent recombination in Crohn's patients but not controls, as shown in FIG. 3.

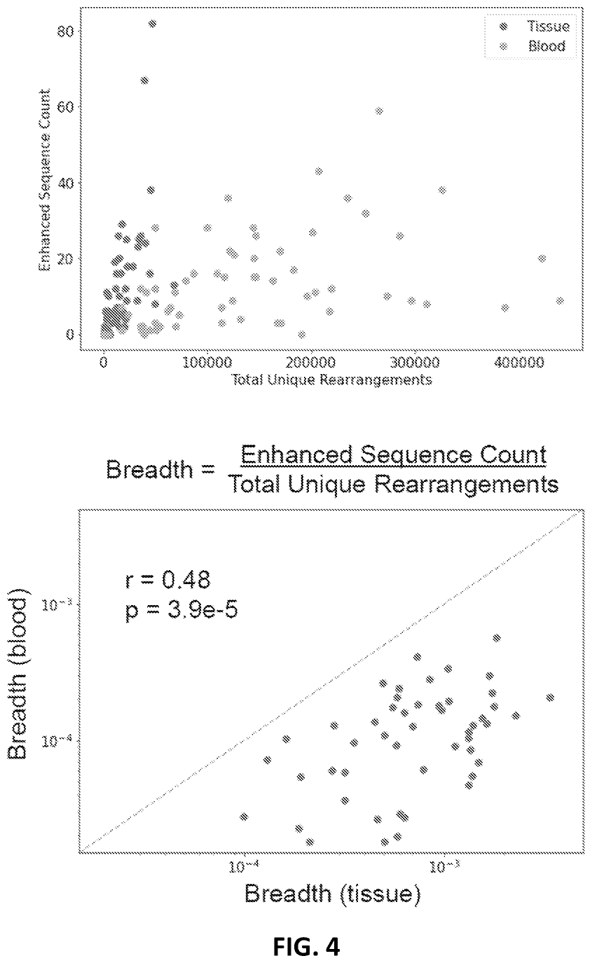

As additional biological validation of these TCR sequences, tissue samples from Crohn's patients at the Hopital Saint Louis were also sequenced using the immunoSEQ Assay. From these tissue-derived repertoires, it was determined that Crohn's disease-associated TCRβ sequences identified in blood are also present and enriched in gut tissue samples compared to blood samples from the same patients. As shown in FIG. 4, a similar number of Crohn's disease-associated TCRβ sequences were observed in blood and gut tissue. The total sequences in tissue samples is small, thus a significantly greater fraction of the sequences in tissue are enhanced sequences. The signal in blood and gut tissue of matched samples was found to be strongly correlated.

A large number of additional clinical samples were then collected and immunosequenced using the ImmunoSEQ Assay to build an improved model. These samples included additional samples from the Hopital Saint Louis as well as from multiple studies performed by the University of Kiel (Germany) and from the Crohn's and Colitis Foundation. A total of 3,890 subjects with Crohn's disease were collected and 80% of these subject samples were used in training (20% left in holdout) to develop a new classifier for Crohn's Disease. Over 4,000 negative controls from other studies of adults (without inflammatory bowel diseases) were also used in training. 1,281 unique TCRβ CDR3 sequences significantly associated with Crohn's Disease were identified (Table 1 herein). This list showed a high degree of overlap from sequences identified in the initial model but added several hundred additional TCRβ sequences. Other models were trained in Crohn's Disease, including different selected cohorts and location-based models for ileal versus colonic disease, that identified other Crohn's associated sequences (see Table 2 herein).

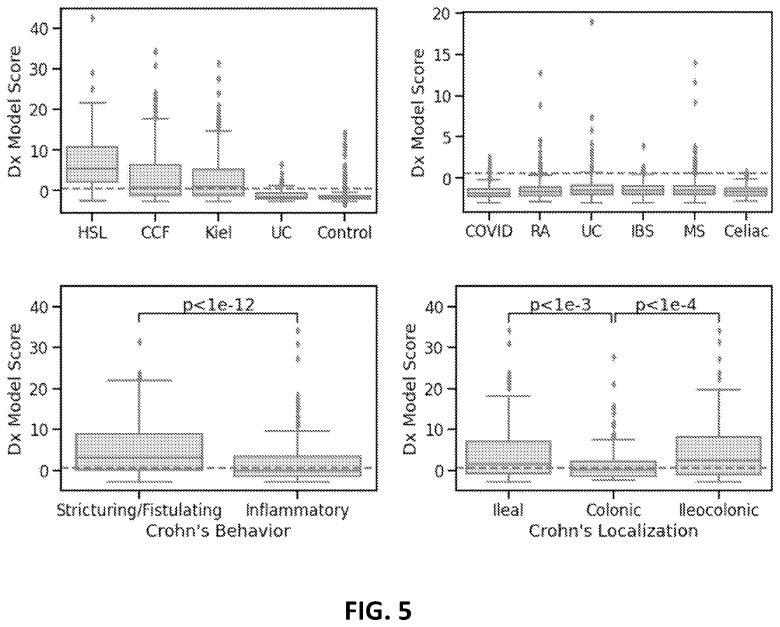

The holdout data from this larger model was tested for performance similar to prior description. As shown in FIG. 5, the sensitivity observed in the holdout data is high across the three main Crohn's cohorts with high specificity against other diseases including the conditions irritable bowel syndrome and celiac disease that also lead to potential gut issues (FIG. 5, upper-right). The model demonstrates significantly higher signal in individuals with more complicated disease with fistulation or stricturing (FIG. 5, Lower-left), and is also elevated in ileal/ileocolonic disease over colonic disease (FIG. 5, Lower-right). Different TCRβ sequences appear to contribute to the ileal versus colonic signals.

Accordingly, the preceding merely illustrates the principles of the present disclosure. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein.

What is claimed is:

1. A computer-implemented method for assessing T cell receptor β chain complementary determining region 3 (TCRβ CDR3) sequences, the method comprising:
assessing TCRβ CDR3 sequences determined from a sample obtained from a subject for the presence or absence of one or more TCRβ CDR3 sequences set forth in SEQ ID Nos:1-1996.

2. The computer-implemented method of claim 1, comprising assessing TCRβ CDR3 sequences determined from the sample obtained from the subject for the presence or absence of one or more TCRβ CDR3 sequences set forth in SEQ ID Nos:1-1281.

3. The computer-implemented method of claim 1 or claim 2, wherein prior to the assessing, the subject has been identified as having, or is suspected of having, inflammatory bowel disease (IBD).

4. The computer-implemented method of any one of claims 1 to 3, wherein the subject has one or more non-specific symptoms consistent with Crohn's disease at the time of the assessing.

5. The computer-implemented method of claim 4, wherein the one or more non-specific symptoms are selected from the group consisting of: diarrhea, fatigue, abdominal pain, abdominal cramping, rectal bleeding, unintended weight loss, and any combination thereof.

6. The computer-implemented method of any one of claims 1 to 5, wherein the TCRβ CDR3 sequences determined from the sample obtained from the subject comprise 10,000 or more TCRβ CDR3 sequences.

7. The computer-implemented method of any one of claims 1 to 6, wherein the TCRβ CDR3 sequences determined from the sample obtained from the subject were determined by performing amplification and high throughput sequencing of genomic DNA present in the sample obtained from the subject.

8. The computer-implemented method of any one of claims 1 to 7, wherein the sample obtained from the subject is a peripheral blood sample.

9. The computer-implemented method of any one of claims 1 to 7, wherein the sample obtained from the subject is a gut tissue sample.

10. A method comprising administering a Crohn's disease therapy to a subject identified as comprising T cells that express a T cell receptor β chain (TCRβ) comprising a TCRβ CDR3 sequence set forth in SEQ ID Nos:1-1996.

11. The method of claim 10, comprising administering the Crohn's disease therapy to a subject identified as comprising T cells that express a TCRβ comprising a TCRβ CDR3 sequence set forth in SEQ ID Nos:1-1281.

12. The method of claim 10 or claim 11, wherein the Crohn's disease therapy comprises administering a therapeutically effective amount of an anti-inflammatory drug to the subject.

13. The method of claim 12, wherein the Crohn's disease therapy comprises administering a therapeutically effective amount of a corticosteroid to the subject.

14. The method of claim 13, wherein the corticosteroid is prednisone, budesonide, or a combination thereof.

15. The method of any one of claims 10 to 14, wherein the Crohn's disease therapy comprises administering a therapeutically effective amount of a 5-aminosalicylate to the subject.

16. The method of claim 15, wherein the 5-aminosalicylate is sulfasalazine, mesalamine, or a combination thereof.

17. The method of any one of claims 10 to 16, wherein the Crohn's disease therapy comprises administering a therapeutically effective amount of an immunosuppressant to the subject.

18. The method of claim 17, wherein the immunosuppressant is azathioprine, mercaptopurine, methotrexate, or any combination thereof.

19. The method of any one of claims 10 to 18, wherein the Crohn's disease therapy comprises administering a therapeutically effective amount of a monoclonal antibody to the subject.

20. The method of claim 19, wherein the monoclonal antibody is natalizumab, vedolizumab, infliximab, adalimumab, certolizumab pegol, ustekinumab, or any combination thereof.

21. The method of any one of claims 10 to 20, wherein the Crohn's disease therapy comprises administering a therapeutically effective amount of an antibiotic to the subject.

22. The method of claim 10 or claim 11, further comprising, prior to administering the Crohn's disease therapy to the subject, identifying the subject as having Crohn's disease and not ulcerative colitis based upon the subject being identified as comprising T cells that express one or more TCRβ comprising one or more TCRβ CDR3 sequences set forth in SEQ ID Nos:1-1996 or SEQ ID Nos:1-1281.

23. The method of claim 10 or claim 11, further comprising, prior to administering the Crohn's disease therapy to the subject, identifying the subject as having Crohn's disease and not irritable bowel syndrome based upon the subject being identified as comprising T cells that express one or more TCRβ comprising one or more TCRβ CDR3 sequences set forth in SEQ ID Nos:1-1996 or SEQ ID Nos:1-1281.

24. The method of claim 10 or claim 11, further comprising, prior to administering the Crohn's disease therapy to the subject, identifying the subject as having Crohn's disease and not celiac disease based upon the subject being identified as comprising T cells that express one or more TCRβ comprising one or more TCRβ CDR3 sequences set forth in SEQ ID Nos:1-1996 or SEQ ID Nos:1-1281.

25. The method according to any one of claims 22 to 24, wherein the Crohn's disease therapy is a therapy adapted for Crohn's disease and not ulcerative colitis, irritable bowel syndrome, and/or celiac disease.

26. The method of claim 10 or claim 11, further comprising, prior to administering the Crohn's disease therapy to the subject, identifying the subject as having Crohn's disease with fistulation or structuring and not Crohn's disease without fistulation or structuring based upon the subject being identified as comprising T cells that express one or more TCRβ comprising one or more TCRβ CDR3 sequences set forth in SEQ ID Nos:1-1996 or SEQ ID Nos:1-1281.

27. The method of claim 26, wherein the Crohn's disease therapy is a therapy adapted for Crohn's disease with fistulation or structuring and not Crohn's disease without fistulation or structuring.

28. The method of claim 10 or claim 11, further comprising, prior to administering the Crohn's disease therapy to the subject, identifying the subject as having ileal/ileocolonic Crohn's disease and not colonic Crohn's disease based upon the subject being identified as comprising T cells that express one or more TCRβ comprising one or more TCRβ CDR3 sequences set forth in SEQ ID Nos:1-1996 or SEQ ID Nos:1-1281.

29. The method of claim 28, wherein the Crohn's disease therapy is a therapy adapted for ileal/ileocolonic Crohn's disease and not colonic Crohn's disease.

30. A non-transitory computer readable medium having stored thereon one or more TCRβ CDR3 sequences set forth in SEQ ID Nos:1-1996 or SEQ ID Nos:1-1281.

31. The non-transitory computer readable medium of claim 30 having stored thereon 5 or more TCRβ CDR3 sequences set forth in SEQ ID Nos:1-1996 or SEQ ID Nos:1-1281.

32. The non-transitory computer readable medium of claim 30 having stored thereon 10 or more TCRβ CDR3 sequences set forth in SEQ ID Nos:1-1996 or SEQ ID Nos:1-1281.

33. The non-transitory computer readable medium of claim 30 having stored thereon 100 or more TCRβ CDR3 sequences set forth in SEQ ID Nos:1-1996 or SEQ ID Nos:1-1281.

34. A system for assessing TCRβ CDR3 sequences, comprising:
one or more processors; and
one or more non-transitory computer-readable media comprising instructions stored thereon, which when executed by the one or more processors, cause the one or more processors to assess TCRβ CDR3 sequences determined from a sample obtained from a subject for the presence or absence of one or more TCRβ CDR3 sequences set forth in SEQ ID Nos:1-1996 or SEQ ID Nos:1-1281.

35. The system of claim 34, wherein the TCRβ CDR3 sequences determined from the sample obtained from the subject comprise 10,000 or more TCRβ CDR3 sequences.

36. The system of claim 34 or claim 35, wherein the instructions, when executed by the one or more processors, cause the one or more processors to assess the determined TCRβ sequences for the presence or absence of 5 or more TCRβ CDR3 sequences set forth in SEQ ID Nos:1-1996 or SEQ ID Nos:1-1281.

37. The system of claim 34 or claim 35, wherein the instructions, when executed by the one or more processors, cause the one or more processors to assess the determined TCRβ sequences for the presence or absence of 10 or more TCRβ CDR3 sequences set forth in SEQ ID Nos:1-1996 or SEQ ID Nos:1-1281.

38. The system of claim 34 or claim 35, wherein the instructions, when executed by the one or more processors, cause the one or more processors to assess the determined TCRβ sequences for the presence or absence of 100 or more TCRβ CDR3 sequences set forth in SEQ ID Nos:1-1996 or SEQ ID Nos:1-1281.


SEQUENCE LISTING (1996 sequences)

SEQ ID NO: 1 - protein, 13 aa, Artificial sequence
  synthetic sequence
CASSTDRAGELFF

SEQ ID NO: 2 - protein, 13 aa, Artificial sequence
  synthetic sequence
CSAPGLGDEKLFF

SEQ ID NO: 3 - protein, 15 aa, Artificial sequence
  synthetic sequence
CATSRDLGTSYEQYF

SEQ ID NO: 4 - protein, 12 aa, Artificial sequence
  synthetic sequence
CASSFSQETQYF

SEQ ID NO: 5 - protein, 15 aa, Artificial sequence
  synthetic sequence
CASSSAGTANQPQHF

SEQ ID NO: 6 - protein, 13 aa, Artificial sequence
  synthetic sequence
CASSSDRAGELFF

SEQ ID NO: 7 - protein, 15 aa, Artificial sequence
  synthetic sequence
CASSSTGTANQPQHF

SEQ ID NO: 8 - protein, 14 aa, Artificial sequence
  synthetic sequence
CAISRGGNQETQYF

SEQ ID NO: 9 - protein, 13 aa, Artificial sequence
  synthetic sequence
CASSSDSPGELFF

SEQ ID NO: 10 - protein, 13 aa, Artificial sequence
  synthetic sequence
CASSSDRPGELFF

SEQ ID NO: 11 - protein, 15 aa, Artificial sequence
  synthetic sequence
CASSPTGTTNQPQHF

SEQ ID NO: 12 - protein, 15 aa, Artificial sequence
  synthetic sequence
CASSQEPSGTYEQYF

SEQ ID NO: 13 - protein, 15 aa, Artificial sequence
  synthetic sequence
CASSIGGGWGQPQHF

SEQ ID NO: 14 - protein, 15 aa, Artificial sequence
  synthetic sequence
CASSQGQGEGYEQYF

SEQ ID NO: 15 - protein, 15 aa, Artificial sequence
  synthetic sequence
CASSATGTANQPQHF

SEQ ID NO: 16 - protein, 14 aa, Artificial sequence
  synthetic sequence
CASRTGLNTGELFF

SEQ ID NO: 17 - protein, 13 aa, Artificial sequence
  synthetic sequence
CSVEGGRPGELFF

SEQ ID NO: 18 - protein, 14 aa, Artificial sequence
  synthetic sequence
CASSQGTEGYGYTF

SEQ ID NO: 19 - protein, 13 aa, Artificial sequence
  synthetic sequence
CASRGEGNTEAFF

SEQ ID NO: 20 - protein, 13 aa, Artificial sequence
  synthetic sequence
CASSLNPPGGYTF

SEQ ID NO: 21 - protein, 17 aa, Artificial sequence
  synthetic sequence
CSATGLAGDSGANVLTF

SEQ ID NO: 22 - protein, 14 aa, Artificial sequence
  synthetic sequence
CASSRRDQYNEQFF

SEQ ID NO: 23 - protein, 15 aa, Artificial sequence
  synthetic sequence
CASSFDRGENYGYTF

SEQ ID NO: 24 - protein, 15 aa, Artificial sequence
  synthetic sequence
CASSLGDRVWETQYF

SEQ ID NO: 25 - protein, 14 aa, Artificial sequence
  synthetic sequence
CASSDRDREGKLFF

SEQ ID NO: 26 - protein, 15 aa, Artificial sequence
  synthetic sequence
CASSVDGTETYEQYF

SEQ ID NO: 27 - protein, 15 aa, Artificial sequence
  synthetic sequence
CASSPPATVNTEAFF

SEQ ID NO: 28 - protein, 16 aa, Artificial sequence
  synthetic sequence
CASSLAVGHLNTEAFF

SEQ ID NO: 29 - protein, 16 aa, Artificial sequence
  synthetic sequence
CASSQEGQGFGNTIYF

SEQ ID NO: 30 - protein, 16 aa, Artificial sequence
  synthetic sequence
CASSLALGHLNTEAFF

SEQ ID NO: 31 - protein, 14 aa, Artificial sequence
  synthetic sequence
CASSLDGTGSEQYF

SEQ ID NO: 32 - protein, 14 aa, Artificial sequence
  synthetic sequence
CASSPGAVNTEAFF

SEQ ID NO: 33 - protein, 16 aa, Artificial sequence
  synthetic sequence
CASSVGGRALYNEQFF

SEQ ID NO: 34 - protein, 14 aa, Artificial sequence
  synthetic sequence
CASRGGQGMTEAFF

SEQ ID NO: 35 - protein, 15 aa, Artificial sequence
  synthetic sequence
CASSYGQGRNYGYTF

SEQ ID NO: 36 - protein, 11 aa, Artificial sequence
  synthetic sequence
CASSEYSPLHF

SEQ ID NO: 37 - protein, 14 aa, Artificial sequence
  synthetic sequence
CASSSGPIQETQYF

SEQ ID NO: 38 - protein, 15 aa, Artificial sequence
  synthetic sequence
CASSSSGTANQPQHF

SEQ ID NO: 39 - protein, 15 aa, Artificial sequence
  synthetic sequence
CASSSRQGRTGELFF

SEQ ID NO: 40 - protein, 12 aa, Artificial sequence
  synthetic sequence
CSALGGVAEAFF

SEQ ID NO: 41 - protein, 13 aa, Artificial sequence
  synthetic sequence
CASSTDSPGELFF

SEQ ID NO: 42 - protein, 13 aa, Artificial sequence
  synthetic sequence
CASSGDRPGELFF

SEQ ID NO: 43 - protein, 14 aa, Artificial sequence
  synthetic sequence
CAISAGDSTDTQYF

SEQ ID NO: 44 - protein, 15 aa, Artificial sequence
  synthetic sequence
CSAPGGGRDNSPLHF

SEQ ID NO: 45 - protein, 14 aa, Artificial sequence
  synthetic sequence
CASSLGSVNTEAFF

SEQ ID NO: 46 - protein, 15 aa, Artificial sequence
  synthetic sequence
CSARDVASGANVLTF

SEQ ID NO: 47 - protein, 15 aa, Artificial sequence
  synthetic sequence
CASSLGTVDNSPLHF

SEQ ID NO: 48 - protein, 13 aa, Artificial sequence
  synthetic sequence
CASSTDRPGELFF

SEQ ID NO: 49 - protein, 17 aa, Artificial sequence
  synthetic sequence
CASSLAGLAGGPDTQYF

SEQ ID NO: 50 - protein, 14 aa, Artificial sequence
  synthetic sequence
CASSLGAVNTEAFF

SEQ ID NO: 51 - protein, 13 aa, Artificial sequence
  synthetic sequence
CSVWTGATDTQYF

SEQ ID NO: 52 - protein, 14 aa, Artificial sequence
  synthetic sequence
CASRSTGYNQPQHF

SEQ ID NO: 53 - protein, 14 aa, Artificial sequence
  synthetic sequence
CASSYGDRNSPLHF

SEQ ID NO: 54 - protein, 13 aa, Artificial sequence
  synthetic sequence
CASRDVDTGELFF

SEQ ID NO: 55 - protein, 15 aa, Artificial sequence
  synthetic sequence
CASSYGEGWNQPQHF

SEQ ID NO: 56 - protein, 15 aa, Artificial sequence
  synthetic sequence
CASSYRQANNQPQHF

SEQ ID NO: 57 - protein, 13 aa, Artificial sequence
  synthetic sequence
CASSPQINYGYTF

SEQ ID NO: 58 - protein, 13 aa, Artificial sequence
  synthetic sequence
CASRDIDTGELFF

SEQ ID NO: 59 - protein, 13 aa, Artificial sequence
  synthetic sequence
CASSLGDRAEAFF

SEQ ID NO: 60 - protein, 14 aa, Artificial sequence
  synthetic sequence
CASRTGVNTGELFF

SEQ ID NO: 61 - protein, 15 aa, Artificial sequence
  synthetic sequence
CASSQEGRSNTEAFF

SEQ ID NO: 62 - protein, 13 aa, Artificial sequence
  synthetic sequence
CASSTDRTGELFF

SEQ ID NO: 63 - protein, 13 aa, Artificial sequence
  synthetic sequence
CASSRGEDSPLHF

SEQ ID NO: 64 - protein, 15 aa, Artificial sequence
  synthetic sequence
CASSLRGGGDYGYTF

SEQ ID NO: 65 - protein, 14 aa, Artificial sequence
  synthetic sequence
CASRGSSGNQPQHF

SEQ ID NO: 66 - protein, 15 aa, Artificial sequence
  synthetic sequence
CASSFNDRGNTEAFF

SEQ ID NO: 67 - protein, 12 aa, Artificial sequence
  synthetic sequence
CASSLGDGEQFF

SEQ ID NO: 68 - protein, 13 aa, Artificial sequence
  synthetic sequence
CASSRLSSYEQYF

SEQ ID NO: 69 - protein, 15 aa, Artificial sequence
  synthetic sequence
CASSLVFGQSYEQYF

SEQ ID NO: 70 - protein, 13 aa, Artificial sequence
  synthetic sequence
CASSPWGGETQYF

SEQ ID NO: 71 - protein, 15 aa, Artificial sequence
  synthetic sequence
CASSPKDRGGEKLFF

SEQ ID NO: 72 - protein, 13 aa, Artificial sequence
  synthetic sequence
CASSQWGGETQYF

SEQ ID NO: 73 - protein, 15 aa, Artificial sequence
  synthetic sequence
CASSYGDRGNQPQHF

SEQ ID NO: 74 - protein, 13 aa, Artificial sequence
  synthetic sequence
CASSQDPQETQYF

SEQ ID NO: 75 - protein, 13 aa, Artificial sequence
  synthetic sequence
CSAWTASYNEQFF

SEQ ID NO: 76 - protein, 14 aa, Artificial sequence
  synthetic sequence
CASSQADSTDTQYF

SEQ ID NO: 77 - protein, 14 aa, Artificial sequence
  synthetic sequence
CASSPGSEGQPQHF

SEQ ID NO: 78 - protein, 12 aa, Artificial sequence
  synthetic sequence
CASRIGEETQYF

SEQ ID NO: 79 - protein, 15 aa, Artificial sequence
  synthetic sequence
CASSQEPSGIYEQYF

SEQ ID NO: 80 - protein, 14 aa, Artificial sequence
  synthetic sequence
CASSERDRGYEQYF

SEQ ID NO: 81 - protein, 15 aa, Artificial sequence
  synthetic sequence
CASSELDRAGNTIYF

SEQ ID NO: 82 - protein, 14 aa, Artificial sequence
  synthetic sequence
CASSYGDKNSPLHF

SEQ ID NO: 83 - protein, 17 aa, Artificial sequence
  synthetic sequence
CASSLAGLAGSPDTQYF

SEQ ID NO: 84 - protein, 12 aa, Artificial sequence
  synthetic sequence
CASSGTGDGYTF

SEQ ID NO: 85 - protein, 15 aa, Artificial sequence
  synthetic sequence
CASSIGSGWGQPQHF

SEQ ID NO: 86 - protein, 13 aa, Artificial sequence
  synthetic sequence
CASRSEGNTEAFF

SEQ ID NO: 87 - protein, 15 aa, Artificial sequence
  synthetic sequence
CASSRQGVGNQPQHF

SEQ ID NO: 88 - protein, 13 aa, Artificial sequence
  synthetic sequence
CASRQGKGTEAFF

SEQ ID NO: 89 - protein, 15 aa, Artificial sequence
  synthetic sequence
CASSPSGGQGDTQYF

SEQ ID NO: 90 - protein, 16 aa, Artificial sequence
  synthetic sequence
CASSSGTGRSNQPQHF

SEQ ID NO: 91 - protein, 15 aa, Artificial sequence
  synthetic sequence
CASSLDLKNTGELFF

SEQ ID NO: 92 - protein, 15 aa, Artificial sequence
  synthetic sequence
CASSYPSRGLETQYF

SEQ ID NO: 93 - protein, 14 aa, Artificial sequence
  synthetic sequence
CASSNSSGANVLTF

SEQ ID NO: 94 - protein, 16 aa, Artificial sequence
  synthetic sequence
CATSDLGVRGTGELFF

SEQ ID NO: 95 - protein, 14 aa, Artificial sequence
  synthetic sequence
CASSFHRDRETQYF

SEQ ID NO: 96 - protein, 13 aa, Artificial sequence
  synthetic sequence
CASSLIRADTQYF

SEQ ID NO: 97 - protein, 15 aa, Artificial sequence
  synthetic sequence
CASSPSGGGEETQYF

SEQ ID NO: 98 - protein, 17 aa, Artificial sequence
  synthetic sequence
CASSPPSGGRNTGELFF

SEQ ID NO: 99 - protein, 16 aa, Artificial sequence
  synthetic sequence
CASSLDRGGSYNEQFF

SEQ ID NO: 100 - protein, 15 aa, Artificial sequence
  synthetic sequence
CASSGRDGNTGELFF

SEQ ID NO: 101 - protein, 16 aa, Artificial sequence
  synthetic sequence
CASSFRLAGPQETQYF

SEQ ID NO: 102 - protein, 13 aa, Artificial sequence
  synthetic sequence
CASSEGGLYGYTF

SEQ ID NO: 103 - protein, 13 aa, Artificial sequence
  synthetic sequence
CASSGDSPGELFF

SEQ ID NO: 104 - protein, 15 aa, Artificial sequence
  synthetic sequence
CASSPSGGLEETQYF

SEQ ID NO: 105 - protein, 13 aa, Artificial sequence
  synthetic sequence
CASSTDRVGEAFF

SEQ ID NO: 106 - protein, 15 aa, Artificial sequence
  synthetic sequence
CASSYSPTGATEAFF

SEQ ID NO: 107 - protein, 16 aa, Artificial sequence
  synthetic sequence
CASSLGRSGSSYEQYF

SEQ ID NO: 108 - protein, 13 aa, Artificial sequence
  synthetic sequence
CASSTDRSGELFF

SEQ ID NO: 109 - protein, 14 aa, Artificial sequence
  synthetic sequence
CASRPDGYNQPQHF

SEQ ID NO: 110 - protein, 11 aa, Artificial sequence
  synthetic sequence
CASHGNSPLHF

SEQ ID NO: 111 - protein, 14 aa, Artificial sequence
  synthetic sequence
CASSLVPGEYGYTF

SEQ ID NO: 112 - protein, 15 aa, Artificial sequence
  synthetic sequence
CASSPRTGSAETQYF

SEQ ID NO: 113 - protein, 15 aa, Artificial sequence
  synthetic sequence
CSARDPREDTGELFF

SEQ ID NO: 114 - protein, 15 aa, Artificial sequence
  synthetic sequence
CASSPSPTANTEAFF

SEQ ID NO: 115 - protein, 15 aa, Artificial sequence
  synthetic sequence
CASSEGDRGNQPQHF

SEQ ID NO: 116 - protein, 16 aa, Artificial sequence
  synthetic sequence
CASSLTGTSGYNEQFF

SEQ ID NO: 117 - protein, 15 aa, Artificial sequence
  synthetic sequence
CASSIGGGQYNEQFF

SEQ ID NO: 118 - protein, 15 aa, Artificial sequence
  synthetic sequence
CASSLSRRYQETQYF

SEQ ID NO: 119 - protein, 17 aa, Artificial sequence
  synthetic sequence
CASSPRTGGSYNSPLHF

SEQ ID NO: 120 - protein, 15 aa, Artificial sequence
  synthetic sequence
CASSESKGGRNEQFF

SEQ ID NO: 121 - protein, 17 aa, Artificial sequence
  synthetic sequence
CASSLARAGGPGDTQYF

SEQ ID NO: 122 - protein, 16 aa, Artificial sequence
  synthetic sequence
CASSVGLAGGGNEQFF

SEQ ID NO: 123 - protein, 16 aa, Artificial sequence
  synthetic sequence
CASSLALAGGPDTQYF

SEQ ID NO: 124 - protein, 16 aa, Artificial sequence
  synthetic sequence
CASSSRLAGAPDTQYF

SEQ ID NO: 125 - protein, 16 aa, Artificial sequence
  synthetic sequence
CSARASGGPTYNEQFF

SEQ ID NO: 126 - protein, 14 aa, Artificial sequence
  synthetic sequence
CASSFRDRATEAFF

SEQ ID NO: 127 - protein, 15 aa, Artificial sequence
  synthetic sequence
CATSFGTGGAGELFF

SEQ ID NO: 128 - protein, 12 aa, Artificial sequence
  synthetic sequence
CASSFGANVLTF

SEQ ID NO: 129 - protein, 15 aa, Artificial sequence
  synthetic sequence
CASSLADRVWETQYF

SEQ ID NO: 130 - protein, 15 aa, Artificial sequence
  synthetic sequence
CASSLGGNSYQPQHF

SEQ ID NO: 131 - protein, 14 aa, Artificial sequence
  synthetic sequence
CAISGGDSTDTQYF

SEQ ID NO: 132 - protein, 15 aa, Artificial sequence
  synthetic sequence
CASSQDRAYNQPQHF

SEQ ID NO: 133 - protein, 14 aa, Artificial sequence
  synthetic sequence
CASSPRGAGDTQYF

SEQ ID NO: 134 - protein, 15 aa, Artificial sequence
  synthetic sequence
CASSPRTGTAETQYF

SEQ ID NO: 135 - protein, 16 aa, Artificial sequence
  synthetic sequence
CASSPPDSPTNEKLFF

SEQ ID NO: 136 - protein, 14 aa, Artificial sequence
  synthetic sequence
CSARGQGETGELFF

SEQ ID NO: 137 - protein, 16 aa, Artificial sequence
  synthetic sequence
CASSPQGTGKGNEQFF

SEQ ID NO: 138 - protein, 15 aa, Artificial sequence
  synthetic sequence
CASRSPRGRNQPQHF

SEQ ID NO: 139 - protein, 15 aa, Artificial sequence
  synthetic sequence
CASSYRQGARQPQHF

SEQ ID NO: 140 - protein, 15 aa, Artificial sequence
  synthetic sequence
CASSLDRGENYGYTF

SEQ ID NO: 141 - protein, 16 aa, Artificial sequence
  synthetic sequence
CASSLVSSGQYNEQFF

SEQ ID NO: 142 - protein, 15 aa, Artificial sequence
  synthetic sequence
CSARDPGEDTGELFF

SEQ ID NO: 143 - protein, 13 aa, Artificial sequence
  synthetic sequence
CASRDADTGELFF

SEQ ID NO: 144 - protein, 13 aa, Artificial sequence
  synthetic sequence
CSARNRSTDTQYF

SEQ ID NO: 145 - protein, 13 aa, Artificial sequence
  synthetic sequence
CSAIGTGSDEQYF

SEQ ID NO: 146 - protein, 15 aa, Artificial sequence
  synthetic sequence
CASRPPTGRNQPQHF

SEQ ID NO: 147 - protein, 14 aa, Artificial sequence
  synthetic sequence
CASSPGSVNTEAFF

SEQ ID NO: 148 - protein, 14 aa, Artificial sequence
  synthetic sequence
CSAPPPGVGNTIYF

SEQ ID NO: 149 - protein, 13 aa, Artificial sequence
  synthetic sequence
CSATGAQADTQYF

SEQ ID NO: 150 - protein, 13 aa, Artificial sequence
  synthetic sequence
CSVKYGSGNTIYF

SEQ ID NO: 151 - protein, 16 aa, Artificial sequence
  synthetic sequence
CASSLGGQGRNYGYTF

SEQ ID NO: 152 - protein, 14 aa, Artificial sequence
  synthetic sequence
CASSLDRAETEAFF

SEQ ID NO: 153 - protein, 13 aa, Artificial sequence
  synthetic sequence
CSVRGQLNTEAFF

SEQ ID NO: 154 - protein, 15 aa, Artificial sequence
  synthetic sequence
CASSPSGTTNQPQHF

SEQ ID NO: 155 - protein, 14 aa, Artificial sequence
  synthetic sequence
CASSPAGGRNEQFF

SEQ ID NO: 156 - protein, 13 aa, Artificial sequence
  synthetic sequence
CSVEGGRPGEQYF

SEQ ID NO: 157 - protein, 13 aa, Artificial sequence
  synthetic sequence
CATKTASTDTQYF

SEQ ID NO: 158 - protein, 12 aa, Artificial sequence
  synthetic sequence
CASSLGDGEQYF

SEQ ID NO: 159 - protein, 15 aa, Artificial sequence
  synthetic sequence
CASSLAPGGHTEAFF

SEQ ID NO: 160 - protein, 14 aa, Artificial sequence
  synthetic sequence
CSVEFLGGADTQYF

SEQ ID NO: 161 - protein, 13 aa, Artificial sequence
  synthetic sequence
CSATGTSDREQYF

SEQ ID NO: 162 - protein, 15 aa, Artificial sequence
  synthetic sequence
CASSYSPTANTEAFF

SEQ ID NO: 163 - protein, 18 aa, Artificial sequence
  synthetic sequence
CASSLGGTGASGANVLTF

SEQ ID NO: 164 - protein, 16 aa, Artificial sequence
  synthetic sequence
CASSFGRAANTGELFF

SEQ ID NO: 165 - protein, 15 aa, Artificial sequence
  synthetic sequence
CASSPSAANNEKLFF

SEQ ID NO: 166 - protein, 14 aa, Artificial sequence
  synthetic sequence
CASSFWQGRNTIYF

SEQ ID NO: 167 - protein, 12 aa, Artificial sequence
  synthetic sequence
CASTSSGNTIYF

SEQ ID NO: 168 - protein, 14 aa, Artificial sequence
  synthetic sequence
CSARWESFYNEQFF

SEQ ID NO: 169 - protein, 15 aa, Artificial sequence
  synthetic sequence
CASSYPSSGRETQYF

SEQ ID NO: 170 - protein, 15 aa, Artificial sequence
  synthetic sequence
CSVEHRGRGKNIQYF

SEQ ID NO: 171 - protein, 14 aa, Artificial sequence
  synthetic sequence
CSARGQGDGQPQHF

SEQ ID NO: 172 - protein, 14 aa, Artificial sequence
  synthetic sequence
CASSLVGRVYGYTF

SEQ ID NO: 173 - protein, 14 aa, Artificial sequence
  synthetic sequence
CAISLGGNQETQYF

SEQ ID NO: 174 - protein, 13 aa, Artificial sequence
  synthetic sequence
CASSGDRAGELFF

SEQ ID NO: 175 - protein, 15 aa, Artificial sequence
  synthetic sequence
CASSQTGTANQPQHF

SEQ ID NO: 176 - protein, 14 aa, Artificial sequence
  synthetic sequence
CASRTGINTGELFF

SEQ ID NO: 177 - protein, 15 aa, Artificial sequence
  synthetic sequence
CSARVGTGNNSPLHF

SEQ ID NO: 178 - protein, 15 aa, Artificial sequence
  synthetic sequence
CASSLKDRDGNTIYF

SEQ ID NO: 179 - protein, 14 aa, Artificial sequence
  synthetic sequence
CASSYKGGADTQYF

SEQ ID NO: 180 - protein, 13 aa, Artificial sequence
  synthetic sequence
CASSTDRAGEAFF

SEQ ID NO: 181 - protein, 15 aa, Artificial sequence
  synthetic sequence
CASSHLRGSNEKLFF

SEQ ID NO: 182 - protein, 18 aa, Artificial sequence
  synthetic sequence
CASSVGSRRLAGAYEQYF

SEQ ID NO: 183 - protein, 14 aa, Artificial sequence
  synthetic sequence
CASSRDRNQETQYF

SEQ ID NO: 184 - protein, 16 aa, Artificial sequence
  synthetic sequence
CASSWRLAGPQETQYF

SEQ ID NO: 185 - protein, 13 aa, Artificial sequence
  synthetic sequence
CASSLEGFGELFF

SEQ ID NO: 186 - protein, 16 aa, Artificial sequence
  synthetic sequence
CATSRDGGTVNSPLHF

SEQ ID NO: 187 - protein, 13 aa, Artificial sequence
  synthetic sequence
CASRAEGNTEAFF

SEQ ID NO: 188 - protein, 14 aa, Artificial sequence
  synthetic sequence
CASSYRAFYNEQFF

SEQ ID NO: 189 - protein, 12 aa, Artificial sequence
  synthetic sequence
CAIQRGGDTQYF

SEQ ID NO: 190 - protein, 11 aa, Artificial sequence
  synthetic sequence
CASSTRHEQYF

SEQ ID NO: 191 - protein, 15 aa, Artificial sequence
  synthetic sequence
CSARVLAGGAGELFF

SEQ ID NO: 192 - protein, 16 aa, Artificial sequence
  synthetic sequence
CASSLDRGGGYNEQFF

SEQ ID NO: 193 - protein, 16 aa, Artificial sequence
  synthetic sequence
CASSLAWTGMNTEAFF

SEQ ID NO: 194 - protein, 15 aa, Artificial sequence
  synthetic sequence
CASSMGLGGSQPQHF

SEQ ID NO: 195 - protein, 16 aa, Artificial sequence
  synthetic sequence
CASSPGRDSSYNEQFF

SEQ ID NO: 196 - protein, 13 aa, Artificial sequence
  synthetic sequence
CASSTDRVGELFF

SEQ ID NO: 197 - protein, 18 aa, Artificial sequence
  synthetic sequence
CASSLKGQGFSGANVLTF

SEQ ID NO: 198 - protein, 13 aa, Artificial sequence
  synthetic sequence
CASRWTGIYGYTF

SEQ ID NO: 199 - protein, 17 aa, Artificial sequence
  synthetic sequence
CASSVALRGALNTEAFF

SEQ ID NO: 200 - protein, 14 aa, Artificial sequence
  synthetic sequence
CASSPGTVGNTIYF

SEQ ID NO: 201 - protein, 11 aa, Artificial sequence
  synthetic sequence
CASSSRHEQFF

SEQ ID NO: 202 - protein, 16 aa, Artificial sequence
  synthetic sequence
CASSESRGRRNTEAFF

SEQ ID NO: 203 - protein, 16 aa, Artificial sequence
  synthetic sequence
CASSPPGDRNTDTQYF

SEQ ID NO: 204 - protein, 13 aa, Artificial sequence
  synthetic sequence
CSATPGRIYGYTF

SEQ ID NO: 205 - protein, 13 aa, Artificial sequence
  synthetic sequence
CSARNRGPYEQYF

SEQ ID NO: 206 - protein, 15 aa, Artificial sequence
  synthetic sequence
CASSIRTGTAETQYF

SEQ ID NO: 207 - protein, 14 aa, Artificial sequence
  synthetic sequence
CASSPRDREGKLFF

SEQ ID NO: 208 - protein, 14 aa, Artificial sequence
  synthetic sequence
CAISKGGNQETQYF

SEQ ID NO: 209 - protein, 14 aa, Artificial sequence
  synthetic sequence
CASSRGASGNTIYF

SEQ ID NO: 210 - protein, 16 aa, Artificial sequence
  synthetic sequence
CASSPGTGRLNQPQHF

SEQ ID NO: 211 - protein, 15 aa, Artificial sequence
  synthetic sequence
CSARDPRVDTGELFF

SEQ ID NO: 212 - protein, 13 aa, Artificial sequence
  synthetic sequence
CSAAGAQADTQYF

SEQ ID NO: 213 - protein, 13 aa, Artificial sequence
  synthetic sequence
CASSLEIQETQYF

SEQ ID NO: 214 - protein, 15 aa, Artificial sequence
  synthetic sequence
CASSTRTGRNEKLFF

SEQ ID NO: 215 - protein, 15 aa, Artificial sequence
  synthetic sequence
CASSASGTANQPQHF

SEQ ID NO: 216 - protein, 14 aa, Artificial sequence
  synthetic sequence
CSARAGVAYNEQFF

SEQ ID NO: 217 - protein, 15 aa, Artificial sequence
  synthetic sequence
CASSVKDRGGEKLFF

SEQ ID NO: 218 - protein, 13 aa, Artificial sequence
  synthetic sequence
CASSSDRSGELFF

SEQ ID NO: 219 - protein, 15 aa, Artificial sequence
  synthetic sequence
CASSTRQGRTGELFF

SEQ ID NO: 220 - protein, 15 aa, Artificial sequence
  synthetic sequence
CASSSRTGLGTEAFF

SEQ ID NO: 221 - protein, 15 aa, Artificial sequence
  synthetic sequence
CASSLGGQKNQPQHF

SEQ ID NO: 222 - protein, 14 aa, Artificial sequence
  synthetic sequence
CASSLQQGRTEAFF

SEQ ID NO: 223 - protein, 16 aa, Artificial sequence
  synthetic sequence
CASSLLSGGNYNEQFF

SEQ ID NO: 224 - protein, 15 aa, Artificial sequence
  synthetic sequence
CASSLRDRDGNTIYF

SEQ ID NO: 225 - protein, 20 aa, Artificial sequence
  synthetic sequence
CASSPPRGLAGGPGTDTQYF

SEQ ID NO: 226 - protein, 14 aa, Artificial sequence
  synthetic sequence
CASSNRDREGKLFF

SEQ ID NO: 227 - protein, 18 aa, Artificial sequence
  synthetic sequence
CSARDRVTGGSYNSPLHF

SEQ ID NO: 228 - protein, 15 aa, Artificial sequence
  synthetic sequence
CASSEKGDSGNTIYF

SEQ ID NO: 229 - protein, 15 aa, Artificial sequence
  synthetic sequence
CASRLRDPSTDTQYF

SEQ ID NO: 230 - protein, 13 aa, Artificial sequence
  synthetic sequence
CASSPRLYNEQFF

SEQ ID NO: 231 - protein, 15 aa, Artificial sequence
  synthetic sequence
CSARDPRTDTGELFF

SEQ ID NO: 232 - protein, 16 aa, Artificial sequence
  synthetic sequence
CASSLVTGTMNTEAFF

SEQ ID NO: 233 - protein, 15 aa, Artificial sequence
  synthetic sequence
CASSPSPTGNTEAFF

SEQ ID NO: 234 - protein, 16 aa, Artificial sequence
  synthetic sequence
CASSLASGSRQETQYF

SEQ ID NO: 235 - protein, 14 aa, Artificial sequence
  synthetic sequence
CASKRGEGSYEQYF

SEQ ID NO: 236 - protein, 13 aa, Artificial sequence
  synthetic sequence
CASSFSVDSPLHF

SEQ ID NO: 237 - protein, 15 aa, Artificial sequence
  synthetic sequence
CSVEGGQGNTDTQYF

SEQ ID NO: 238 - protein, 15 aa, Artificial sequence
  synthetic sequence
CASSESAVYNSPLHF

SEQ ID NO: 239 - protein, 15 aa, Artificial sequence
  synthetic sequence
CASSLAGQKNQPQHF

SEQ ID NO: 240 - protein, 13 aa, Artificial sequence
  synthetic sequence
CASSPHLKETQYF

SEQ ID NO: 241 - protein, 14 aa, Artificial sequence
  synthetic sequence
CASSERDRPYEQYF

SEQ ID NO: 242 - protein, 18 aa, Artificial sequence
  synthetic sequence
CASSVAPRGGRSTDTQYF

SEQ ID NO: 243 - protein, 15 aa, Artificial sequence
  synthetic sequence
CASSSMTSGTDTQYF

SEQ ID NO: 244 - protein, 14 aa, Artificial sequence
  synthetic sequence
CASRTGGGNTEAFF

SEQ ID NO: 245 - protein, 12 aa, Artificial sequence
  synthetic sequence
CASSATGNTIYF

SEQ ID NO: 246 - protein, 16 aa, Artificial sequence
  synthetic sequence
CASSLAWIGMNTEAFF

SEQ ID NO: 247 - protein, 15 aa, Artificial sequence
  synthetic sequence
CSARVLAGGSGELFF

SEQ ID NO: 248 - protein, 15 aa, Artificial sequence
  synthetic sequence
CASSIRTGSAETQYF

SEQ ID NO: 249 - protein, 13 aa, Artificial sequence
  synthetic sequence
CASSTDRAGEQFF

SEQ ID NO: 250 - protein, 15 aa, Artificial sequence
  synthetic sequence
CASSAQGVGNQPQHF

SEQ ID NO: 251 - protein, 17 aa, Artificial sequence
  synthetic sequence
CSARLAGGPPSYNEQFF

SEQ ID NO: 252 - protein, 17 aa, Artificial sequence
  synthetic sequence
CASSPLAGGPRTDTQYF

SEQ ID NO: 253 - protein, 15 aa, Artificial sequence
  synthetic sequence
CASSPLGGSNEKLFF

SEQ ID NO: 254 - protein, 13 aa, Artificial sequence
  synthetic sequence
CASSHEGGETQYF

SEQ ID NO: 255 - protein, 14 aa, Artificial sequence
  synthetic sequence
CASSPLLARETQYF

SEQ ID NO: 256 - protein, 15 aa, Artificial sequence
  synthetic sequence
CASSFRGGGDYGYTF

SEQ ID NO: 257 - protein, 16 aa, Artificial sequence
  synthetic sequence
CASSFAGPRSYNEQFF

SEQ ID NO: 258 - protein, 17 aa, Artificial sequence
  synthetic sequence
CASSPKTGGSYNSPLHF

SEQ ID NO: 259 - protein, 16 aa, Artificial sequence
  synthetic sequence
CASSSRLAGEVDTQYF

SEQ ID NO: 260 - protein, 14 aa, Artificial sequence
  synthetic sequence
CASSRLAGNNEQFF

SEQ ID NO: 261 - protein, 14 aa, Artificial sequence
  synthetic sequence
CASSPTSYSYEQYF

SEQ ID NO: 262 - protein, 11 aa, Artificial sequence
  synthetic sequence
CASSHRHEQFF

SEQ ID NO: 263 - protein, 13 aa, Artificial sequence
  synthetic sequence
CSVPRQGTDTQYF

SEQ ID NO: 264 - protein, 16 aa, Artificial sequence
  synthetic sequence
CASSFLSGGNYNEQFF

SEQ ID NO: 265 - protein, 13 aa, Artificial sequence
  synthetic sequence
CSATGTSDREQFF

SEQ ID NO: 266 - protein, 15 aa, Artificial sequence
  synthetic sequence
CASSYSVSPNQPQHF

SEQ ID NO: 267 - protein, 14 aa, Artificial sequence
  synthetic sequence
CASSLTEGGQPQHF

SEQ ID NO: 268 - protein, 15 aa, Artificial sequence
  synthetic sequence
CASSLGDRRGNTIYF

SEQ ID NO: 269 - protein, 14 aa, Artificial sequence
  synthetic sequence
CASSGGQGETEAFF

SEQ ID NO: 270 - protein, 12 aa, Artificial sequence
  synthetic sequence
CASSPRDYEQYF

SEQ ID NO: 271 - protein, 16 aa, Artificial sequence
  synthetic sequence
CASSPPGDRNSYEQYF

SEQ ID NO: 272 - protein, 14 aa, Artificial sequence
  synthetic sequence
CASSPGTEGYGYTF

SEQ ID NO: 273 - protein, 15 aa, Artificial sequence
  synthetic sequence
CASSLVFGENYGYTF

SEQ ID NO: 274 - protein, 15 aa, Artificial sequence
  synthetic sequence
CASSLGTSYTDTQYF

SEQ ID NO: 275 - protein, 12 aa, Artificial sequence
  synthetic sequence
CSAIRGSGELFF

SEQ ID NO: 276 - protein, 15 aa, Artificial sequence
  synthetic sequence
CASSLGQGEGYEQYF

SEQ ID NO: 277 - protein, 12 aa, Artificial sequence
  synthetic sequence
CSARGVRREQYF

SEQ ID NO: 278 - protein, 15 aa, Artificial sequence
  synthetic sequence
CASRTPRGRNQPQHF

SEQ ID NO: 279 - protein, 17 aa, Artificial sequence
  synthetic sequence
CASSQEGAGLATDTQYF

SEQ ID NO: 280 - protein, 17 aa, Artificial sequence
  synthetic sequence
CASSFRLAGAPGYEQYF

SEQ ID NO: 281 - protein, 14 aa, Artificial sequence
  synthetic sequence
CASTPGQGADGYTF

SEQ ID NO: 282 - protein, 17 aa, Artificial sequence
  synthetic sequence
CASSPGTSGGARETQYF

SEQ ID NO: 283 - protein, 14 aa, Artificial sequence
  synthetic sequence
CASRRDREGNEQFF

SEQ ID NO: 284 - protein, 16 aa, Artificial sequence
  synthetic sequence
CAIRGLAGGPTDTQYF

SEQ ID NO: 285 - protein, 14 aa, Artificial sequence
  synthetic sequence
CAIGTGDSTDTQYF

SEQ ID NO: 286 - protein, 13 aa, Artificial sequence
  synthetic sequence
CATSPTSNYGYTF

SEQ ID NO: 287 - protein, 17 aa, Artificial sequence
  synthetic sequence
CASSPPAGGRNTGELFF

SEQ ID NO: 288 - protein, 14 aa, Artificial sequence
  synthetic sequence
CASSPYTNTGELFF

SEQ ID NO: 289 - protein, 13 aa, Artificial sequence
  synthetic sequence
CASSNRDSSPLHF

SEQ ID NO: 290 - protein, 14 aa, Artificial sequence
  synthetic sequence
CASSPTSFSYEQYF

SEQ ID NO: 291 - protein, 16 aa, Artificial sequence
  synthetic sequence
CSATGLADSGANVLTF

SEQ ID NO: 292 - protein, 13 aa, Artificial sequence
  synthetic sequence
CASSSGPTGELFF

SEQ ID NO: 293 - protein, 16 aa, Artificial sequence
  synthetic sequence
CASSPQGTGKGETQYF

SEQ ID NO: 294 - protein, 13 aa, Artificial sequence
  synthetic sequence
CASRRVADQPQHF

SEQ ID NO: 295 - protein, 14 aa, Artificial sequence
  synthetic sequence
CASSFAGEDYEQYF

SEQ ID NO: 296 - protein, 15 aa, Artificial sequence
  synthetic sequence
CASSPETTVNTEAFF

SEQ ID NO: 297 - protein, 14 aa, Artificial sequence
  synthetic sequence
CASSPTPSLNEQFF

SEQ ID NO: 298 - protein, 17 aa, Artificial sequence
  synthetic sequence
CASSARDRGSGANVLTF

SEQ ID NO: 299 - protein, 15 aa, Artificial sequence
  synthetic sequence
CASSLFGGIQETQYF

SEQ ID NO: 300 - protein, 12 aa, Artificial sequence
  synthetic sequence
CASSLPPNEQFF

SEQ ID NO: 301 - protein, 16 aa, Artificial sequence
  synthetic sequence
CASSPKGGVNTGELFF

SEQ ID NO: 302 - protein, 15 aa, Artificial sequence
  synthetic sequence
CASSESKGGRNEQYF

SEQ ID NO: 303 - protein, 16 aa, Artificial sequence
  synthetic sequence
CASSPQGQGKGQPQHF

SEQ ID NO: 304 - protein, 15 aa, Artificial sequence
  synthetic sequence
CASSPFGTSYNEQFF

SEQ ID NO: 305 - protein, 14 aa, Artificial sequence
  synthetic sequence
CASKAGGPGNEQFF

SEQ ID NO: 306 - protein, 15 aa, Artificial sequence
  synthetic sequence
CASSYLGEQGNEQFF

SEQ ID NO: 307 - protein, 16 aa, Artificial sequence
  synthetic sequence
CASSEGRGGPKETQYF

SEQ ID NO: 308 - protein, 17 aa, Artificial sequence
  synthetic sequence
CSARRAGGPPSYNEQFF

SEQ ID NO: 309 - protein, 14 aa, Artificial sequence
  synthetic sequence
CASSTQQGRTEAFF

SEQ ID NO: 310 - protein, 13 aa, Artificial sequence
  synthetic sequence
CSARSQGTDTQYF

SEQ ID NO: 311 - protein, 15 aa, Artificial sequence
  synthetic sequence
CASSKGLAGNYEQYF

SEQ ID NO: 312 - protein, 15 aa, Artificial sequence
  synthetic sequence
CASSYGQGWNTEAFF

SEQ ID NO: 313 - protein, 15 aa, Artificial sequence
  synthetic sequence
CASSYLGEQGYGYTF

SEQ ID NO: 314 - protein, 16 aa, Artificial sequence
  synthetic sequence
CASSLAVGHMNTEAFF

SEQ ID NO: 315 - protein, 13 aa, Artificial sequence
  synthetic sequence
CASSELRTGELFF

SEQ ID NO: 316 - protein, 15 aa, Artificial sequence
  synthetic sequence
CASSYSVAPNQPQHF

SEQ ID NO: 317 - protein, 13 aa, Artificial sequence
  synthetic sequence
CASSADRPGELFF

SEQ ID NO: 318 - protein, 14 aa, Artificial sequence
  synthetic sequence
CSARWESYYNEQFF

SEQ ID NO: 319 - protein, 13 aa, Artificial sequence
  synthetic sequence
CASSRELADTQYF

SEQ ID NO: 320 - protein, 16 aa, Artificial sequence
  synthetic sequence
CASSISPQGGPYEQYF

SEQ ID NO: 321 - protein, 14 aa, Artificial sequence
  synthetic sequence
CASSIKRTGTEAFF

SEQ ID NO: 322 - protein, 15 aa, Artificial sequence
  synthetic sequence
CAWSRTGRNTGELFF

SEQ ID NO: 323 - protein, 15 aa, Artificial sequence
  synthetic sequence
CASSQETGVQETQYF

SEQ ID NO: 324 - protein, 16 aa, Artificial sequence
  synthetic sequence
CASSVRTASSTDTQYF

SEQ ID NO: 325 - protein, 13 aa, Artificial sequence
  synthetic sequence
CASRRHRNTEAFF

SEQ ID NO: 326 - protein, 14 aa, Artificial sequence
  synthetic sequence
CASSFWQGRYEQYF

SEQ ID NO: 327 - protein, 14 aa, Artificial sequence
  synthetic sequence
CASSQDIAGETQYF

SEQ ID NO: 328 - protein, 15 aa, Artificial sequence
  synthetic sequence
CASRPGRDSGNTIYF

SEQ ID NO: 329 - protein, 11 aa, Artificial sequence
  synthetic sequence
CSAHTGEKLFF

SEQ ID NO: 330 - protein, 14 aa, Artificial sequence
  synthetic sequence
CAIGGGDSTDTQYF

SEQ ID NO: 331 - protein, 13 aa, Artificial sequence
  synthetic sequence
CASSLEIADTQYF

SEQ ID NO: 332 - protein, 15 aa, Artificial sequence
  synthetic sequence
CASSPTGTANQPQHF

SEQ ID NO: 333 - protein, 14 aa, Artificial sequence
  synthetic sequence
CASSQGRERQPQHF

SEQ ID NO: 334 - protein, 15 aa, Artificial sequence
  synthetic sequence
CASSLRGGDDYGYTF

SEQ ID NO: 335 - protein, 13 aa, Artificial sequence
  synthetic sequence
CASSREGAGEQYF

SEQ ID NO: 336 - protein, 16 aa, Artificial sequence
  synthetic sequence
CASSLVSQGQYNEQFF

SEQ ID NO: 337 - protein, 16 aa, Artificial sequence
  synthetic sequence
CASSQESGGPLDTQYF

SEQ ID NO: 338 - protein, 14 aa, Artificial sequence
  synthetic sequence
CASSPPWGTDTQYF

SEQ ID NO: 339 - protein, 16 aa, Artificial sequence
  synthetic sequence
CASSLGGDSSYNEQFF

SEQ ID NO: 340 - protein, 16 aa, Artificial sequence
  synthetic sequence
CASSFTWTGMNTEAFF

SEQ ID NO: 341 - protein, 16 aa, Artificial sequence
  synthetic sequence
CASSQEGQGFGYGYTF

SEQ ID NO: 342 - protein, 16 aa, Artificial sequence
  synthetic sequence
CASSQEGQGFGNEQFF

SEQ ID NO: 343 - protein, 12 aa, Artificial sequence
  synthetic sequence
CASSYGANVLTF

SEQ ID NO: 344 - protein, 15 aa, Artificial sequence
  synthetic sequence
CASSLLSTVNQPQHF

SEQ ID NO: 345 - protein, 15 aa, Artificial sequence
  synthetic sequence
CSARLLAGGAGELFF

SEQ ID NO: 346 - protein, 15 aa, Artificial sequence
  synthetic sequence
CASSYGQGWNQPQHF

SEQ ID NO: 347 - protein, 15 aa, Artificial sequence
  synthetic sequence
CAWRDLGGHTDTQYF

SEQ ID NO: 348 - protein, 13 aa, Artificial sequence
  synthetic sequence
CASSLNQDNEQFF

SEQ ID NO: 349 - protein, 16 aa, Artificial sequence
  synthetic sequence
CASSQEGQGWGNTIYF

SEQ ID NO: 350 - protein, 16 aa, Artificial sequence
  synthetic sequence
CASSQESQGFGNTIYF

SEQ ID NO: 351 - protein, 15 aa, Artificial sequence
  synthetic sequence
CAISVEGGGSYEQYF

SEQ ID NO: 352 - protein, 17 aa, Artificial sequence
  synthetic sequence
CSARGTSGGGLTDTQYF

SEQ ID NO: 353 - protein, 15 aa, Artificial sequence
  synthetic sequence
CASSFRGSGQETQYF

SEQ ID NO: 354 - protein, 14 aa, Artificial sequence
  synthetic sequence
CASSQALSGNTIYF

SEQ ID NO: 355 - protein, 16 aa, Artificial sequence
  synthetic sequence
CSAHRGAGGPQETQYF

SEQ ID NO: 356 - protein, 15 aa, Artificial sequence
  synthetic sequence
CASSLGDRGRDGYTF

SEQ ID NO: 357 - protein, 13 aa, Artificial sequence
  synthetic sequence
CSAKWGTDQPQHF

SEQ ID NO: 358 - protein, 14 aa, Artificial sequence
  synthetic sequence
CASSLDGTGSEQFF

SEQ ID NO: 359 - protein, 13 aa, Artificial sequence
  synthetic sequence
CASSTDTPGELFF

SEQ ID NO: 360 - protein, 13 aa, Artificial sequence
  synthetic sequence
CATRDTDTGELFF

SEQ ID NO: 361 - protein, 15 aa, Artificial sequence
  synthetic sequence
CASSPSTANNEKLFF

SEQ ID NO: 362 - protein, 15 aa, Artificial sequence
  synthetic sequence
CASIRLAGGADTQYF

SEQ ID NO: 363 - protein, 13 aa, Artificial sequence
  synthetic sequence
CASSQWGGEKLFF

SEQ ID NO: 364 - protein, 14 aa, Artificial sequence
  synthetic sequence
CASSQQGVNYGYTF

SEQ ID NO: 365 - protein, 13 aa, Artificial sequence
  synthetic sequence
CASSQNLGREQYF

SEQ ID NO: 366 - protein, 17 aa, Artificial sequence
  synthetic sequence
CASSVGPGQGVNTEAFF

SEQ ID NO: 367 - protein, 12 aa, Artificial sequence
  synthetic sequence
CASTPTGNTIYF

SEQ ID NO: 368 - protein, 15 aa, Artificial sequence
  synthetic sequence
CASSLNPGGGDGYTF

SEQ ID NO: 369 - protein, 15 aa, Artificial sequence
  synthetic sequence
CSARDPSGDTGELFF

SEQ ID NO: 370 - protein, 15 aa, Artificial sequence
  synthetic sequence
CASSPTGTGVDGYTF

SEQ ID NO: 371 - protein, 12 aa, Artificial sequence
  synthetic sequence
CASSRADFEAFF

SEQ ID NO: 372 - protein, 16 aa, Artificial sequence
  synthetic sequence
CASSPPVGARNQPQHF

SEQ ID NO: 373 - protein, 16 aa, Artificial sequence
  synthetic sequence
CASSEAYRDSSYEQYF

SEQ ID NO: 374 - protein, 15 aa, Artificial sequence
  synthetic sequence
CASSLTDQSYNEQFF

SEQ ID NO: 375 - protein, 12 aa, Artificial sequence
  synthetic sequence
CASSSENSPLHF

SEQ ID NO: 376 - protein, 14 aa, Artificial sequence
  synthetic sequence
CASSPAGARNEQFF

SEQ ID NO: 377 - protein, 15 aa, Artificial sequence
  synthetic sequence
CASSSQTGLNQPQHF

SEQ ID NO: 378 - protein, 16 aa, Artificial sequence
  synthetic sequence
CASSQDLQRIYNEQFF

SEQ ID NO: 379 - protein, 14 aa, Artificial sequence
  synthetic sequence
CASSPDGTGGEQYF

SEQ ID NO: 380 - protein, 13 aa, Artificial sequence
  synthetic sequence
CASSGHPNTEAFF

SEQ ID NO: 381 - protein, 13 aa, Artificial sequence
  synthetic sequence
CASSFNPSYEQYF

SEQ ID NO: 382 - protein, 12 aa, Artificial sequence
  synthetic sequence
CASSSSGARAFF

SEQ ID NO: 383 - protein, 14 aa, Artificial sequence
  synthetic sequence
CSATGGSGNNEQFF

SEQ ID NO: 384 - protein, 15 aa, Artificial sequence
  synthetic sequence
CASSMGGGWGQPQHF

SEQ ID NO: 385 - protein, 13 aa, Artificial sequence
  synthetic sequence
CAWTRGLSYEQYF

SEQ ID NO: 386 - protein, 15 aa, Artificial sequence
  synthetic sequence
CASSYGDGWNQPQHF

SEQ ID NO: 387 - protein, 12 aa, Artificial sequence
  synthetic sequence
CASSDSQNIQYF

SEQ ID NO: 388 - protein, 16 aa, Artificial sequence
  synthetic sequence
CASSLTLGHLNTEAFF

SEQ ID NO: 389 - protein, 17 aa, Artificial sequence
  synthetic sequence
CASSYSPSGRVGNIQYF

SEQ ID NO: 390 - protein, 14 aa, Artificial sequence
  synthetic sequence
CASSQGRGEQPQHF

SEQ ID NO: 391 - protein, 15 aa, Artificial sequence
  synthetic sequence
CASSLGIGDNQPQHF

SEQ ID NO: 392 - protein, 16 aa, Artificial sequence
  synthetic sequence
CASSQGGTGPDNEQFF

SEQ ID NO: 393 - protein, 17 aa, Artificial sequence
  synthetic sequence
CASSISPMTGSNQPQHF

SEQ ID NO: 394 - protein, 13 aa, Artificial sequence
  synthetic sequence
CASSSDKAGELFF

SEQ ID NO: 395 - protein, 16 aa, Artificial sequence
  synthetic sequence
CASSLSGLAGERELFF

SEQ ID NO: 396 - protein, 15 aa, Artificial sequence
  synthetic sequence
CASSGRQEINEKLFF

SEQ ID NO: 397 - protein, 14 aa, Artificial sequence
  synthetic sequence
CASSFRAEGEKLFF

SEQ ID NO: 398 - protein, 15 aa, Artificial sequence
  synthetic sequence
CASSLAPGQGGEQYF

SEQ ID NO: 399 - protein, 16 aa, Artificial sequence
  synthetic sequence
CASSLASGTGTYGYTF

SEQ ID NO: 400 - protein, 14 aa, Artificial sequence
  synthetic sequence
CASSLGQHTGELFF

SEQ ID NO: 401 - protein, 13 aa, Artificial sequence
  synthetic sequence
CASSADSPGELFF

SEQ ID NO: 402 - protein, 15 aa, Artificial sequence
  synthetic sequence
CASSEDGRGADTQYF

SEQ ID NO: 403 - protein, 14 aa, Artificial sequence
  synthetic sequence
CASSRSTAQETQYF

SEQ ID NO: 404 - protein, 15 aa, Artificial sequence
  synthetic sequence
CASRAPRGRNQPQHF

SEQ ID NO: 405 - protein, 14 aa, Artificial sequence
  synthetic sequence
CASTRTRRSYEQYF

SEQ ID NO: 406 - protein, 17 aa, Artificial sequence
  synthetic sequence
CASSVALRGALNNEQFF

SEQ ID NO: 407 - protein, 11 aa, Artificial sequence
  synthetic sequence
CASSTGGEQYF

SEQ ID NO: 408 - protein, 13 aa, Artificial sequence
  synthetic sequence
CSVEGTRPGELFF

SEQ ID NO: 409 - protein, 14 aa, Artificial sequence
  synthetic sequence
CASSRDFAYNEQFF

SEQ ID NO: 410 - protein, 16 aa, Artificial sequence
  synthetic sequence
CASSPPTGAGTGELFF

SEQ ID NO: 411 - protein, 12 aa, Artificial sequence
  synthetic sequence
CATSRDSKTQYF

SEQ ID NO: 412 - protein, 13 aa, Artificial sequence
  synthetic sequence
CASMDRAGEKLFF

SEQ ID NO: 413 - protein, 16 aa, Artificial sequence
  synthetic sequence
CASSLRTTGLNTEAFF

SEQ ID NO: 414 - protein, 15 aa, Artificial sequence
  synthetic sequence
CASSPRTGRAETQYF

SEQ ID NO: 415 - protein, 14 aa, Artificial sequence
  synthetic sequence
CSARRGVAYNEQFF

SEQ ID NO: 416 - protein, 15 aa, Artificial sequence
  synthetic sequence
CSARELAGGAGELFF

SEQ ID NO: 417 - protein, 13 aa, Artificial sequence
  synthetic sequence
CASSWKTDYEQYF

SEQ ID NO: 418 - protein, 14 aa, Artificial sequence
  synthetic sequence
CASSLYPSLYEQYF

SEQ ID NO: 419 - protein, 15 aa, Artificial sequence
  synthetic sequence
CASSEWGDDNQPQHF

SEQ ID NO: 420 - protein, 15 aa, Artificial sequence
  synthetic sequence
CSARDPRQDTGELFF

SEQ ID NO: 421 - protein, 14 aa, Artificial sequence
  synthetic sequence
CSARGQGDHQPQHF

SEQ ID NO: 422 - protein, 17 aa, Artificial sequence
  synthetic sequence
CASSPPSAGRNTGELFF

SEQ ID NO: 423 - protein, 14 aa, Artificial sequence
  synthetic sequence
CSARVGVAYNEQFF

SEQ ID NO: 424 - protein, 15 aa, Artificial sequence
  synthetic sequence
CASSLVFGESYEQYF

SEQ ID NO: 425 - protein, 14 aa, Artificial sequence
  synthetic sequence
CASSTQGRDSPLHF

SEQ ID NO: 426 - protein, 15 aa, Artificial sequence
  synthetic sequence
CASRRPQGRNQPQHF

SEQ ID NO: 427 - protein, 12 aa, Artificial sequence
  synthetic sequence
CSATTSWNEQFF

SEQ ID NO: 428 - protein, 15 aa, Artificial sequence
  synthetic sequence
CASSLTGLIQETQYF

SEQ ID NO: 429 - protein, 15 aa, Artificial sequence
  synthetic sequence
CASSSGTAGGSPLHF

SEQ ID NO: 430 - protein, 13 aa, Artificial sequence
  synthetic sequence
CSVWGRATDTQYF

SEQ ID NO: 431 - protein, 14 aa, Artificial sequence
  synthetic sequence
CAISQGDSTDTQYF

SEQ ID NO: 432 - protein, 15 aa, Artificial sequence
  synthetic sequence
CASSAKDRGGEKLFF

SEQ ID NO: 433 - protein, 14 aa, Artificial sequence
  synthetic sequence
CSVEFSGGADTQYF

SEQ ID NO: 434 - protein, 15 aa, Artificial sequence
  synthetic sequence
CASSPKAGLTGELFF

SEQ ID NO: 435 - protein, 13 aa, Artificial sequence
  synthetic sequence
CASSSELQETQYF

SEQ ID NO: 436 - protein, 12 aa, Artificial sequence
  synthetic sequence
CSLEEGTDTQYF

SEQ ID NO: 437 - protein, 15 aa, Artificial sequence
  synthetic sequence
CASSLRGSGQETQYF

SEQ ID NO: 438 - protein, 14 aa, Artificial sequence
  synthetic sequence
CASSLGASLDTQYF

SEQ ID NO: 439 - protein, 16 aa, Artificial sequence
  synthetic sequence
CASSLITGTLNTEAFF

SEQ ID NO: 440 - protein, 13 aa, Artificial sequence
  synthetic sequence
CSATGAGSDEQYF

SEQ ID NO: 441 - protein, 14 aa, Artificial sequence
  synthetic sequence
CSANLGQENTEAFF

SEQ ID NO: 442 - protein, 14 aa, Artificial sequence
  synthetic sequence
CSARPQGDNQPQHF

SEQ ID NO: 443 - protein, 11 aa, Artificial sequence
  synthetic sequence
CASRQAHGYTF

SEQ ID NO: 444 - protein, 15 aa, Artificial sequence
  synthetic sequence
CASSPQGQLNTEAFF

SEQ ID NO: 445 - protein, 13 aa, Artificial sequence
  synthetic sequence
CASRTGGARGYTF

SEQ ID NO: 446 - protein, 16 aa, Artificial sequence
  synthetic sequence
CASSGGSGGPQETQYF

SEQ ID NO: 447 - protein, 15 aa, Artificial sequence
  synthetic sequence
CSVEGGTGNTDTQYF

SEQ ID NO: 448 - protein, 15 aa, Artificial sequence
  synthetic sequence
CASSERASGYNEQFF

SEQ ID NO: 449 - protein, 13 aa, Artificial sequence
  synthetic sequence
CASSPQTGYEQYF

SEQ ID NO: 450 - protein, 15 aa, Artificial sequence
  synthetic sequence
CASSTGTSYNSPLHF

SEQ ID NO: 451 - protein, 15 aa, Artificial sequence
  synthetic sequence
CASSLEGKGDQPQHF

SEQ ID NO: 452 - protein, 13 aa, Artificial sequence
  synthetic sequence
CASSGDLPGELFF

SEQ ID NO: 453 - protein, 13 aa, Artificial sequence
  synthetic sequence
CASSSDEPEKLFF

SEQ ID NO: 454 - protein, 16 aa, Artificial sequence
  synthetic sequence
CASSLAWTGMSYEQYF

SEQ ID NO: 455 - protein, 13 aa, Artificial sequence
  synthetic sequence
CASSGDSAGELFF

SEQ ID NO: 456 - protein, 20 aa, Artificial sequence
  synthetic sequence
CASSPPRGTSGGPNTGELFF

SEQ ID NO: 457 - protein, 15 aa, Artificial sequence
  synthetic sequence
CSARALAGGAGELFF

SEQ ID NO: 458 - protein, 15 aa, Artificial sequence
  synthetic sequence
CASSPTGGARYEQYF

SEQ ID NO: 459 - protein, 15 aa, Artificial sequence
  synthetic sequence
CASSTRTGSAETQYF

SEQ ID NO: 460 - protein, 15 aa, Artificial sequence
  synthetic sequence
CASSTRTGTAETQYF

SEQ ID NO: 461 - protein, 12 aa, Artificial sequence
  synthetic sequence
CASSQTFDEQYF

SEQ ID NO: 462 - protein, 15 aa, Artificial sequence
  synthetic sequence
CATSRDYPQNSPLHF

SEQ ID NO: 463 - protein, 13 aa, Artificial sequence
  synthetic sequence
CASSSDKPGELFF

SEQ ID NO: 464 - protein, 16 aa, Artificial sequence
  synthetic sequence
CASSFVWTGMNTEAFF

SEQ ID NO: 465 - protein, 13 aa, Artificial sequence
  synthetic sequence
CSARGNDYQPQHF

SEQ ID NO: 466 - protein, 16 aa, Artificial sequence
  synthetic sequence
CASSLISGTVNTEAFF

SEQ ID NO: 467 - protein, 13 aa, Artificial sequence
  synthetic sequence
CASSSDNPGELFF

SEQ ID NO: 468 - protein, 16 aa, Artificial sequence
  synthetic sequence
CASSPPGDRNTYEQYF

SEQ ID NO: 469 - protein, 14 aa, Artificial sequence
  synthetic sequence
CASSLWQGRYEQYF

SEQ ID NO: 470 - protein, 15 aa, Artificial sequence
  synthetic sequence
CASSLDAGPQETQYF

SEQ ID NO: 471 - protein, 15 aa, Artificial sequence
  synthetic sequence
CASSRIAGDYNEQFF

SEQ ID NO: 472 - protein, 15 aa, Artificial sequence
  synthetic sequence
CASSESGTRKETQYF

SEQ ID NO: 473 - protein, 15 aa, Artificial sequence
  synthetic sequence
CASSEVFGGRETQYF

SEQ ID NO: 474 - protein, 16 aa, Artificial sequence
  synthetic sequence
CASSYRLAGPQETQYF

SEQ ID NO: 475 - protein, 14 aa, Artificial sequence
  synthetic sequence
CASSLDLKAGELFF

SEQ ID NO: 476 - protein, 14 aa, Artificial sequence
  synthetic sequence
CASSLDGTGTEQYF

SEQ ID NO: 477 - protein, 15 aa, Artificial sequence
  synthetic sequence
CASSLVRGHNSPLHF

SEQ ID NO: 478 - protein, 16 aa, Artificial sequence
  synthetic sequence
CASSGDPPSSYNEQFF

SEQ ID NO: 479 - protein, 12 aa, Artificial sequence
  synthetic sequence
CASSPDQNGYTF

SEQ ID NO: 480 - protein, 15 aa, Artificial sequence
  synthetic sequence
CSAPPDSQSYNEQFF

SEQ ID NO: 481 - protein, 15 aa, Artificial sequence
  synthetic sequence
CASSVAQGDGGELFF

SEQ ID NO: 482 - protein, 18 aa, Artificial sequence
  synthetic sequence
CASSVAPRGGRGTDTQYF

SEQ ID NO: 483 - protein, 13 aa, Artificial sequence
  synthetic sequence
CASSSDLPGELFF

SEQ ID NO: 484 - protein, 14 aa, Artificial sequence
  synthetic sequence
CASSLPGLLNEQFF

SEQ ID NO: 485 - protein, 14 aa, Artificial sequence
  synthetic sequence
CASSLLGQRRGYTF

SEQ ID NO: 486 - protein, 14 aa, Artificial sequence
  synthetic sequence
CASSLDLKVTEAFF

SEQ ID NO: 487 - protein, 15 aa, Artificial sequence
  synthetic sequence
CASSEVFGGREKLFF

SEQ ID NO: 488 - protein, 15 aa, Artificial sequence
  synthetic sequence
CASRGPRGRNQPQHF

SEQ ID NO: 489 - protein, 16 aa, Artificial sequence
  synthetic sequence
CASSLKSGLGTGELFF

SEQ ID NO: 490 - protein, 16 aa, Artificial sequence
  synthetic sequence
CASSESKGRRNTEAFF

SEQ ID NO: 491 - protein, 12 aa, Artificial sequence
  synthetic sequence
CSATGWGTEAFF

SEQ ID NO: 492 - protein, 14 aa, Artificial sequence
  synthetic sequence
CASSLQRDSYEQYF

SEQ ID NO: 493 - protein, 16 aa, Artificial sequence
  synthetic sequence
CASSLGNRVYQETQYF

SEQ ID NO: 494 - protein, 12 aa, Artificial sequence
  synthetic sequence
CASSWGDEGYTF

SEQ ID NO: 495 - protein, 17 aa, Artificial sequence
  synthetic sequence
CASSLAGSRSGANVLTF

SEQ ID NO: 496 - protein, 15 aa, Artificial sequence
  synthetic sequence
CASSFRRNSGNTIYF

SEQ ID NO: 497 - protein, 16 aa, Artificial sequence
  synthetic sequence
CASSLGGLAGDNEQFF

SEQ ID NO: 498 - protein, 14 aa, Artificial sequence
  synthetic sequence
CASSRGREDQPQHF

SEQ ID NO: 499 - protein, 15 aa, Artificial sequence
  synthetic sequence
CATSRDWGQNSPLHF

SEQ ID NO: 500 - protein, 15 aa, Artificial sequence
  synthetic sequence
CASSLTAQSYNEQFF

SEQ ID NO: 501 - protein, 15 aa, Artificial sequence
  synthetic sequence
CASSYGQGRSYGYTF

SEQ ID NO: 502 - protein, 15 aa, Artificial sequence
  synthetic sequence
CAISEGSSGLETQYF

SEQ ID NO: 503 - protein, 15 aa, Artificial sequence
  synthetic sequence
CASSRGGQGKNIQYF

SEQ ID NO: 504 - protein, 15 aa, Artificial sequence
  synthetic sequence
CASSYGVTRGTEAFF

SEQ ID NO: 505 - protein, 16 aa, Artificial sequence
  synthetic sequence
CASSLVRDRAQETQYF

SEQ ID NO: 506 - protein, 15 aa, Artificial sequence
  synthetic sequence
CASSQGGQGIYEQYF

SEQ ID NO: 507 - protein, 16 aa, Artificial sequence
  synthetic sequence
CASSPGQGRLNQPQHF

SEQ ID NO: 508 - protein, 12 aa, Artificial sequence
  synthetic sequence
CAWRVQYNEQFF

SEQ ID NO: 509 - protein, 15 aa, Artificial sequence
  synthetic sequence
CASSSGGGPGSPLHF

SEQ ID NO: 510 - protein, 14 aa, Artificial sequence
  synthetic sequence
CASSLYPSVYEQYF

SEQ ID NO: 511 - protein, 15 aa, Artificial sequence
  synthetic sequence
CASSFNGLMNTEAFF

SEQ ID NO: 512 - protein, 13 aa, Artificial sequence
  synthetic sequence
CAWARGLSYEQYF

SEQ ID NO: 513 - protein, 15 aa, Artificial sequence
  synthetic sequence
CASSLYGTGPYEQYF

SEQ ID NO: 514 - protein, 14 aa, Artificial sequence
  synthetic sequence
CASSLRGIGDTQYF

SEQ ID NO: 515 - protein, 14 aa, Artificial sequence
  synthetic sequence
CSARGQGDDQPQHF

SEQ ID NO: 516 - protein, 17 aa, Artificial sequence
  synthetic sequence
CASSLVGSRSGANVLTF

SEQ ID NO: 517 - protein, 13 aa, Artificial sequence
  synthetic sequence
CASSGERPGELFF

SEQ ID NO: 518 - protein, 17 aa, Artificial sequence
  synthetic sequence
CASSSGTSGRAPDTQYF

SEQ ID NO: 519 - protein, 17 aa, Artificial sequence
  synthetic sequence
CASSLRGRGDLNTEAFF

SEQ ID NO: 520 - protein, 16 aa, Artificial sequence
  synthetic sequence
CASSADQGVSGNTIYF

SEQ ID NO: 521 - protein, 16 aa, Artificial sequence
  synthetic sequence
CASSLARTSGRNEQFF

SEQ ID NO: 522 - protein, 12 aa, Artificial sequence
  synthetic sequence
CASSVPPDTQYF

SEQ ID NO: 523 - protein, 15 aa, Artificial sequence
  synthetic sequence
CASRIPRGRNQPQHF

SEQ ID NO: 524 - protein, 14 aa, Artificial sequence
  synthetic sequence
CASSSGSVNTEAFF

SEQ ID NO: 525 - protein, 17 aa, Artificial sequence
  synthetic sequence
CASSLVPTGGGYNEQFF

SEQ ID NO: 526 - protein, 15 aa, Artificial sequence
  synthetic sequence
CASRGSRGRNQPQHF

SEQ ID NO: 527 - protein, 15 aa, Artificial sequence
  synthetic sequence
CASSLKWPGSYEQYF

SEQ ID NO: 528 - protein, 14 aa, Artificial sequence
  synthetic sequence
CASSFSRGQETQYF

SEQ ID NO: 529 - protein, 13 aa, Artificial sequence
  synthetic sequence
CASSWERGTEAFF

SEQ ID NO: 530 - protein, 15 aa, Artificial sequence
  synthetic sequence
CASSPGDRADQPQHF

SEQ ID NO: 531 - protein, 16 aa, Artificial sequence
  synthetic sequence
CASSPPTSGEYNEQFF

SEQ ID NO: 532 - protein, 18 aa, Artificial sequence
  synthetic sequence
CASSPPNSGRVGTDTQYF

SEQ ID NO: 533 - protein, 17 aa, Artificial sequence
  synthetic sequence
CASSQEGGRASTDTQYF

SEQ ID NO: 534 - protein, 15 aa, Artificial sequence
  synthetic sequence
CASSDHLGSGNTIYF

SEQ ID NO: 535 - protein, 15 aa, Artificial sequence
  synthetic sequence
CSAIRLAGGKDTQYF

SEQ ID NO: 536 - protein, 11 aa, Artificial sequence
  synthetic sequence
CASRNNEKLFF

SEQ ID NO: 537 - protein, 14 aa, Artificial sequence
  synthetic sequence
CASSFWQGRNIQYF

SEQ ID NO: 538 - protein, 15 aa, Artificial sequence
  synthetic sequence
CSARALAGGSGELFF

SEQ ID NO: 539 - protein, 12 aa, Artificial sequence
  synthetic sequence
CSARVVLGTQYF

SEQ ID NO: 540 - protein, 13 aa, Artificial sequence
  synthetic sequence
CASSTGEGNTIYF

SEQ ID NO: 541 - protein, 16 aa, Artificial sequence
  synthetic sequence
CASSSGLSDSNQPQHF

SEQ ID NO: 542 - protein, 15 aa, Artificial sequence
  synthetic sequence
CSARELAGGSGELFF

SEQ ID NO: 543 - protein, 13 aa, Artificial sequence
  synthetic sequence
CSARGRGVEKLFF

SEQ ID NO: 544 - protein, 16 aa, Artificial sequence
  synthetic sequence
CASSLRGRRNQETQYF

SEQ ID NO: 545 - protein, 15 aa, Artificial sequence
  synthetic sequence
CSAPVGTAPSYEQYF

SEQ ID NO: 546 - protein, 12 aa, Artificial sequence
  synthetic sequence
CSAQGNNQPQHF

SEQ ID NO: 547 - protein, 15 aa, Artificial sequence
  synthetic sequence
CATSRDREKGNTIYF

SEQ ID NO: 548 - protein, 14 aa, Artificial sequence
  synthetic sequence
CASSNRDRGHEQYF

SEQ ID NO: 549 - protein, 13 aa, Artificial sequence
  synthetic sequence
CASSTDKAGELFF

SEQ ID NO: 550 - protein, 16 aa, Artificial sequence
  synthetic sequence
CASSPGTGRINQPQHF

SEQ ID NO: 551 - protein, 16 aa, Artificial sequence
  synthetic sequence
CASSPTLAGGRGEQFF

SEQ ID NO: 552 - protein, 17 aa, Artificial sequence
  synthetic sequence
CAISESRGGNSNQPQHF

SEQ ID NO: 553 - protein, 15 aa, Artificial sequence
  synthetic sequence
CASRLPRGRNQPQHF

SEQ ID NO: 554 - protein, 15 aa, Artificial sequence
  synthetic sequence
CASSPVGGARYEQYF

SEQ ID NO: 555 - protein, 13 aa, Artificial sequence
  synthetic sequence
CASGRLSSYEQYF

SEQ ID NO: 556 - protein, 14 aa, Artificial sequence
  synthetic sequence
CASSPGTSRKTQYF

SEQ ID NO: 557 - protein, 16 aa, Artificial sequence
  synthetic sequence
CSATGLAASGANVLTF

SEQ ID NO: 558 - protein, 16 aa, Artificial sequence
  synthetic sequence
CASSLAIGHLNTEAFF

SEQ ID NO: 559 - protein, 15 aa, Artificial sequence
  synthetic sequence
CASSLFGGLQETQYF

SEQ ID NO: 560 - protein, 13 aa, Artificial sequence
  synthetic sequence
CASSESQGSPLHF

SEQ ID NO: 561 - protein, 14 aa, Artificial sequence
  synthetic sequence
CASSGQGRTYEQYF

SEQ ID NO: 562 - protein, 17 aa, Artificial sequence
  synthetic sequence
CASSLSGLAGNPDTQYF

SEQ ID NO: 563 - protein, 15 aa, Artificial sequence
  synthetic sequence
CASSENRWSNSPLHF

SEQ ID NO: 564 - protein, 18 aa, Artificial sequence
  synthetic sequence
CSARDRITGGSYNSPLHF

SEQ ID NO: 565 - protein, 17 aa, Artificial sequence
  synthetic sequence
CASSLARAGGPEDTQYF

SEQ ID NO: 566 - protein, 14 aa, Artificial sequence
  synthetic sequence
CASSERDRGHEQYF

SEQ ID NO: 567 - protein, 15 aa, Artificial sequence
  synthetic sequence
CASSPKTGDNTEAFF

SEQ ID NO: 568 - protein, 18 aa, Artificial sequence
  synthetic sequence
CASSVAPRGGRATDTQYF

SEQ ID NO: 569 - protein, 15 aa, Artificial sequence
  synthetic sequence
CASSQGGQEQETQYF

SEQ ID NO: 570 - protein, 14 aa, Artificial sequence
  synthetic sequence
CATSNAVLNTEAFF

SEQ ID NO: 571 - protein, 12 aa, Artificial sequence
  synthetic sequence
CASSFAKREAFF

SEQ ID NO: 572 - protein, 15 aa, Artificial sequence
  synthetic sequence
CASSPGPPPNNEQFF

SEQ ID NO: 573 - protein, 16 aa, Artificial sequence
  synthetic sequence
CASSLALAGGPDEQYF

SEQ ID NO: 574 - protein, 14 aa, Artificial sequence
  synthetic sequence
CASRGQDPQETQYF

SEQ ID NO: 575 - protein, 16 aa, Artificial sequence
  synthetic sequence
CSADRTSGGRYNEQFF

SEQ ID NO: 576 - protein, 13 aa, Artificial sequence
  synthetic sequence
CASSGQGVGGYTF

SEQ ID NO: 577 - protein, 14 aa, Artificial sequence
  synthetic sequence
CASSQGSVNTEAFF

SEQ ID NO: 578 - protein, 13 aa, Artificial sequence
  synthetic sequence
CASHRLSNYGYTF

SEQ ID NO: 579 - protein, 15 aa, Artificial sequence
  synthetic sequence
CASSESRSSYNEQFF

SEQ ID NO: 580 - protein, 10 aa, Artificial sequence
  synthetic sequence
CSAGGTGGFF

SEQ ID NO: 581 - protein, 17 aa, Artificial sequence
  synthetic sequence
CASSVGPGQGIYNEQFF

SEQ ID NO: 582 - protein, 13 aa, Artificial sequence
  synthetic sequence
CASILRQDYGYTF

SEQ ID NO: 583 - protein, 14 aa, Artificial sequence
  synthetic sequence
CASSPGTSLNIQYF

SEQ ID NO: 584 - protein, 12 aa, Artificial sequence
  synthetic sequence
CASSTPPDTQYF

SEQ ID NO: 585 - protein, 18 aa, Artificial sequence
  synthetic sequence
CASSQEGLAGGISYEQYF

SEQ ID NO: 586 - protein, 15 aa, Artificial sequence
  synthetic sequence
CASSSTGGRSGELFF

SEQ ID NO: 587 - protein, 15 aa, Artificial sequence
  synthetic sequence
CAWSTRTGVTYEQYF

SEQ ID NO: 588 - protein, 13 aa, Artificial sequence
  synthetic sequence
CASRGLARHEQYF

SEQ ID NO: 589 - protein, 14 aa, Artificial sequence
  synthetic sequence
CASSMIHMNTEAFF

SEQ ID NO: 590 - protein, 15 aa, Artificial sequence
  synthetic sequence
CASSLAPGQGGGYTF

SEQ ID NO: 591 - protein, 13 aa, Artificial sequence
  synthetic sequence
CARVLAGGGEQFF

SEQ ID NO: 592 - protein, 16 aa, Artificial sequence
  synthetic sequence
CASSYGNPGQSYEQYF

SEQ ID NO: 593 - protein, 14 aa, Artificial sequence
  synthetic sequence
CSAREGVAYNEQFF

SEQ ID NO: 594 - protein, 15 aa, Artificial sequence
  synthetic sequence
CASSIRAGTAETQYF

SEQ ID NO: 595 - protein, 15 aa, Artificial sequence
  synthetic sequence
CASSIRSGTAETQYF

SEQ ID NO: 596 - protein, 14 aa, Artificial sequence
  synthetic sequence
CAIRRPESTDTQYF

SEQ ID NO: 597 - protein, 14 aa, Artificial sequence
  synthetic sequence
CASSLREVGETQYF

SEQ ID NO: 598 - protein, 15 aa, Artificial sequence
  synthetic sequence
CASSSETSGRNEQFF

SEQ ID NO: 599 - protein, 14 aa, Artificial sequence
  synthetic sequence
CASRPDRRNTEAFF

SEQ ID NO: 600 - protein, 16 aa, Artificial sequence
  synthetic sequence
CASSYQDRGANYGYTF

SEQ ID NO: 601 - protein, 12 aa, Artificial sequence
  synthetic sequence
CASSPRDYEQFF

SEQ ID NO: 602 - protein, 15 aa, Artificial sequence
  synthetic sequence
CASSLESQRTDTQYF

SEQ ID NO: 603 - protein, 16 aa, Artificial sequence
  synthetic sequence
CSARATGGPTYNEQFF

SEQ ID NO: 604 - protein, 15 aa, Artificial sequence
  synthetic sequence
CASSLGGTERYEQYF

SEQ ID NO: 605 - protein, 15 aa, Artificial sequence
  synthetic sequence
CATSRDNSLQETQYF

SEQ ID NO: 606 - protein, 16 aa, Artificial sequence
  synthetic sequence
CATSREGTGRNTEAFF

SEQ ID NO: 607 - protein, 15 aa, Artificial sequence
  synthetic sequence
CASSLSWTGYEKLFF

SEQ ID NO: 608 - protein, 17 aa, Artificial sequence
  synthetic sequence
CASSYSTGTGFRETQYF

SEQ ID NO: 609 - protein, 14 aa, Artificial sequence
  synthetic sequence
CASSQETGLDGYTF

SEQ ID NO: 610 - protein, 13 aa, Artificial sequence
  synthetic sequence
CASTRGRGETQYF

SEQ ID NO: 611 - protein, 16 aa, Artificial sequence
  synthetic sequence
CASSSRTGDSGNTIYF

SEQ ID NO: 612 - protein, 16 aa, Artificial sequence
  synthetic sequence
CASSTRPDSNTGELFF

SEQ ID NO: 613 - protein, 15 aa, Artificial sequence
  synthetic sequence
CASSRGQGEGYEQYF

SEQ ID NO: 614 - protein, 13 aa, Artificial sequence
  synthetic sequence
CASGPTRPYEQYF

SEQ ID NO: 615 - protein, 15 aa, Artificial sequence
  synthetic sequence
CASSQGQGEGYEQFF

SEQ ID NO: 616 - protein, 13 aa, Artificial sequence
  synthetic sequence
CASSTDKTGELFF

SEQ ID NO: 617 - protein, 13 aa, Artificial sequence
  synthetic sequence
CASSLEWDQPQHF

SEQ ID NO: 618 - protein, 15 aa, Artificial sequence
  synthetic sequence
CASSPQGSAGNTIYF

SEQ ID NO: 619 - protein, 15 aa, Artificial sequence
  synthetic sequence
CASSGRLADTGELFF

SEQ ID NO: 620 - protein, 16 aa, Artificial sequence
  synthetic sequence
CASSPQGTGKGYEQYF

SEQ ID NO: 621 - protein, 14 aa, Artificial sequence
  synthetic sequence
CASRTGVGNQPQHF

SEQ ID NO: 622 - protein, 13 aa, Artificial sequence
  synthetic sequence
CASSDRDSSPLHF

SEQ ID NO: 623 - protein, 15 aa, Artificial sequence
  synthetic sequence
CASSPRQGTAETQYF

SEQ ID NO: 624 - protein, 14 aa, Artificial sequence
  synthetic sequence
CASSASSGYYEQYF

SEQ ID NO: 625 - protein, 14 aa, Artificial sequence
  synthetic sequence
CSAPLGTIQETQYF

SEQ ID NO: 626 - protein, 17 aa, Artificial sequence
  synthetic sequence
CASSVAARGALNTEAFF

SEQ ID NO: 627 - protein, 11 aa, Artificial sequence
  synthetic sequence
CASSTGGEQFF

SEQ ID NO: 628 - protein, 16 aa, Artificial sequence
  synthetic sequence
CASSLFGQNSGNTIYF

SEQ ID NO: 629 - protein, 15 aa, Artificial sequence
  synthetic sequence
CASSLVFGESYGYTF

SEQ ID NO: 630 - protein, 14 aa, Artificial sequence
  synthetic sequence
CASSFVRDRETQYF

SEQ ID NO: 631 - protein, 17 aa, Artificial sequence
  synthetic sequence
CASSPPAAGRNTGELFF

SEQ ID NO: 632 - protein, 15 aa, Artificial sequence
  synthetic sequence
CATSRDSSNQETQYF

SEQ ID NO: 633 - protein, 14 aa, Artificial sequence
  synthetic sequence
CASSQGRDKNIQYF

SEQ ID NO: 634 - protein, 17 aa, Artificial sequence
  synthetic sequence
CASSQDTSGSYTGELFF

SEQ ID NO: 635 - protein, 13 aa, Artificial sequence
  synthetic sequence
CASKGGTNEKLFF

SEQ ID NO: 636 - protein, 14 aa, Artificial sequence
  synthetic sequence
CASSLQQGRSEAFF

SEQ ID NO: 637 - protein, 15 aa, Artificial sequence
  synthetic sequence
CASSRTGGRGGELFF

SEQ ID NO: 638 - protein, 15 aa, Artificial sequence
  synthetic sequence
CASSQGEREGHEQFF

SEQ ID NO: 639 - protein, 13 aa, Artificial sequence
  synthetic sequence
CASRQQGPETQYF

SEQ ID NO: 640 - protein, 14 aa, Artificial sequence
  synthetic sequence
CASSPPGARNEQFF

SEQ ID NO: 641 - protein, 16 aa, Artificial sequence
  synthetic sequence
CASSLVGGISSYEQYF

SEQ ID NO: 642 - protein, 15 aa, Artificial sequence
  synthetic sequence
CASSASGQGGYEQYF

SEQ ID NO: 643 - protein, 15 aa, Artificial sequence
  synthetic sequence
CSAPPGGRDNSPLHF

SEQ ID NO: 644 - protein, 17 aa, Artificial sequence
  synthetic sequence
CASSYSGTGTLNQPQHF

SEQ ID NO: 645 - protein, 16 aa, Artificial sequence
  synthetic sequence
CASSLGGRQGRTEAFF

SEQ ID NO: 646 - protein, 15 aa, Artificial sequence
  synthetic sequence
CSARTGTPNQETQYF

SEQ ID NO: 647 - protein, 13 aa, Artificial sequence
  synthetic sequence
CASSTDRPGEQFF

SEQ ID NO: 648 - protein, 16 aa, Artificial sequence
  synthetic sequence
CASSLSYSHSGNTIYF

SEQ ID NO: 649 - protein, 15 aa, Artificial sequence
  synthetic sequence
CASSLGGLIDQPQHF

SEQ ID NO: 650 - protein, 13 aa, Artificial sequence
  synthetic sequence
CSGWGLATDTQYF

SEQ ID NO: 651 - protein, 14 aa, Artificial sequence
  synthetic sequence
CASSPGTQKNIQYF

SEQ ID NO: 652 - protein, 14 aa, Artificial sequence
  synthetic sequence
CASSGSGGNTEAFF

SEQ ID NO: 653 - protein, 15 aa, Artificial sequence
  synthetic sequence
CASSPVGGGRYEQYF

SEQ ID NO: 654 - protein, 12 aa, Artificial sequence
  synthetic sequence
CASSAFDQPQHF

SEQ ID NO: 655 - protein, 15 aa, Artificial sequence
  synthetic sequence
CSARDPTGDTGELFF

SEQ ID NO: 656 - protein, 12 aa, Artificial sequence
  synthetic sequence
CASSIPPGEQYF

SEQ ID NO: 657 - protein, 13 aa, Artificial sequence
  synthetic sequence
CASSPMKGYEQYF

SEQ ID NO: 658 - protein, 14 aa, Artificial sequence
  synthetic sequence
CASRGDGSNTEAFF

SEQ ID NO: 659 - protein, 16 aa, Artificial sequence
  synthetic sequence
CAWSVTGGLRNQPQHF

SEQ ID NO: 660 - protein, 15 aa, Artificial sequence
  synthetic sequence
CASSIRGGTAETQYF

SEQ ID NO: 661 - protein, 16 aa, Artificial sequence
  synthetic sequence
CASSARTGGAGQPQHF

SEQ ID NO: 662 - protein, 13 aa, Artificial sequence
  synthetic sequence
CASREHSTDTQYF

SEQ ID NO: 663 - protein, 17 aa, Artificial sequence
  synthetic sequence
CASSSRGAGGSYNEQFF

SEQ ID NO: 664 - protein, 15 aa, Artificial sequence
  synthetic sequence
CASRGARGRNQPQHF

SEQ ID NO: 665 - protein, 17 aa, Artificial sequence
  synthetic sequence
CASSQRTGGSYNSPLHF

SEQ ID NO: 666 - protein, 15 aa, Artificial sequence
  synthetic sequence
CASRTARGRNQPQHF

SEQ ID NO: 667 - protein, 14 aa, Artificial sequence
  synthetic sequence
CASSQLLARETQYF

SEQ ID NO: 668 - protein, 15 aa, Artificial sequence
  synthetic sequence
CSARDAVSGTDTQYF

SEQ ID NO: 669 - protein, 15 aa, Artificial sequence
  synthetic sequence
CSARLGTGNNSPLHF

SEQ ID NO: 670 - protein, 14 aa, Artificial sequence
  synthetic sequence
CSVEFAGGADTQYF

SEQ ID NO: 671 - protein, 16 aa, Artificial sequence
  synthetic sequence
CATSRDGGTINSPLHF

SEQ ID NO: 672 - protein, 15 aa, Artificial sequence
  synthetic sequence
CSVGGPGGSNTEAFF

SEQ ID NO: 673 - protein, 15 aa, Artificial sequence
  synthetic sequence
CASSYSGEQGNGYTF

SEQ ID NO: 674 - protein, 14 aa, Artificial sequence
  synthetic sequence
CSALSGSSYNEQFF

SEQ ID NO: 675 - protein, 17 aa, Artificial sequence
  synthetic sequence
CASSPPQRGSYNSPLHF

SEQ ID NO: 676 - protein, 14 aa, Artificial sequence
  synthetic sequence
CSATSGTDDSPLHF

SEQ ID NO: 677 - protein, 14 aa, Artificial sequence
  synthetic sequence
CASSNPGDYNEQFF

SEQ ID NO: 678 - protein, 15 aa, Artificial sequence
  synthetic sequence
CASSLQGRAGNTIYF

SEQ ID NO: 679 - protein, 14 aa, Artificial sequence
  synthetic sequence
CATLQVGSYNEQFF

SEQ ID NO: 680 - protein, 15 aa, Artificial sequence
  synthetic sequence
CASSLFGTGPYEQYF

SEQ ID NO: 681 - protein, 15 aa, Artificial sequence
  synthetic sequence
CASSQVLAGVDEQFF

SEQ ID NO: 682 - protein, 13 aa, Artificial sequence
  synthetic sequence
CASSSWGGETQYF

SEQ ID NO: 683 - protein, 13 aa, Artificial sequence
  synthetic sequence
CASSFGSTETQYF

SEQ ID NO: 684 - protein, 15 aa, Artificial sequence
  synthetic sequence
CASSLTTGGNSPLHF

SEQ ID NO: 685 - protein, 15 aa, Artificial sequence
  synthetic sequence
CSARVRAGGPGELFF

SEQ ID NO: 686 - protein, 13 aa, Artificial sequence
  synthetic sequence
CASSARHLYGYTF

SEQ ID NO: 687 - protein, 15 aa, Artificial sequence
  synthetic sequence
CASSEGGRTAYEQYF

SEQ ID NO: 688 - protein, 15 aa, Artificial sequence
  synthetic sequence
CSAPKGASAYNEQFF

SEQ ID NO: 689 - protein, 14 aa, Artificial sequence
  synthetic sequence
CASSLGRPGGEQYF

SEQ ID NO: 690 - protein, 15 aa, Artificial sequence
  synthetic sequence
CASSVEGSTHTEAFF

SEQ ID NO: 691 - protein, 12 aa, Artificial sequence
  synthetic sequence
CRISGGVTEAFF

SEQ ID NO: 692 - protein, 16 aa, Artificial sequence
  synthetic sequence
CASSQGAQGFGNTIYF

SEQ ID NO: 693 - protein, 12 aa, Artificial sequence
  synthetic sequence
CASSYDVVEQYF

SEQ ID NO: 694 - protein, 15 aa, Artificial sequence
  synthetic sequence
CASSLGGGPGSPLHF

SEQ ID NO: 695 - protein, 16 aa, Artificial sequence
  synthetic sequence
CASSYLPTGSNQPQHF

SEQ ID NO: 696 - protein, 16 aa, Artificial sequence
  synthetic sequence
CASSSRLAGNPDTQYF

SEQ ID NO: 697 - protein, 15 aa, Artificial sequence
  synthetic sequence
CSARDFTADNSPLHF

SEQ ID NO: 698 - protein, 13 aa, Artificial sequence
  synthetic sequence
CASSQELADTQYF

SEQ ID NO: 699 - protein, 15 aa, Artificial sequence
  synthetic sequence
CASSSETSGANVLTF

SEQ ID NO: 700 - protein, 13 aa, Artificial sequence
  synthetic sequence
CSVLRTSNTEAFF

SEQ ID NO: 701 - protein, 14 aa, Artificial sequence
  synthetic sequence
CASGRTSGGETQYF

SEQ ID NO: 702 - protein, 10 aa, Artificial sequence
  synthetic sequence
CSEDTDTQYF

SEQ ID NO: 703 - protein, 16 aa, Artificial sequence
  synthetic sequence
CASSQVPVSSGNTIYF

SEQ ID NO: 704 - protein, 16 aa, Artificial sequence
  synthetic sequence
CASSLAWTGLNTEAFF

SEQ ID NO: 705 - protein, 17 aa, Artificial sequence
  synthetic sequence
CASSVGFRSSYNSPLHF

SEQ ID NO: 706 - protein, 17 aa, Artificial sequence
  synthetic sequence
CASSVALRGALNYGYTF

SEQ ID NO: 707 - protein, 15 aa, Artificial sequence
  synthetic sequence
CASSESDRSSQPQHF

SEQ ID NO: 708 - protein, 15 aa, Artificial sequence
  synthetic sequence
CASSLDVSRNSPLHF

SEQ ID NO: 709 - protein, 14 aa, Artificial sequence
  synthetic sequence
CASSALGDNSPLHF

SEQ ID NO: 710 - protein, 13 aa, Artificial sequence
  synthetic sequence
CASSLEPIQPQHF

SEQ ID NO: 711 - protein, 15 aa, Artificial sequence
  synthetic sequence
CASSLDRIHNSPLHF

SEQ ID NO: 712 - protein, 16 aa, Artificial sequence
  synthetic sequence
CASSRDRGGSYNEQFF

SEQ ID NO: 713 - protein, 15 aa, Artificial sequence
  synthetic sequence
CASSLDSGRPDTQYF

SEQ ID NO: 714 - protein, 13 aa, Artificial sequence
  synthetic sequence
CASSLDQADTQYF

SEQ ID NO: 715 - protein, 13 aa, Artificial sequence
  synthetic sequence
CASSHRQSETQYF

SEQ ID NO: 716 - protein, 11 aa, Artificial sequence
  synthetic sequence
CASSNNEKLFF

SEQ ID NO: 717 - protein, 18 aa, Artificial sequence
  synthetic sequence
CASSLTSGRAGSSYEQYF

SEQ ID NO: 718 - protein, 16 aa, Artificial sequence
  synthetic sequence
CASSIGLAGGGNEQFF

SEQ ID NO: 719 - protein, 15 aa, Artificial sequence
  synthetic sequence
CAWSTRTGTTYEQYF

SEQ ID NO: 720 - protein, 13 aa, Artificial sequence
  synthetic sequence
CASSLHAGNTIYF

SEQ ID NO: 721 - protein, 16 aa, Artificial sequence
  synthetic sequence
CASSPPPDRPTGELFF

SEQ ID NO: 722 - protein, 14 aa, Artificial sequence
  synthetic sequence
CASSGQLSGNTIYF

SEQ ID NO: 723 - protein, 15 aa, Artificial sequence
  synthetic sequence
CASSYGTSYNSPLHF

SEQ ID NO: 724 - protein, 14 aa, Artificial sequence
  synthetic sequence
CASRSQGASYEQYF

SEQ ID NO: 725 - protein, 14 aa, Artificial sequence
  synthetic sequence
CASSPDVVLETQYF

SEQ ID NO: 726 - protein, 14 aa, Artificial sequence
  synthetic sequence
CASSSMTGGEKLFF

SEQ ID NO: 727 - protein, 11 aa, Artificial sequence
  synthetic sequence
CASSTGAEQYF

SEQ ID NO: 728 - protein, 15 aa, Artificial sequence
  synthetic sequence
CASSPREGRTDTQYF

SEQ ID NO: 729 - protein, 14 aa, Artificial sequence
  synthetic sequence
CASSLTSSLETQYF

SEQ ID NO: 730 - protein, 14 aa, Artificial sequence
  synthetic sequence
CASSVMQANTEAFF

SEQ ID NO: 731 - protein, 13 aa, Artificial sequence
  synthetic sequence
CSAPDLRSYEQYF

SEQ ID NO: 732 - protein, 15 aa, Artificial sequence
  synthetic sequence
CASSYLGEQGNEQYF

SEQ ID NO: 733 - protein, 16 aa, Artificial sequence
  synthetic sequence
CASSVRQDFSGNTIYF

SEQ ID NO: 734 - protein, 14 aa, Artificial sequence
  synthetic sequence
CSASQELSNQPQHF

SEQ ID NO: 735 - protein, 14 aa, Artificial sequence
  synthetic sequence
CSARRQGETGELFF

SEQ ID NO: 736 - protein, 14 aa, Artificial sequence
  synthetic sequence
CASSPPGARNEQYF

SEQ ID NO: 737 - protein, 12 aa, Artificial sequence
  synthetic sequence
CASSYDVVEAFF

SEQ ID NO: 738 - protein, 13 aa, Artificial sequence
  synthetic sequence
CSARRTPPTEAFF

SEQ ID NO: 739 - protein, 15 aa, Artificial sequence
  synthetic sequence
CASSLTQANNYGYTF

SEQ ID NO: 740 - protein, 12 aa, Artificial sequence
  synthetic sequence
CSVAGWGEKLFF

SEQ ID NO: 741 - protein, 15 aa, Artificial sequence
  synthetic sequence
CSARTLAGGAGELFF

SEQ ID NO: 742 - protein, 16 aa, Artificial sequence
  synthetic sequence
CASSLQGQGAPGELFF

SEQ ID NO: 743 - protein, 14 aa, Artificial sequence
  synthetic sequence
CASSPTNTGVEQYF

SEQ ID NO: 744 - protein, 15 aa, Artificial sequence
  synthetic sequence
CSAKPLGGGYNEQFF

SEQ ID NO: 745 - protein, 15 aa, Artificial sequence
  synthetic sequence
CASSLPDRVNSPLHF

SEQ ID NO: 746 - protein, 14 aa, Artificial sequence
  synthetic sequence
CASNNPGDTGELFF

SEQ ID NO: 747 - protein, 15 aa, Artificial sequence
  synthetic sequence
CASSYGQGWTQPQHF

SEQ ID NO: 748 - protein, 15 aa, Artificial sequence
  synthetic sequence
CASSQPGQGAREQYF

SEQ ID NO: 749 - protein, 16 aa, Artificial sequence
  synthetic sequence
CASSLAGGGRENEQFF

SEQ ID NO: 750 - protein, 16 aa, Artificial sequence
  synthetic sequence
CASSLAWRDYTDTQYF

SEQ ID NO: 751 - protein, 15 aa, Artificial sequence
  synthetic sequence
CASSYSGEQGNEQFF

SEQ ID NO: 752 - protein, 14 aa, Artificial sequence
  synthetic sequence
CSVRGVGENTEAFF

SEQ ID NO: 753 - protein, 15 aa, Artificial sequence
  synthetic sequence
CASSEGVRGGYGYTF

SEQ ID NO: 754 - protein, 14 aa, Artificial sequence
  synthetic sequence
CASSISRTENIQYF

SEQ ID NO: 755 - protein, 16 aa, Artificial sequence
  synthetic sequence
CASSFRGQGARQPQHF

SEQ ID NO: 756 - protein, 13 aa, Artificial sequence
  synthetic sequence
CATSTMGGTEAFF

SEQ ID NO: 757 - protein, 15 aa, Artificial sequence
  synthetic sequence
CASSLDLKSTGELFF

SEQ ID NO: 758 - protein, 15 aa, Artificial sequence
  synthetic sequence
CASSVVGAGEEKLFF

SEQ ID NO: 759 - protein, 16 aa, Artificial sequence
  synthetic sequence
CATSREQGFSSYEQYF

SEQ ID NO: 760 - protein, 15 aa, Artificial sequence
  synthetic sequence
CASSVVPTEGEKLFF

SEQ ID NO: 761 - protein, 14 aa, Artificial sequence
  synthetic sequence
CASSPHKQNTEAFF

SEQ ID NO: 762 - protein, 13 aa, Artificial sequence
  synthetic sequence
CASSLEMADTQYF

SEQ ID NO: 763 - protein, 16 aa, Artificial sequence
  synthetic sequence
CASSQVLGGLQETQYF

SEQ ID NO: 764 - protein, 14 aa, Artificial sequence
  synthetic sequence
CASSPGSNSQPQHF

SEQ ID NO: 765 - protein, 14 aa, Artificial sequence
  synthetic sequence
CASSPILARETQYF

SEQ ID NO: 766 - protein, 14 aa, Artificial sequence
  synthetic sequence
CASSPGAQSQPQHF

SEQ ID NO: 767 - protein, 15 aa, Artificial sequence
  synthetic sequence
CASSRSAGVGDTQYF

SEQ ID NO: 768 - protein, 12 aa, Artificial sequence
  synthetic sequence
CASSFGREKLFF

SEQ ID NO: 769 - protein, 15 aa, Artificial sequence
  synthetic sequence
CASSDHTGPGNTIYF

SEQ ID NO: 770 - protein, 12 aa, Artificial sequence
  synthetic sequence
CSVEEGTDTQYF

SEQ ID NO: 771 - protein, 14 aa, Artificial sequence
  synthetic sequence
CASSSTGNSYEQYF

SEQ ID NO: 772 - protein, 14 aa, Artificial sequence
  synthetic sequence
CASRRLAGDYEQYF

SEQ ID NO: 773 - protein, 16 aa, Artificial sequence
  synthetic sequence
CASSPPDGPTNEKLFF

SEQ ID NO: 774 - protein, 15 aa, Artificial sequence
  synthetic sequence
CSARSPGRYTGELFF

SEQ ID NO: 775 - protein, 15 aa, Artificial sequence
  synthetic sequence
CASSSRDGRTDTQYF

SEQ ID NO: 776 - protein, 15 aa, Artificial sequence
  synthetic sequence
CASSQDFGGAGGYTF

SEQ ID NO: 777 - protein, 14 aa, Artificial sequence
  synthetic sequence
CASSRTGQGNTIYF

SEQ ID NO: 778 - protein, 15 aa, Artificial sequence
  synthetic sequence
CASSQGGARGETQYF

SEQ ID NO: 779 - protein, 17 aa, Artificial sequence
  synthetic sequence
CASSLLGSRSGANVLTF

SEQ ID NO: 780 - protein, 13 aa, Artificial sequence
  synthetic sequence
CASSSEVQETQYF

SEQ ID NO: 781 - protein, 14 aa, Artificial sequence
  synthetic sequence
CASSYRVSNSPLHF

SEQ ID NO: 782 - protein, 15 aa, Artificial sequence
  synthetic sequence
CASSLYGSGPYEQYF

SEQ ID NO: 783 - protein, 15 aa, Artificial sequence
  synthetic sequence
CSARDNRADTGELFF

SEQ ID NO: 784 - protein, 14 aa, Artificial sequence
  synthetic sequence
CASSGAGGGETQYF

SEQ ID NO: 785 - protein, 14 aa, Artificial sequence
  synthetic sequence
CASSLYPQVYEQYF

SEQ ID NO: 786 - protein, 17 aa, Artificial sequence
  synthetic sequence
CASSHRTSGGPTGELFF

SEQ ID NO: 787 - protein, 13 aa, Artificial sequence
  synthetic sequence
CASSPYTNQPQHF

SEQ ID NO: 788 - protein, 15 aa, Artificial sequence
  synthetic sequence
CASSDWTSGYNEQFF

SEQ ID NO: 789 - protein, 15 aa, Artificial sequence
  synthetic sequence
CSANGLAGVVGEQFF

SEQ ID NO: 790 - protein, 14 aa, Artificial sequence
  synthetic sequence
CASSRQREDQPQHF

SEQ ID NO: 791 - protein, 15 aa, Artificial sequence
  synthetic sequence
CASRSSTGRNQPQHF

SEQ ID NO: 792 - protein, 17 aa, Artificial sequence
  synthetic sequence
CASSGLTSGSRTDTQYF

SEQ ID NO: 793 - protein, 14 aa, Artificial sequence
  synthetic sequence
CASSQEGFTDTQYF

SEQ ID NO: 794 - protein, 16 aa, Artificial sequence
  synthetic sequence
CASSLAGVAGVDEQFF

SEQ ID NO: 795 - protein, 14 aa, Artificial sequence
  synthetic sequence
CASSPLDTRYEQYF

SEQ ID NO: 796 - protein, 14 aa, Artificial sequence
  synthetic sequence
CASSNPGDRNTIYF

SEQ ID NO: 797 - protein, 15 aa, Artificial sequence
  synthetic sequence
CASSPGQISNTEAFF

SEQ ID NO: 798 - protein, 15 aa, Artificial sequence
  synthetic sequence
CSVEGGRGNTDTQYF

SEQ ID NO: 799 - protein, 17 aa, Artificial sequence
  synthetic sequence
CASSLLGGLSSGNTIYF

SEQ ID NO: 800 - protein, 15 aa, Artificial sequence
  synthetic sequence
CASSSPGHWNTEAFF

SEQ ID NO: 801 - protein, 14 aa, Artificial sequence
  synthetic sequence
CASSFWQGRHEQYF

SEQ ID NO: 802 - protein, 14 aa, Artificial sequence
  synthetic sequence
CASSLYPSTYEQYF

SEQ ID NO: 803 - protein, 15 aa, Artificial sequence
  synthetic sequence
CASSLWGARYYGYTF

SEQ ID NO: 804 - protein, 15 aa, Artificial sequence
  synthetic sequence
CASSLGGGPLSEQFF

SEQ ID NO: 805 - protein, 15 aa, Artificial sequence
  synthetic sequence
CASSYLGEQGQPQHF

SEQ ID NO: 806 - protein, 15 aa, Artificial sequence
  synthetic sequence
CASSEGQGEGYEQYF

SEQ ID NO: 807 - protein, 15 aa, Artificial sequence
  synthetic sequence
CASSYLGEQGNGYTF

SEQ ID NO: 808 - protein, 15 aa, Artificial sequence
  synthetic sequence
CASSPQWAGSYEQYF

SEQ ID NO: 809 - protein, 13 aa, Artificial sequence
  synthetic sequence
CASSSGTGEGYTF

SEQ ID NO: 810 - protein, 15 aa, Artificial sequence
  synthetic sequence
CASSLWGGPDNEQFF

SEQ ID NO: 811 - protein, 13 aa, Artificial sequence
  synthetic sequence
CASSAPSDEKLFF

SEQ ID NO: 812 - protein, 15 aa, Artificial sequence
  synthetic sequence
CSARILAGGSGELFF

SEQ ID NO: 813 - protein, 16 aa, Artificial sequence
  synthetic sequence
CASSLSSSFPYNEQFF

SEQ ID NO: 814 - protein, 13 aa, Artificial sequence
  synthetic sequence
CASSLGVRRGYTF

SEQ ID NO: 815 - protein, 16 aa, Artificial sequence
  synthetic sequence
CATSRDLAESSYEQYF

SEQ ID NO: 816 - protein, 15 aa, Artificial sequence
  synthetic sequence
CASSFWQGRNQPQHF

SEQ ID NO: 817 - protein, 14 aa, Artificial sequence
  synthetic sequence
CASSPRWGTDTQYF

SEQ ID NO: 818 - protein, 17 aa, Artificial sequence
  synthetic sequence
CASSQGSTSGGRETQYF

SEQ ID NO: 819 - protein, 16 aa, Artificial sequence
  synthetic sequence
CSARPGTGGLTDTQYF

SEQ ID NO: 820 - protein, 15 aa, Artificial sequence
  synthetic sequence
CASSLFGGVQETQYF

SEQ ID NO: 821 - protein, 15 aa, Artificial sequence
  synthetic sequence
CASSLYGGLQETQYF

SEQ ID NO: 822 - protein, 15 aa, Artificial sequence
  synthetic sequence
CASSLYGSLTDTQYF

SEQ ID NO: 823 - protein, 13 aa, Artificial sequence
  synthetic sequence
CATKGGTNEKLFF

SEQ ID NO: 824 - protein, 15 aa, Artificial sequence
  synthetic sequence
CASSQGRATGGGYTF

SEQ ID NO: 825 - protein, 15 aa, Artificial sequence
  synthetic sequence
CASSSTGQGANEQFF

SEQ ID NO: 826 - protein, 13 aa, Artificial sequence
  synthetic sequence
CASSPRLFNEQFF

SEQ ID NO: 827 - protein, 15 aa, Artificial sequence
  synthetic sequence
CAISLEGGGSYEQYF

SEQ ID NO: 828 - protein, 15 aa, Artificial sequence
  synthetic sequence
CASSLRRGVNTEAFF

SEQ ID NO: 829 - protein, 11 aa, Artificial sequence
  synthetic sequence
CAWGPAGELFF

SEQ ID NO: 830 - protein, 16 aa, Artificial sequence
  synthetic sequence
CASSYGNPGQTYEQYF

SEQ ID NO: 831 - protein, 16 aa, Artificial sequence
  synthetic sequence
CASSLDRGAGYNEQFF

SEQ ID NO: 832 - protein, 16 aa, Artificial sequence
  synthetic sequence
CATSREGTGVGTEAFF

SEQ ID NO: 833 - protein, 15 aa, Artificial sequence
  synthetic sequence
CATSRDSSFQETQYF

SEQ ID NO: 834 - protein, 13 aa, Artificial sequence
  synthetic sequence
CASSYGTVTEAFF

SEQ ID NO: 835 - protein, 13 aa, Artificial sequence
  synthetic sequence
CSAVTRTYNEQFF

SEQ ID NO: 836 - protein, 13 aa, Artificial sequence
  synthetic sequence
CASSREIADTQYF

SEQ ID NO: 837 - protein, 13 aa, Artificial sequence
  synthetic sequence
CASSFKGRRGYTF

SEQ ID NO: 838 - protein, 13 aa, Artificial sequence
  synthetic sequence
CSVGEQGTDTQYF

SEQ ID NO: 839 - protein, 14 aa, Artificial sequence
  synthetic sequence
CASSQGSLQETQYF

SEQ ID NO: 840 - protein, 14 aa, Artificial sequence
  synthetic sequence
CASSPPGGRNEQFF

SEQ ID NO: 841 - protein, 15 aa, Artificial sequence
  synthetic sequence
CASSLTGLVQETQYF

SEQ ID NO: 842 - protein, 16 aa, Artificial sequence
  synthetic sequence
CASSESRGRAANEQFF

SEQ ID NO: 843 - protein, 18 aa, Artificial sequence
  synthetic sequence
CASSLYVQASSYNSPLHF

SEQ ID NO: 844 - protein, 16 aa, Artificial sequence
  synthetic sequence
CASSWRVAGPQETQYF

SEQ ID NO: 845 - protein, 17 aa, Artificial sequence
  synthetic sequence
CASSERTGGSYNSPLHF

SEQ ID NO: 846 - protein, 15 aa, Artificial sequence
  synthetic sequence
CSARDNLADTGELFF

SEQ ID NO: 847 - protein, 15 aa, Artificial sequence
  synthetic sequence
CSAIRTGGYQETQYF

SEQ ID NO: 848 - protein, 14 aa, Artificial sequence
  synthetic sequence
CASSQPGQVNEQFF

SEQ ID NO: 849 - protein, 15 aa, Artificial sequence
  synthetic sequence
CASSRLAGAGETQYF

SEQ ID NO: 850 - protein, 14 aa, Artificial sequence
  synthetic sequence
CASSPGTSLNTQYF

SEQ ID NO: 851 - protein, 17 aa, Artificial sequence
  synthetic sequence
CASSLVGGSSGANVLTF

SEQ ID NO: 852 - protein, 15 aa, Artificial sequence
  synthetic sequence
CASSYRQGVRETQYF

SEQ ID NO: 853 - protein, 15 aa, Artificial sequence
  synthetic sequence
CASSPLGAGTDTQYF

SEQ ID NO: 854 - protein, 12 aa, Artificial sequence
  synthetic sequence
CASSPRGNEQFF

SEQ ID NO: 855 - protein, 14 aa, Artificial sequence
  synthetic sequence
CASRSPGDYNEQFF

SEQ ID NO: 856 - protein, 16 aa, Artificial sequence
  synthetic sequence
CASSESTGPLTDTQYF

SEQ ID NO: 857 - protein, 15 aa, Artificial sequence
  synthetic sequence
CASSYGLAGVRAQYF

SEQ ID NO: 858 - protein, 14 aa, Artificial sequence
  synthetic sequence
CASTPGQGSDGYTF

SEQ ID NO: 859 - protein, 15 aa, Artificial sequence
  synthetic sequence
CAWSPGQAAGEKLFF

SEQ ID NO: 860 - protein, 16 aa, Artificial sequence
  synthetic sequence
CASSLGLAGGRDIQYF

SEQ ID NO: 861 - protein, 15 aa, Artificial sequence
  synthetic sequence
CASSIDSGTGDEAFF

SEQ ID NO: 862 - protein, 14 aa, Artificial sequence
  synthetic sequence
CASSLNRINTEAFF

SEQ ID NO: 863 - protein, 16 aa, Artificial sequence
  synthetic sequence
CASSLDGGRAPDTQYF

SEQ ID NO: 864 - protein, 12 aa, Artificial sequence
  synthetic sequence
CASSPRDHEQYF

SEQ ID NO: 865 - protein, 15 aa, Artificial sequence
  synthetic sequence
CSARKLAGGSGELFF

SEQ ID NO: 866 - protein, 18 aa, Artificial sequence
  synthetic sequence
CASSQEGLAGGITYEQYF

SEQ ID NO: 867 - protein, 15 aa, Artificial sequence
  synthetic sequence
CASSPYGGGRYEQYF

SEQ ID NO: 868 - protein, 15 aa, Artificial sequence
  synthetic sequence
CSAKDLGGPYNEQFF

SEQ ID NO: 869 - protein, 12 aa, Artificial sequence
  synthetic sequence
CATSSDTKTQYF

SEQ ID NO: 870 - protein, 12 aa, Artificial sequence
  synthetic sequence
CASKGEEETQYF

SEQ ID NO: 871 - protein, 14 aa, Artificial sequence
  synthetic sequence
CASSPQGAQDTQYF

SEQ ID NO: 872 - protein, 14 aa, Artificial sequence
  synthetic sequence
CASRGQDPNEKLFF

SEQ ID NO: 873 - protein, 12 aa, Artificial sequence
  synthetic sequence
CASTLTGNTIYF

SEQ ID NO: 874 - protein, 13 aa, Artificial sequence
  synthetic sequence
CSAPRQGTDTQYF

SEQ ID NO: 875 - protein, 14 aa, Artificial sequence
  synthetic sequence
CSARAQGETGELFF

SEQ ID NO: 876 - protein, 15 aa, Artificial sequence
  synthetic sequence
CASRGGRDSGNTIYF

SEQ ID NO: 877 - protein, 14 aa, Artificial sequence
  synthetic sequence
CASSDVGRNNEQFF

SEQ ID NO: 878 - protein, 15 aa, Artificial sequence
  synthetic sequence
CASSFLPGLGNEQFF

SEQ ID NO: 879 - protein, 16 aa, Artificial sequence
  synthetic sequence
CASSPPDNPTNEKLFF

SEQ ID NO: 880 - protein, 12 aa, Artificial sequence
  synthetic sequence
CSALRPGPGYTF

SEQ ID NO: 881 - protein, 12 aa, Artificial sequence
  synthetic sequence
CSVGQGADEAFF

SEQ ID NO: 882 - protein, 14 aa, Artificial sequence
  synthetic sequence
CASSMGNGPYEQYF

SEQ ID NO: 883 - protein, 15 aa, Artificial sequence
  synthetic sequence
CASRSPAGRNQPQHF

SEQ ID NO: 884 - protein, 15 aa, Artificial sequence
  synthetic sequence
CASSLVGVPPDTQYF

SEQ ID NO: 885 - protein, 14 aa, Artificial sequence
  synthetic sequence
CASSPGTSRRTQYF

SEQ ID NO: 886 - protein, 15 aa, Artificial sequence
  synthetic sequence
CASTLGGGWGQPQHF

SEQ ID NO: 887 - protein, 14 aa, Artificial sequence
  synthetic sequence
CASSLVNGNYGYTF

SEQ ID NO: 888 - protein, 13 aa, Artificial sequence
  synthetic sequence
CASSTERAGELFF

SEQ ID NO: 889 - protein, 14 aa, Artificial sequence
  synthetic sequence
CASSFVREGEKLFF

SEQ ID NO: 890 - protein, 13 aa, Artificial sequence
  synthetic sequence
CASSAYTNQPQHF

SEQ ID NO: 891 - protein, 16 aa, Artificial sequence
  synthetic sequence
CSASLTSGRAADTQYF

SEQ ID NO: 892 - protein, 14 aa, Artificial sequence
  synthetic sequence
CASSPVDSNYGYTF

SEQ ID NO: 893 - protein, 12 aa, Artificial sequence
  synthetic sequence
CSAADWQETQYF

SEQ ID NO: 894 - protein, 15 aa, Artificial sequence
  synthetic sequence
CASSTRTGVRETQYF

SEQ ID NO: 895 - protein, 12 aa, Artificial sequence
  synthetic sequence
CSAKGPGAEAFF

SEQ ID NO: 896 - protein, 14 aa, Artificial sequence
  synthetic sequence
CASGPSTSTDTQYF

SEQ ID NO: 897 - protein, 14 aa, Artificial sequence
  synthetic sequence
CSAPSSGGPYEQYF

SEQ ID NO: 898 - protein, 13 aa, Artificial sequence
  synthetic sequence
CASSFEIQETQYF

SEQ ID NO: 899 - protein, 15 aa, Artificial sequence
  synthetic sequence
CATSRDNSQQETQYF

SEQ ID NO: 900 - protein, 13 aa, Artificial sequence
  synthetic sequence
CSAITRTYNEQFF

SEQ ID NO: 901 - protein, 15 aa, Artificial sequence
  synthetic sequence
CASSLAGGGLYEQYF

SEQ ID NO: 902 - protein, 15 aa, Artificial sequence
  synthetic sequence
CASSSETGGRNEQFF

SEQ ID NO: 903 - protein, 16 aa, Artificial sequence
  synthetic sequence
CASSDRTGTSYNEQFF

SEQ ID NO: 904 - protein, 17 aa, Artificial sequence
  synthetic sequence
CASSLLGQSSGANVLTF

SEQ ID NO: 905 - protein, 15 aa, Artificial sequence
  synthetic sequence
CASSLGTDPRETQYF

SEQ ID NO: 906 - protein, 12 aa, Artificial sequence
  synthetic sequence
CAIRQDPYEQYF

SEQ ID NO: 907 - protein, 15 aa, Artificial sequence
  synthetic sequence
CASSARGQGSYEQYF

SEQ ID NO: 908 - protein, 15 aa, Artificial sequence
  synthetic sequence
CAWSPIDSLNTEAFF

SEQ ID NO: 909 - protein, 12 aa, Artificial sequence
  synthetic sequence
CASSGTGDDYTF

SEQ ID NO: 910 - protein, 12 aa, Artificial sequence
  synthetic sequence
CATTTTGDEQYF

SEQ ID NO: 911 - protein, 16 aa, Artificial sequence
  synthetic sequence
CASSIIAGKGNQPQHF

SEQ ID NO: 912 - protein, 13 aa, Artificial sequence
  synthetic sequence
CASNRWGQETQYF

SEQ ID NO: 913 - protein, 14 aa, Artificial sequence
  synthetic sequence
CASSYGSAGTEAFF

SEQ ID NO: 914 - protein, 12 aa, Artificial sequence
  synthetic sequence
CASRPQRNEQFF

SEQ ID NO: 915 - protein, 16 aa, Artificial sequence
  synthetic sequence
CASSQGDRGATDTQYF

SEQ ID NO: 916 - protein, 13 aa, Artificial sequence
  synthetic sequence
CASSQVSGFEQFF

SEQ ID NO: 917 - protein, 14 aa, Artificial sequence
  synthetic sequence
CSARSQGETGELFF

SEQ ID NO: 918 - protein, 15 aa, Artificial sequence
  synthetic sequence
CASSLGSGGVGEQYF

SEQ ID NO: 919 - protein, 14 aa, Artificial sequence
  synthetic sequence
CASRRDRGLYGYTF

SEQ ID NO: 920 - protein, 14 aa, Artificial sequence
  synthetic sequence
CASRPGQGGDEQYF

SEQ ID NO: 921 - protein, 14 aa, Artificial sequence
  synthetic sequence
CASSIDSRDNEQFF

SEQ ID NO: 922 - protein, 14 aa, Artificial sequence
  synthetic sequence
CASSGPAANEKLFF

SEQ ID NO: 923 - protein, 14 aa, Artificial sequence
  synthetic sequence
CASRGTGSVNEQFF

SEQ ID NO: 924 - protein, 15 aa, Artificial sequence
  synthetic sequence
CASGGTSSGANVLTF

SEQ ID NO: 925 - protein, 14 aa, Artificial sequence
  synthetic sequence
CAISAGGNRETQYF

SEQ ID NO: 926 - protein, 14 aa, Artificial sequence
  synthetic sequence
CASSSAVSGTEAFF

SEQ ID NO: 927 - protein, 14 aa, Artificial sequence
  synthetic sequence
CASSPQDRYYGYTF

SEQ ID NO: 928 - protein, 15 aa, Artificial sequence
  synthetic sequence
CASSLAATLYNEQFF

SEQ ID NO: 929 - protein, 16 aa, Artificial sequence
  synthetic sequence
CATSRDLPESSYEQYF

SEQ ID NO: 930 - protein, 16 aa, Artificial sequence
  synthetic sequence
CASSPLSGTVSYEQYF

SEQ ID NO: 931 - protein, 16 aa, Artificial sequence
  synthetic sequence
CASSLDHRGRVTEAFF

SEQ ID NO: 932 - protein, 14 aa, Artificial sequence
  synthetic sequence
CASSYLDRAHEQYF

SEQ ID NO: 933 - protein, 13 aa, Artificial sequence
  synthetic sequence
CATHRLANYGYTF

SEQ ID NO: 934 - protein, 15 aa, Artificial sequence
  synthetic sequence
CASSQSVGDTGELFF

SEQ ID NO: 935 - protein, 16 aa, Artificial sequence
  synthetic sequence
CASSQRTSGIQETQYF

SEQ ID NO: 936 - protein, 13 aa, Artificial sequence
  synthetic sequence
CASSPVASNEQFF

SEQ ID NO: 937 - protein, 17 aa, Artificial sequence
  synthetic sequence
CASSLSGLAGAPDTQYF

SEQ ID NO: 938 - protein, 14 aa, Artificial sequence
  synthetic sequence
CASSLGGAKDEQYF

SEQ ID NO: 939 - protein, 14 aa, Artificial sequence
  synthetic sequence
CASSPGTSMKTQYF

SEQ ID NO: 940 - protein, 14 aa, Artificial sequence
  synthetic sequence
CSAFQGAAGNTIYF

SEQ ID NO: 941 - protein, 14 aa, Artificial sequence
  synthetic sequence
CASRRGRDPDTQYF

SEQ ID NO: 942 - protein, 15 aa, Artificial sequence
  synthetic sequence
CASSVWGDDNQPQHF

SEQ ID NO: 943 - protein, 15 aa, Artificial sequence
  synthetic sequence
CASSLAGVDTDTQYF

SEQ ID NO: 944 - protein, 14 aa, Artificial sequence
  synthetic sequence
CSARDQGETGELFF

SEQ ID NO: 945 - protein, 15 aa, Artificial sequence
  synthetic sequence
CASSEFWGSTDTQYF

SEQ ID NO: 946 - protein, 14 aa, Artificial sequence
  synthetic sequence
CASSDPAGANVLTF

SEQ ID NO: 947 - protein, 18 aa, Artificial sequence
  synthetic sequence
CASSQDRTGGPLGNEQFF

SEQ ID NO: 948 - protein, 13 aa, Artificial sequence
  synthetic sequence
CATRREIADTQYF

SEQ ID NO: 949 - protein, 14 aa, Artificial sequence
  synthetic sequence
CASSSYTNTGELFF

SEQ ID NO: 950 - protein, 11 aa, Artificial sequence
  synthetic sequence
CASSSGARQYF

SEQ ID NO: 951 - protein, 15 aa, Artificial sequence
  synthetic sequence
CSARDRAGGPGELFF

SEQ ID NO: 952 - protein, 14 aa, Artificial sequence
  synthetic sequence
CASSLALSGNTIYF

SEQ ID NO: 953 - protein, 13 aa, Artificial sequence
  synthetic sequence
CASSLEGGSGYTF

SEQ ID NO: 954 - protein, 15 aa, Artificial sequence
  synthetic sequence
CSARVGGRPEETQYF

SEQ ID NO: 955 - protein, 14 aa, Artificial sequence
  synthetic sequence
CASSAPGDNYGYTF

SEQ ID NO: 956 - protein, 13 aa, Artificial sequence
  synthetic sequence
CASSYRGVYEQYF

SEQ ID NO: 957 - protein, 13 aa, Artificial sequence
  synthetic sequence
CSVPLQGTDTQYF

SEQ ID NO: 958 - protein, 14 aa, Artificial sequence
  synthetic sequence
CASSGDRVSDGYTF

SEQ ID NO: 959 - protein, 14 aa, Artificial sequence
  synthetic sequence
CASSFWQGRNEQFF

SEQ ID NO: 960 - protein, 13 aa, Artificial sequence
  synthetic sequence
CASRYSGTGELFF

SEQ ID NO: 961 - protein, 15 aa, Artificial sequence
  synthetic sequence
CASSGGDREDQPQHF

SEQ ID NO: 962 - protein, 15 aa, Artificial sequence
  synthetic sequence
CASSIRTGVRETQYF

SEQ ID NO: 963 - protein, 15 aa, Artificial sequence
  synthetic sequence
CASSPLQGRAGELFF

SEQ ID NO: 964 - protein, 14 aa, Artificial sequence
  synthetic sequence
CSASVSRGSYEQYF

SEQ ID NO: 965 - protein, 15 aa, Artificial sequence
  synthetic sequence
CASSSETSGRNEQYF

SEQ ID NO: 966 - protein, 13 aa, Artificial sequence
  synthetic sequence
CASSSHSTYEQYF

SEQ ID NO: 967 - protein, 16 aa, Artificial sequence
  synthetic sequence
CASSVEEDRGLNEQFF

SEQ ID NO: 968 - protein, 14 aa, Artificial sequence
  synthetic sequence
CASSSQTGLYGYTF

SEQ ID NO: 969 - protein, 14 aa, Artificial sequence
  synthetic sequence
CASSQVGEKNIQYF

SEQ ID NO: 970 - protein, 13 aa, Artificial sequence
  synthetic sequence
CSVEGGRVGELFF

SEQ ID NO: 971 - protein, 15 aa, Artificial sequence
  synthetic sequence
CASSLRTGVREKLFF

SEQ ID NO: 972 - protein, 14 aa, Artificial sequence
  synthetic sequence
CASSLWQARYEQYF

SEQ ID NO: 973 - protein, 14 aa, Artificial sequence
  synthetic sequence
CASSLAGAPSEQFF

SEQ ID NO: 974 - protein, 16 aa, Artificial sequence
  synthetic sequence
CASSARTVSGANVLTF

SEQ ID NO: 975 - protein, 16 aa, Artificial sequence
  synthetic sequence
CASSPGTGFGNTEAFF

SEQ ID NO: 976 - protein, 13 aa, Artificial sequence
  synthetic sequence
CASRREIADTQYF

SEQ ID NO: 977 - protein, 17 aa, Artificial sequence
  synthetic sequence
CASSLKGAGSGANVLTF

SEQ ID NO: 978 - protein, 15 aa, Artificial sequence
  synthetic sequence
CASSLAGGSLYEQYF

SEQ ID NO: 979 - protein, 14 aa, Artificial sequence
  synthetic sequence
CASRPGGAPTEAFF

SEQ ID NO: 980 - protein, 15 aa, Artificial sequence
  synthetic sequence
CASSLELVGGYEQYF

SEQ ID NO: 981 - protein, 15 aa, Artificial sequence
  synthetic sequence
CASSSDRDSPYEQYF

SEQ ID NO: 982 - protein, 14 aa, Artificial sequence
  synthetic sequence
CASSLLGVSYEQYF

SEQ ID NO: 983 - protein, 15 aa, Artificial sequence
  synthetic sequence
CASSETWAGNTEAFF

SEQ ID NO: 984 - protein, 14 aa, Artificial sequence
  synthetic sequence
CASSQETGLNEQYF

SEQ ID NO: 985 - protein, 13 aa, Artificial sequence
  synthetic sequence
CASTPLNTGELFF

SEQ ID NO: 986 - protein, 13 aa, Artificial sequence
  synthetic sequence
CASRRLVNTEAFF

SEQ ID NO: 987 - protein, 14 aa, Artificial sequence
  synthetic sequence
CASSLGVSGGEQFF

SEQ ID NO: 988 - protein, 15 aa, Artificial sequence
  synthetic sequence
CASSLELTGGYEQYF

SEQ ID NO: 989 - protein, 14 aa, Artificial sequence
  synthetic sequence
CASSAQQGRTEAFF

SEQ ID NO: 990 - protein, 16 aa, Artificial sequence
  synthetic sequence
CASSLITSGDTDTQYF

SEQ ID NO: 991 - protein, 13 aa, Artificial sequence
  synthetic sequence
CASRWGFQETQYF

SEQ ID NO: 992 - protein, 15 aa, Artificial sequence
  synthetic sequence
CASSFGGPGGQPQHF

SEQ ID NO: 993 - protein, 14 aa, Artificial sequence
  synthetic sequence
CASGTWGSNQPQHF

SEQ ID NO: 994 - protein, 14 aa, Artificial sequence
  synthetic sequence
CASSERDRGFEQYF

SEQ ID NO: 995 - protein, 15 aa, Artificial sequence
  synthetic sequence
CASSRSKQGDTEAFF

SEQ ID NO: 996 - protein, 14 aa, Artificial sequence
  synthetic sequence
CSVWTGSSYNEQFF

SEQ ID NO: 997 - protein, 13 aa, Artificial sequence
  synthetic sequence
CASSLDSFTEAFF

SEQ ID NO: 998 - protein, 15 aa, Artificial sequence
  synthetic sequence
CASSLFGAGPYEQYF

SEQ ID NO: 999 - protein, 15 aa, Artificial sequence
  synthetic sequence
CSARILAGGAGELFF

SEQ ID NO: 1000 - protein, 13 aa, Artificial sequence
  synthetic sequence
CAWNRGLDYEQYF

SEQ ID NO: 1001 - protein, 14 aa, Artificial sequence
  synthetic sequence
CASSKTGQGNTIYF

SEQ ID NO: 1002 - protein, 13 aa, Artificial sequence
  synthetic sequence
CASSPFTNQPQHF

SEQ ID NO: 1003 - protein, 14 aa, Artificial sequence
  synthetic sequence
CASSSTGNTYEQYF

SEQ ID NO: 1004 - protein, 15 aa, Artificial sequence
  synthetic sequence
CASSYGQGRYNEQFF

SEQ ID NO: 1005 - protein, 15 aa, Artificial sequence
  synthetic sequence
CASSLFGSGPYEQYF

SEQ ID NO: 1006 - protein, 13 aa, Artificial sequence
  synthetic sequence
CASSLLQRREQYF

SEQ ID NO: 1007 - protein, 14 aa, Artificial sequence
  synthetic sequence
CASIQIGDTGELFF

SEQ ID NO: 1008 - protein, 14 aa, Artificial sequence
  synthetic sequence
CASSIDSRDHEQYF

SEQ ID NO: 1009 - protein, 11 aa, Artificial sequence
  synthetic sequence
CASSSGAQQFF

SEQ ID NO: 1010 - protein, 14 aa, Artificial sequence
  synthetic sequence
CSASPRSGNQPQHF

SEQ ID NO: 1011 - protein, 16 aa, Artificial sequence
  synthetic sequence
CASSLLSGTVSYEQYF

SEQ ID NO: 1012 - protein, 15 aa, Artificial sequence
  synthetic sequence
CASSVRQGIREKLFF

SEQ ID NO: 1013 - protein, 15 aa, Artificial sequence
  synthetic sequence
CASREGGVGNQPQHF

SEQ ID NO: 1014 - protein, 14 aa, Artificial sequence
  synthetic sequence
CASSPPWGADTQYF

SEQ ID NO: 1015 - protein, 15 aa, Artificial sequence
  synthetic sequence
CASSLLGQGRDGYTF

SEQ ID NO: 1016 - protein, 15 aa, Artificial sequence
  synthetic sequence
CASSYRTGVRETQYF

SEQ ID NO: 1017 - protein, 15 aa, Artificial sequence
  synthetic sequence
CASSDRDSGTGELFF

SEQ ID NO: 1018 - protein, 14 aa, Artificial sequence
  synthetic sequence
CASSLREIGETQYF

SEQ ID NO: 1019 - protein, 14 aa, Artificial sequence
  synthetic sequence
CASSLYPSIYEQYF

SEQ ID NO: 1020 - protein, 15 aa, Artificial sequence
  synthetic sequence
CASSLSGAGAGEQYF

SEQ ID NO: 1021 - protein, 17 aa, Artificial sequence
  synthetic sequence
CASSLESGTSGYNEQFF

SEQ ID NO: 1022 - protein, 15 aa, Artificial sequence
  synthetic sequence
CASSQGEREGHEQYF

SEQ ID NO: 1023 - protein, 16 aa, Artificial sequence
  synthetic sequence
CASSLYGGMNTGELFF

SEQ ID NO: 1024 - protein, 15 aa, Artificial sequence
  synthetic sequence
CASSIRTGIRETQYF

SEQ ID NO: 1025 - protein, 15 aa, Artificial sequence
  synthetic sequence
CASSYSAGEAQPQHF

SEQ ID NO: 1026 - protein, 15 aa, Artificial sequence
  synthetic sequence
CASSLIFGQSYEQYF

SEQ ID NO: 1027 - protein, 14 aa, Artificial sequence
  synthetic sequence
CSAHRQPGGETQYF

SEQ ID NO: 1028 - protein, 14 aa, Artificial sequence
  synthetic sequence
CASSSQTGLGGYTF

SEQ ID NO: 1029 - protein, 15 aa, Artificial sequence
  synthetic sequence
CSAQPPSGGSYEQYF

SEQ ID NO: 1030 - protein, 18 aa, Artificial sequence
  synthetic sequence
CASSSLRDRSSYNSPLHF

SEQ ID NO: 1031 - protein, 15 aa, Artificial sequence
  synthetic sequence
CASTRAPGSSYEQYF

SEQ ID NO: 1032 - protein, 16 aa, Artificial sequence
  synthetic sequence
CASSLDRGRGYNEQFF

SEQ ID NO: 1033 - protein, 14 aa, Artificial sequence
  synthetic sequence
CSARGQGEVGELFF

SEQ ID NO: 1034 - protein, 15 aa, Artificial sequence
  synthetic sequence
CASRSPQGRNQPQHF

SEQ ID NO: 1035 - protein, 16 aa, Artificial sequence
  synthetic sequence
CASSLDSTLAKNIQYF

SEQ ID NO: 1036 - protein, 15 aa, Artificial sequence
  synthetic sequence
CAWSPPDSLNEKLFF

SEQ ID NO: 1037 - protein, 14 aa, Artificial sequence
  synthetic sequence
CSARAQGEAGELFF

SEQ ID NO: 1038 - protein, 15 aa, Artificial sequence
  synthetic sequence
CASSTRQGVRETQYF

SEQ ID NO: 1039 - protein, 17 aa, Artificial sequence
  synthetic sequence
CASSQDRGGAGNQPQHF

SEQ ID NO: 1040 - protein, 12 aa, Artificial sequence
  synthetic sequence
CASSFRDNEQFF

SEQ ID NO: 1041 - protein, 13 aa, Artificial sequence
  synthetic sequence
CASSQNLNYGYTF

SEQ ID NO: 1042 - protein, 14 aa, Artificial sequence
  synthetic sequence
CASSYAGENNEQFF

SEQ ID NO: 1043 - protein, 15 aa, Artificial sequence
  synthetic sequence
CASSQRQGVREKLFF

SEQ ID NO: 1044 - protein, 18 aa, Artificial sequence
  synthetic sequence
CASSLTSGTSGSSYEQYF

SEQ ID NO: 1045 - protein, 13 aa, Artificial sequence
  synthetic sequence
CASSRKGDEKLFF

SEQ ID NO: 1046 - protein, 15 aa, Artificial sequence
  synthetic sequence
CSANRLSSYNSPLHF

SEQ ID NO: 1047 - protein, 13 aa, Artificial sequence
  synthetic sequence
CASSELPVTEAFF

SEQ ID NO: 1048 - protein, 14 aa, Artificial sequence
  synthetic sequence
CATSDLTDRDGYTF

SEQ ID NO: 1049 - protein, 15 aa, Artificial sequence
  synthetic sequence
CASSWRTGVRETQYF

SEQ ID NO: 1050 - protein, 16 aa, Artificial sequence
  synthetic sequence
CASSLGTVASNQPQHF

SEQ ID NO: 1051 - protein, 14 aa, Artificial sequence
  synthetic sequence
CASSFWQGRNTQYF

SEQ ID NO: 1052 - protein, 14 aa, Artificial sequence
  synthetic sequence
CASSPLLERETQYF

SEQ ID NO: 1053 - protein, 14 aa, Artificial sequence
  synthetic sequence
CASSFWQGRHIQYF

SEQ ID NO: 1054 - protein, 14 aa, Artificial sequence
  synthetic sequence
CSARGQGEAGELFF

SEQ ID NO: 1055 - protein, 15 aa, Artificial sequence
  synthetic sequence
CASSIRQGVRETQYF

SEQ ID NO: 1056 - protein, 15 aa, Artificial sequence
  synthetic sequence
CSAQKLSSYNSPLHF

SEQ ID NO: 1057 - protein, 14 aa, Artificial sequence
  synthetic sequence
CASSDHGQNTEAFF

SEQ ID NO: 1058 - protein, 15 aa, Artificial sequence
  synthetic sequence
CASSPLAGSTNEQFF

SEQ ID NO: 1059 - protein, 16 aa, Artificial sequence
  synthetic sequence
CATSRDPQGAKNEQFF

SEQ ID NO: 1060 - protein, 15 aa, Artificial sequence
  synthetic sequence
CASSPPDREDQPQHF

SEQ ID NO: 1061 - protein, 15 aa, Artificial sequence
  synthetic sequence
CSARPGGGVNQPQHF

SEQ ID NO: 1062 - protein, 15 aa, Artificial sequence
  synthetic sequence
CASSVRTGVRETQYF

SEQ ID NO: 1063 - protein, 16 aa, Artificial sequence
  synthetic sequence
CASSRQLAGGAYEQYF

SEQ ID NO: 1064 - protein, 15 aa, Artificial sequence
  synthetic sequence
CASSLGGLVKETQYF

SEQ ID NO: 1065 - protein, 15 aa, Artificial sequence
  synthetic sequence
CASSLLTSGTYEQYF

SEQ ID NO: 1066 - protein, 13 aa, Artificial sequence
  synthetic sequence
CASSLDRRREQYF

SEQ ID NO: 1067 - protein, 14 aa, Artificial sequence
  synthetic sequence
CASRGTGSTNEQFF

SEQ ID NO: 1068 - protein, 16 aa, Artificial sequence
  synthetic sequence
CASSLGLAGSHNEQFF

SEQ ID NO: 1069 - protein, 11 aa, Artificial sequence
  synthetic sequence
CASATGAEQYF

SEQ ID NO: 1070 - protein, 14 aa, Artificial sequence
  synthetic sequence
CASSFKPVNTEAFF

SEQ ID NO: 1071 - protein, 15 aa, Artificial sequence
  synthetic sequence
CASSLRTGVRETQYF

SEQ ID NO: 1072 - protein, 15 aa, Artificial sequence
  synthetic sequence
CASSLAQGGQYEQYF

SEQ ID NO: 1073 - protein, 18 aa, Artificial sequence
  synthetic sequence
CASSLTPGTSGSSYEQYF

SEQ ID NO: 1074 - protein, 14 aa, Artificial sequence
  synthetic sequence
CSAGPPRVNTEAFF

SEQ ID NO: 1075 - protein, 17 aa, Artificial sequence
  synthetic sequence
CASSVEGGRGSTDTQYF

SEQ ID NO: 1076 - protein, 15 aa, Artificial sequence
  synthetic sequence
CASSLRQGVREKLFF

SEQ ID NO: 1077 - protein, 15 aa, Artificial sequence
  synthetic sequence
CASSLVFGTSYEQYF

SEQ ID NO: 1078 - protein, 17 aa, Artificial sequence
  synthetic sequence
CASSQEGGRPSTDTQYF

SEQ ID NO: 1079 - protein, 14 aa, Artificial sequence
  synthetic sequence
CASSSGSEGQPQHF

SEQ ID NO: 1080 - protein, 18 aa, Artificial sequence
  synthetic sequence
CASSLTSGTSGTPYEQYF

SEQ ID NO: 1081 - protein, 12 aa, Artificial sequence
  synthetic sequence
CASQGSHNEQFF

SEQ ID NO: 1082 - protein, 15 aa, Artificial sequence
  synthetic sequence
CSARTLAGGSGELFF

SEQ ID NO: 1083 - protein, 13 aa, Artificial sequence
  synthetic sequence
CASSSYTNQPQHF

SEQ ID NO: 1084 - protein, 14 aa, Artificial sequence
  synthetic sequence
CASSEGAPWQPQHF

SEQ ID NO: 1085 - protein, 15 aa, Artificial sequence
  synthetic sequence
CASSLRQGIREKLFF

SEQ ID NO: 1086 - protein, 14 aa, Artificial sequence
  synthetic sequence
CASSLARTGETQYF

SEQ ID NO: 1087 - protein, 16 aa, Artificial sequence
  synthetic sequence
CASSGRTGGNTGELFF

SEQ ID NO: 1088 - protein, 15 aa, Artificial sequence
  synthetic sequence
CASSSRGGVRETQYF

SEQ ID NO: 1089 - protein, 12 aa, Artificial sequence
  synthetic sequence
CASTRVGSEAFF

SEQ ID NO: 1090 - protein, 14 aa, Artificial sequence
  synthetic sequence
CASSLASVTDTQYF

SEQ ID NO: 1091 - protein, 16 aa, Artificial sequence
  synthetic sequence
CASRTSGLSGANVLTF

SEQ ID NO: 1092 - protein, 14 aa, Artificial sequence
  synthetic sequence
CASGGTGGPYEQYF

SEQ ID NO: 1093 - protein, 12 aa, Artificial sequence
  synthetic sequence
CASSLWSREQFF

SEQ ID NO: 1094 - protein, 13 aa, Artificial sequence
  synthetic sequence
CASSSKDRGEQYF

SEQ ID NO: 1095 - protein, 16 aa, Artificial sequence
  synthetic sequence
CASSLWGREANEKLFF

SEQ ID NO: 1096 - protein, 16 aa, Artificial sequence
  synthetic sequence
CASSLRRGGMNTEAFF

SEQ ID NO: 1097 - protein, 11 aa, Artificial sequence
  synthetic sequence
CASSSGAQQYF

SEQ ID NO: 1098 - protein, 14 aa, Artificial sequence
  synthetic sequence
CASSDVGRSDEQFF

SEQ ID NO: 1099 - protein, 16 aa, Artificial sequence
  synthetic sequence
CASSTKTGVRAGELFF

SEQ ID NO: 1100 - protein, 12 aa, Artificial sequence
  synthetic sequence
CASTNTNYGYTF

SEQ ID NO: 1101 - protein, 15 aa, Artificial sequence
  synthetic sequence
CASSVRQGVREKLFF

SEQ ID NO: 1102 - protein, 15 aa, Artificial sequence
  synthetic sequence
CASSTRTGIRETQYF

SEQ ID NO: 1103 - protein, 15 aa, Artificial sequence
  synthetic sequence
CASSLRTGIRETQYF

SEQ ID NO: 1104 - protein, 13 aa, Artificial sequence
  synthetic sequence
CSVGQQGTDTQYF

SEQ ID NO: 1105 - protein, 11 aa, Artificial sequence
  synthetic sequence
CASSSGARQFF

SEQ ID NO: 1106 - protein, 13 aa, Artificial sequence
  synthetic sequence
CATTRGRGETQYF

SEQ ID NO: 1107 - protein, 16 aa, Artificial sequence
  synthetic sequence
CASSLGLAGGGNTIYF

SEQ ID NO: 1108 - protein, 14 aa, Artificial sequence
  synthetic sequence
CASSAPWGTDTQYF

SEQ ID NO: 1109 - protein, 15 aa, Artificial sequence
  synthetic sequence
CASSLYGFRTDTQYF

SEQ ID NO: 1110 - protein, 14 aa, Artificial sequence
  synthetic sequence
CASSEAGGSNTQYF

SEQ ID NO: 1111 - protein, 17 aa, Artificial sequence
  synthetic sequence
CSAREWRGRSSYNEQFF

SEQ ID NO: 1112 - protein, 16 aa, Artificial sequence
  synthetic sequence
CASSLTGTEGNSPLHF

SEQ ID NO: 1113 - protein, 16 aa, Artificial sequence
  synthetic sequence
CASSGRTGPNTGELFF

SEQ ID NO: 1114 - protein, 15 aa, Artificial sequence
  synthetic sequence
CASSSRQGVRETQYF

SEQ ID NO: 1115 - protein, 13 aa, Artificial sequence
  synthetic sequence
CASSYGPREPQHF

SEQ ID NO: 1116 - protein, 11 aa, Artificial sequence
  synthetic sequence
CASSSGAKQYF

SEQ ID NO: 1117 - protein, 15 aa, Artificial sequence
  synthetic sequence
CASSRGTDPRETQYF

SEQ ID NO: 1118 - protein, 12 aa, Artificial sequence
  synthetic sequence
CASSLWSREQYF

SEQ ID NO: 1119 - protein, 12 aa, Artificial sequence
  synthetic sequence
CATSDKKNIQYF

SEQ ID NO: 1120 - protein, 12 aa, Artificial sequence
  synthetic sequence
CASSEGGHTQYF

SEQ ID NO: 1121 - protein, 14 aa, Artificial sequence
  synthetic sequence
CASKRTSSYNEQFF

SEQ ID NO: 1122 - protein, 13 aa, Artificial sequence
  synthetic sequence
CASSRGSTETQYF

SEQ ID NO: 1123 - protein, 13 aa, Artificial sequence
  synthetic sequence
CASSAGPTGELFF

SEQ ID NO: 1124 - protein, 14 aa, Artificial sequence
  synthetic sequence
CASNNRDRGYEQYF

SEQ ID NO: 1125 - protein, 16 aa, Artificial sequence
  synthetic sequence
CASSPRGGRGSYEQYF

SEQ ID NO: 1126 - protein, 15 aa, Artificial sequence
  synthetic sequence
CASSRYREGPYEQYF

SEQ ID NO: 1127 - protein, 17 aa, Artificial sequence
  synthetic sequence
CATSRDPDRAGANVLTF

SEQ ID NO: 1128 - protein, 13 aa, Artificial sequence
  synthetic sequence
CASQGASTDTQYF

SEQ ID NO: 1129 - protein, 12 aa, Artificial sequence
  synthetic sequence
CATSDRQNIQYF

SEQ ID NO: 1130 - protein, 16 aa, Artificial sequence
  synthetic sequence
CASSRDSTLAKNIQYF

SEQ ID NO: 1131 - protein, 18 aa, Artificial sequence
  synthetic sequence
CASSLTTGLAGSSYEQYF

SEQ ID NO: 1132 - protein, 17 aa, Artificial sequence
  synthetic sequence
CASSQARTGRFGNTIYF

SEQ ID NO: 1133 - protein, 12 aa, Artificial sequence
  synthetic sequence
CASKDRENIQYF

SEQ ID NO: 1134 - protein, 14 aa, Artificial sequence
  synthetic sequence
CSATGQGSGQPQHF

SEQ ID NO: 1135 - protein, 14 aa, Artificial sequence
  synthetic sequence
CSARRQGEAGELFF

SEQ ID NO: 1136 - protein, 15 aa, Artificial sequence
  synthetic sequence
CSARGKREGLGEQFF

SEQ ID NO: 1137 - protein, 14 aa, Artificial sequence
  synthetic sequence
CASSPSLVRDEQFF

SEQ ID NO: 1138 - protein, 15 aa, Artificial sequence
  synthetic sequence
CASSLTGGWGNEQFF

SEQ ID NO: 1139 - protein, 15 aa, Artificial sequence
  synthetic sequence
CASSWRQGVRETQYF

SEQ ID NO: 1140 - protein, 15 aa, Artificial sequence
  synthetic sequence
CASSTRTGVREKLFF

SEQ ID NO: 1141 - protein, 15 aa, Artificial sequence
  synthetic sequence
CASSPSGRADNEQFF

SEQ ID NO: 1142 - protein, 13 aa, Artificial sequence
  synthetic sequence
CASKDRDNSPLHF

SEQ ID NO: 1143 - protein, 15 aa, Artificial sequence
  synthetic sequence
CSARSRGGGPGELFF

SEQ ID NO: 1144 - protein, 17 aa, Artificial sequence
  synthetic sequence
CATSRDPDRSGANVLTF

SEQ ID NO: 1145 - protein, 14 aa, Artificial sequence
  synthetic sequence
CSARQQGETGELFF

SEQ ID NO: 1146 - protein, 12 aa, Artificial sequence
  synthetic sequence
CASRDTRGEQYF

SEQ ID NO: 1147 - protein, 15 aa, Artificial sequence
  synthetic sequence
CASSVRTGIREKLFF

SEQ ID NO: 1148 - protein, 15 aa, Artificial sequence
  synthetic sequence
CASSFRTGVRETQYF

SEQ ID NO: 1149 - protein, 11 aa, Artificial sequence
  synthetic sequence
CSVDSPNEQFF

SEQ ID NO: 1150 - protein, 16 aa, Artificial sequence
  synthetic sequence
CASSLDRQGRDYGYTF

SEQ ID NO: 1151 - protein, 10 aa, Artificial sequence
  synthetic sequence
CASRLNEAFF

SEQ ID NO: 1152 - protein, 14 aa, Artificial sequence
  synthetic sequence
CASISAGEKNIQYF

SEQ ID NO: 1153 - protein, 14 aa, Artificial sequence
  synthetic sequence
CSANPSGVGETQYF

SEQ ID NO: 1154 - protein, 13 aa, Artificial sequence
  synthetic sequence
CASSPLADNEQFF

SEQ ID NO: 1155 - protein, 14 aa, Artificial sequence
  synthetic sequence
CSARSQGEAGELFF

SEQ ID NO: 1156 - protein, 16 aa, Artificial sequence
  synthetic sequence
CASSVDSTLAKNIQYF

SEQ ID NO: 1157 - protein, 15 aa, Artificial sequence
  synthetic sequence
CASSHRTGVRETQYF

SEQ ID NO: 1158 - protein, 15 aa, Artificial sequence
  synthetic sequence
CASSLYGAGPYEQYF

SEQ ID NO: 1159 - protein, 15 aa, Artificial sequence
  synthetic sequence
CASSQESGNGETQYF

SEQ ID NO: 1160 - protein, 15 aa, Artificial sequence
  synthetic sequence
CASSNRQGIRETQYF

SEQ ID NO: 1161 - protein, 12 aa, Artificial sequence
  synthetic sequence
CASRDTRGTQYF

SEQ ID NO: 1162 - protein, 15 aa, Artificial sequence
  synthetic sequence
CASSNRQGVRETQYF

SEQ ID NO: 1163 - protein, 15 aa, Artificial sequence
  synthetic sequence
CASSQASAAQETQYF

SEQ ID NO: 1164 - protein, 11 aa, Artificial sequence
  synthetic sequence
CASRSFAEAFF

SEQ ID NO: 1165 - protein, 17 aa, Artificial sequence
  synthetic sequence
CASSLASQRYSNQPQHF

SEQ ID NO: 1166 - protein, 14 aa, Artificial sequence
  synthetic sequence
CASSPGVQSQPQHF

SEQ ID NO: 1167 - protein, 16 aa, Artificial sequence
  synthetic sequence
CASSQGLRGGGETQYF

SEQ ID NO: 1168 - protein, 17 aa, Artificial sequence
  synthetic sequence
CASSLMGSSSGANVLTF

SEQ ID NO: 1169 - protein, 15 aa, Artificial sequence
  synthetic sequence
CASSIRQGVREKLFF

SEQ ID NO: 1170 - protein, 15 aa, Artificial sequence
  synthetic sequence
CASSLGHPMNTEAFF

SEQ ID NO: 1171 - protein, 14 aa, Artificial sequence
  synthetic sequence
CSAGPRSGNQPQHF

SEQ ID NO: 1172 - protein, 15 aa, Artificial sequence
  synthetic sequence
CASSWGRGTTGELFF

SEQ ID NO: 1173 - protein, 15 aa, Artificial sequence
  synthetic sequence
CASSLGDYFTGELFF

SEQ ID NO: 1174 - protein, 15 aa, Artificial sequence
  synthetic sequence
CSAIRTGGFQETQYF

SEQ ID NO: 1175 - protein, 15 aa, Artificial sequence
  synthetic sequence
CASSIRTGVREKLFF

SEQ ID NO: 1176 - protein, 16 aa, Artificial sequence
  synthetic sequence
CASSQQGVGSNQPQHF

SEQ ID NO: 1177 - protein, 15 aa, Artificial sequence
  synthetic sequence
CASSPLGGGGETQYF

SEQ ID NO: 1178 - protein, 16 aa, Artificial sequence
  synthetic sequence
CASSALAGETTDTQYF

SEQ ID NO: 1179 - protein, 14 aa, Artificial sequence
  synthetic sequence
CASSSDRGLAPLHF

SEQ ID NO: 1180 - protein, 15 aa, Artificial sequence
  synthetic sequence
CASSIRTGIREKLFF

SEQ ID NO: 1181 - protein, 15 aa, Artificial sequence
  synthetic sequence
CASSLAPGQGGEQFF

SEQ ID NO: 1182 - protein, 14 aa, Artificial sequence
  synthetic sequence
CASSLTPTSGEQYF

SEQ ID NO: 1183 - protein, 14 aa, Artificial sequence
  synthetic sequence
CASSTGTRGETQYF

SEQ ID NO: 1184 - protein, 15 aa, Artificial sequence
  synthetic sequence
CASSLSGQGGNTIYF

SEQ ID NO: 1185 - protein, 14 aa, Artificial sequence
  synthetic sequence
CASSRTGKEDGYTF

SEQ ID NO: 1186 - protein, 15 aa, Artificial sequence
  synthetic sequence
CASSPSGRDTDTQYF

SEQ ID NO: 1187 - protein, 15 aa, Artificial sequence
  synthetic sequence
CASSFRQGVRETQYF

SEQ ID NO: 1188 - protein, 16 aa, Artificial sequence
  synthetic sequence
CASSLDSSLAKNIQYF

SEQ ID NO: 1189 - protein, 14 aa, Artificial sequence
  synthetic sequence
CSARAQGEVGELFF

SEQ ID NO: 1190 - protein, 16 aa, Artificial sequence
  synthetic sequence
CASSVDRGGSYNEQFF

SEQ ID NO: 1191 - protein, 14 aa, Artificial sequence
  synthetic sequence
CASSDRTALEKLFF

SEQ ID NO: 1192 - protein, 13 aa, Artificial sequence
  synthetic sequence
CSVPAQGTDTQYF

SEQ ID NO: 1193 - protein, 15 aa, Artificial sequence
  synthetic sequence
CASSTRTGIREKLFF

SEQ ID NO: 1194 - protein, 14 aa, Artificial sequence
  synthetic sequence
CSAGRQGTTGELFF

SEQ ID NO: 1195 - protein, 15 aa, Artificial sequence
  synthetic sequence
CASSIRQGIRETQYF

SEQ ID NO: 1196 - protein, 14 aa, Artificial sequence
  synthetic sequence
CSARGPGETGELFF

SEQ ID NO: 1197 - protein, 16 aa, Artificial sequence
  synthetic sequence
CASSSDTTLAKNIQYF

SEQ ID NO: 1198 - protein, 13 aa, Artificial sequence
  synthetic sequence
CAISRGRGYEQYF

SEQ ID NO: 1199 - protein, 11 aa, Artificial sequence
  synthetic sequence
CASRYQNEQFF

SEQ ID NO: 1200 - protein, 16 aa, Artificial sequence
  synthetic sequence
CASSIDSTLAKNIQYF

SEQ ID NO: 1201 - protein, 14 aa, Artificial sequence
  synthetic sequence
CASSLYGSADTQYF

SEQ ID NO: 1202 - protein, 14 aa, Artificial sequence
  synthetic sequence
CASSKRGANYGYTF

SEQ ID NO: 1203 - protein, 15 aa, Artificial sequence
  synthetic sequence
CASSLREGSGETQYF

SEQ ID NO: 1204 - protein, 14 aa, Artificial sequence
  synthetic sequence
CASSKGPTGNTIYF

SEQ ID NO: 1205 - protein, 15 aa, Artificial sequence
  synthetic sequence
CASSVRQGIRETQYF

SEQ ID NO: 1206 - protein, 14 aa, Artificial sequence
  synthetic sequence
CATLQAGGHNEQFF

SEQ ID NO: 1207 - protein, 15 aa, Artificial sequence
  synthetic sequence
CSARSRAGGPGELFF

SEQ ID NO: 1208 - protein, 16 aa, Artificial sequence
  synthetic sequence
CASGLAGGPGTGELFF

SEQ ID NO: 1209 - protein, 15 aa, Artificial sequence
  synthetic sequence
CASSASGGRYNEQFF

SEQ ID NO: 1210 - protein, 14 aa, Artificial sequence
  synthetic sequence
CASSLRRDSQPQHF

SEQ ID NO: 1211 - protein, 15 aa, Artificial sequence
  synthetic sequence
CASSERAGVRETQYF

SEQ ID NO: 1212 - protein, 15 aa, Artificial sequence
  synthetic sequence
CSARKLAGGAGELFF

SEQ ID NO: 1213 - protein, 15 aa, Artificial sequence
  synthetic sequence
CSARSKREGLGEQFF

SEQ ID NO: 1214 - protein, 15 aa, Artificial sequence
  synthetic sequence
CASSPYGQGGDEQYF

SEQ ID NO: 1215 - protein, 14 aa, Artificial sequence
  synthetic sequence
CSARPQGDDQPQHF

SEQ ID NO: 1216 - protein, 15 aa, Artificial sequence
  synthetic sequence
CASSYRGGIRETQYF

SEQ ID NO: 1217 - protein, 12 aa, Artificial sequence
  synthetic sequence
CATSDRKNIQYF

SEQ ID NO: 1218 - protein, 14 aa, Artificial sequence
  synthetic sequence
CSARGQGEGGELFF

SEQ ID NO: 1219 - protein, 15 aa, Artificial sequence
  synthetic sequence
CASSRLAGMEETQYF

SEQ ID NO: 1220 - protein, 15 aa, Artificial sequence
  synthetic sequence
CASSLRGGVREKLFF

SEQ ID NO: 1221 - protein, 16 aa, Artificial sequence
  synthetic sequence
CASRRKDSYSNQPQHF

SEQ ID NO: 1222 - protein, 14 aa, Artificial sequence
  synthetic sequence
CASSSDVGGQPQHF

SEQ ID NO: 1223 - protein, 14 aa, Artificial sequence
  synthetic sequence
CSGRGQGETGELFF

SEQ ID NO: 1224 - protein, 14 aa, Artificial sequence
  synthetic sequence
CSVEGEGNTDTQYF

SEQ ID NO: 1225 - protein, 13 aa, Artificial sequence
  synthetic sequence
CASSPDGGGEQYF

SEQ ID NO: 1226 - protein, 17 aa, Artificial sequence
  synthetic sequence
CASSSPGRGGGNQPQHF

SEQ ID NO: 1227 - protein, 12 aa, Artificial sequence
  synthetic sequence
CASRDSKNIQYF

SEQ ID NO: 1228 - protein, 14 aa, Artificial sequence
  synthetic sequence
CATSVTGVQETQYF

SEQ ID NO: 1229 - protein, 12 aa, Artificial sequence
  synthetic sequence
CASSPRTNEQYF

SEQ ID NO: 1230 - protein, 15 aa, Artificial sequence
  synthetic sequence
CASSYRTGIRETQYF

SEQ ID NO: 1231 - protein, 11 aa, Artificial sequence
  synthetic sequence
CASSSGALQYF

SEQ ID NO: 1232 - protein, 15 aa, Artificial sequence
  synthetic sequence
CASSTRSGVRETQYF

SEQ ID NO: 1233 - protein, 15 aa, Artificial sequence
  synthetic sequence
CASSYRGGVRETQYF

SEQ ID NO: 1234 - protein, 15 aa, Artificial sequence
  synthetic sequence
CASSSGVLAKNIQYF

SEQ ID NO: 1235 - protein, 13 aa, Artificial sequence
  synthetic sequence
CASKAGVTDTQYF

SEQ ID NO: 1236 - protein, 15 aa, Artificial sequence
  synthetic sequence
CASSGRGGNTGELFF

SEQ ID NO: 1237 - protein, 15 aa, Artificial sequence
  synthetic sequence
CASSLIFGESYEQYF

SEQ ID NO: 1238 - protein, 15 aa, Artificial sequence
  synthetic sequence
CASSQDTTAYNEQFF

SEQ ID NO: 1239 - protein, 15 aa, Artificial sequence
  synthetic sequence
CASSIRQGIREKLFF

SEQ ID NO: 1240 - protein, 13 aa, Artificial sequence
  synthetic sequence
CSATPGGRDGYTF

SEQ ID NO: 1241 - protein, 13 aa, Artificial sequence
  synthetic sequence
CASSTELQETQYF

SEQ ID NO: 1242 - protein, 16 aa, Artificial sequence
  synthetic sequence
CASSLVGGGTRYGYTF

SEQ ID NO: 1243 - protein, 14 aa, Artificial sequence
  synthetic sequence
CASSNPGDRDTQYF

SEQ ID NO: 1244 - protein, 15 aa, Artificial sequence
  synthetic sequence
CASSTRQGIRETQYF

SEQ ID NO: 1245 - protein, 15 aa, Artificial sequence
  synthetic sequence
CSARERAGGPGELFF

SEQ ID NO: 1246 - protein, 14 aa, Artificial sequence
  synthetic sequence
CASSWDSGANVLTF

SEQ ID NO: 1247 - protein, 13 aa, Artificial sequence
  synthetic sequence
CASSPDQSNEQFF

SEQ ID NO: 1248 - protein, 15 aa, Artificial sequence
  synthetic sequence
CASSLRGGVRETQYF

SEQ ID NO: 1249 - protein, 11 aa, Artificial sequence
  synthetic sequence
CASSSGALQFF

SEQ ID NO: 1250 - protein, 16 aa, Artificial sequence
  synthetic sequence
CASSLSTSFPYNEQFF

SEQ ID NO: 1251 - protein, 16 aa, Artificial sequence
  synthetic sequence
CSARVPSGSGTGELFF

SEQ ID NO: 1252 - protein, 15 aa, Artificial sequence
  synthetic sequence
CASSYRSGVRETQYF

SEQ ID NO: 1253 - protein, 11 aa, Artificial sequence
  synthetic sequence
CASGRGNEQFF

SEQ ID NO: 1254 - protein, 17 aa, Artificial sequence
  synthetic sequence
CSARAWGGRSSYNEQFF

SEQ ID NO: 1255 - protein, 14 aa, Artificial sequence
  synthetic sequence
CASSSPGGPYEQYF

SEQ ID NO: 1256 - protein, 15 aa, Artificial sequence
  synthetic sequence
CASSYRQGIRETQYF

SEQ ID NO: 1257 - protein, 14 aa, Artificial sequence
  synthetic sequence
CAWSGLGVGETQYF

SEQ ID NO: 1258 - protein, 14 aa, Artificial sequence
  synthetic sequence
CASTQQGAGEKLFF

SEQ ID NO: 1259 - protein, 15 aa, Artificial sequence
  synthetic sequence
CASSLRQGIRETQYF

SEQ ID NO: 1260 - protein, 15 aa, Artificial sequence
  synthetic sequence
CASSLRQGVRETQYF

SEQ ID NO: 1261 - protein, 14 aa, Artificial sequence
  synthetic sequence
CSARDQGEGGELFF

SEQ ID NO: 1262 - protein, 15 aa, Artificial sequence
  synthetic sequence
CASSVRQGVRETQYF

SEQ ID NO: 1263 - protein, 14 aa, Artificial sequence
  synthetic sequence
CSARKQGETGELFF

SEQ ID NO: 1264 - protein, 15 aa, Artificial sequence
  synthetic sequence
CASSTRGGVRETQYF

SEQ ID NO: 1265 - protein, 13 aa, Artificial sequence
  synthetic sequence
CSASIGYTDTQYF

SEQ ID NO: 1266 - protein, 17 aa, Artificial sequence
  synthetic sequence
CASSLRRGESYNSPLHF

SEQ ID NO: 1267 - protein, 15 aa, Artificial sequence
  synthetic sequence
CASSQRQGVRETQYF

SEQ ID NO: 1268 - protein, 17 aa, Artificial sequence
  synthetic sequence
CASSPKTGGLMNTEAFF

SEQ ID NO: 1269 - protein, 13 aa, Artificial sequence
  synthetic sequence
CASSYWDRYEQYF

SEQ ID NO: 1270 - protein, 15 aa, Artificial sequence
  synthetic sequence
CASSLEAGFQETQYF

SEQ ID NO: 1271 - protein, 15 aa, Artificial sequence
  synthetic sequence
CASSYRPGVRETQYF

SEQ ID NO: 1272 - protein, 14 aa, Artificial sequence
  synthetic sequence
CSVTGGAGNTEAFF

SEQ ID NO: 1273 - protein, 17 aa, Artificial sequence
  synthetic sequence
CASSLAAGTGGNQPQHF

SEQ ID NO: 1274 - protein, 15 aa, Artificial sequence
  synthetic sequence
CASSLVDSGNYEQYF

SEQ ID NO: 1275 - protein, 13 aa, Artificial sequence
  synthetic sequence
CASSLSTGEPQHF

SEQ ID NO: 1276 - protein, 16 aa, Artificial sequence
  synthetic sequence
CASSPRVAGDLNEQFF

SEQ ID NO: 1277 - protein, 13 aa, Artificial sequence
  synthetic sequence
CASSLFTKNIQYF

SEQ ID NO: 1278 - protein, 15 aa, Artificial sequence
  synthetic sequence
CASSLGGFSGNTIYF

SEQ ID NO: 1279 - protein, 14 aa, Artificial sequence
  synthetic sequence
CASNTWGAGNTIYF

SEQ ID NO: 1280 - protein, 15 aa, Artificial sequence
  synthetic sequence
CASSNRGGVRETQYF

SEQ ID NO: 1281 - protein, 15 aa, Artificial sequence
  synthetic sequence
CASSLRTGIREKLFF

SEQ ID NO: 1282 - protein, 15 aa, Artificial sequence
  synthetic sequence
CASSPTGGTDTEAFF

SEQ ID NO: 1283 - protein, 14 aa, Artificial sequence
  synthetic sequence
CASSNPGEKNIQYF

SEQ ID NO: 1284 - protein, 15 aa, Artificial sequence
  synthetic sequence
CASRPPRGRNQPQHF

SEQ ID NO: 1285 - protein, 17 aa, Artificial sequence
  synthetic sequence
CASSLGTAGSYNSPLHF

SEQ ID NO: 1286 - protein, 14 aa, Artificial sequence
  synthetic sequence
CASSLDLRGAEAFF

SEQ ID NO: 1287 - protein, 17 aa, Artificial sequence
  synthetic sequence
CASSYSGLAGVYNEQFF

SEQ ID NO: 1288 - protein, 15 aa, Artificial sequence
  synthetic sequence
CSARLLAGGSGELFF

SEQ ID NO: 1289 - protein, 17 aa, Artificial sequence
  synthetic sequence
CASSLGRAGSYNSPLHF

SEQ ID NO: 1290 - protein, 14 aa, Artificial sequence
  synthetic sequence
CASRGGQGNTEAFF

SEQ ID NO: 1291 - protein, 15 aa, Artificial sequence
  synthetic sequence
CASSLVGGQADTQYF

SEQ ID NO: 1292 - protein, 15 aa, Artificial sequence
  synthetic sequence
CAISDSDRGYQPQHF

SEQ ID NO: 1293 - protein, 14 aa, Artificial sequence
  synthetic sequence
CSARGQGDNQPQHF

SEQ ID NO: 1294 - protein, 13 aa, Artificial sequence
  synthetic sequence
CASSLEGNSPLHF

SEQ ID NO: 1295 - protein, 17 aa, Artificial sequence
  synthetic sequence
CASSWTSGRAGYNEQFF

SEQ ID NO: 1296 - protein, 15 aa, Artificial sequence
  synthetic sequence
CASSEADRAGNTIYF

SEQ ID NO: 1297 - protein, 14 aa, Artificial sequence
  synthetic sequence
CASSSPGDKNIQYF

SEQ ID NO: 1298 - protein, 14 aa, Artificial sequence
  synthetic sequence
CASSMPGEKNIQYF

SEQ ID NO: 1299 - protein, 14 aa, Artificial sequence
  synthetic sequence
CASSLGGGRSGYTF

SEQ ID NO: 1300 - protein, 17 aa, Artificial sequence
  synthetic sequence
CASSSGQAGSYNSPLHF

SEQ ID NO: 1301 - protein, 13 aa, Artificial sequence
  synthetic sequence
CSARVIDSYEQYF

SEQ ID NO: 1302 - protein, 14 aa, Artificial sequence
  synthetic sequence
CASSQPGDKNIQYF

SEQ ID NO: 1303 - protein, 17 aa, Artificial sequence
  synthetic sequence
CASSYSPPSGIYNEQFF

SEQ ID NO: 1304 - protein, 12 aa, Artificial sequence
  synthetic sequence
CASSQEFTEAFF

SEQ ID NO: 1305 - protein, 15 aa, Artificial sequence
  synthetic sequence
CASSLDQGSNQPQHF

SEQ ID NO: 1306 - protein, 17 aa, Artificial sequence
  synthetic sequence
CASSYSGLAGIYNEQFF

SEQ ID NO: 1307 - protein, 15 aa, Artificial sequence
  synthetic sequence
CASRPSTGRNQPQHF

SEQ ID NO: 1308 - protein, 11 aa, Artificial sequence
  synthetic sequence
CASSSRHEQYF

SEQ ID NO: 1309 - protein, 15 aa, Artificial sequence
  synthetic sequence
CASSIDRVHNSPLHF

SEQ ID NO: 1310 - protein, 15 aa, Artificial sequence
  synthetic sequence
CASSEGRADTYEQYF

SEQ ID NO: 1311 - protein, 14 aa, Artificial sequence
  synthetic sequence
CAISEKGQGYEQYF

SEQ ID NO: 1312 - protein, 15 aa, Artificial sequence
  synthetic sequence
CASSQDGGYQETQYF

SEQ ID NO: 1313 - protein, 17 aa, Artificial sequence
  synthetic sequence
CASSYSPLAGVYNEQFF

SEQ ID NO: 1314 - protein, 16 aa, Artificial sequence
  synthetic sequence
CASSLDRGSGYNEQFF

SEQ ID NO: 1315 - protein, 15 aa, Artificial sequence
  synthetic sequence
CASRTGTGNGNTIYF

SEQ ID NO: 1316 - protein, 16 aa, Artificial sequence
  synthetic sequence
CASSYGRLAGGYEQYF

SEQ ID NO: 1317 - protein, 14 aa, Artificial sequence
  synthetic sequence
CASSNPGDKNIQYF

SEQ ID NO: 1318 - protein, 14 aa, Artificial sequence
  synthetic sequence
CASSDVGRTDTQYF

SEQ ID NO: 1319 - protein, 14 aa, Artificial sequence
  synthetic sequence
CASSLRDKNYGYTF

SEQ ID NO: 1320 - protein, 15 aa, Artificial sequence
  synthetic sequence
CASSLDRVHNSPLHF

SEQ ID NO: 1321 - protein, 18 aa, Artificial sequence
  synthetic sequence
CASSQEGIRGLMNTEAFF

SEQ ID NO: 1322 - protein, 17 aa, Artificial sequence
  synthetic sequence
CASSWTSGRLSYNEQFF

SEQ ID NO: 1323 - protein, 13 aa, Artificial sequence
  synthetic sequence
CASSRELQETQYF

SEQ ID NO: 1324 - protein, 14 aa, Artificial sequence
  synthetic sequence
CSATSGTDNSPLHF

SEQ ID NO: 1325 - protein, 16 aa, Artificial sequence
  synthetic sequence
CASSLYVAASTDTQYF

SEQ ID NO: 1326 - protein, 13 aa, Artificial sequence
  synthetic sequence
CASSLELQETQYF

SEQ ID NO: 1327 - protein, 15 aa, Artificial sequence
  synthetic sequence
CASSLGDRADQPQHF

SEQ ID NO: 1328 - protein, 15 aa, Artificial sequence
  synthetic sequence
CASSPGGGPGSPLHF

SEQ ID NO: 1329 - protein, 14 aa, Artificial sequence
  synthetic sequence
CSVEGQGAVETQYF

SEQ ID NO: 1330 - protein, 14 aa, Artificial sequence
  synthetic sequence
CASSYSRVYNEQFF

SEQ ID NO: 1331 - protein, 14 aa, Artificial sequence
  synthetic sequence
CASSLRPDNTEAFF

SEQ ID NO: 1332 - protein, 16 aa, Artificial sequence
  synthetic sequence
CASSLVWTGRSYEQYF

SEQ ID NO: 1333 - protein, 15 aa, Artificial sequence
  synthetic sequence
CASSQGTGDTYEQYF

SEQ ID NO: 1334 - protein, 13 aa, Artificial sequence
  synthetic sequence
CSAPDLSSYEQYF

SEQ ID NO: 1335 - protein, 14 aa, Artificial sequence
  synthetic sequence
CASRASSGNQPQHF

SEQ ID NO: 1336 - protein, 13 aa, Artificial sequence
  synthetic sequence
CASRDTDTGELFF

SEQ ID NO: 1337 - protein, 17 aa, Artificial sequence
  synthetic sequence
CASSIRGGSYSNQPQHF

SEQ ID NO: 1338 - protein, 16 aa, Artificial sequence
  synthetic sequence
CASSPLTSGPYNEQFF

SEQ ID NO: 1339 - protein, 15 aa, Artificial sequence
  synthetic sequence
CASSQDWVSNQPQHF

SEQ ID NO: 1340 - protein, 15 aa, Artificial sequence
  synthetic sequence
CASSETGTGNEKLFF

SEQ ID NO: 1341 - protein, 15 aa, Artificial sequence
  synthetic sequence
CASSSSGTGYQPQHF

SEQ ID NO: 1342 - protein, 13 aa, Artificial sequence
  synthetic sequence
CASSAFTNQPQHF

SEQ ID NO: 1343 - protein, 14 aa, Artificial sequence
  synthetic sequence
CASRTSGGLETQYF

SEQ ID NO: 1344 - protein, 15 aa, Artificial sequence
  synthetic sequence
CASSLGPPPNNEQFF

SEQ ID NO: 1345 - protein, 14 aa, Artificial sequence
  synthetic sequence
CSALEGAAKNIQYF

SEQ ID NO: 1346 - protein, 16 aa, Artificial sequence
  synthetic sequence
CASSQDPAGGVNEQFF

SEQ ID NO: 1347 - protein, 12 aa, Artificial sequence
  synthetic sequence
CASSTREGEQYF

SEQ ID NO: 1348 - protein, 15 aa, Artificial sequence
  synthetic sequence
CASSRVRGGNTEAFF

SEQ ID NO: 1349 - protein, 15 aa, Artificial sequence
  synthetic sequence
CASSLGTGNNEKLFF

SEQ ID NO: 1350 - protein, 14 aa, Artificial sequence
  synthetic sequence
CASRTPGLHNEQFF

SEQ ID NO: 1351 - protein, 14 aa, Artificial sequence
  synthetic sequence
CASRSQGDGQPQHF

SEQ ID NO: 1352 - protein, 15 aa, Artificial sequence
  synthetic sequence
CASSYFGRGTDEQYF

SEQ ID NO: 1353 - protein, 13 aa, Artificial sequence
  synthetic sequence
CASGGHSGNTIYF

SEQ ID NO: 1354 - protein, 13 aa, Artificial sequence
  synthetic sequence
CASSFTVDSPLHF

SEQ ID NO: 1355 - protein, 17 aa, Artificial sequence
  synthetic sequence
CASSLGKTSGGQETQYF

SEQ ID NO: 1356 - protein, 15 aa, Artificial sequence
  synthetic sequence
CSARDTRTGGSEQYF

SEQ ID NO: 1357 - protein, 14 aa, Artificial sequence
  synthetic sequence
CASSRTGSEQPQHF

SEQ ID NO: 1358 - protein, 14 aa, Artificial sequence
  synthetic sequence
CASRTSGDTGELFF

SEQ ID NO: 1359 - protein, 15 aa, Artificial sequence
  synthetic sequence
CASSPVAGGSQPQHF

SEQ ID NO: 1360 - protein, 14 aa, Artificial sequence
  synthetic sequence
CASSEDPGGQPQHF

SEQ ID NO: 1361 - protein, 14 aa, Artificial sequence
  synthetic sequence
CASSQGDIRWGYTF

SEQ ID NO: 1362 - protein, 12 aa, Artificial sequence
  synthetic sequence
CASRVRRDEAFF

SEQ ID NO: 1363 - protein, 13 aa, Artificial sequence
  synthetic sequence
CASSYTPREKLFF

SEQ ID NO: 1364 - protein, 14 aa, Artificial sequence
  synthetic sequence
CASSPRDRADGYTF

SEQ ID NO: 1365 - protein, 17 aa, Artificial sequence
  synthetic sequence
CASSLGEAGSYNSPLHF

SEQ ID NO: 1366 - protein, 13 aa, Artificial sequence
  synthetic sequence
CSAEQGGVHEQYF

SEQ ID NO: 1367 - protein, 15 aa, Artificial sequence
  synthetic sequence
CASSLWGGRAYEQYF

SEQ ID NO: 1368 - protein, 14 aa, Artificial sequence
  synthetic sequence
CASSSMGDNQPQHF

SEQ ID NO: 1369 - protein, 15 aa, Artificial sequence
  synthetic sequence
CASSQEVTYNQPQHF

SEQ ID NO: 1370 - protein, 14 aa, Artificial sequence
  synthetic sequence
CASSVGDGLGPQHF

SEQ ID NO: 1371 - protein, 14 aa, Artificial sequence
  synthetic sequence
CASRTSGGTGELFF

SEQ ID NO: 1372 - protein, 13 aa, Artificial sequence
  synthetic sequence
CASSFQQGAGYTF

SEQ ID NO: 1373 - protein, 17 aa, Artificial sequence
  synthetic sequence
CASSPGQAGSYNSPLHF

SEQ ID NO: 1374 - protein, 14 aa, Artificial sequence
  synthetic sequence
CASRGAGRYNEQFF

SEQ ID NO: 1375 - protein, 15 aa, Artificial sequence
  synthetic sequence
CAISDGGFSNQPQHF

SEQ ID NO: 1376 - protein, 15 aa, Artificial sequence
  synthetic sequence
CAISDSGRGVQPQHF

SEQ ID NO: 1377 - protein, 15 aa, Artificial sequence
  synthetic sequence
CASSFTGNSNYGYTF

SEQ ID NO: 1378 - protein, 14 aa, Artificial sequence
  synthetic sequence
CASSLMRGGNTIYF

SEQ ID NO: 1379 - protein, 15 aa, Artificial sequence
  synthetic sequence
CASSSSGRRTDTQYF

SEQ ID NO: 1380 - protein, 14 aa, Artificial sequence
  synthetic sequence
CASRAGRGTDTQYF

SEQ ID NO: 1381 - protein, 14 aa, Artificial sequence
  synthetic sequence
CASSVDGPNTEAFF

SEQ ID NO: 1382 - protein, 15 aa, Artificial sequence
  synthetic sequence
CAWSAIDSLNQPQHF

SEQ ID NO: 1383 - protein, 12 aa, Artificial sequence
  synthetic sequence
LSSFEKPLPAVP

SEQ ID NO: 1384 - protein, 13 aa, Artificial sequence
  synthetic sequence
CASSSTVDSPLHF

SEQ ID NO: 1385 - protein, 14 aa, Artificial sequence
  synthetic sequence
CSANLAGAVNEQFF

SEQ ID NO: 1386 - protein, 13 aa, Artificial sequence
  synthetic sequence
CASSQELQETQYF

SEQ ID NO: 1387 - protein, 14 aa, Artificial sequence
  synthetic sequence
CASSPRLGNEKLFF

SEQ ID NO: 1388 - protein, 15 aa, Artificial sequence
  synthetic sequence
CASSLTGGRTDTQYF

SEQ ID NO: 1389 - protein, 15 aa, Artificial sequence
  synthetic sequence
CASSPTGGTQETQYF

SEQ ID NO: 1390 - protein, 13 aa, Artificial sequence
  synthetic sequence
CASTRGQGNGYTF

SEQ ID NO: 1391 - protein, 14 aa, Artificial sequence
  synthetic sequence
CASRSDRLNYGYTF

SEQ ID NO: 1392 - protein, 14 aa, Artificial sequence
  synthetic sequence
CSARDDRTGSEAFF

SEQ ID NO: 1393 - protein, 16 aa, Artificial sequence
  synthetic sequence
CASSLVVGGDNSPLHF

SEQ ID NO: 1394 - protein, 15 aa, Artificial sequence
  synthetic sequence
CASSQGGGRGSPLHF

SEQ ID NO: 1395 - protein, 15 aa, Artificial sequence
  synthetic sequence
CASSLNDRGNTEAFF

SEQ ID NO: 1396 - protein, 15 aa, Artificial sequence
  synthetic sequence
CASSIRSGSAETQYF

SEQ ID NO: 1397 - protein, 15 aa, Artificial sequence
  synthetic sequence
CASSFDPSSYNEQFF

SEQ ID NO: 1398 - protein, 15 aa, Artificial sequence
  synthetic sequence
CSASLGRGAYNEQFF

SEQ ID NO: 1399 - protein, 12 aa, Artificial sequence
  synthetic sequence
CASSHFPTEAFF

SEQ ID NO: 1400 - protein, 13 aa, Artificial sequence
  synthetic sequence
CASSVEGNNEQFF

SEQ ID NO: 1401 - protein, 15 aa, Artificial sequence
  synthetic sequence
CASSLAGDLLYGYTF

SEQ ID NO: 1402 - protein, 16 aa, Artificial sequence
  synthetic sequence
CASSLANGNSGNTIYF

SEQ ID NO: 1403 - protein, 17 aa, Artificial sequence
  synthetic sequence
CASSPGRAGSYNSPLHF

SEQ ID NO: 1404 - protein, 14 aa, Artificial sequence
  synthetic sequence
CASRRRGRTDTQYF

SEQ ID NO: 1405 - protein, 15 aa, Artificial sequence
  synthetic sequence
CASSEEAGGPETQYF

SEQ ID NO: 1406 - protein, 13 aa, Artificial sequence
  synthetic sequence
CASSEDRVGGYTF

SEQ ID NO: 1407 - protein, 15 aa, Artificial sequence
  synthetic sequence
CASSASGRVNNEQFF

SEQ ID NO: 1408 - protein, 15 aa, Artificial sequence
  synthetic sequence
CASTYLDRGNYGYTF

SEQ ID NO: 1409 - protein, 15 aa, Artificial sequence
  synthetic sequence
CSARDQTSGRDTQYF

SEQ ID NO: 1410 - protein, 14 aa, Artificial sequence
  synthetic sequence
CASSSASTNEKLFF

SEQ ID NO: 1411 - protein, 12 aa, Artificial sequence
  synthetic sequence
CAWTLQYNEQFF

SEQ ID NO: 1412 - protein, 14 aa, Artificial sequence
  synthetic sequence
CASSAATTNEKLFF

SEQ ID NO: 1413 - protein, 13 aa, Artificial sequence
  synthetic sequence
CASSAPQGRGYTF

SEQ ID NO: 1414 - protein, 16 aa, Artificial sequence
  synthetic sequence
CASSPRTGGAGQPQHF

SEQ ID NO: 1415 - protein, 13 aa, Artificial sequence
  synthetic sequence
CASSQARDAEAFF

SEQ ID NO: 1416 - protein, 14 aa, Artificial sequence
  synthetic sequence
CASSLLNKNTEAFF

SEQ ID NO: 1417 - protein, 13 aa, Artificial sequence
  synthetic sequence
CASSRLVRDTQYF

SEQ ID NO: 1418 - protein, 14 aa, Artificial sequence
  synthetic sequence
CSAGDRVYNSPLHF

SEQ ID NO: 1419 - protein, 14 aa, Artificial sequence
  synthetic sequence
CASSEYRQNTEAFF

SEQ ID NO: 1420 - protein, 13 aa, Artificial sequence
  synthetic sequence
CASSFQPSYEQYF

SEQ ID NO: 1421 - protein, 13 aa, Artificial sequence
  synthetic sequence
CASSRLVTDTQYF

SEQ ID NO: 1422 - protein, 12 aa, Artificial sequence
  synthetic sequence
CASSPGQWGYTF

SEQ ID NO: 1423 - protein, 14 aa, Artificial sequence
  synthetic sequence
CASSQPGQTYEQYF

SEQ ID NO: 1424 - protein, 7 aa, Artificial sequence
  synthetic sequence
CSNGYTF

SEQ ID NO: 1425 - protein, 14 aa, Artificial sequence
  synthetic sequence
CASSNPGQVNIQYF

SEQ ID NO: 1426 - protein, 13 aa, Artificial sequence
  synthetic sequence
CASSPPGGSPLHF

SEQ ID NO: 1427 - protein, 14 aa, Artificial sequence
  synthetic sequence
CASSPMGRTDTQYF

SEQ ID NO: 1428 - protein, 14 aa, Artificial sequence
  synthetic sequence
CAISVTGEDTEAFF

SEQ ID NO: 1429 - protein, 14 aa, Artificial sequence
  synthetic sequence
CASSLGAVNEKLFF

SEQ ID NO: 1430 - protein, 15 aa, Artificial sequence
  synthetic sequence
CASSPEDSGTDTQYF

SEQ ID NO: 1431 - protein, 14 aa, Artificial sequence
  synthetic sequence
CASSLQGAVTEAFF

SEQ ID NO: 1432 - protein, 14 aa, Artificial sequence
  synthetic sequence
CASSYDREHSPLHF

SEQ ID NO: 1433 - protein, 13 aa, Artificial sequence
  synthetic sequence
CATSDRGGTEAFF

SEQ ID NO: 1434 - protein, 13 aa, Artificial sequence
  synthetic sequence
CASSFRERYEQYF

SEQ ID NO: 1435 - protein, 14 aa, Artificial sequence
  synthetic sequence
CASSSWDRSYGYTF

SEQ ID NO: 1436 - protein, 15 aa, Artificial sequence
  synthetic sequence
CSARVRPLSTDTQYF

SEQ ID NO: 1437 - protein, 13 aa, Artificial sequence
  synthetic sequence
CSTKSSYNSPLHF

SEQ ID NO: 1438 - protein, 13 aa, Artificial sequence
  synthetic sequence
CASSFSTGGEQYF

SEQ ID NO: 1439 - protein, 16 aa, Artificial sequence
  synthetic sequence
CASSPRGDSSGNTIYF

SEQ ID NO: 1440 - protein, 15 aa, Artificial sequence
  synthetic sequence
CASSRTSGSRYEQYF

SEQ ID NO: 1441 - protein, 14 aa, Artificial sequence
  synthetic sequence
CASSGRDGGTEAFF

SEQ ID NO: 1442 - protein, 15 aa, Artificial sequence
  synthetic sequence
CASSLGAGYLDTQYF

SEQ ID NO: 1443 - protein, 13 aa, Artificial sequence
  synthetic sequence
CASTGPSGNTIYF

SEQ ID NO: 1444 - protein, 16 aa, Artificial sequence
  synthetic sequence
CSARKSGTSSYNEQFF

SEQ ID NO: 1445 - protein, 13 aa, Artificial sequence
  synthetic sequence
CSGDRATNTEAFF

SEQ ID NO: 1446 - protein, 15 aa, Artificial sequence
  synthetic sequence
CASSLGGGWGQPQHF

SEQ ID NO: 1447 - protein, 14 aa, Artificial sequence
  synthetic sequence
CASTGRTENTEAFF

SEQ ID NO: 1448 - protein, 13 aa, Artificial sequence
  synthetic sequence
CASLDRDTGELFF

SEQ ID NO: 1449 - protein, 12 aa, Artificial sequence
  synthetic sequence
CAGGPSSYEQYF

SEQ ID NO: 1450 - protein, 15 aa, Artificial sequence
  synthetic sequence
CASSPLGVDNSPLHF

SEQ ID NO: 1451 - protein, 16 aa, Artificial sequence
  synthetic sequence
CASSGVGYLAKNIQYF

SEQ ID NO: 1452 - protein, 17 aa, Artificial sequence
  synthetic sequence
CASSPGTSGSYNSPLHF

SEQ ID NO: 1453 - protein, 13 aa, Artificial sequence
  synthetic sequence
CASSYSTGQEQYF

SEQ ID NO: 1454 - protein, 13 aa, Artificial sequence
  synthetic sequence
CSVAMDSNTEAFF

SEQ ID NO: 1455 - protein, 17 aa, Artificial sequence
  synthetic sequence
CASSRGQGAAGANVLTF

SEQ ID NO: 1456 - protein, 11 aa, Artificial sequence
  synthetic sequence
CSVGLVGEAFF

SEQ ID NO: 1457 - protein, 15 aa, Artificial sequence
  synthetic sequence
CASRAPTGRNQPQHF

SEQ ID NO: 1458 - protein, 15 aa, Artificial sequence
  synthetic sequence
CASSLARRANTEAFF

SEQ ID NO: 1459 - protein, 13 aa, Artificial sequence
  synthetic sequence
CSVVSSRGQPQHF

SEQ ID NO: 1460 - protein, 16 aa, Artificial sequence
  synthetic sequence
CASSGAGFLAKNIQYF

SEQ ID NO: 1461 - protein, 13 aa, Artificial sequence
  synthetic sequence
CSARDTGGKKLFF

SEQ ID NO: 1462 - protein, 15 aa, Artificial sequence
  synthetic sequence
CASRDQGAGNTEAFF

SEQ ID NO: 1463 - protein, 15 aa, Artificial sequence
  synthetic sequence
CASSLARGLGGELFF

SEQ ID NO: 1464 - protein, 15 aa, Artificial sequence
  synthetic sequence
CATSRDNSNNEKLFF

SEQ ID NO: 1465 - protein, 16 aa, Artificial sequence
  synthetic sequence
CASSLDLAASNYGYTF

SEQ ID NO: 1466 - protein, 18 aa, Artificial sequence
  synthetic sequence
CASSLAPGVAGSSYEQYF

SEQ ID NO: 1467 - protein, 12 aa, Artificial sequence
  synthetic sequence
CASSWQGGGYTF

SEQ ID NO: 1468 - protein, 16 aa, Artificial sequence
  synthetic sequence
CASSPLAGALSYEQYF

SEQ ID NO: 1469 - protein, 15 aa, Artificial sequence
  synthetic sequence
CATSRDYSQNSPLHF

SEQ ID NO: 1470 - protein, 15 aa, Artificial sequence
  synthetic sequence
CATSRERVSNTEAFF

SEQ ID NO: 1471 - protein, 14 aa, Artificial sequence
  synthetic sequence
CASSPLADRYEQYF

SEQ ID NO: 1472 - protein, 14 aa, Artificial sequence
  synthetic sequence
CAISVEAAGNEQFF

SEQ ID NO: 1473 - protein, 15 aa, Artificial sequence
  synthetic sequence
CASSLDGRLTDTQYF

SEQ ID NO: 1474 - protein, 13 aa, Artificial sequence
  synthetic sequence
CASSPRFYNEQFF

SEQ ID NO: 1475 - protein, 15 aa, Artificial sequence
  synthetic sequence
CASSPPGRPNTEAFF

SEQ ID NO: 1476 - protein, 17 aa, Artificial sequence
  synthetic sequence
CASSFRRGESYNSPLHF

SEQ ID NO: 1477 - protein, 15 aa, Artificial sequence
  synthetic sequence
CASSFPTGANTEAFF

SEQ ID NO: 1478 - protein, 14 aa, Artificial sequence
  synthetic sequence
CASSQPGEKNIQYF

SEQ ID NO: 1479 - protein, 12 aa, Artificial sequence
  synthetic sequence
CAWLGTNYGYTF

SEQ ID NO: 1480 - protein, 14 aa, Artificial sequence
  synthetic sequence
CSVGAGGTNEKLFF

SEQ ID NO: 1481 - protein, 13 aa, Artificial sequence
  synthetic sequence
CASSLRFNTEAFF

SEQ ID NO: 1482 - protein, 15 aa, Artificial sequence
  synthetic sequence
CSAKDRGESNQPQHF

SEQ ID NO: 1483 - protein, 12 aa, Artificial sequence
  synthetic sequence
CSARQGSNGYTF

SEQ ID NO: 1484 - protein, 15 aa, Artificial sequence
  synthetic sequence
CASSYPGNSGNTIYF

SEQ ID NO: 1485 - protein, 16 aa, Artificial sequence
  synthetic sequence
CASSYGAPNSGNTIYF

SEQ ID NO: 1486 - protein, 15 aa, Artificial sequence
  synthetic sequence
CASTPGDRGNQPQHF

SEQ ID NO: 1487 - protein, 16 aa, Artificial sequence
  synthetic sequence
CASSPLVGGSYNEQFF

SEQ ID NO: 1488 - protein, 14 aa, Artificial sequence
  synthetic sequence
CASSTGYSGNTIYF

SEQ ID NO: 1489 - protein, 15 aa, Artificial sequence
  synthetic sequence
CASSHRQGAYEKLFF

SEQ ID NO: 1490 - protein, 15 aa, Artificial sequence
  synthetic sequence
CATSSRGRGNQPQHF

SEQ ID NO: 1491 - protein, 13 aa, Artificial sequence
  synthetic sequence
CASSRRAITEAFF

SEQ ID NO: 1492 - protein, 13 aa, Artificial sequence
  synthetic sequence
CASRVGAGDTQYF

SEQ ID NO: 1493 - protein, 14 aa, Artificial sequence
  synthetic sequence
CASSNPGVKNIQYF

SEQ ID NO: 1494 - protein, 15 aa, Artificial sequence
  synthetic sequence
CASSVSGTANQPQHF

SEQ ID NO: 1495 - protein, 16 aa, Artificial sequence
  synthetic sequence
CASSQDKRQGGTEAFF

SEQ ID NO: 1496 - protein, 13 aa, Artificial sequence
  synthetic sequence
CSAPGRSDTEAFF

SEQ ID NO: 1497 - protein, 14 aa, Artificial sequence
  synthetic sequence
CASSLWDTNSPLHF

SEQ ID NO: 1498 - protein, 15 aa, Artificial sequence
  synthetic sequence
CASSEGRADSYEQYF

SEQ ID NO: 1499 - protein, 13 aa, Artificial sequence
  synthetic sequence
CASSPWGGEKLFF

SEQ ID NO: 1500 - protein, 15 aa, Artificial sequence
  synthetic sequence
CSARQLAGGSGELFF

SEQ ID NO: 1501 - protein, 15 aa, Artificial sequence
  synthetic sequence
CASSVQKGANQPQHF

SEQ ID NO: 1502 - protein, 15 aa, Artificial sequence
  synthetic sequence
CASSLLPGGADTQYF

SEQ ID NO: 1503 - protein, 14 aa, Artificial sequence
  synthetic sequence
CASREPGGLTEAFF

SEQ ID NO: 1504 - protein, 14 aa, Artificial sequence
  synthetic sequence
CSVLLAGGKETQYF

SEQ ID NO: 1505 - protein, 12 aa, Artificial sequence
  synthetic sequence
CASSHTGYGYTF

SEQ ID NO: 1506 - protein, 14 aa, Artificial sequence
  synthetic sequence
CASSLGVSGGEQYF

SEQ ID NO: 1507 - protein, 13 aa, Artificial sequence
  synthetic sequence
CATRDGDTGELFF

SEQ ID NO: 1508 - protein, 12 aa, Artificial sequence
  synthetic sequence
CSVGRQETEAFF

SEQ ID NO: 1509 - protein, 14 aa, Artificial sequence
  synthetic sequence
CASSLARSRTEAFF

SEQ ID NO: 1510 - protein, 14 aa, Artificial sequence
  synthetic sequence
CASSLAGAPSEQYF

SEQ ID NO: 1511 - protein, 12 aa, Artificial sequence
  synthetic sequence
CASSLGYEGYTF

SEQ ID NO: 1512 - protein, 16 aa, Artificial sequence
  synthetic sequence
CASSLGLGRPTGELFF

SEQ ID NO: 1513 - protein, 15 aa, Artificial sequence
  synthetic sequence
CASSLRGPRDTEAFF

SEQ ID NO: 1514 - protein, 13 aa, Artificial sequence
  synthetic sequence
CSARLGTVSGYTF

SEQ ID NO: 1515 - protein, 14 aa, Artificial sequence
  synthetic sequence
CASSLWDRDTEAFF

SEQ ID NO: 1516 - protein, 14 aa, Artificial sequence
  synthetic sequence
CATLQVGAKNIQYF

SEQ ID NO: 1517 - protein, 13 aa, Artificial sequence
  synthetic sequence
CSARVLDSYEQYF

SEQ ID NO: 1518 - protein, 12 aa, Artificial sequence
  synthetic sequence
CAWSRQAGGYTF

SEQ ID NO: 1519 - protein, 14 aa, Artificial sequence
  synthetic sequence
CASRGQGDGQPQHF

SEQ ID NO: 1520 - protein, 14 aa, Artificial sequence
  synthetic sequence
CASSSPGAKNIQYF

SEQ ID NO: 1521 - protein, 15 aa, Artificial sequence
  synthetic sequence
CASSQPKRGTYEQYF

SEQ ID NO: 1522 - protein, 15 aa, Artificial sequence
  synthetic sequence
CASSRAGGGQETQYF

SEQ ID NO: 1523 - protein, 12 aa, Artificial sequence
  synthetic sequence
CASSRTGTPLHF

SEQ ID NO: 1524 - protein, 14 aa, Artificial sequence
  synthetic sequence
CASSLDLIAYEQYF

SEQ ID NO: 1525 - protein, 15 aa, Artificial sequence
  synthetic sequence
CASSPDRGQGDTQYF

SEQ ID NO: 1526 - protein, 16 aa, Artificial sequence
  synthetic sequence
CASSIDPGQTYYGYTF

SEQ ID NO: 1527 - protein, 15 aa, Artificial sequence
  synthetic sequence
CSAPRPSGREETQYF

SEQ ID NO: 1528 - protein, 14 aa, Artificial sequence
  synthetic sequence
CASSYREGNSPLHF

SEQ ID NO: 1529 - protein, 13 aa, Artificial sequence
  synthetic sequence
CSVRAGVRNEQFF

SEQ ID NO: 1530 - protein, 14 aa, Artificial sequence
  synthetic sequence
CASSVTGGQAEAFF

SEQ ID NO: 1531 - protein, 13 aa, Artificial sequence
  synthetic sequence
CAISQSSNYGYTF

SEQ ID NO: 1532 - protein, 15 aa, Artificial sequence
  synthetic sequence
CSALPRTGGGNTIYF

SEQ ID NO: 1533 - protein, 14 aa, Artificial sequence
  synthetic sequence
CASSRTGLDNEQFF

SEQ ID NO: 1534 - protein, 15 aa, Artificial sequence
  synthetic sequence
CSAREGTSEHNEQFF

SEQ ID NO: 1535 - protein, 16 aa, Artificial sequence
  synthetic sequence
CASSLTPTGGGQPQHF

SEQ ID NO: 1536 - protein, 16 aa, Artificial sequence
  synthetic sequence
CASSLSPGGPTGELFF

SEQ ID NO: 1537 - protein, 15 aa, Artificial sequence
  synthetic sequence
CASSRTGTTTDTQYF

SEQ ID NO: 1538 - protein, 15 aa, Artificial sequence
  synthetic sequence
CSARDAGRYNEKLFF

SEQ ID NO: 1539 - protein, 15 aa, Artificial sequence
  synthetic sequence
CASSPTAFYNSPLHF

SEQ ID NO: 1540 - protein, 15 aa, Artificial sequence
  synthetic sequence
CASSLDSLGNTEAFF

SEQ ID NO: 1541 - protein, 12 aa, Artificial sequence
  synthetic sequence
CATRDLGEKLFF

SEQ ID NO: 1542 - protein, 14 aa, Artificial sequence
  synthetic sequence
CASSRTEYNSPLHF

SEQ ID NO: 1543 - protein, 16 aa, Artificial sequence
  synthetic sequence
CSARDPRQGRSYGYTF

SEQ ID NO: 1544 - protein, 15 aa, Artificial sequence
  synthetic sequence
CASSTRQGVREKLFF

SEQ ID NO: 1545 - protein, 14 aa, Artificial sequence
  synthetic sequence
CASSRKGRNTEAFF

SEQ ID NO: 1546 - protein, 15 aa, Artificial sequence
  synthetic sequence
CASSPGGGTAYEQYF

SEQ ID NO: 1547 - protein, 14 aa, Artificial sequence
  synthetic sequence
CASSPDREAHEQYF

SEQ ID NO: 1548 - protein, 13 aa, Artificial sequence
  synthetic sequence
CSASMDRDQPQHF

SEQ ID NO: 1549 - protein, 15 aa, Artificial sequence
  synthetic sequence
CASSRAGTKYNEQFF

SEQ ID NO: 1550 - protein, 14 aa, Artificial sequence
  synthetic sequence
CASSQAGRETEAFF

SEQ ID NO: 1551 - protein, 13 aa, Artificial sequence
  synthetic sequence
CASTRQDGNTIYF

SEQ ID NO: 1552 - protein, 13 aa, Artificial sequence
  synthetic sequence
CASSYGEQYGYTF

SEQ ID NO: 1553 - protein, 15 aa, Artificial sequence
  synthetic sequence
CAIRGSSSSYNEQFF

SEQ ID NO: 1554 - protein, 13 aa, Artificial sequence
  synthetic sequence
CASSQGEDYGYTF

SEQ ID NO: 1555 - protein, 15 aa, Artificial sequence
  synthetic sequence
CASKRGASGANVLTF

SEQ ID NO: 1556 - protein, 14 aa, Artificial sequence
  synthetic sequence
CASTPGQLNTEAFF

SEQ ID NO: 1557 - protein, 14 aa, Artificial sequence
  synthetic sequence
CASRDRGESQPQHF

SEQ ID NO: 1558 - protein, 17 aa, Artificial sequence
  synthetic sequence
CATSDSGLAGGAETQYF

SEQ ID NO: 1559 - protein, 16 aa, Artificial sequence
  synthetic sequence
CASSSGVAGVQETQYF

SEQ ID NO: 1560 - protein, 11 aa, Artificial sequence
  synthetic sequence
CSASDHSPLHF

SEQ ID NO: 1561 - protein, 16 aa, Artificial sequence
  synthetic sequence
CASSLEPTSGGETQYF

SEQ ID NO: 1562 - protein, 14 aa, Artificial sequence
  synthetic sequence
CSARADHPRETQYF

SEQ ID NO: 1563 - protein, 13 aa, Artificial sequence
  synthetic sequence
CASQRLANYGYTF

SEQ ID NO: 1564 - protein, 13 aa, Artificial sequence
  synthetic sequence
CASSRSPREGYTF

SEQ ID NO: 1565 - protein, 14 aa, Artificial sequence
  synthetic sequence
CASSQVFGQETQYF

SEQ ID NO: 1566 - protein, 16 aa, Artificial sequence
  synthetic sequence
CASSLPGTGGNTEAFF

SEQ ID NO: 1567 - protein, 13 aa, Artificial sequence
  synthetic sequence
CASSYLNYYGYTF

SEQ ID NO: 1568 - protein, 14 aa, Artificial sequence
  synthetic sequence
CASNTQGANSPLHF

SEQ ID NO: 1569 - protein, 14 aa, Artificial sequence
  synthetic sequence
CASSLYPALYEQYF

SEQ ID NO: 1570 - protein, 16 aa, Artificial sequence
  synthetic sequence
CASSRQRGSSYNEQFF

SEQ ID NO: 1571 - protein, 13 aa, Artificial sequence
  synthetic sequence
CATSDLTRSEQFF

SEQ ID NO: 1572 - protein, 16 aa, Artificial sequence
  synthetic sequence
CASSPAGVGLTDTQYF

SEQ ID NO: 1573 - protein, 14 aa, Artificial sequence
  synthetic sequence
CASSFGDKNYGYTF

SEQ ID NO: 1574 - protein, 14 aa, Artificial sequence
  synthetic sequence
CASSQDPGRGTQYF

SEQ ID NO: 1575 - protein, 13 aa, Artificial sequence
  synthetic sequence
CASTIQGIQPQHF

SEQ ID NO: 1576 - protein, 14 aa, Artificial sequence
  synthetic sequence
CASSQAGNIYGYTF

SEQ ID NO: 1577 - protein, 14 aa, Artificial sequence
  synthetic sequence
CASSRVGPNYGYTF

SEQ ID NO: 1578 - protein, 14 aa, Artificial sequence
  synthetic sequence
CASRIRGGAYEQYF

SEQ ID NO: 1579 - protein, 15 aa, Artificial sequence
  synthetic sequence
CASSPSGAAYSPLHF

SEQ ID NO: 1580 - protein, 13 aa, Artificial sequence
  synthetic sequence
CASREVSGNTIYF

SEQ ID NO: 1581 - protein, 16 aa, Artificial sequence
  synthetic sequence
CASSPAGDRNSYEQYF

SEQ ID NO: 1582 - protein, 14 aa, Artificial sequence
  synthetic sequence
CASTQGGSGTEAFF

SEQ ID NO: 1583 - protein, 15 aa, Artificial sequence
  synthetic sequence
CASSQELGLYNEQFF

SEQ ID NO: 1584 - protein, 14 aa, Artificial sequence
  synthetic sequence
CSASWRTGGTEAFF

SEQ ID NO: 1585 - protein, 15 aa, Artificial sequence
  synthetic sequence
CASSPPAGGNTEAFF

SEQ ID NO: 1586 - protein, 14 aa, Artificial sequence
  synthetic sequence
CASKGTQGNQPQHF

SEQ ID NO: 1587 - protein, 13 aa, Artificial sequence
  synthetic sequence
CAISDRALQPQHF

SEQ ID NO: 1588 - protein, 14 aa, Artificial sequence
  synthetic sequence
CASSKDRDYNEQFF

SEQ ID NO: 1589 - protein, 13 aa, Artificial sequence
  synthetic sequence
CSVGRTGGFGYTF

SEQ ID NO: 1590 - protein, 13 aa, Artificial sequence
  synthetic sequence
CSGKGTTNEKLFF

SEQ ID NO: 1591 - protein, 14 aa, Artificial sequence
  synthetic sequence
CAISAPGPNYGYTF

SEQ ID NO: 1592 - protein, 14 aa, Artificial sequence
  synthetic sequence
CASSFGDSSYGYTF

SEQ ID NO: 1593 - protein, 14 aa, Artificial sequence
  synthetic sequence
CASSLWQARNTIYF

SEQ ID NO: 1594 - protein, 14 aa, Artificial sequence
  synthetic sequence
CASTGSFSGNTIYF

SEQ ID NO: 1595 - protein, 16 aa, Artificial sequence
  synthetic sequence
CASSQVRGRDTDTQYF

SEQ ID NO: 1596 - protein, 13 aa, Artificial sequence
  synthetic sequence
CSASWASNEKLFF

SEQ ID NO: 1597 - protein, 15 aa, Artificial sequence
  synthetic sequence
CASSLGGNTYQPQHF

SEQ ID NO: 1598 - protein, 12 aa, Artificial sequence
  synthetic sequence
CAWNRRRETQYF

SEQ ID NO: 1599 - protein, 12 aa, Artificial sequence
  synthetic sequence
CASRKLIYGYTF

SEQ ID NO: 1600 - protein, 14 aa, Artificial sequence
  synthetic sequence
CATSRDRQVPGYTF

SEQ ID NO: 1601 - protein, 17 aa, Artificial sequence
  synthetic sequence
CSARDPDPSSYNSPLHF

SEQ ID NO: 1602 - protein, 16 aa, Artificial sequence
  synthetic sequence
CASSLELLAGVETQYF

SEQ ID NO: 1603 - protein, 16 aa, Artificial sequence
  synthetic sequence
CASSQVRTGANEKLFF

SEQ ID NO: 1604 - protein, 15 aa, Artificial sequence
  synthetic sequence
CASSIRTGIVYEQYF

SEQ ID NO: 1605 - protein, 12 aa, Artificial sequence
  synthetic sequence
CASSTPYNEQFF

SEQ ID NO: 1606 - protein, 15 aa, Artificial sequence
  synthetic sequence
CASSPPQREGETQYF

SEQ ID NO: 1607 - protein, 13 aa, Artificial sequence
  synthetic sequence
CSVEEFRGDGYTF

SEQ ID NO: 1608 - protein, 12 aa, Artificial sequence
  synthetic sequence
CASSVGLYEQYF

SEQ ID NO: 1609 - protein, 14 aa, Artificial sequence
  synthetic sequence
CASNLAGDADTQYF

SEQ ID NO: 1610 - protein, 13 aa, Artificial sequence
  synthetic sequence
CSAPSGSGNTQYF

SEQ ID NO: 1611 - protein, 14 aa, Artificial sequence
  synthetic sequence
CASRPPGQKNIQYF

SEQ ID NO: 1612 - protein, 15 aa, Artificial sequence
  synthetic sequence
CASSLAYYSNQPQHF

SEQ ID NO: 1613 - protein, 14 aa, Artificial sequence
  synthetic sequence
CASRSTAGSYEQYF

SEQ ID NO: 1614 - protein, 14 aa, Artificial sequence
  synthetic sequence
CASSGAAGTDTQYF

SEQ ID NO: 1615 - protein, 10 aa, Artificial sequence
  synthetic sequence
CASGGLPQHF

SEQ ID NO: 1616 - protein, 15 aa, Artificial sequence
  synthetic sequence
CASSTTGGENYGYTF

SEQ ID NO: 1617 - protein, 15 aa, Artificial sequence
  synthetic sequence
CASSRWTGLNTEAFF

SEQ ID NO: 1618 - protein, 12 aa, Artificial sequence
  synthetic sequence
CASSTTANEQFF

SEQ ID NO: 1619 - protein, 16 aa, Artificial sequence
  synthetic sequence
CASSYAGQGFNQPQHF

SEQ ID NO: 1620 - protein, 15 aa, Artificial sequence
  synthetic sequence
CASSRPGQKNTEAFF

SEQ ID NO: 1621 - protein, 14 aa, Artificial sequence
  synthetic sequence
CASSLYSRVYEQYF

SEQ ID NO: 1622 - protein, 14 aa, Artificial sequence
  synthetic sequence
CASRGTGSTQPQHF

SEQ ID NO: 1623 - protein, 15 aa, Artificial sequence
  synthetic sequence
CASSLGASNNQPQHF

SEQ ID NO: 1624 - protein, 15 aa, Artificial sequence
  synthetic sequence
CASSRGTGEIYGYTF

SEQ ID NO: 1625 - protein, 13 aa, Artificial sequence
  synthetic sequence
CASSPDGGIQAFF

SEQ ID NO: 1626 - protein, 15 aa, Artificial sequence
  synthetic sequence
CASGLERDSNQPQHF

SEQ ID NO: 1627 - protein, 15 aa, Artificial sequence
  synthetic sequence
CASSDSLAGAGEQFF

SEQ ID NO: 1628 - protein, 14 aa, Artificial sequence
  synthetic sequence
CSASALVSGNTIYF

SEQ ID NO: 1629 - protein, 17 aa, Artificial sequence
  synthetic sequence
CSARDRGLGTNTGELFF

SEQ ID NO: 1630 - protein, 15 aa, Artificial sequence
  synthetic sequence
CASSRLGEAGNTIYF

SEQ ID NO: 1631 - protein, 14 aa, Artificial sequence
  synthetic sequence
CASSLEVYGTEAFF

SEQ ID NO: 1632 - protein, 15 aa, Artificial sequence
  synthetic sequence
CASSSPGTLNYGYTF

SEQ ID NO: 1633 - protein, 14 aa, Artificial sequence
  synthetic sequence
CASRRQGAVTEAFF

SEQ ID NO: 1634 - protein, 15 aa, Artificial sequence
  synthetic sequence
CASSPETAPSYEQYF

SEQ ID NO: 1635 - protein, 12 aa, Artificial sequence
  synthetic sequence
CSAMTGPYGYTF

SEQ ID NO: 1636 - protein, 14 aa, Artificial sequence
  synthetic sequence
CASSLSAWNYGYTF

SEQ ID NO: 1637 - protein, 15 aa, Artificial sequence
  synthetic sequence
CASSPLPGGDEKLFF

SEQ ID NO: 1638 - protein, 14 aa, Artificial sequence
  synthetic sequence
CASSYEVTNYGYTF

SEQ ID NO: 1639 - protein, 14 aa, Artificial sequence
  synthetic sequence
CASSQGTSVGELFF

SEQ ID NO: 1640 - protein, 14 aa, Artificial sequence
  synthetic sequence
CASSSQGTSTEAFF

SEQ ID NO: 1641 - protein, 17 aa, Artificial sequence
  synthetic sequence
CASSQGLAGVLSDTQYF

SEQ ID NO: 1642 - protein, 16 aa, Artificial sequence
  synthetic sequence
CASSLIGGRITGELFF

SEQ ID NO: 1643 - protein, 15 aa, Artificial sequence
  synthetic sequence
CASSFGGDMNTEAFF

SEQ ID NO: 1644 - protein, 13 aa, Artificial sequence
  synthetic sequence
CASGPGGGRGYTF

SEQ ID NO: 1645 - protein, 17 aa, Artificial sequence
  synthetic sequence
CASSASEGSSYNSPLHF

SEQ ID NO: 1646 - protein, 13 aa, Artificial sequence
  synthetic sequence
CASSLGQGEEAFF

SEQ ID NO: 1647 - protein, 14 aa, Artificial sequence
  synthetic sequence
CASGKGGADEKLFF

SEQ ID NO: 1648 - protein, 14 aa, Artificial sequence
  synthetic sequence
CASSFQGAGDGYTF

SEQ ID NO: 1649 - protein, 13 aa, Artificial sequence
  synthetic sequence
CASSSSGLLEAFF

SEQ ID NO: 1650 - protein, 15 aa, Artificial sequence
  synthetic sequence
CASSLGAGGEGGYTF

SEQ ID NO: 1651 - protein, 15 aa, Artificial sequence
  synthetic sequence
CASSFWDSLNYGYTF

SEQ ID NO: 1652 - protein, 13 aa, Artificial sequence
  synthetic sequence
CASSAGRLEKLFF

SEQ ID NO: 1653 - protein, 16 aa, Artificial sequence
  synthetic sequence
CASSRRTGGSTDTQYF

SEQ ID NO: 1654 - protein, 13 aa, Artificial sequence
  synthetic sequence
CASSYRVWTEAFF

SEQ ID NO: 1655 - protein, 12 aa, Artificial sequence
  synthetic sequence
CASSQSNYGYTF

SEQ ID NO: 1656 - protein, 15 aa, Artificial sequence
  synthetic sequence
CASSFNRGHSYEQYF

SEQ ID NO: 1657 - protein, 14 aa, Artificial sequence
  synthetic sequence
CASSLRPPGDTQYF

SEQ ID NO: 1658 - protein, 14 aa, Artificial sequence
  synthetic sequence
CASSFPVGNSPLHF

SEQ ID NO: 1659 - protein, 16 aa, Artificial sequence
  synthetic sequence
CASSRISGPSSYEQYF

SEQ ID NO: 1660 - protein, 14 aa, Artificial sequence
  synthetic sequence
CATRSGLGSNEQFF

SEQ ID NO: 1661 - protein, 16 aa, Artificial sequence
  synthetic sequence
CSASPNRGGGNQPQHF

SEQ ID NO: 1662 - protein, 12 aa, Artificial sequence
  synthetic sequence
CATSREKAPQHF

SEQ ID NO: 1663 - protein, 17 aa, Artificial sequence
  synthetic sequence
CASSAGTAGSYNSPLHF

SEQ ID NO: 1664 - protein, 13 aa, Artificial sequence
  synthetic sequence
CASSYSGYYEAFF

SEQ ID NO: 1665 - protein, 15 aa, Artificial sequence
  synthetic sequence
CASTPIDSLNTEAFF

SEQ ID NO: 1666 - protein, 16 aa, Artificial sequence
  synthetic sequence
CASSQGRQGDTGELFF

SEQ ID NO: 1667 - protein, 13 aa, Artificial sequence
  synthetic sequence
CSARWRGIYEQYF

SEQ ID NO: 1668 - protein, 14 aa, Artificial sequence
  synthetic sequence
CASQIQGLNTEAFF

SEQ ID NO: 1669 - protein, 14 aa, Artificial sequence
  synthetic sequence
CASSYHRDRETQYF

SEQ ID NO: 1670 - protein, 13 aa, Artificial sequence
  synthetic sequence
CASKRDGGEKLFF

SEQ ID NO: 1671 - protein, 15 aa, Artificial sequence
  synthetic sequence
CASSLPRTGGNTIYF

SEQ ID NO: 1672 - protein, 17 aa, Artificial sequence
  synthetic sequence
CASSYSNPRSYNSPLHF

SEQ ID NO: 1673 - protein, 14 aa, Artificial sequence
  synthetic sequence
CASSLWGEGYGYTF

SEQ ID NO: 1674 - protein, 14 aa, Artificial sequence
  synthetic sequence
CAWTQQGTNEKLFF

SEQ ID NO: 1675 - protein, 14 aa, Artificial sequence
  synthetic sequence
CASSSGQFNQPQHF

SEQ ID NO: 1676 - protein, 13 aa, Artificial sequence
  synthetic sequence
CASSLGTGNIQYF

SEQ ID NO: 1677 - protein, 14 aa, Artificial sequence
  synthetic sequence
CASSNPGANTEAFF

SEQ ID NO: 1678 - protein, 15 aa, Artificial sequence
  synthetic sequence
CASSSWTSGRYEQYF

SEQ ID NO: 1679 - protein, 16 aa, Artificial sequence
  synthetic sequence
CASSRGGQRYNSPLHF

SEQ ID NO: 1680 - protein, 16 aa, Artificial sequence
  synthetic sequence
CASSEYLAGDTGELFF

SEQ ID NO: 1681 - protein, 14 aa, Artificial sequence
  synthetic sequence
CASSPTGLIYEQYF

SEQ ID NO: 1682 - protein, 14 aa, Artificial sequence
  synthetic sequence
CASSLSGAASPLHF

SEQ ID NO: 1683 - protein, 15 aa, Artificial sequence
  synthetic sequence
CASSLRVEGADTQYF

SEQ ID NO: 1684 - protein, 15 aa, Artificial sequence
  synthetic sequence
CASRTGGGLNSPLHF

SEQ ID NO: 1685 - protein, 13 aa, Artificial sequence
  synthetic sequence
CSARRNLNNEQFF

SEQ ID NO: 1686 - protein, 15 aa, Artificial sequence
  synthetic sequence
CASSQVGRGPNEQFF

SEQ ID NO: 1687 - protein, 15 aa, Artificial sequence
  synthetic sequence
CATSRNRGGYQPQHF

SEQ ID NO: 1688 - protein, 13 aa, Artificial sequence
  synthetic sequence
CASSKGLHYGYTF

SEQ ID NO: 1689 - protein, 14 aa, Artificial sequence
  synthetic sequence
CSAKGDRMNTEAFF

SEQ ID NO: 1690 - protein, 12 aa, Artificial sequence
  synthetic sequence
CASNMGSYGYTF

SEQ ID NO: 1691 - protein, 13 aa, Artificial sequence
  synthetic sequence
CSVEVRGYTEAFF

SEQ ID NO: 1692 - protein, 12 aa, Artificial sequence
  synthetic sequence
CASSEQTGEAFF

SEQ ID NO: 1693 - protein, 15 aa, Artificial sequence
  synthetic sequence
CASSPYRTGPYEQYF

SEQ ID NO: 1694 - protein, 14 aa, Artificial sequence
  synthetic sequence
CASRGTSGGGTQYF

SEQ ID NO: 1695 - protein, 15 aa, Artificial sequence
  synthetic sequence
CASSSEGGYNQPQHF

SEQ ID NO: 1696 - protein, 14 aa, Artificial sequence
  synthetic sequence
CASSFLGHNSPLHF

SEQ ID NO: 1697 - protein, 15 aa, Artificial sequence
  synthetic sequence
CASSYSTALQETQYF

SEQ ID NO: 1698 - protein, 16 aa, Artificial sequence
  synthetic sequence
CASSSPETGTNSPLHF

SEQ ID NO: 1699 - protein, 15 aa, Artificial sequence
  synthetic sequence
CASSPGAGPPYEQYF

SEQ ID NO: 1700 - protein, 14 aa, Artificial sequence
  synthetic sequence
CATSQGGYNSPLHF

SEQ ID NO: 1701 - protein, 14 aa, Artificial sequence
  synthetic sequence
CASSPGQNHNEQFF

SEQ ID NO: 1702 - protein, 14 aa, Artificial sequence
  synthetic sequence
CASRRDTLASPLHF

SEQ ID NO: 1703 - protein, 14 aa, Artificial sequence
  synthetic sequence
CASRSEGASTEAFF

SEQ ID NO: 1704 - protein, 11 aa, Artificial sequence
  synthetic sequence
CASSRPREQYF

SEQ ID NO: 1705 - protein, 9 aa, Artificial sequence
  synthetic sequence
CASVREQYF

SEQ ID NO: 1706 - protein, 15 aa, Artificial sequence
  synthetic sequence
CASSYRDRDAYEQYF

SEQ ID NO: 1707 - protein, 15 aa, Artificial sequence
  synthetic sequence
CASSGGDRGRSPLHF

SEQ ID NO: 1708 - protein, 14 aa, Artificial sequence
  synthetic sequence
CAISADRAGAEAFF

SEQ ID NO: 1709 - protein, 15 aa, Artificial sequence
  synthetic sequence
CASSPTTGLNQPQHF

SEQ ID NO: 1710 - protein, 14 aa, Artificial sequence
  synthetic sequence
CASSRQFNTGELFF

SEQ ID NO: 1711 - protein, 15 aa, Artificial sequence
  synthetic sequence
CASSQDAGKGYGYTF

SEQ ID NO: 1712 - protein, 17 aa, Artificial sequence
  synthetic sequence
CASSPGTAGSYNSPLHF

SEQ ID NO: 1713 - protein, 15 aa, Artificial sequence
  synthetic sequence
CASSQYSASTDTQYF

SEQ ID NO: 1714 - protein, 14 aa, Artificial sequence
  synthetic sequence
CASRLDTSTDTQYF

SEQ ID NO: 1715 - protein, 14 aa, Artificial sequence
  synthetic sequence
CASSFRPGDYGYTF

SEQ ID NO: 1716 - protein, 14 aa, Artificial sequence
  synthetic sequence
CASSQALTSDEQFF

SEQ ID NO: 1717 - protein, 16 aa, Artificial sequence
  synthetic sequence
CASSFGDRGGNYGYTF

SEQ ID NO: 1718 - protein, 13 aa, Artificial sequence
  synthetic sequence
CASHRGLGTEAFF

SEQ ID NO: 1719 - protein, 15 aa, Artificial sequence
  synthetic sequence
CATSDRGLLGETQYF

SEQ ID NO: 1720 - protein, 14 aa, Artificial sequence
  synthetic sequence
CASSTQGVTYEQYF

SEQ ID NO: 1721 - protein, 14 aa, Artificial sequence
  synthetic sequence
CASRAGTSTGELFF

SEQ ID NO: 1722 - protein, 17 aa, Artificial sequence
  synthetic sequence
CASSPPLRDRAYNEQFF

SEQ ID NO: 1723 - protein, 16 aa, Artificial sequence
  synthetic sequence
CASNRPAGSGANVLTF

SEQ ID NO: 1724 - protein, 14 aa, Artificial sequence
  synthetic sequence
CASSPLRGSYGYTF

SEQ ID NO: 1725 - protein, 15 aa, Artificial sequence
  synthetic sequence
CSAPRGGAYYNEQFF

SEQ ID NO: 1726 - protein, 15 aa, Artificial sequence
  synthetic sequence
CASSLDPSPNTEAFF

SEQ ID NO: 1727 - protein, 15 aa, Artificial sequence
  synthetic sequence
CASSLSGQLNYGYTF

SEQ ID NO: 1728 - protein, 12 aa, Artificial sequence
  synthetic sequence
CASRDHNYGYTF

SEQ ID NO: 1729 - protein, 16 aa, Artificial sequence
  synthetic sequence
CASSPLSSSGANVLTF

SEQ ID NO: 1730 - protein, 12 aa, Artificial sequence
  synthetic sequence
CASSSPNAEAFF

SEQ ID NO: 1731 - protein, 15 aa, Artificial sequence
  synthetic sequence
CASSQDPSGVTEAFF

SEQ ID NO: 1732 - protein, 15 aa, Artificial sequence
  synthetic sequence
CASSTSGTDNQPQHF

SEQ ID NO: 1733 - protein, 12 aa, Artificial sequence
  synthetic sequence
CASSLGPQGQYF

SEQ ID NO: 1734 - protein, 15 aa, Artificial sequence
  synthetic sequence
CASSLGQGGSQPQHF

SEQ ID NO: 1735 - protein, 15 aa, Artificial sequence
  synthetic sequence
CASSEWGLRSYEQYF

SEQ ID NO: 1736 - protein, 17 aa, Artificial sequence
  synthetic sequence
CSAPRTSGGRSTDTQYF

SEQ ID NO: 1737 - protein, 16 aa, Artificial sequence
  synthetic sequence
CASSYLRAGNTDTQYF

SEQ ID NO: 1738 - protein, 14 aa, Artificial sequence
  synthetic sequence
CASSRTADNSPLHF

SEQ ID NO: 1739 - protein, 15 aa, Artificial sequence
  synthetic sequence
CASSLSFGGNSPLHF

SEQ ID NO: 1740 - protein, 14 aa, Artificial sequence
  synthetic sequence
CASSELAGLYGYTF

SEQ ID NO: 1741 - protein, 15 aa, Artificial sequence
  synthetic sequence
CASSFPGTNTDTQYF

SEQ ID NO: 1742 - protein, 13 aa, Artificial sequence
  synthetic sequence
CASSPPVNYGYTF

SEQ ID NO: 1743 - protein, 12 aa, Artificial sequence
  synthetic sequence
CASKDTNTEAFF

SEQ ID NO: 1744 - protein, 15 aa, Artificial sequence
  synthetic sequence
CASSPRTGINSPLHF

SEQ ID NO: 1745 - protein, 12 aa, Artificial sequence
  synthetic sequence
CASSPRLAEAFF

SEQ ID NO: 1746 - protein, 15 aa, Artificial sequence
  synthetic sequence
CASSQELQGLYGYTF

SEQ ID NO: 1747 - protein, 15 aa, Artificial sequence
  synthetic sequence
CASSPRLDRGYGYTF

SEQ ID NO: 1748 - protein, 13 aa, Artificial sequence
  synthetic sequence
CASISTGTYGYTF

SEQ ID NO: 1749 - protein, 13 aa, Artificial sequence
  synthetic sequence
CASSESPGYGYTF

SEQ ID NO: 1750 - protein, 15 aa, Artificial sequence
  synthetic sequence
CAWSVQGSRNQPQHF

SEQ ID NO: 1751 - protein, 16 aa, Artificial sequence
  synthetic sequence
CASSMGQGAGAGELFF

SEQ ID NO: 1752 - protein, 17 aa, Artificial sequence
  synthetic sequence
CASSYSPPSGVYNEQFF

SEQ ID NO: 1753 - protein, 15 aa, Artificial sequence
  synthetic sequence
CAWSVRPGRNTEAFF

SEQ ID NO: 1754 - protein, 14 aa, Artificial sequence
  synthetic sequence
CASSLGVGWETQYF

SEQ ID NO: 1755 - protein, 14 aa, Artificial sequence
  synthetic sequence
CASSLTSGRHEQYF

SEQ ID NO: 1756 - protein, 14 aa, Artificial sequence
  synthetic sequence
CASSLDRGVTEAFF

SEQ ID NO: 1757 - protein, 15 aa, Artificial sequence
  synthetic sequence
CATSRRTGRNSPLHF

SEQ ID NO: 1758 - protein, 16 aa, Artificial sequence
  synthetic sequence
CASSSGTGGSGNTIYF

SEQ ID NO: 1759 - protein, 16 aa, Artificial sequence
  synthetic sequence
CASSLDTTLAKNIQYF

SEQ ID NO: 1760 - protein, 13 aa, Artificial sequence
  synthetic sequence
CASSERLTGELFF

SEQ ID NO: 1761 - protein, 11 aa, Artificial sequence
  synthetic sequence
CASSQHQKLFF

SEQ ID NO: 1762 - protein, 12 aa, Artificial sequence
  synthetic sequence
CASTKQGQPQHF

SEQ ID NO: 1763 - protein, 12 aa, Artificial sequence
  synthetic sequence
CSAPGESTEAFF

SEQ ID NO: 1764 - protein, 14 aa, Artificial sequence
  synthetic sequence
CASSPAIRDTEAFF

SEQ ID NO: 1765 - protein, 14 aa, Artificial sequence
  synthetic sequence
CSVEETGGNQPQHF

SEQ ID NO: 1766 - protein, 14 aa, Artificial sequence
  synthetic sequence
CASSQEGGIYGYTF

SEQ ID NO: 1767 - protein, 17 aa, Artificial sequence
  synthetic sequence
CASSQDRGSSSGNTIYF

SEQ ID NO: 1768 - protein, 12 aa, Artificial sequence
  synthetic sequence
CSATQGLETQYF

SEQ ID NO: 1769 - protein, 17 aa, Artificial sequence
  synthetic sequence
CASSYGASGGLTDTQYF

SEQ ID NO: 1770 - protein, 14 aa, Artificial sequence
  synthetic sequence
CSVDGQGNTDTQYF

SEQ ID NO: 1771 - protein, 15 aa, Artificial sequence
  synthetic sequence
CAWSVRDSLNEKLFF

SEQ ID NO: 1772 - protein, 15 aa, Artificial sequence
  synthetic sequence
CASSPTGVAGNTIYF

SEQ ID NO: 1773 - protein, 15 aa, Artificial sequence
  synthetic sequence
CASSPPVGDNSPLHF

SEQ ID NO: 1774 - protein, 14 aa, Artificial sequence
  synthetic sequence
CASRRDRGNEKLFF

SEQ ID NO: 1775 - protein, 15 aa, Artificial sequence
  synthetic sequence
CATSREKGPNTEAFF

SEQ ID NO: 1776 - protein, 15 aa, Artificial sequence
  synthetic sequence
CASSLRRGLGYGYTF

SEQ ID NO: 1777 - protein, 16 aa, Artificial sequence
  synthetic sequence
CASSVEDSGGYNEQFF

SEQ ID NO: 1778 - protein, 14 aa, Artificial sequence
  synthetic sequence
CASSASGGYYEQYF

SEQ ID NO: 1779 - protein, 17 aa, Artificial sequence
  synthetic sequence
CASSLRSSGGVDNEQFF

SEQ ID NO: 1780 - protein, 14 aa, Artificial sequence
  synthetic sequence
CASSQSPTSYEQYF

SEQ ID NO: 1781 - protein, 16 aa, Artificial sequence
  synthetic sequence
CASSLAPLAGGNEQFF

SEQ ID NO: 1782 - protein, 14 aa, Artificial sequence
  synthetic sequence
CASSQDRRSYEQYF

SEQ ID NO: 1783 - protein, 16 aa, Artificial sequence
  synthetic sequence
CASSLAGTGRADTQYF

SEQ ID NO: 1784 - protein, 12 aa, Artificial sequence
  synthetic sequence
CASSFAQETQYF

SEQ ID NO: 1785 - protein, 14 aa, Artificial sequence
  synthetic sequence
CASSSNRVAYEQYF

SEQ ID NO: 1786 - protein, 15 aa, Artificial sequence
  synthetic sequence
CSASLAGVAKNIQYF

SEQ ID NO: 1787 - protein, 15 aa, Artificial sequence
  synthetic sequence
CASSTMTSGSYEQYF

SEQ ID NO: 1788 - protein, 13 aa, Artificial sequence
  synthetic sequence
CASSQQLAGEQYF

SEQ ID NO: 1789 - protein, 11 aa, Artificial sequence
  synthetic sequence
CASRGLEKLFF

SEQ ID NO: 1790 - protein, 15 aa, Artificial sequence
  synthetic sequence
CASSYPTGATYEQYF

SEQ ID NO: 1791 - protein, 14 aa, Artificial sequence
  synthetic sequence
CASSYRESNQPQHF

SEQ ID NO: 1792 - protein, 15 aa, Artificial sequence
  synthetic sequence
CSVELGTGGNYGYTF

SEQ ID NO: 1793 - protein, 14 aa, Artificial sequence
  synthetic sequence
CASSRQGQGNTIYF

SEQ ID NO: 1794 - protein, 12 aa, Artificial sequence
  synthetic sequence
CASSLGQRKAFF

SEQ ID NO: 1795 - protein, 13 aa, Artificial sequence
  synthetic sequence
CSARFQVDQPQHF

SEQ ID NO: 1796 - protein, 14 aa, Artificial sequence
  synthetic sequence
CASSLDVYNQPQHF

SEQ ID NO: 1797 - protein, 14 aa, Artificial sequence
  synthetic sequence
CASSDSDSQETQYF

SEQ ID NO: 1798 - protein, 15 aa, Artificial sequence
  synthetic sequence
CASSLAATNNNEQFF

SEQ ID NO: 1799 - protein, 16 aa, Artificial sequence
  synthetic sequence
CSAGRGTGGVNQPQHF

SEQ ID NO: 1800 - protein, 13 aa, Artificial sequence
  synthetic sequence
CASSRLGPDTQYF

SEQ ID NO: 1801 - protein, 14 aa, Artificial sequence
  synthetic sequence
CASSLSEVNTEAFF

SEQ ID NO: 1802 - protein, 14 aa, Artificial sequence
  synthetic sequence
CASSQNRENEKLFF

SEQ ID NO: 1803 - protein, 12 aa, Artificial sequence
  synthetic sequence
CSAHRSNTEAFF

SEQ ID NO: 1804 - protein, 16 aa, Artificial sequence
  synthetic sequence
CASSQESTASGNTIYF

SEQ ID NO: 1805 - protein, 12 aa, Artificial sequence
  synthetic sequence
CASRDVGETQYF

SEQ ID NO: 1806 - protein, 14 aa, Artificial sequence
  synthetic sequence
CASVQGASNYGYTF

SEQ ID NO: 1807 - protein, 14 aa, Artificial sequence
  synthetic sequence
CASSNPGDRNEQFF

SEQ ID NO: 1808 - protein, 14 aa, Artificial sequence
  synthetic sequence
CASRHRGHNQPQHF

SEQ ID NO: 1809 - protein, 12 aa, Artificial sequence
  synthetic sequence
CSAGQGKGEQYF

SEQ ID NO: 1810 - protein, 14 aa, Artificial sequence
  synthetic sequence
CASSPLAGVYEQYF

SEQ ID NO: 1811 - protein, 14 aa, Artificial sequence
  synthetic sequence
CASTPPPGNQPQHF

SEQ ID NO: 1812 - protein, 13 aa, Artificial sequence
  synthetic sequence
CASSDNWGTEAFF

SEQ ID NO: 1813 - protein, 13 aa, Artificial sequence
  synthetic sequence
CASRDGDTGELFF

SEQ ID NO: 1814 - protein, 12 aa, Artificial sequence
  synthetic sequence
CAWSRQHNEQFF

SEQ ID NO: 1815 - protein, 16 aa, Artificial sequence
  synthetic sequence
CASSPRQVFSGNTIYF

SEQ ID NO: 1816 - protein, 16 aa, Artificial sequence
  synthetic sequence
CSVEDLAASSYNEQFF

SEQ ID NO: 1817 - protein, 10 aa, Artificial sequence
  synthetic sequence
CARGGQPQHF

SEQ ID NO: 1818 - protein, 14 aa, Artificial sequence
  synthetic sequence
CASSPTAYSYEQYF

SEQ ID NO: 1819 - protein, 15 aa, Artificial sequence
  synthetic sequence
CASSLRTGLRETQYF

SEQ ID NO: 1820 - protein, 13 aa, Artificial sequence
  synthetic sequence
CASSQEILDTQYF

SEQ ID NO: 1821 - protein, 13 aa, Artificial sequence
  synthetic sequence
CASSMEGSRGYTF

SEQ ID NO: 1822 - protein, 16 aa, Artificial sequence
  synthetic sequence
CAISEWDRGMNTEAFF

SEQ ID NO: 1823 - protein, 15 aa, Artificial sequence
  synthetic sequence
CSARSQGRYTGELFF

SEQ ID NO: 1824 - protein, 15 aa, Artificial sequence
  synthetic sequence
CASSPYRVNTGELFF

SEQ ID NO: 1825 - protein, 15 aa, Artificial sequence
  synthetic sequence
CASSPQDRVNQPQHF

SEQ ID NO: 1826 - protein, 14 aa, Artificial sequence
  synthetic sequence
CSAREQGETGELFF

SEQ ID NO: 1827 - protein, 14 aa, Artificial sequence
  synthetic sequence
CASSDSAEGGELFF

SEQ ID NO: 1828 - protein, 13 aa, Artificial sequence
  synthetic sequence
CASRVDSHQPQHF

SEQ ID NO: 1829 - protein, 17 aa, Artificial sequence
  synthetic sequence
CASSSGTAGSYNSPLHF

SEQ ID NO: 1830 - protein, 15 aa, Artificial sequence
  synthetic sequence
CASSPRSGRGETQYF

SEQ ID NO: 1831 - protein, 12 aa, Artificial sequence
  synthetic sequence
CASRGAVQPQHF

SEQ ID NO: 1832 - protein, 13 aa, Artificial sequence
  synthetic sequence
CASRADRRNTIYF

SEQ ID NO: 1833 - protein, 16 aa, Artificial sequence
  synthetic sequence
CATSRGTGASTGELFF

SEQ ID NO: 1834 - protein, 13 aa, Artificial sequence
  synthetic sequence
CASSSGQDSPLHF

SEQ ID NO: 1835 - protein, 16 aa, Artificial sequence
  synthetic sequence
CASSQDRRGGNYEQFF

SEQ ID NO: 1836 - protein, 16 aa, Artificial sequence
  synthetic sequence
CASSQVDSPNTGELFF

SEQ ID NO: 1837 - protein, 14 aa, Artificial sequence
  synthetic sequence
CASRRQGGAYEQYF

SEQ ID NO: 1838 - protein, 15 aa, Artificial sequence
  synthetic sequence
CASRRGTVSTDTQYF

SEQ ID NO: 1839 - protein, 17 aa, Artificial sequence
  synthetic sequence
CASSLGDRGQTNEKLFF

SEQ ID NO: 1840 - protein, 13 aa, Artificial sequence
  synthetic sequence
CASSGQVDNEQFF

SEQ ID NO: 1841 - protein, 15 aa, Artificial sequence
  synthetic sequence
CASSEARKGNTEAFF

SEQ ID NO: 1842 - protein, 16 aa, Artificial sequence
  synthetic sequence
CSARDHEAGGYNEQFF

SEQ ID NO: 1843 - protein, 14 aa, Artificial sequence
  synthetic sequence
CASSQEHEGYGYTF

SEQ ID NO: 1844 - protein, 14 aa, Artificial sequence
  synthetic sequence
CSVGTGLASYEQYF

SEQ ID NO: 1845 - protein, 15 aa, Artificial sequence
  synthetic sequence
CSARPLRDRTYGYTF

SEQ ID NO: 1846 - protein, 15 aa, Artificial sequence
  synthetic sequence
CASSIEGTENEKLFF

SEQ ID NO: 1847 - protein, 15 aa, Artificial sequence
  synthetic sequence
CASSVEGASREKLFF

SEQ ID NO: 1848 - protein, 12 aa, Artificial sequence
  synthetic sequence
CASSPPSHEQFF

SEQ ID NO: 1849 - protein, 15 aa, Artificial sequence
  synthetic sequence
CASSDPGTGGYGYTF

SEQ ID NO: 1850 - protein, 15 aa, Artificial sequence
  synthetic sequence
CASSQERSTNYGYTF

SEQ ID NO: 1851 - protein, 15 aa, Artificial sequence
  synthetic sequence
CASSGRTGGNYEQYF

SEQ ID NO: 1852 - protein, 14 aa, Artificial sequence
  synthetic sequence
CASSDLTSGNTIYF

SEQ ID NO: 1853 - protein, 14 aa, Artificial sequence
  synthetic sequence
CASSLVDSSYEQYF

SEQ ID NO: 1854 - protein, 14 aa, Artificial sequence
  synthetic sequence
CASSFLARTGELFF

SEQ ID NO: 1855 - protein, 15 aa, Artificial sequence
  synthetic sequence
CASSLGHRGTGELFF

SEQ ID NO: 1856 - protein, 14 aa, Artificial sequence
  synthetic sequence
CASSPRLAGETQYF

SEQ ID NO: 1857 - protein, 14 aa, Artificial sequence
  synthetic sequence
CASSPLGRQETQYF

SEQ ID NO: 1858 - protein, 14 aa, Artificial sequence
  synthetic sequence
CASSLKGRHYGYTF

SEQ ID NO: 1859 - protein, 15 aa, Artificial sequence
  synthetic sequence
CASSSPTGGGQPQHF

SEQ ID NO: 1860 - protein, 14 aa, Artificial sequence
  synthetic sequence
CASSPRGEGETQYF

SEQ ID NO: 1861 - protein, 14 aa, Artificial sequence
  synthetic sequence
CASSLLATAYEQYF

SEQ ID NO: 1862 - protein, 13 aa, Artificial sequence
  synthetic sequence
CASSHGVDTEAFF

SEQ ID NO: 1863 - protein, 13 aa, Artificial sequence
  synthetic sequence
CSATGLPNSPLHF

SEQ ID NO: 1864 - protein, 18 aa, Artificial sequence
  synthetic sequence
CASSLVGTGGLAKNIQYF

SEQ ID NO: 1865 - protein, 13 aa, Artificial sequence
  synthetic sequence
CASSWSRSYEQYF

SEQ ID NO: 1866 - protein, 14 aa, Artificial sequence
  synthetic sequence
CASTLGLYNSPLHF

SEQ ID NO: 1867 - protein, 12 aa, Artificial sequence
  synthetic sequence
CASVGQGNGYTF

SEQ ID NO: 1868 - protein, 14 aa, Artificial sequence
  synthetic sequence
CASSSRYLNTEAFF

SEQ ID NO: 1869 - protein, 16 aa, Artificial sequence
  synthetic sequence
CASSIFPGQGLETQYF

SEQ ID NO: 1870 - protein, 15 aa, Artificial sequence
  synthetic sequence
CASSIGKQGDQPQHF

SEQ ID NO: 1871 - protein, 15 aa, Artificial sequence
  synthetic sequence
CASSLEGTVTDTQYF

SEQ ID NO: 1872 - protein, 16 aa, Artificial sequence
  synthetic sequence
CASSPTRTGMNTEAFF

SEQ ID NO: 1873 - protein, 13 aa, Artificial sequence
  synthetic sequence
CASRAGGSSPLHF

SEQ ID NO: 1874 - protein, 16 aa, Artificial sequence
  synthetic sequence
CASSPRQGQGNQPQHF

SEQ ID NO: 1875 - protein, 14 aa, Artificial sequence
  synthetic sequence
CASSSPGGNNEQFF

SEQ ID NO: 1876 - protein, 12 aa, Artificial sequence
  synthetic sequence
CSARPGRNGYTF

SEQ ID NO: 1877 - protein, 11 aa, Artificial sequence
  synthetic sequence
CASGPGDIQYF

SEQ ID NO: 1878 - protein, 13 aa, Artificial sequence
  synthetic sequence
CATSLFSYNEQFF

SEQ ID NO: 1879 - protein, 12 aa, Artificial sequence
  synthetic sequence
CASSVQGNIQYF

SEQ ID NO: 1880 - protein, 14 aa, Artificial sequence
  synthetic sequence
CASSSDRLLSPLHF

SEQ ID NO: 1881 - protein, 15 aa, Artificial sequence
  synthetic sequence
CASKRGTANQETQYF

SEQ ID NO: 1882 - protein, 12 aa, Artificial sequence
  synthetic sequence
CASNPPGGGYTF

SEQ ID NO: 1883 - protein, 13 aa, Artificial sequence
  synthetic sequence
CASNRGIQETQYF

SEQ ID NO: 1884 - protein, 14 aa, Artificial sequence
  synthetic sequence
CASSSPGEKNIQYF

SEQ ID NO: 1885 - protein, 14 aa, Artificial sequence
  synthetic sequence
CASSYSRGNHEQYF

SEQ ID NO: 1886 - protein, 17 aa, Artificial sequence
  synthetic sequence
CASSYSPPAGIYNEQFF

SEQ ID NO: 1887 - protein, 15 aa, Artificial sequence
  synthetic sequence
CASSFGGSGGETQYF

SEQ ID NO: 1888 - protein, 17 aa, Artificial sequence
  synthetic sequence
CASSYSPLAGIYNEQFF

SEQ ID NO: 1889 - protein, 13 aa, Artificial sequence
  synthetic sequence
CASSPQQGYEQYF

SEQ ID NO: 1890 - protein, 16 aa, Artificial sequence
  synthetic sequence
CASSYSGAATNEKLFF

SEQ ID NO: 1891 - protein, 14 aa, Artificial sequence
  synthetic sequence
CASRLGASNQPQHF

SEQ ID NO: 1892 - protein, 14 aa, Artificial sequence
  synthetic sequence
CASSLEQYNSPLHF

SEQ ID NO: 1893 - protein, 15 aa, Artificial sequence
  synthetic sequence
CASSLVMGPWNEQFF

SEQ ID NO: 1894 - protein, 14 aa, Artificial sequence
  synthetic sequence
CAIRYSDVYNEQFF

SEQ ID NO: 1895 - protein, 13 aa, Artificial sequence
  synthetic sequence
CASSKTDNYGYTF

SEQ ID NO: 1896 - protein, 12 aa, Artificial sequence
  synthetic sequence
CASGLGAYEQYF

SEQ ID NO: 1897 - protein, 14 aa, Artificial sequence
  synthetic sequence
CASRTGTTSYEQYF

SEQ ID NO: 1898 - protein, 14 aa, Artificial sequence
  synthetic sequence
CASSLSADYQPQHF

SEQ ID NO: 1899 - protein, 16 aa, Artificial sequence
  synthetic sequence
CASSEAQRGSYNEQFF

SEQ ID NO: 1900 - protein, 15 aa, Artificial sequence
  synthetic sequence
CASSYSRDGQETQYF

SEQ ID NO: 1901 - protein, 14 aa, Artificial sequence
  synthetic sequence
CSARDDRVGNTIYF

SEQ ID NO: 1902 - protein, 14 aa, Artificial sequence
  synthetic sequence
CASRGTLRNTEAFF

SEQ ID NO: 1903 - protein, 13 aa, Artificial sequence
  synthetic sequence
CSARDQAYQPQHF

SEQ ID NO: 1904 - protein, 14 aa, Artificial sequence
  synthetic sequence
CASSQPGDTDTQYF

SEQ ID NO: 1905 - protein, 13 aa, Artificial sequence
  synthetic sequence
CASSSGPAGGYTF

SEQ ID NO: 1906 - protein, 15 aa, Artificial sequence
  synthetic sequence
CASSAWTATNEKLFF

SEQ ID NO: 1907 - protein, 15 aa, Artificial sequence
  synthetic sequence
CASSYDRAGGTEAFF

SEQ ID NO: 1908 - protein, 14 aa, Artificial sequence
  synthetic sequence
CASSPTSTNEKLFF

SEQ ID NO: 1909 - protein, 15 aa, Artificial sequence
  synthetic sequence
CASSYGQPSNYGYTF

SEQ ID NO: 1910 - protein, 11 aa, Artificial sequence
  synthetic sequence
CASSRVNEQYF

SEQ ID NO: 1911 - protein, 13 aa, Artificial sequence
  synthetic sequence
CASSAWTDQPQHF

SEQ ID NO: 1912 - protein, 13 aa, Artificial sequence
  synthetic sequence
CASSPTTTDTQYF

SEQ ID NO: 1913 - protein, 15 aa, Artificial sequence
  synthetic sequence
CASSPLGTGGDTQYF

SEQ ID NO: 1914 - protein, 13 aa, Artificial sequence
  synthetic sequence
CAWMRGLSYEQYF

SEQ ID NO: 1915 - protein, 15 aa, Artificial sequence
  synthetic sequence
CASSQDRQSGQPQHF

SEQ ID NO: 1916 - protein, 12 aa, Artificial sequence
  synthetic sequence
CAWRLQYNEQFF

SEQ ID NO: 1917 - protein, 13 aa, Artificial sequence
  synthetic sequence
CASSLSGHNGYTF

SEQ ID NO: 1918 - protein, 14 aa, Artificial sequence
  synthetic sequence
CAISERGARDTQYF

SEQ ID NO: 1919 - protein, 14 aa, Artificial sequence
  synthetic sequence
CASTTAGGPRELFF

SEQ ID NO: 1920 - protein, 15 aa, Artificial sequence
  synthetic sequence
CASRKDRGSNQPQHF

SEQ ID NO: 1921 - protein, 16 aa, Artificial sequence
  synthetic sequence
CASSLSGTGTNYGYTF

SEQ ID NO: 1922 - protein, 12 aa, Artificial sequence
  synthetic sequence
CASSPVVTEAFF

SEQ ID NO: 1923 - protein, 15 aa, Artificial sequence
  synthetic sequence
CASSSSGGGNEKLFF

SEQ ID NO: 1924 - protein, 14 aa, Artificial sequence
  synthetic sequence
CASSALDSNYGYTF

SEQ ID NO: 1925 - protein, 11 aa, Artificial sequence
  synthetic sequence
CASSLQEKLFF

SEQ ID NO: 1926 - protein, 13 aa, Artificial sequence
  synthetic sequence
CASSGTGYYGYTF

SEQ ID NO: 1927 - protein, 17 aa, Artificial sequence
  synthetic sequence
CASSPPQGSSYNSPLHF

SEQ ID NO: 1928 - protein, 12 aa, Artificial sequence
  synthetic sequence
CSALRGSDTQYF

SEQ ID NO: 1929 - protein, 13 aa, Artificial sequence
  synthetic sequence
CSATSGRARELFF

SEQ ID NO: 1930 - protein, 13 aa, Artificial sequence
  synthetic sequence
CASSPRTGNTIYF

SEQ ID NO: 1931 - protein, 14 aa, Artificial sequence
  synthetic sequence
CASSHGLMNTEAFF

SEQ ID NO: 1932 - protein, 15 aa, Artificial sequence
  synthetic sequence
CASSRGLAGNYEQYF

SEQ ID NO: 1933 - protein, 15 aa, Artificial sequence
  synthetic sequence
CASSLKRGMNTEAFF

SEQ ID NO: 1934 - protein, 16 aa, Artificial sequence
  synthetic sequence
CASSQVPGYSGNTIYF

SEQ ID NO: 1935 - protein, 14 aa, Artificial sequence
  synthetic sequence
CASSPTAGSTEAFF

SEQ ID NO: 1936 - protein, 14 aa, Artificial sequence
  synthetic sequence
CASSKGQGNTEAFF

SEQ ID NO: 1937 - protein, 14 aa, Artificial sequence
  synthetic sequence
CASSQGTGTSGYTF

SEQ ID NO: 1938 - protein, 14 aa, Artificial sequence
  synthetic sequence
CSARDGRGGDGYTF

SEQ ID NO: 1939 - protein, 13 aa, Artificial sequence
  synthetic sequence
CASSLMGANVLTF

SEQ ID NO: 1940 - protein, 15 aa, Artificial sequence
  synthetic sequence
CSAPTGRGSYNEQFF

SEQ ID NO: 1941 - protein, 15 aa, Artificial sequence
  synthetic sequence
CASSEDRAGNTEAFF

SEQ ID NO: 1942 - protein, 13 aa, Artificial sequence
  synthetic sequence
CASSEDRVDGYTF

SEQ ID NO: 1943 - protein, 13 aa, Artificial sequence
  synthetic sequence
CASSQEPPYEQYF

SEQ ID NO: 1944 - protein, 15 aa, Artificial sequence
  synthetic sequence
CASSRDRGRSTEAFF

SEQ ID NO: 1945 - protein, 16 aa, Artificial sequence
  synthetic sequence
CASSSTGRPNTGELFF

SEQ ID NO: 1946 - protein, 14 aa, Artificial sequence
  synthetic sequence
CASSDRGKNQPQHF

SEQ ID NO: 1947 - protein, 13 aa, Artificial sequence
  synthetic sequence
CASSKEVQETQYF

SEQ ID NO: 1948 - protein, 14 aa, Artificial sequence
  synthetic sequence
CASSPNLGGYEQYF

SEQ ID NO: 1949 - protein, 13 aa, Artificial sequence
  synthetic sequence
CASSAHGNTEAFF

SEQ ID NO: 1950 - protein, 13 aa, Artificial sequence
  synthetic sequence
CASSLAIGYGYTF

SEQ ID NO: 1951 - protein, 12 aa, Artificial sequence
  synthetic sequence
CASSFASETQYF

SEQ ID NO: 1952 - protein, 14 aa, Artificial sequence
  synthetic sequence
CASSSATGNQPQHF

SEQ ID NO: 1953 - protein, 13 aa, Artificial sequence
  synthetic sequence
CASSLEFNEKLFF

SEQ ID NO: 1954 - protein, 15 aa, Artificial sequence
  synthetic sequence
CASSLRAASNTEAFF

SEQ ID NO: 1955 - protein, 14 aa, Artificial sequence
  synthetic sequence
CASSQASEGYGYTF

SEQ ID NO: 1956 - protein, 14 aa, Artificial sequence
  synthetic sequence
CASSQGTRNSPLHF

SEQ ID NO: 1957 - protein, 13 aa, Artificial sequence
  synthetic sequence
CASRETGNTEAFF

SEQ ID NO: 1958 - protein, 14 aa, Artificial sequence
  synthetic sequence
CSAGGSSGYNEQFF

SEQ ID NO: 1959 - protein, 14 aa, Artificial sequence
  synthetic sequence
CASSIGVLRTEAFF

SEQ ID NO: 1960 - protein, 15 aa, Artificial sequence
  synthetic sequence
CASSFRGKANYGYTF

SEQ ID NO: 1961 - protein, 13 aa, Artificial sequence
  synthetic sequence
CASSSQVGYEQYF

SEQ ID NO: 1962 - protein, 13 aa, Artificial sequence
  synthetic sequence
CASSPSGPNEQFF

SEQ ID NO: 1963 - protein, 15 aa, Artificial sequence
  synthetic sequence
CASSETGGSNQPQHF

SEQ ID NO: 1964 - protein, 14 aa, Artificial sequence
  synthetic sequence
CASSLRQGDSEQYF

SEQ ID NO: 1965 - protein, 13 aa, Artificial sequence
  synthetic sequence
CSASGDLYNEQFF

SEQ ID NO: 1966 - protein, 14 aa, Artificial sequence
  synthetic sequence
CASSFGGRWETQYF

SEQ ID NO: 1967 - protein, 14 aa, Artificial sequence
  synthetic sequence
CASSQVGDKNIQYF

SEQ ID NO: 1968 - protein, 14 aa, Artificial sequence
  synthetic sequence
CASSLVTGGLGYTF

SEQ ID NO: 1969 - protein, 14 aa, Artificial sequence
  synthetic sequence
CASSRQPSGNTIYF

SEQ ID NO: 1970 - protein, 16 aa, Artificial sequence
  synthetic sequence
CASSLDPGGRAYEQYF

SEQ ID NO: 1971 - protein, 14 aa, Artificial sequence
  synthetic sequence
CASSPLDRVTEAFF

SEQ ID NO: 1972 - protein, 15 aa, Artificial sequence
  synthetic sequence
CASSGEGSYNSPLHF

SEQ ID NO: 1973 - protein, 15 aa, Artificial sequence
  synthetic sequence
CSALRTSGRANEQFF

SEQ ID NO: 1974 - protein, 13 aa, Artificial sequence
  synthetic sequence
CSAQFGGNQPQHF

SEQ ID NO: 1975 - protein, 15 aa, Artificial sequence
  synthetic sequence
CASSPDRGYNSPLHF

SEQ ID NO: 1976 - protein, 14 aa, Artificial sequence
  synthetic sequence
CSARVDRLNYGYTF

SEQ ID NO: 1977 - protein, 14 aa, Artificial sequence
  synthetic sequence
CASSIQGSHYEQYF

SEQ ID NO: 1978 - protein, 15 aa, Artificial sequence
  synthetic sequence
CASSLTGLGNQPQHF

SEQ ID NO: 1979 - protein, 13 aa, Artificial sequence
  synthetic sequence
CSAVRDSLYGYTF

SEQ ID NO: 1980 - protein, 15 aa, Artificial sequence
  synthetic sequence
CSARDRAGFNSPLHF

SEQ ID NO: 1981 - protein, 14 aa, Artificial sequence
  synthetic sequence
CASSPIFSNQPQHF

SEQ ID NO: 1982 - protein, 15 aa, Artificial sequence
  synthetic sequence
CASSLERGQGQPQHF

SEQ ID NO: 1983 - protein, 13 aa, Artificial sequence
  synthetic sequence
CSARGAAHQPQHF

SEQ ID NO: 1984 - protein, 14 aa, Artificial sequence
  synthetic sequence
CASSEGPPAGELFF

SEQ ID NO: 1985 - protein, 17 aa, Artificial sequence
  synthetic sequence
CASSPGQSGSYNSPLHF

SEQ ID NO: 1986 - protein, 14 aa, Artificial sequence
  synthetic sequence
CASSLGGDTYEQYF

SEQ ID NO: 1987 - protein, 13 aa, Artificial sequence
  synthetic sequence
CASSPVTNYGYTF

SEQ ID NO: 1988 - protein, 14 aa, Artificial sequence
  synthetic sequence
CASSQQLSGNTIYF

SEQ ID NO: 1989 - protein, 15 aa, Artificial sequence
  synthetic sequence
CASSLTDRRDSPLHF

SEQ ID NO: 1990 - protein, 13 aa, Artificial sequence
  synthetic sequence
CAWSFRGSYEQYF

SEQ ID NO: 1991 - protein, 16 aa, Artificial sequence
  synthetic sequence
CASSSRQGGSNQPQHF

SEQ ID NO: 1992 - protein, 14 aa, Artificial sequence
  synthetic sequence
CASRSEYSNQPQHF

SEQ ID NO: 1993 - protein, 12 aa, Artificial sequence
  synthetic sequence
CSVLAGVETQYF

SEQ ID NO: 1994 - protein, 15 aa, Artificial sequence
  synthetic sequence
CASSPPAGDHEKLFF

SEQ ID NO: 1995 - protein, 14 aa, Artificial sequence
  synthetic sequence
CASSEHHSNQPQHF

SEQ ID NO: 1996 - protein, 15 aa, Artificial sequence
  synthetic sequence
CASSPDRGFNQPQHF